(19) United States
(12) Patent Application Publication
(10) Pub. No.: US 2026/0327199 A1
(43) Pub. Date: Oct. 22, 2026

(54) HYDROXY AND ALKOXY COUMARINS AS MODULATORS OF POLRMT

(71) Applicant: Pretzel Therapeutics, Inc., Waltham, MA (US)

(72) Inventors: Gabriel Martinez Botella, Wayland, MA (US); Jeremy Green, Waltham, MA (US); Paul S. Charifson, Carrabassett Valley, ME (US); Simon Giroux, Cambridge, MA (US); Andrew Griffin, L'Île Bizard (CA)

(73) Assignee: Pretzel Therapeutics, Inc., Waltham, MA (US)

(21) Appl. No.: 18/687,736

(22) PCT Filed: Aug. 30, 2022
(86) PCT No.: PCT/US2022/042092

§ 371 Date: Feb. 28, 2024

Related U.S. Application Data
(60) Provisional application No. 63/238,754, filed on Aug. 30, 2021.
(60) Provisional application No. 63/276,278, filed on Nov. 5, 2021.
(60) Provisional application No. 63/284,950, filed on Dec. 1, 2021.
(60) Provisional application No. 63/331,430, filed on Apr. 15, 2022.
(60) Provisional application No. 63/358,700, filed on Jul. 6, 2022.

(51) Int. Cl. C07D 311/16; A61K 31/37; A61K 31/381; A61K 31/397; A61K 31/4025; A61K 31/422; A61K 31/4433; A61K 31/453; A61K 31/541; C07D 405/04; C07D 405/12; C07D 407/04; C07D 407/12; C07D 409/04; C07D 413/04

(52) U.S. Cl. CPC C07D 311/16; A61K 31/37; A61K 31/381; A61K 31/397; A61K 31/4025; A61K 31/422; A61K 31/4433; A61K 31/453; A61K 31/541; C07D 405/04; C07D 405/12; C07D 407/04; C07D 407/12; C07D 409/04; C07D 413/04

(57) ABSTRACT

The present invention provides novel hydroxy and alkoxy coumarin compounds that are inhibitors of mitochondrial RNA polymerase for treating various diseases such as cancer and others associated with metabolic disorders and mitochondrial dysfunction.

### CROSS-REFERENCE TO RELATED APPLICATIONS

This application is the National Stage entry under 35 U.S.C. § 371 of International Application No. PCT/US2022/042092, filed on Aug. 30, 2022, published on Mar. 9, 2023 under Publication Number WO 2023/034339 A1, which claims priority from U.S. Provisional Application No. 63/238,754, filed Aug. 30, 2021, U.S. Provisional Application No. 63/276,278, filed Nov. 5, 2021, U.S. Provisional Application No. 63/284,950, filed Dec. 1, 2021, U.S. Provisional Application No. 63/331,430, filed Apr. 15, 2022, and U.S. Provisional Application No. 63/358,700, filed Jul. 6, 2022 all of the applications identified above are incorporated herein by reference in their entirety.

### FIELD OF THE INVENTION

The present invention relates to novel POLRMT modulators, their prodrugs, their pharmaceutically acceptable salts, and pharmaceutical compositions thereof. The present invention also relates to methods of using such compounds and compositions, including to inhibit or promote POLRMT, and to treat various neurodegenerative and metabolic disorders, cancer, and also disorders related to aging and mitochondrial diseases.

### BACKGROUND OF THE INVENTION

Human mitochondrial RNA polymerase, POLRMT (also referred to as h-mtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368). See, e.g., Arnold, J. J., et al., “Human mitochondrial RNA polymerase: Structure-function, mechanism and inhibition,” Biochim. Biophys. Acta, 1819, 948-960 (2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Regarding the POLRMT NTD, structurally it resembles the N-terminal domain (also called the promoter-binding domain) of T7 RNA polymerase. However, for promoter-specific transcription initiation POLRMT requires assistance from additional transcription factors, whereas T7 RNA polymerase does not.

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site. See, e.g., Basu, U., et al., “Structure, mechanism, and regulation of mitochondrial DNA transcription initiation,” J. Biol. Chem., 295(52), 18406-425 (2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id.

The mitochondrial genome encodes the various subunits of the electron transport chain. See, e.g., Shokolenko, I. N., et al., “Maintenance and expression of mammalian mitochondrial DNA,” Annu. Rev. Biochem., 85, 133-160 (2016). Specifically, transcription of the mitochondrial genome is necessary for the expression of 13 subunits of the oxidative phosphorylation (OXPHOS) system, as well as two rRNAs and 22 tRNAs. See, e.g., Shokolenko, I. N., et al., “Mitochondrial transcription in mammalian cells,” Frontiers in Bioscience, Landmark, 22, 835-853 (2017). Thus, POLRMT is essential for biogenesis of the OXPHOS system, resulting in ATP production. This, in turn, is vital for energy homeostasis in the cell.

Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer. See Siegel, R. L. et al., “Cancer statistics 2022.” CA Cancer J. Clin. (72) 7-33 (2022). High rates of OXPHOS have been shown to support growth in cancer cell lines, including in a subset of diffuse large B cell lymphoma cells. See, e.g., DeBeradinis, R. J., “A mitochondrial power play in lymphoma,” Cancer Cell, 22, 423-24 (2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity. Rodrigues, M. F., et al., “Enhanced OXPHOS, glutaminolysis and β-oxidation constitute the metastatic phenotype of melanoma cells,” Biochem. J. 473: 703-715 (2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown AML cells demonstrated a reduction in POLRMT levels, decreased oxidative phosphorylation, and increased cell death as compared to control AML cells. See Bralha, F. N., et al., “Targeting mitochondrial RNA polymerase in acute myeloid leukemia,” Oncotarget, 6(35), 37216-228 (2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene. Salem, A. F., et al. “Mitochondrial biogenesis in epithelial cancer cells promotes breast cancer tumor growth and confers autophagy resistance,” Cell Cycle, 11(22), 4174-80 (2012). Recently, the expression of POLRMT in non-small cell lung cancer (NSCLC) has been examined. See Zhou, T. et al., “The requirement of mitochondrial RNA polymerase for non-small cell lung cancer cell growth,” Cell Death and Disease, 12, 751 (2021). While POLRMT mRNA and protein were detected in normal human lung tissue, their levels were significantly higher in cancer tissue. Similar results were obtained when comparing primary lung epithelial cells to NSCLC cells. Using short hairpin RNA (shRNA) to silence POLRMT mRNA and downregulate POLRMT protein resulted in inhibition of NSCLC cell viability, proliferation, migration, and invasion. Moreover, silencing of POLRMT significantly induced apoptosis activation in both primary and established NSCLC cells. Injection of POLRMT shRNA in an adeno-associated virus construct into tumors effectively inhibited NSCLC xenograft growth in mice. Taken together, these data suggest that POLRMT could be an oncogenic gene for NSCLC.

The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy. See Yu, H.-J., “Targeting mitochondrial metabolism and RNA polymerase POLRMT to overcome multidrug resistance in cancer,” Front. Chem., 9:775226 (2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small-molecule inhibitors of POLRMT have been developed. See, e.g., EP 3 598 972 A1; WO 2019/057821 A1; and WO 2020/188049 A1. Some of these inhibitors have been shown to be useful in inhibiting cancer cell proliferation without affecting control cells. See Bonekamp, N. A., et al., “Small-molecule inhibitors of human mitochondrial DNA transcription,” Nature, 588, 712-716 (2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals. Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated. See Mennuni, M. et al., “Metabolic resistance to the inhibition of mitochondrial transcription revealed by CRISPER-Cas9 screen,” EMBO reports, 23: e53054 1-18 (2022). Using a CRISPR-Cas9 whole-genome screen, loss of genes belonging to von Hippel-Lindau (VHL) and mammalian target of rapamycin complex 1 (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity. See Pospisilik, J. A., et al., “Targeted deletion of AIF decreases mitochondrial oxidative phosphorylation and protects from obesity and diabetes,” Cell, 131, 476-91 (2007). Correlated with these phenotypic changes were the metabolic alterations of increased glucose uptake and enhanced fuel utilization. Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component. See, e.g., Filograna, R., et al., “Mitochondrial DNA copy number in human disease: the more the better?” FEBS Letters, 595, 976-1002 (2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POLγ) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurogenerative disorders, cancer, and aging. See e.g., Filograna, R., et al., “Mitochondrial DNA copy number in human disease: the more the better?” FEBS Letters, 595, 976-1002 (2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease. See Oláhová, M., et al., “POLRMT mutations impair mitochondrial transcription causing neurological disease.” Nat. Commun., 12, 1135 (2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Pharmacological inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat various neurodegenerative and metabolic disorders, cancer, and also disorders related to aging and mitochondrial diseases. The compounds and their pharmaceutically acceptable salts are particularly useful as modulators of POLRMT.

In one embodiment, the present invention is directed to a compound, a prodrug thereof, or a pharmaceutically acceptable salt thereof, represented by formula (I):

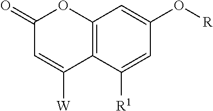

wherein:
W is C₆-C₁₀ aryl, C₆-C₁₀ cycloaryl, or 5-10 membered heteroaryl, any of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, CH₂CH₂R², 5-or 6-membered heterocyclyl optionally substituted with C₁-C₄ alkyl or C(O)R³, or C₁-C₆ alkyl optionally substituted with one or more groups, each independently selected from the group consisting of C₁-C₄ alkyl, 5-or 6-membered heterocyclyl, hydroxyl, cyano, fluoro, C₁-C₄ alkoxyl, C₁-C₄ haloalkoxyl, and NR³R⁴,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and C(O)R³,
provided that when R is hydrogen, then R¹ is methyl and W is substituted, and further provided that when R is methyl, then W is disubstituted;
R¹ is hydrogen, fluoro, chloro, hydroxyl, cyano, C₃-C₄ cycloalkyl, C₁-C₂ alkoxyl, C₁-C₂ haloalkoxyl, or C₁-C₃ alkyl optionally substituted with one or more fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl, OR³, NR³R⁴, C(O)R³, or C(O)NR³R⁴;
each R³ is independently hydrogen or C₁-C₄ alkyl optionally substituted with one or more groups selected from the group consisting of fluoro, hydroxyl, C₁-C₄ haloalkoxyl, and C₁-C₄ alkoxyl;
each R⁴ is independently R³, C(O)C₁-C₄ alkyl optionally substituted with one or more fluoro groups, or C(O)NHC₁-C₄ alkyl optionally substituted with one or more fluoro groups;
or if R³ and R⁴ are attached to the same nitrogen atom, R³ and R⁴ together with their connecting nitrogen form a 5-or 6-membered heterocyclic ring optionally containing another heteroatom that is N, O, S, S(O), or S(O)NR³, and such heterocyclic ring is optionally substituted with one or more groups each independently selected from the group consisting of fluoro, chloro, C₁-C₄ alkyl, C₁-C₄ alkoxyl, C₁-C₄ haloalkoxyl, carboxyl, and C₁-C₄ alkylcarboxylate; and
R⁵ is hydrogen or C₁-C₄ alkyl.

In certain embodiments, the present invention is directed to a compound, a prodrug thereof, or a pharmaceutically acceptable salt thereof, represented by formula (I):

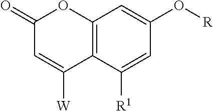

wherein:
W is C₆-C₁₀ aryl, C₆-C₁₀ cycloaryl, or 5-10 membered heteroaryl, any of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl, provided that at least one substituent is at an ortho position relative to the attachment point with the central ring if R¹ is hydrogen;
R is C₁-C₆ alkyl substituted with one or more groups selected from the group consisting of carboxyl, C(O)OR⁵, and C(O)NR³R⁴, and such C₁-C₆ alkyl is optionally further substituted with one or more groups, each independently selected from the group consisting of C₁-C₄ alkyl, 5-or 6-membered heterocyclyl, hydroxyl, cyano, fluoro, C₁-C₄ alkoxyl, C₁-C₄ haloalkoxyl, and NR³R⁴;
R¹ is hydrogen, fluoro, chloro, hydroxyl, cyano, C₃-C₄ cycloalkyl, C₁-C₂ alkoxyl, C₁-C₂ haloalkoxyl, or C₁-C₃ alkyl optionally substituted with one or more fluoro;
each R³ is independently hydrogen or C₁-C₄ alkyl optionally substituted with one or more groups selected from the group consisting of fluoro, hydroxyl, C₁-C₄ haloalkoxyl, and C₁-C₄ alkoxyl;
each R⁴ is independently R³, C(O)C₁-C₄ alkyl optionally substituted with one or more fluoro groups, or C(O)NHC₁-C₄ alkyl optionally substituted with one or more fluoro groups;
or if R³ and R⁴ are attached to the same nitrogen atom, R³ and R⁴ together with their connecting nitrogen form a 4-to 6-membered heterocyclic ring optionally containing one or more heteroatoms that is N, O, S, S(O), SO₂, or S(O)NR³, and such heterocyclic ring is optionally substituted with one or more groups each independently selected from the group consisting of fluoro, chloro, C₁-C₄ alkyl, C₁-C₄ alkoxyl, C₁-C₄ haloalkoxyl, carboxyl, oxo, and C₁-C₄ alkylcarboxylate; and
R⁵ is hydrogen or C₁-C₄ alkyl.

Further embodiments of the present invention are compounds of the invention (that is, compounds of formula (I)), their pharmaceutically acceptable salts, or prodrugs of the compounds wherein one or more hydrogen is substituted with a deuterium atom.

Additional embodiments of the invention are pharmaceutical compositions comprising a compound of the invention, a pharmaceutically acceptable salt thereof, or a prodrug thereof and one or more pharmaceutically acceptable excipients.

Further embodiments of the invention are methods of treating a disease, such methods comprising administering to a subject in need thereof a therapeutically effective amount of a compound of the invention, a prodrug thereof, or a pharmaceutically acceptable salt thereof. In some embodiments, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, choriocarcinoma, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, polycythemia vera, prostate cancer, rhabdomyosarcoma, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, non-alcoholic steatohepatitis (NASH), and related metabolic syndromes such as non-alcoholic fatty liver disease (NAFLD). In some embodiments, the disease is related to aging or a mitochondrial disorder.

Additional embodiments of the invention are methods of treating neurodegenerative disorders and metabolic disorders, such as those identified in Bonekamp, N. A. et al. “Small-molecule inhibitors of human mitochondrial DNA transcription,” Nature, 588, 712-716 (2020), Filograna, R. et al, “Mitochondrial DNA copy number in human disease: the more the better?” FEBS Lett., 595, 976-1002 (2021), Wrendenber, A. et al. “Respiratory chain dysfunction in skeletal muscle does not cause insulin resistance,” Biochem. Biophys. Res. Comm., 350, 202-207 (2006), Pospililik, J. A. et al. “Targeted deletion of AIF decreases mitochondrial oxidative phosphorylation and protects from obesity and diabetes,” Cell, 131, 476-491 (2007), and PCT International Publication No. WO 2019/057821 A1 and references therein.

Further embodiments of the invention are methods of treating disease of aging.

### DETAILED DESCRIPTION OF THE INVENTION

Modulators of POLRMT are useful in compositions and methods suitable for treating many disorders, such as cancer, neurodegenerative disorders, metabolic disorders, as well as diseases related to aging and mitochondrial diseases. Provided herein are compounds of formula (I), pharmaceutically acceptable salts thereof, prodrugs thereof, and pharmaceutical compositions comprising such compounds, their salts, or their prodrugs that are useful in treating a condition or disease, such as cancer, neurodegenerative disorders, and metabolic disorders.

### Definitions

The term “alkyl” as used herein refers to both branched- and straight-chain saturated aliphatic hydrocarbon groups having the specified number of carbon atoms in a specified range. For example the term “C₁-C₆ alkyl” means linear or branched chain alkyl groups, including all possible isomers, having 1, 2, 3, 4, 5, or 6 carbon atoms. Furthermore, alkyl groups allow for substituents to be located on any of the carbon atoms. For example, a substituted C₃ alkyl group allows for the substituent to be located on any of the three carbon atoms.

The term “alkoxy” or “alkoxyl” as used herein refers to an —O-alkyl group. For example, the term “C₁-C₄ alkoxyl” means —O—C₁-C₄ alkyl. Examples of alkoxyl include methoxyl, ethoxyl, propoxyl (e.g., n-propoxyl and isopropoxyl), and the like.

The term “haloalkoxy” or “haloalkoxyl” as used herein refers to an —O-alkyl group in which at least one of the hydrogen atoms of the alkyl group is replaced with a halogen atom. Examples of haloalkoxyl include trifluoromethoxyl, 2,2,2-trifluoroethoxyl, and the like.

The term “alkanoyl” or “acyl” as used herein refers to an —C(O)-alkyl group. For example, the term “C₁-C₆ alkanoyl” means —C(O)—C₁-C₆ alkyl. Examples of alkanoyl include acetyl, propionyl, butyryl, and the like.

The term “bicyclic” as used herein refers to a saturated or unsaturated 6-to 12-membered ring consisting of two joined cyclic substructures, and includes fused, bridged, and spiro bicyclic rings.

The term “heterobicyclic” as used herein refers to a bicyclic ring that contains 1 or more heteroatom(s) in one or more rings that are optionally substituted or oxidized, and may be selected from nitrogen (including N-oxides), oxygen, sulfur (including oxidized forms such as sulfones and sulfonates), phosphorus (including oxidized forms such as phosphates), boron, etc. Examples of heterobicyclic rings include, but are not limited to, 8-azabicyclo[3.2.1]octan-8-yl, 3-oxa-8-azabicyclo[3.2.1]octan-8-yl, 8-oxa-3-azabicyclo[3.2.1]octan-3-yl, and 5-methyl-2,5-diazabicyclo[2.2.1]heptan-2-yl.

The term “cycloalkyl” as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term “aryl” as used herein refers to a monocyclic or fused bicyclic ring system having the characteristics of aromaticity, wherein at least one ring contains a completely conjugated pi-electron system. Typically, aryl groups contain 6 to 14 carbon atoms (“C₆-C₁₄ aryl”) or preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom of the aromatic portion or a C or N atom of the non-aromatic portion of the ring system. Examples, without limitation, of aryl groups include phenyl, biphenyl, naphthyl, anthracenyl, indanyl, indenyl, and tetrahydronaphthyl.

The term “cycloaryl” herein refers to a polycyclic group wherein an aryl group is fused to a 5-or 6-membered aliphatic or heterocyclic ring. For example, “C₆-C₁₂ cycloaryl” means a C₆-C₁₂ aryl fused to a 5-or 6-membered aliphatic or heterocyclic ring. One example of C₆ cycloaryl is 2,3-dihydrobenzo[b][1,4]dioxine.

The term “heteroaryl” as used herein refers to (i) a 5-or 6-membered ring having the characteristics of aromaticity containing at least one heteroatom selected from N, O and S, wherein each N is optionally in the form of an oxide, and (ii) a 9-or 10-membered bicyclic fused ring system, wherein the fused ring system of (ii) contains at least one heteroatom independently selected from N, O and S, wherein each ring in the fused ring system contains zero, one or more than one heteroatoms, at least one ring is aromatic, each N is optionally in the form of an oxide, and each S in a ring which is not aromatic is optionally S(O) or S(O)₂. Typically, heteroaryl groups contain 5 to 14 ring atoms (“5-14 membered heteroaryl”), and preferably 5 to 12 ring atoms (“5-12 membered heteroaryl”). Heteroaryl rings are attached to the base molecule via a ring atom of the heteroaromatic ring, such that aromaticity is maintained. Suitable 5-and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9-and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term “heteroaryloxy” or “heteroaryloxyl” as used herein refers to an —O— heteroaryl group.

The term “heterocycle”, “heterocyclyl”, or “heterocyclic” as used herein represents a stable 3-to 10-membered monocyclic, non-aromatic ring that is either saturated or unsaturated, and that consists of carbon atoms and from one to two heteroatoms selected from the group consisting of N, O, and S. Examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, and oxazepanyl.

The term “oxo” as used herein refers to a group which consists of oxygen which is double bonded to carbon or any other element.

The term “imine” as used herein refers to a group containing a carbon-nitrogen double bond.

The term “carboxyl” as used herein refers to a combination of two functional groups attached to a single carbon atom, namely, hydroxyl (OH) and carbonyl (O).

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” mean that the groups may be the same or different.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen”, the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (T Higuchi and W Stella) and “Bioreversible Carriers in Drug Design,” Pergamon Press, 1987 (ed. E B Roche, American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety. Some non-limiting examples of prodrugs in accordance with the invention include: (i) where the compound contains a carboxylic acid functionality —(COOH), an ester thereof, for example, replacement of the hydrogen with (C₁-C₆)alkyl; (ii) where the compound contains an alcohol functionality (—OH), an ether thereof, for example, replacement of the hydrogen with (C₁-C₆)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₆ alkanoyl. Further examples of replacement groups in accordance with the foregoing examples and examples of other prodrug types may be found in the aforementioned references.

The terms “treatment”, “treating” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

### Compounds

In one embodiment, the present invention is directed to a compound, a prodrug thereof, or a pharmaceutically acceptable salt thereof, represented by formula (I):

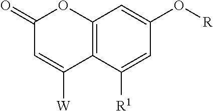

wherein:
W is C₆-C₁₀ aryl, C₆-C₁₀ cycloaryl, or 5-10 membered heteroaryl, any of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, CH₂CH₂R², 5-or 6-membered heterocyclyl optionally substituted with C₁-C₄ alkyl or C(O)R³, or C₁-C₆ alkyl optionally substituted with one or more groups, each independently selected from the group consisting of C₁-C₄ alkyl, 5-or 6-membered heterocyclyl, hydroxyl, cyano, fluoro, C₁-C₄ alkoxyl, C₁-C₄ haloalkoxyl, and NR³R⁴,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and C(O)R³,
provided that when R is hydrogen, then R¹ is methyl and W is substituted, and further provided that when R is methyl, then W is disubstituted;
R¹ is hydrogen, fluoro, chloro, hydroxyl, cyano, C₃-C₄ cycloalkyl, C₁-C₂ alkoxyl, C₁-C₂ haloalkoxyl, or C₁-C₃ alkyl optionally substituted with one or more fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl, OR³, NR³R⁴, C(O)R³, or C(O)NR³R⁴;
each R³ is independently hydrogen or C₁-C₄ alkyl optionally substituted with one or more groups selected from the group consisting of fluoro, hydroxyl, C₁-C₄ haloalkoxyl, and C₁-C₄ alkoxyl;
each R⁴ is independently R³, C(O)C₁-C₄ alkyl optionally substituted with one or more fluoro groups, or C(O)NHC₁-C₄ alkyl optionally substituted with one or more fluoro groups;
or if R³ and R⁴ are attached to the same nitrogen atom, R³ and R⁴ together with their connecting nitrogen form a 5-or 6-membered heterocyclic ring optionally containing another heteroatom that is N, O, S, S(O), or S(O)NR³, and such heterocyclic ring is optionally substituted with one or more groups each independently selected from the group consisting of fluoro, chloro, C₁-C₄ alkyl, C₁-C₄ alkoxyl, C₁-C₄ haloalkoxyl, carboxyl, and C₁-C₄ alkylcarboxylate; and
R⁵ is hydrogen or C₁-C₄ alkyl.

In certain embodiments, the present invention is directed to a compound, a prodrug thereof, or a pharmaceutically acceptable salt thereof, represented by formula (I):

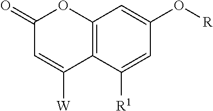

wherein:
W is C₆ aryl, C₆ cycloaryl, or a 5-or 6-membered heteroaryl, any of which is optionally substituted with one or more groups, each independently selected from the group consisting of fluoro, chloro, C₁ alkyl, trifluoromethyl, hydroxyl, and C₁ alkoxyl;
R is hydrogen, CH₂CH₂R², C₆ aryl, 5-or 6-membered heterocyclyl optionally substituted with C₁ alkyl or C(O)R³, or C₁-C₄ alkyl optionally substituted with one or more groups, each independently selected from the group consisting of 6-membered heterocyclyl, hydroxyl, cyano, fluoro, C₁ alkoxyl, and NR³R⁴;
R¹ is hydrogen or C₁ alkyl;
R² is C₆ aryl;
each R³ is independently hydrogen or C₁ alkyl; and
each R⁴ is independently R³.

In certain embodiments, the present invention is directed to a compound, a prodrug thereof, or a pharmaceutically acceptable salt thereof, represented by formula (I):

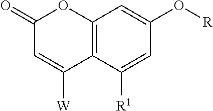

wherein:
W is C₆ aryl or 5-membered heteroaryl, any of which is optionally substituted with one or more groups, each independently selected from the group consisting of methyl, chloro, and fluoro;
R is 6-membered heterocyclyl or C₁-C₄ alkyl optionally substituted with one or more groups independently selected from the group consisting of cyano, fluoro, and methoxyl; and
R¹ is hydrogen.

In certain embodiments, the present invention is directed to a compound, a prodrug thereof, or a pharmaceutically acceptable salt thereof, represented by formula (I):

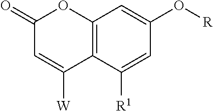

wherein:
W is C₆-C₁₀ aryl, C₆-C₁₀ cycloaryl, or 5-10 membered heteroaryl, any of which is optionally substituted with one or more groups, each independently selected from the group consisting of fluoro, chloro,, trifluoromethyl, difluoromethyl, cyano, hydroxyl, C₁-C₄ alkoxyl, and C₁-C₄ alkyl optionally substituted with one or more deuterium and hydroxyl, provided that at least one substituent is at an ortho position relative to the attachment point with the central ring if R¹ is hydrogen;
R is C₁-C₆ alkyl substituted with one or more groups selected from the group consisting of carboxyl, C(O)OR⁵, and C(O)NR³R⁴, and such C₁-C₆ alkyl is optionally further substituted with one or more groups, each independently selected from the group consisting of C₁-C₄ alkyl, 5-or 6-membered heterocyclyl, hydroxyl, cyano, fluoro, C₁-C₄ alkoxyl, C₁-C₄ haloalkoxyl, and NR³R⁴;
R¹ is hydrogen, fluoro, chloro, hydroxyl, cyano, C₃-C₄ cycloalkyl, C₁-C₂ alkoxyl, C₁-C₂ haloalkoxyl, or C₁-C₃ alkyl optionally substituted with one or more fluoro;
each R³ is independently hydrogen or C₁-C₄ alkyl optionally substituted with one or more groups selected from the group consisting of fluoro, hydroxyl, C₁-C₄ haloalkoxyl, and C₁-C₄ alkoxyl;
each R⁴ is independently R³, C(O)C₁-C₄ alkyl optionally substituted with one or more fluoro groups, or C(O)NHC₁-C₄ alkyl optionally substituted with one or more fluoro groups;
or if R³ and R⁴ are attached to the same nitrogen atom, R³ and R⁴ together with their connecting nitrogen form a 4-to 6-membered heterocyclic ring optionally containing one or more heteroatoms that is N, O, S, S(O), SO₂, or S(O)NR³, and such heterocyclic ring is optionally substituted with one or more groups each independently selected from the group consisting of fluoro, chloro, C₁-C₄ alkyl, C₁-C₄ alkoxyl, C₁-C₄ haloalkoxyl, carboxyl, oxo, and C₁-C₄ alkylcarboxylate; and
R⁵ is hydrogen or C₁-C₄ alkyl.

In certain embodiments, the present invention is directed to a compound, a prodrug thereof, or a pharmaceutically acceptable salt thereof, represented by formula (I):

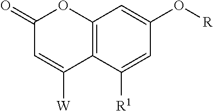

wherein:
W is C₆ aryl, which is optionally substituted with one or more groups, each independently selected from the group consisting of fluoro, chloro, and C₁ alkyl;
R is C₁-C₂ alkyl substituted with a group selected from the group consisting of carboxyl, C(O)OR⁵, and C(O)NR³R⁴, and such C₁-C₂ substituted alkyl is optionally further substituted with one or more groups, each independently selected from the group consisting of hydroxyl, C₁-C₂ alkoxyl, and C₁ haloalkoxyl;
R¹ is hydrogen or C₁ alkyl;
each R³ is independently hydrogen or C₁-C₂ alkyl optionally substituted with one or more groups selected from the group consisting of fluoro, and C₁ alkoxyl;
each R⁴ is independently R³;
or if R³ and R⁴ are attached to the same nitrogen atom, R³ and R⁴ together with their connecting nitrogen form a 4-or 6-membered heterocyclic ring optionally containing another heteroatom that is SO₂; and
R⁵ is C₁ alkyl.

In certain embodiments, the present invention is directed to a compound, a prodrug thereof, or a pharmaceutically acceptable salt thereof, represented by formula (I):

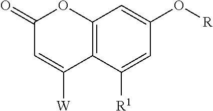

wherein:
W is C₆ aryl substituted with one or more groups, each independently selected from the group consisting of fluoro, chloro, and C₁ alkyl;
R is C₁-C₂ alkyl substituted with a group selected from the group consisting of carboxyl, C(O)OR⁵, and C(O)NR³R⁴, and such C₁-C₂ substituted alkyl is optionally further substituted with one or more groups, each independently selected from the group consisting of hydroxyl, C₁ alkoxyl, and C₁ haloalkoxyl;
R¹ is hydrogen or C₁ alkyl;
each R³ is independently hydrogen or C₁-C₂ alkyl optionally substituted with one or more groups selected from the group consisting of fluoro, and C₁ alkoxyl;
each R⁴ is independently R³;
or if R³ and R⁴ are attached to the same nitrogen atom, R³ and R⁴ together with their connecting nitrogen form a 4-or 6-membered heterocyclic ring optionally containing another heteroatom that is SO₂; and
R⁵ is C₁ alkyl.

In certain embodiments, W is phenyl optionally substituted with one or more groups, each independently selected from the group consisting of fluoro, chloro, C₁-C₄ alkyl, cyano, hydroxyl, and C₁-C₄ alkoxyl, provided that at least one substituent is at an ortho position relative to the attachment point with the central ring.

In certain embodiments, W is phenyl.

In certain embodiments, W is 4-fluorophenyl.

In certain embodiments, W is 2-chlorophenyl.

In certain embodiments, W is 2-trifluoromethylphenyl.

In certain embodiments, W is 3-methylthiophenyl.

In certain embodiments, W is 2-methyl-4-hydroxyphenyl.

In certain embodiments, W is 2-methoxyphenyl.

In certain embodiments, W is 4-trifluoromethylpyridinyl.

In certain embodiments, W is 2,3-dihydrobenzo[b][1,4]dioxinyl.

In certain embodiments, W is 2-chloro-4-fluorophenyl.

In certain embodiments, W is 2-methylphenyl.

In certain embodiments, W is 2,3-dimethylthiophenyl.

In certain embodiments, W is 3-methylpyridinyl.

In certain embodiments, W is 2-methylpyridinyl.

In certain embodiments, W is 3,5-dimethylisoxazolyl.

In certain embodiments, W is 2-chloro-4-methylpyridinyl.

In certain embodiments, W is 2,5-dimethylphenyl.

In certain embodiments, R¹ is hydrogen.

In certain embodiments, R¹ is methyl.

In certain embodiments, R is H.

In certain embodiments, R is a 5-membered heterocyclyl ring.

In certain embodiments, R is a 6-membered heterocyclyl ring.

In certain embodiments, R is C₄ alkyl substituted with methoxy.

In certain embodiments, R is C₂ alkyl substituted with ethoxy and C(O)NH₂.

In certain embodiments, R is C₂ alkyl substituted with methoxy and C(O)NR³R⁴.

In certain embodiments, R is C₂ alkyl substituted with methoxy and C(O)NR³R⁴.

In certain embodiments, R is C₂ alkyl substituted with methoxy and CO₂H.

In certain embodiments, R is C₂ alkyl substituted with methoxy and C(O)OCH₃.

In certain embodiments, R is C₂ alkyl substituted with hydroxy and CO₂H.

In certain embodiments, R is C₂ alkyl substituted with C(O)NR³R⁴.

In certain embodiments, R is C₂ alkyl substituted with CF₃.

In certain embodiments, R is C₃ alkyl substituted with OCH₃.

In certain embodiments, R is C₂ alkyl substituted with CN.

In certain embodiments, R is C₁ alkyl substituted with C(O)NH₂.

In certain embodiments, R is C₂ alkyl substituted with N(CH₃)₂.

In certain embodiments, R is C₂ alkyl substituted with OCH₃.

In certain embodiments, R is C₂ alkyl substituted with OH.

In certain embodiments, R is C₂ alkyl substituted with Ph.

In certain embodiments, R is C₁ alkyl substituted with CN.

In certain embodiments, R is C₁ alkyl substituted with tetrahydropyran.

In certain embodiments, R is CH₃.

In certain embodiments, R is CH(CH₃)₂.

In certain embodiments, R is C(CH₃)₃.

In certain embodiments, R is CH₂CH₂CH₃.

In certain embodiments, R is phenyl.

In certain embodiments, R is piperidine substituted with acetate.

In certain embodiments, R is pyrrolidine substituted with acetate.

In certain embodiments, R is pyrrolidine substituted with CH₃.

In certain embodiments, R is tetrahydropyran.

In certain embodiments, the compounds inhibits POLRMT.

In certain embodiments, the compounds promote POLRMT.

The compounds of the present invention may contain asymmetric carbon atoms (sometimes as the result of a deuterium atom) and thereby can exist as either individual stereoisomers or mixtures of the enantiomers or mixtures of diastereomers.

Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and ketoenol tautomerism is possible, it is understood that the substituent might in fact be present, in whole or in part, in the oxo (═O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention. See, e.g., Gannes, L Z et al., Comp Biochem Physiol Mol Integr Physiol, 119, 725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali et al, J. Med. Chem. 62, 5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances, deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto et al., J. Med. Chem. 34, 2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min. to 48 h., preferably 10 min. to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schutzenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 40 Ed.” (Theodora W. Greene, Peter G. M. Wuts) Wiley-Interscience, 2007; “Protecting Groups 3rd Ed.” (P. J. Kocienski) Thieme, 2004 and the like.

Deuterated POLRMT modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds, a pharmaceutically acceptable salt of said compounds, or prodrugs of said compounds. In addition to the compound of the invention, a salt thereof, or a prodrug thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., Remington: The Science and Practice of Pharmacy, Elsevier Science, 23rd ed. (2020).

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

In another embodiment, a pharmaceutical composition of this invention further comprises a second therapeutic agent. The second therapeutic agent may be selected from any pharmaceutically active compound; preferably the second therapeutic agent is known to treat cancer, neurodegenerative disorders, or metabolic disorders. Alternatively, the compounds of the invention and second therapeutic agent may be administered together (within less than 24 hours of one another, consecutively or simultaneously) but in separate pharmaceutical compositions. In certain embodiments, the compounds on the invention and second therapeutic agent can be administered separately (e.g., more than 24 hours of one another.) If the second therapeutic agent acts synergistically with the compounds of this invention, the therapeutically effective amount of such compounds and/or the second therapeutic agent may be less that such amount required when either is administered alone.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by either mass spectrometry or nuclear magnetic resonance spectroscopy (NMR), where peaks assigned to the characteristic protons in the title compound are presented where appropriate. ¹H NMR shift (δH) are given in parts per million (ppm) down field from an internal reference standard.

The abbreviations used herein are known to a person of ordinary skill in the art. A partial list of abbreviations that may be used herein include: acetonitrile (MeCN), ammonium carbonate (NH₄)₂CO₃, ammonium chloride (NH₄Cl), aqueous (aq.), 1,1′-bis(diphenylphosphino)ferrocene (dppf), 1,3-bis(diphenylphosphino)propane (dppp), bis(pinacolato)diboron (B₂pin₂), N-bromosuccinimide (NBS), bromo-tris-pyrrolidino-phosphonium hexafluorophosphate (PyBroP),boron tribromide (BBr₃), butyl lithium (BuLi), calculated (Calcd.), cesium carbonate (Cs₂CO₃), dichloromethane (DCM, CH₂Cl₂), N,N-dicyclohexylcarbodiimide (DCC), dichloroethane (DCE), diethyl azodicarboxylate (DEAD), diisopropyl azodicarboxylate (DIAD), N,N-diisopropylethylamine (DIPEA), 4-dimethylaminopyridine (DMAP), dimethylformamide (DMF), dimethyl sulfoxide (DMSO), di-tert-butyl decarbonate (Boc₂O), 1-ethyl-3-(3-dimethylaminopropyl)carbodiimide (EDC), electrospray ionization (ESI), enantiomeric excess (ee), ethyl acetate (EtOAc), hour (h.), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), hydroxybenzotriazole (HOBt), isopropyl alcohol (IPA), lithium hydroxide monohydrate (LiOH·H2O), methanol (MeOH), methyl iodide (MeI), minutes (min.), potassium carbonate (K₂CO₃), liquid chromatography-mass spectrometry (LCMS), phenyliodide(III) diacetate (PIDA), propylphosphonic anhydride (T₃P), reverse phase (RP), room/ambient temperature (rt, RT), silver oxide (Ag₂O), sodium hydride (NaH), sodium sulfate (Na₂SO₃), supercritical fluid chromatography (SFC), tetrahydrofuran (THF), triethylamine (Et₃N), thionyl chloride (SOCl₂), triphenylphosphine (PPh₃), dicyclohexyl[2′,4′,6′-tris(propan-2-yl)[1,1′-biphenyl]-2-yl]phosphane (XPhos).

Table 1 provides a listing of example compounds of the present invention and IC₅₀ values for inhibition of POLRMT.

### Examples 1-2: 7-Hydroxy-4-(o-tolyl)-2H-chromen-2-one and 7-propoxy-4-(o-tolyl)-2H-chromen-2-one

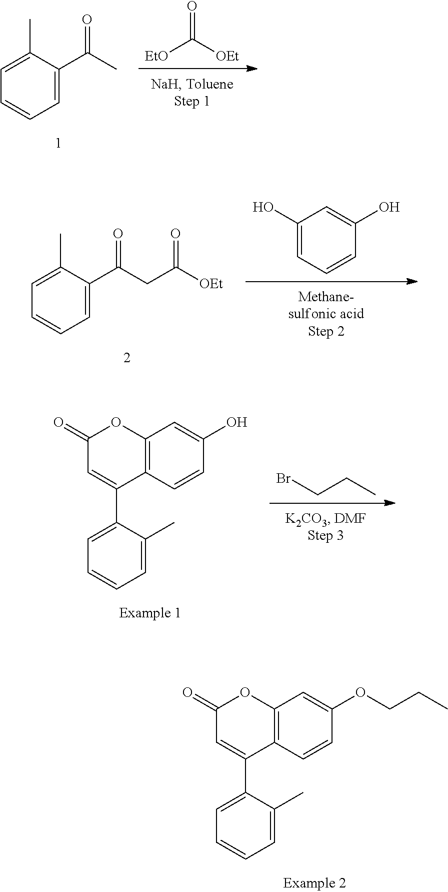

Synthesis of ethyl 3-oxo-3-(o-tolyl)propanoate, 2 [Step 1]: To a suspension of NaH (60% dispersion in mineral oil) (1.28 g, 53.6 mmol) in toluene (30 mL) at ambient temperature was added diethyl carbonate (7.2 mL, 59.62 mmol). The mixture was stirred for 15 min., then 2-methylacetophenone (1, 2 g, 14.9 mmol) was added. The mixture was then gradually warmed to 100° C. and stirred at 100° C. for 4 h. The reaction mixture was then cooled to 0-1° C. and quenched with saturated aqueous NH₄Cl solution and extracted with EtOAc (thrice). The organic layers were combined, washed with water, brine, dried over anhydrous sodium sulfate, filtered, then concentrated under reduced pressure. This product was purified by flash column chromatography to give ethyl 3-oxo-3-(o-tolyl)propanoate (2, 2.5 g). LCMS Calcd. for C₁₂H₁₄O₃: 206, found [M+H]⁺=207.

Synthesis of 7-hydroxy-4-(o-tolyl)-2H-chromen-2-one, Example 1 [Step 2]: Methane sulfonic acid (2 mL) was added drop-wise to a mixture of ethyl 3-oxo-3-(o-tolyl)propanoate (2, 1 g, 4.84 mmol) and resorcinol (0.53 g, 4.84 mmol) at ambient temperature. The resulting mixture was stirred for 16 h, cooled to 0-1° C., and quenched with water resulting in formation of a solid. The solid was filtered, washed with water, pentane, and dried under reduced pressure to give 7-hydroxy-4-(o-tolyl)-2H-chromen-2-one (Example 1, 1 g). ¹H NMR (400 MHz, DMSO-d₆) δ 10.63 (s, 1H), 7.43-7.32 (m, 3H), 7.23 (d, 1H), 6.81-6.71 (m, 3H), 6.09 (s, 1H), 2.16 (s, 3H). LCMS Calcd. for C₁₆H₁₂O₃: 252, found [M+H]⁺=253.

Synthesis of 7-propoxy-4-(o-tolyl)-2H-chromen-2-one, Example 2 [Step 3]: To a stirred solution of 7-hydroxy-4-(o-tolyl)-2H-chromen-2-one (Example 1, 150 mg, mmol) in DMF (2 mL) at ambient temperature was added K₂CO₃ (205 mg, 1.49 mmol) followed by 1-bromopropane (0.11 mL, 1.19 mmol). The mixture was heated to 70° C. for 16 h, cooled to ambient temperature, and partitioned between EtOAc and water. The organic layer was collected, washed with water (four times), brine, dried over anhydrous sodium sulfate, filtered, then concentrated under reduced pressure. The product was purified by flash column chromatography and lyophilized to afford 7-propoxy-4-(o-tolyl)-2H-chromen-2-one (Example 2, 134 mg). ¹H NMR (400 MHz, DMSO-d₆) δ 7.48-7.30 (m, 3H), 7.23 (d, 1H), 7.07 (s, 1H), 6.92-6.80 (m, 2H), 6.17 (s, 1H), 4.04 (t, 2H), 2.10 (s, 3H), 1.83-1.68 (m, 2H), 0.98 (t, 3H). LCMS (ESI) Calcd. for C₁₉H₁₈O₃: 294, found [M+H]⁺=295.

### Example 3: 7-(2-hydroxyethoxy)-4-(o-tolyl)-2H-chromen-2-one

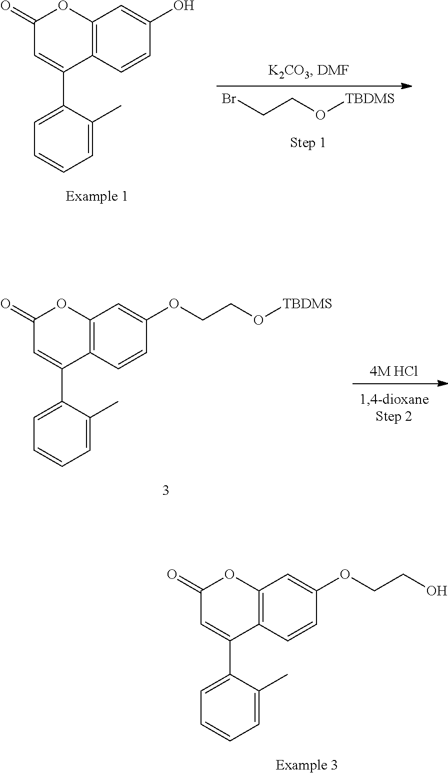

Synthesis of 7-(2-((tert-butyldimethylsilyl)oxy)ethoxy)-4-(o-tolyl)-2H-chromen-2-one, 3 [Step 1]: To a stirred solution of 7-hydroxy-4-(o-tolyl)-2H-chromen-2-one (Example 1, 150 mg, 0.595 mmol) in DMF at ambient temperature and under an inert atmosphere was added potassium carbonate (205 mg, 1.49 mmol), followed by (2-bromoethoxy)(tert-butyl-dimethyl)silane (0.26 mL, 1.19 mmol). The reaction mixture was heated to 70° C. and stirred for 16 h, cooled to ambient temperature, and partitioned between EtOAc and water. The aqueous layer was separated and extracted with EtOAc (twice). The combined organic layers were washed with brine, dried over anhydrous sodium sulfate, filtered, then concentrated under reduced pressure. The product was purified by flash column chromatography to afford 7-(2-((tert-butyldimethylsilyl)oxy)ethoxy)-4-(o-tolyl)-2H-chromen-2-one (3, 235 mg). LCMS (ESI) Calcd. for C₂₄H₃₀O₄Si: 410, found [M+H]⁺=411. ¹H NMR (400 MHz, DMSO-d₆) δ 7.45-7.33 (m, 3H), 7.24 (d, 1H), 7.08 (s, 1H), 6.87 (br s, 2H), 6.17 (s, 1H), 4.16 (br s, 2H), 3.94 (br s, 2H), 2.1 (s, 3H), 0.84 (s, 9H), 0.053 (s, 6H).

Synthesis of 7-(2-hydroxyethoxy)-4-(o-tolyl)-2H-chromen-2-one, Example 3 [Step 2]: To a stirred solution of 7-(2-((tert-butyldimethylsilyl)oxy)ethoxy)-4-(o-tolyl)-2H-chromen-2-one (3, 153 mg, 0.373 mmol) in 1,4-dioxane (2 mL) at 0° C. was added 4M HCl in 1,4-dioxane (1.0 mL) dropwise. The reaction mixture was gradually warmed to ambient temperature and stirred for 16 h. The solvent was removed under reduced pressure and the resulting mixture diluted with water and extracted with 30% IPA in chloroform (four times). The combined organic layers were dried over anhydrous sodium sulfate, filtered, then concentrated under reduced pressure. The product was purified by flash column chromatography to afford 7-(2-hydroxyethoxy)-4-(o-tolyl)-2H-chromen-2-one (Example 3, 37 mg). LCMS (ESI) Calcd. for C₁₈H₁₆O₄: 296, found [M+H]⁺=297. ¹H NMR (400 MHz, DMSO-d₆) δ 7.45-7.33 (m, 3H), 7.25 (d, 1H), 7.08 (br s, 1H), 6.91-6.85 (m, 2H), 6.18 (s, 1H), 4.92 (t, 1H), 4.15 (t, 2H), 3.75-3.72 (m, 2H), 2.11 (s, 3H).

### Example 4: 7-methoyx-4-(o-tolyl)-2H-chromen-2-one

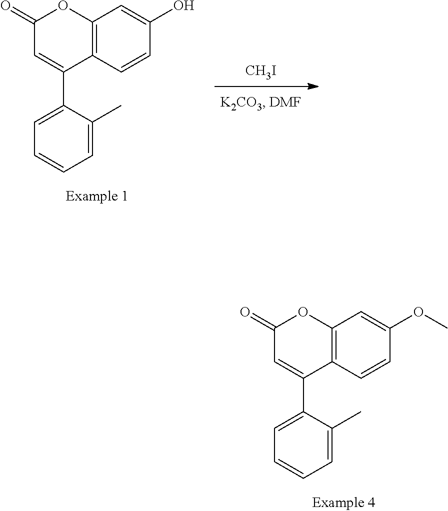 [SMILES: Cc1ccccc1-c1cc(=O)oc2cc(O)ccc12 ; "CH3I" ; COc1ccc2c(-c3ccccc3C)cc(=O)oc2c1]

Synthesis of 7-methoyx-4-(o-tolyl)-2H-chromen-2-one, Example 4: To a stirred solution of 7-hydroxy-4-(o-tolyl)-2H-chromen-2-one (Example 1, 150 mg, 0.595 mmol) in DMF (2.5 mL) at ambient temperature was added K₂CO₃ (82 mg, 0.595 mmol). The reaction mixture was cooled to 0° C. and methyl iodide (422 mg, 2.97 mmol) was added. The mixture was gradually warmed to ambient temperature and stirred for 5 h., at which time it was partitioned between EtOAc and water. The organic layer was isolated, washed with brine, dried over anhydrous sodium sulfate, filtered, then concentrated under reduced pressure. The product was purified by flash column chromatography on silica gel, then RP prep-HPLC followed by lyophilization to give 7-methoxy-4-(o-tolyl)-2H-chromen-2-one (Example 4, 20 mg). ¹H NMR (400 MHz, DMSO-d₆) δ 7.44-7.41 (m, 2H), 7.36 (t, 1H), 7.24 (d, 1H), 7.09 (s, 1H), 6.84 (m, 2H), 6.16 (s, 1H), 3.86 (s, 3H), 2.11 (s, 3H). LCMS Calcd. for C₁₇H₁₄O₃: 266, found [M+H]=267.

### Example 5: 7-isopropoxy-4-(o-tolyl)-2H-chromen-2-one

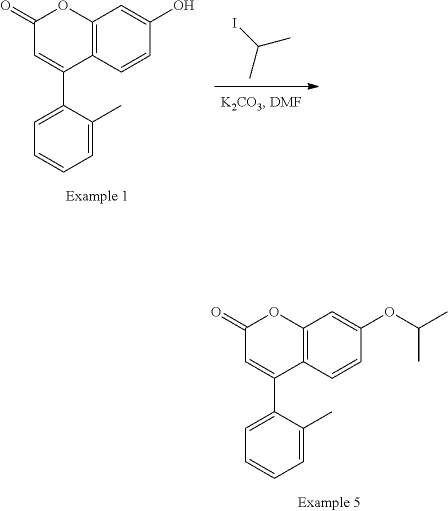

Synthesis of 7-isopropoxy-4-(o-tolyl)-2H-chromen-2-one, Example 5: To a stirred solution of 7-hydroxy-4-(o-tolyl)-2H-chromen-2-one (Example 1, 150 mg, 0.595 mmol) in DMF (2.5 mL) at ambient temperature was added K₂CO₃ (82 mg, 0.595 mmol) followed by 2-iodopropane (202 mg, 1.19 mmol). The reaction mixture was stirred at ambient temperature for 5 h, diluted with EtOAc, and washed with water (five times), brine, dried over anhydrous sodium sulfate, filtered, then concentrated under reduced pressure. The product was purified by flash column chromatography on silica gel, then by RP prep-HPLC to give 7-isopropoxy-4-(o-tolyl)-2H-chromen-2-one (Example 5, 61 mg). ¹H NMR (400 MHz, DMSO-d₆) δ 7.44-7.41 (m, 2H), 7.36 (t, 1H), 7.24 (d, 1H), 7.06 (s, 1H), 6.84 (m, 2H), 6.16 (s, 1H), 4.79-4.73 (m, 1H), 2.11 (s, 3H), 1.30 (d, 6H). LCMS Calcd. for C₁₉H₁₈O₃: 294, found [M+H]⁺=295.

### Example 6: 7-phenoxy-4-(o-tolyl)-2H-chromen-2-one

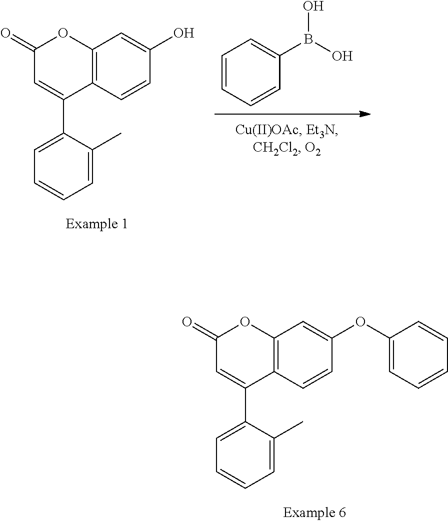 [SMILES: Cc1ccccc1-c1cc(=O)oc2cc(O)ccc12 ; OB(O)c1ccccc1 ; Cc1ccccc1-c1cc(=O)oc2cc(Oc3ccccc3)ccc12]

Synthesis of 7-phenoxy-4-(o-tolyl)-2H-chromen-2-one, Example 6: To a solution of 7-hydroxy-4-(o-tolyl)-2H-chromen-2-one (Example 1, 250 mg, 0.991 mmol), copper(II) acetate (180 mg, 0.991 mmol) and phenylboronic acid (242 mg, 1.98 mmol) in CH₂Cl₂ (10 mL) at ambient temperature and under an oxygen atmosphere was added triethylamine (0.69 mL, 4.96 mmol) and 4 Å molecular sieves. The reaction mixture was stirred at ambient temperature for 24 h., filtered, and the filtrate was concentrated under reduced pressure. Purification by flash column chromatography on silica gel, then by RP prep-HPLC gave 7-phenoxy-4-(o-tolyl)-2H-chromen-2-one (Example 6, 150 mg). ¹H NMR (400 MHz, DMSO-d₆) δ 7.46 (t, 2H), 7.43-7.38 (m, 2H), 7.34 (t, 1H), 7.26 (t, 2H), 7.16 (d, 2H), 7.0-6.96 (m, 2H), 6.91-6.88 (m, 1H), 6.27 (s, 1H), 2.12 (s, 3H). LCMS Calcd. for C₂₂H₁₆O₃: 328, found [M+H]⁺=329.

### Example 7: 7-phenethoxy-4-(o-tolyl)-2H-chromen-2-one

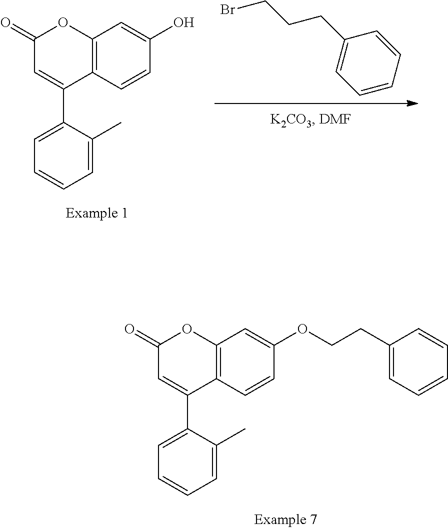 [SMILES: Cc1ccccc1-c1cc(=O)oc2cc(O)ccc12 ; BrCCCc1ccccc1 ; Cc1ccccc1-c1cc(=O)oc2cc(OCCc3ccccc3)ccc12]

Synthesis of 7-phenethoxy-4-(o-tolyl)-2H-chromen-2-one, Example 7: To a stirred solution of 7-hydroxy-4-(o-tolyl)-2H-chromen-2-one (Example 1, 150 mg, 0.595 mmol) in DMF at ambient temperature under an inert atmosphere was added K₂CO₃ (205 mg, 1.49 mmol) followed 2-bromoethylbenzene (0.16 mL, 1.19 mmol). The reaction mixture was heated to 70° C. for 16 h., then cooled to ambient temperature. The mixture was partitioned between EtOAc and water, and the organic layer was separated, washed with brine, dried over anhydrous sodium sulfate, filtered, then concentrated under reduced pressure. The product was purified by flash column chromatography on silica gel, then by RP prep-HPLC to afford 7-phenethoxy-4-(o-tolyl)-2H-chromen-2-one (Example 7, 70 mg). ¹H NMR (400 MHz, DMSO-d₆) δ 7.47-7.36 (m, 2H), 7.39-7.27 (m, 5H), 7.24 (d, 2H), 7.10 (d, 1H), 6.91-6.81 (m, 2H), 6.18 (s, 1H), 4.32 (t, 2H), 3.07 (t, 2H), 2.10 (s, 3H). LCMS (ESI) Calcd. for C₂₄H₂₀O₃: 356, found [M+H]⁺=357.

### Example 8: 7-(2-(dimethylamino)ethoxy)-4-(o-tolyl)-2H-chromen-2-one

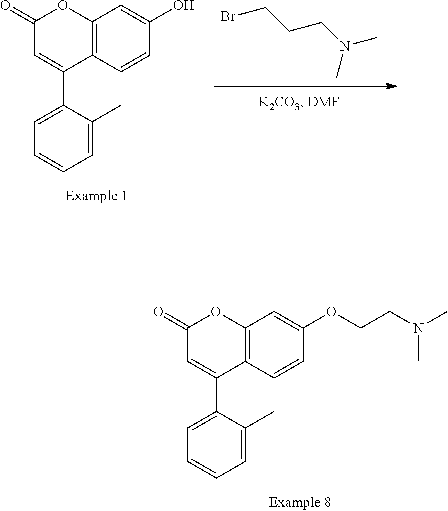

Synthesis of 7-(2-(dimethylamino)ethoxy)-4-(o-tolyl)-2H-chromen-2-one, Example 8: To a solution of 7-hydroxy-4-(o-tolyl)-2H-chromen-2-one (Example 1, 150 mg, 0.595 mmol) in DMF (3 mL) at ambient temperature was added K₂CO₃ (205 mg, 1.49 mmol. Dimethylaminoethyl bromide hydrobromide (152 mg, 0.654 mmol) was added and the mixture heated to 70° C., and stirred overnight. The reaction mixture was cooled to ambient temperature, partitioned between EtOAc and water, and the aqueous layer was separated and extracted with EtOAc. The combined organic layers were washed with water (5×15 mL), brine, dried over anhydrous sodium sulfate, filtered, and then concentrated under reduced pressure. The product was purified by flash column chromatography on silica gel, then by RP prep-HPLC to give 7-(2-(dimethylamino)ethoxy)-4-(o-tolyl)-2H-chromen-2-one (Example 8, 31 mg). ¹H NMR (400 MHz, DMSO-d₆) δ 7.47-7.30 (m, 3H), 7.23 (d, 1H), 7.1 (m, 1H), 6.93-6.82 (m, 2H), 6.17 (s, 1H), 4.19 (t, 2H), 2.68 (t, 2H), 2.2 (s, 6H), 2.1 (s, 3H). LCMS (ESI) Calcd. for C₂₀H₂₁NO₃: 323, found [M+H]⁺=324.

### Example 9: 7-(2-methoxyethoxy)-4-(o-tolyl)-2H-chromen-2-one

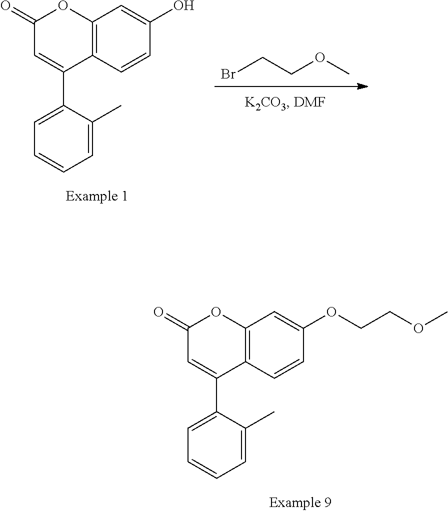

Synthesis of 7-(2-methoxyethoxy)-4-(o-tolyl)-2H-chromen-2-one, Example 9: To a solution of 7-hydroxy-4-(o-tolyl)-2H-chromen-2-one (Example 1, 150 mg, 0.59 mmol) in DMF (3 mL) at ambient temperature was added K₂CO₃ (205 mg, 1.49 mmol). 2-Bromoethyl methyl ether (91 mg, 0.654 mmol) was added and the mixture was heated to 70° C., and stirred overnight. The reaction mixture was cooled to ambient temperature and partitioned between EtOAc and water, and the organic layer was separated and washed with water (four times), brine, dried over anhydrous sodium sulfate, filtered, and then concentrated under reduced pressure. The product was purified by flash column chromatography on silica gel and lyophilized to give 7-(2-methoxyethoxy)-4-(o-tolyl)-2H-chromen-2-one (Example 9, 131 mg). ¹H NMR (400 MHz, DMSO-d₆) δ 7.45-7.38 (m, 2H), 7.35 (t, 1H), 7.25 (d, 1H), 7.10 (d, 1H), 6.90-6.84 (m, 2H), 6.18 (s, 1H), 4.21 (m, 2H), 3.67 (m, 2H), 3.28 (s, 3H), 2.11 (s, 3H). LCMS (ESI) Calcd. for C₁₉H₁₈O₄: 310, found [M+H]⁺=311.

### Examples 10-12: Ethyl 3-((2-oxo-4-(o-tolyl)-2H-chromen-7-yl)oxy)propanoate, 3-((2-oxo-4-(o-tolyl)-2H-chromen-7-yl)oxy)propanoic acid, and N-methyl-3-((2-oxo-4-(o-tolyl)-2H-chromen-7-yl)oxy)propanamide

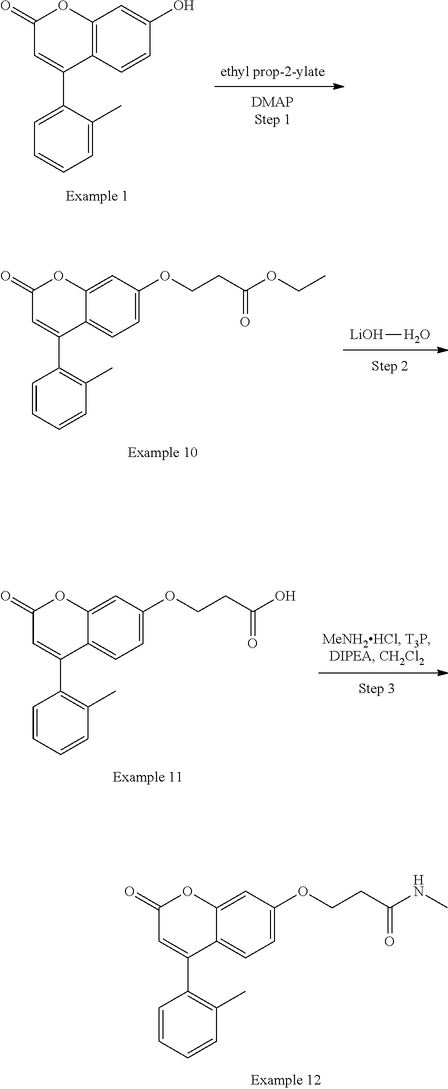

Synthesis of ethyl 3-((2-oxo-4-(o-tolyl)-2H-chromen-7-yl)oxy)propanoate, Example 10 [Step 1]: A mixture of 7-hydroxy-4-(o-tolyl)-2H-chromen-2-one (Example 1, 200 mg, 0.793 mmol), ethyl prop-2-enoate (1.0 mL, 9.51 mmol) and DMAP (19 mg, mmol) was heated to 100° C. for 24 h. The reaction temperature was increased to 120° C. and held there for another 24 h. Reaction was continued for another 48 h. at 130° C. The mixture was cooled to ambient temperature and concentrated under reduced pressure. Purification by flash column chromatography on silica gel gave ethyl 3-((2-oxo-4-(o-tolyl)-2H-chromen-7-yl)oxy)propanoate (Example 10, 48 mg). ¹H NMR (400 MHz, DMSO-d₆) δ 7.45-7.35 (m, 3H), 7.25 (d, 1H), 7.10 (m, 1H), 6.86 (m, 2H), 6.19 (s, 1H), 4.3 (t, 2H), 4.1 (q, 2H), 2.82 (t, 2H), 2.11 (s, 3H), 1.18 (t, 3H). LCMS (ESI) Calcd. for C₂₁H₂₀O₅: 352, found [M+H]⁺=353.

Synthesis of 3-((2-oxo-4-(o-tolyl)-2H-chromen-7-yl)oxy)propanoic acid, Example 11 [Step 2]: To a solution of ethyl 3-((2-oxo-4-(o-tolyl)-2H-chromen-7-yl)oxy)propanoate (Example 10, 45 mg, 0.128 mmol) in THF (4 mL) at ambient temperature was added a solution of LiOH·H₂O (27 mg, 0.639 mmol) in water (1 mL). The reaction mixture was stirred for 5 h., and concentrated under reduced pressure. The resulting mixture was acidified with saturated aqueous citric acid solution and extracted with EtOAc (twice). The combined organic extracts were washed with water, brine, dried over anhydrous sodium sulfate, filtered, and concentrated under reduced pressure to give 3-((2-oxo-4-(o-tolyl)-2H-chromen-7-yl)oxy)propanoic acid (Example 11, 41 mg). LCMS (ESI) Calcd. for C₁₉H₁₆O₅: 324, found [M+H]⁺=325.

Synthesis of N-methyl-3-((2-oxo-4-(o-tolyl)-2H-chromen-7-yl)oxy)propenamide, Example 12 [Step 3]: To a solution of 3-((2-oxo-4-(o-tolyl)-2H-chromen-7-yl)oxy)propanoic acid (Example 11, 41 mg, 0.126 mmol) in CH₂Cl₂ (4 mL) at ambient temperature and under an inert atmosphere was added methyl amine hydrochloride (10 mg, 0.152 mmol). The mixture was cooled to 0° C. and DIPEA (0.044 mL, 0.253 mmol) was added, followed by dropwise addition of T₃P, 50% in EtOAc (0.041 mL, 0.139 mmol). The resulting mixture was gradually warmed to ambient temperature and stirred for 2 h. The mixture was diluted with CH₂Cl₂, washed with water, brine, dried over anhydrous sodium sulfate, filtered, and concentrated under reduced pressure. The product was purified by RP prep-HPLC to produce N-methyl-3-((2-oxo-4-(o-tolyl)-2H-chromen-7-yl)oxy)propanamide (Example 12, 7 mg). ¹H NMR (400 MHz, DMSO-d₆) δ 7.92 (br s, 1H), 7.43-7.33 (m, 3H), 7.23 (d, 1H), 7.08 (s, 1H), 6.87 (m, 2H), 6.18 (s, 1H), 4.33 (t, 2H), 2.59-2.55 (m, 5H), 2.10 (s, 3H). LCMS Calcd. for C₂₀H₁₉NO₄: 337, found [M+H]⁺=338.

### Examples 13-14: 4-(2-chloro-4-fluorophenyl)-7-hydroxy-2H-chromen-2-one and methyl 2-((4-(2-chloro-4-fluorophenyl)-2-oxo-2H-chromen-7-yl)oxy)-3-methoxypropanoate

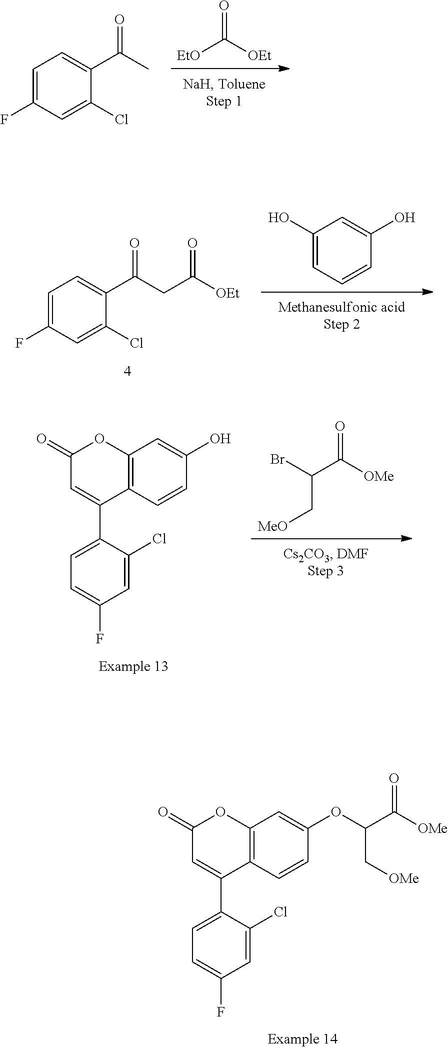

Synthesis of ethyl 3-(2-chloro-4-fluorophenyl)-3-oxopropanoate, 4 [Step 1]: Sodium hydride (60% dispersion in mineral oil) (12.11 g, 303 mmol) was charged in a 1 L round-bottom flask, washed with n-pentane, and dried under flow of nitrogen gas. Dry toluene (150 mL) was added and the resulting suspension was cooled to 0° C. Diethyl carbonate (70 mL, 579 mmol) was then added dropwise, followed by 1-(2-chloro-4-fluorophenyl)ethan-1-one (25.00 g, 145 mmol). The mixture was stirred for 30 min., then gradually warmed to 70° C., where it was stirred for 16 h. The reaction mixture was then cooled to 0° C. and quenched by dropwise addition of saturated aqueous NH₄Cl solution. The resulting mixture was extracted with EtOAc (thrice). The combined organic extracts were washed with brine, dried over anhydrous sodium sulfate, filtered, and concentrated under reduced pressure. The product was purified by flash column chromatography on silica gel to give ethyl 3-(2-chloro-4-fluoro-phenyl)-3-oxo-propanoate (4, 14 g). LCMS (ESI) Calcd. for C₁₁H₁₀ClFO₃: 244, found [M+H]⁺=245. ¹H NMR (400 MHz, DMSO-d₆) δ 7.93-7.89 (m, 1H), 7.61-7.57 (m, 1H), 7.41-7.34 (m, 1H), 4.2 (s, 2H), 4.11 (q, 2H), 1.19 (t, 3H).

Synthesis of 4-(2-chloro-4-fluorophenyl)-7-hydroxy-2H-chromen-2-one, Example 13 [Step 2]: To a mixture of ethyl 3-(2-chloro-4-fluoro-phenyl)-3-oxo-propanoate (4, 5 g, 20.4 mmol) and benzene-1,3-diol (2.25 g, 20.4 mmol) at ambient temperature was added methane sulphonic acid (13 mL, 204 mmol) dropwise. The mixture was stirred at 50° C. overnight, cooled to 10° C., and quenched with water. The product was filtered, washed with water and n-pentane, and dried under reduced pressure to give 4-(2-chloro-4-fluorophenyl)-7-hydroxy-2H-chromen-2-one (Example 13, 5.8 g). LCMS (ESI) Calcd. for C₁₅H₈ClFO₃: 290, found [M+H]⁺=291. ¹H NMR (400 MHz, DMSO-d₆) δ 10.68 (s, 1H), 7.70-7.67 (dd, 1H), 7.56-7.53 (m, 1H), 7.44-7.39 (dt, 1H), 6.87 (d, 1H), 6.80 (d, 1H), 6.75-6.73 (dd, 1H), 6.21 (s, 1H).

Synthesis of methyl 2-((4-(2-chloro-4-fluorophenyl)-2-oxo-2H-chromen-7-yl)oxy)-3-methoxypropanoate, Example 14 [Step 3]: To a stirred solution of 4-(2-chloro-4-fluorophenyl)-7-hydroxy-2H-chromen-2-one (Example 13, 200 mg, 0.688 mmol) in DMF (2 mL) at ambient temperature was added Cs₂CO₃ (336.28 mg, 1.0321 mmol) followed by methyl 2-bromo-3-methoxy-propanoate (149.13 mg, 0.7569 mmol). The reaction mixture was stirred at ambient temperature for 18 h., then diluted with EtOAc, washed with water (thrice), brine, dried over anhydrous sodium sulfate, filtered, and concentrated under reduced pressure. The product was purified by RP prep-HPLC and lyophilized to give methyl 2-((4-(2-chloro-4-fluorophenyl)-2-oxo-2H-chromen-7-yl)oxy)-3-methoxypropanoate (Example 14, 152 mg). ¹H NMR (400 MHz, DMSO-d₆) δ 7.70 (d, 1H), 7.56 (t, 1H), 7.46-7.42 (m, 1H), 7.09 (s, 1H), 6.96-6.90 (m, 2H), 6.33 (s, 1H), 5.36 (s, 1H), 3.89-3.79 (m, 2H), 3.70 (s, 3H), 3.28 (s, 3H). LCMS Calcd. for C₂₀H₁₆ClFO₆: 406, found [M+H]⁺=407.

### Example 15: (R)-4-(2-chloro-4-fluorophenyl)-7-((3-hydroxy-1-oxo-1-(piperidin-1-yl)propan-2-yl)oxy)-2H-chromen-2-one

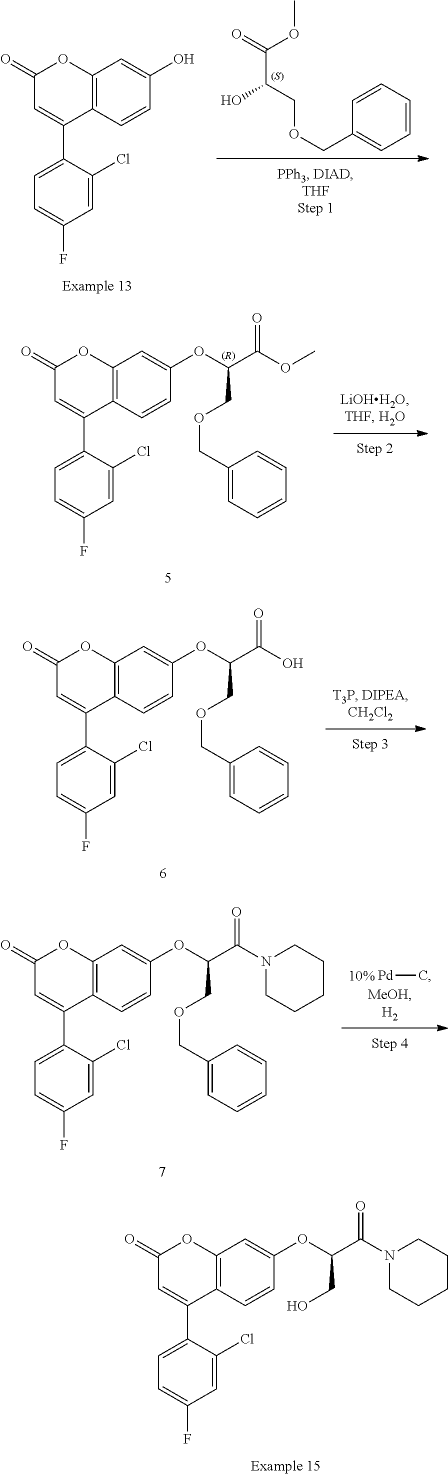

Synthesis of methyl (R)-3-(benzyloxy)-2-((4-(2-chloro-4-fluorophenyl)-2-oxo-2H-chromen-7-yl)oxy)propanoate, 5 [Step 1]: To a stirred solution of 4-(2-chloro-4-fluorophenyl)-7-hydroxy-2H-chromen-2-one (Example 13, 280 mg, 1 mmol) in THF (2 mL) under an inert atmosphere were added triphenylphosphine (379 mg, 1.4 mmol) followed by methyl (S)-3-(benzyloxy)-2-hydroxypropanoate (200 mg, 1 mmol) and molecular sieves (4 Å°). The mixture was cooled to 0° C. and DIAD (0.6 mL, 3 mmol) was added dropwise. The reaction mixture was gradually warmed to 50° C. and stirred for 12 h. The reaction mixture was filtered and the product was purified by flash column chromatography on silica gel to give methyl (R)-3-(benzyloxy)-2-((4-(2-chloro-4-fluorophenyl)-2-oxo-2H-chromen-7-yl)oxy)propanoate (5, 150 mg). LCMS (ESI) Calcd. for C₂₆H₂₀ClFO₆: 482, found [M+H]⁺=483.

Synthesis of (R)-3-(benzyloxy)-2-((4-(2-chloro-4-fluorophenyl)-2-oxo-2H-chromen-7-yl)oxy)propanoic acid, 6 [Step 2]: To a solution of methyl (R)-3-(benzyloxy)-2-((4-(2-chloro-4-fluorophenyl)-2-oxo-2H-chromen-7-yl)oxy)propanoate (5, 100 mg, 0.2 mmol) in THF (3 mL) at ambient temperature was added a solution of LiOH·H₂O (25 mg, 0.6 mmol) in water (0.6 mL). The reaction mixture was stirred for 16 h., then concentrated under reduced pressure. The product was diluted with water and washed with EtOAc. The aqueous layer was acidified (pH=5-6) with 10M aqueous HCl solution and the product precipitated. The product was filtered, washed with water, and dried to give (R)-3-(benzyloxy)-2-((4-(2-chloro-4-fluorophenyl)-2-oxo-2H-chromen-7-yl)oxy)propanoic acid (6, 90 mg). LCMS (ESI) Calcd. for C₂₅H₁₈ClFO₆: 468, found [M+H]⁺=469.

Synthesis of (R)-7-((3-(benzyloxy)-1-oxo-1-(piperidin-1-yl)propan-2-yl)oxy)-4-(2-chloro-4-fluorophenyl)-2H-chromen-2-one, 7 [Step 3]: To a solution of (R)-3-(benzyloxy)-2-((4-(2-chloro-4-fluorophenyl)-2-oxo-2H-chromen-7-yl)oxy)propanoic acid (6, 50 mg, 0.1 mmol) in CH₂Cl₂ (4 mL) at ambient temperature was added piperidine (0.02 mL, 0.2 mmol) followed by DIPEA (0.04 mL, 0.3 mmol). The mixture was cooled 0° C. and T₃P, 50% in EtOAc (0.03 mL, 0.1 mmol) was added. The resulting mixture was stirred for 2 h. at ambient temperature and diluted with CH₂Cl₂. The product was washed with 10% aqueous K₂CO₃ solution, brine, dried over anhydrous sodium sulfate, filtered, and concentrated under reduced pressure. The product was purified by flash column chromatography on silica gel to give (R)-7-((3-(benzyloxy)-1-oxo-1-(piperidin-1-yl)propan-2-yl)oxy)-4-(2-chloro-4-fluorophenyl)-2H-chromen-2-one (7, 45 mg). LCMS (ESI) Calcd. for C₃₀H₂₇ClFNO₅: 535, found [M+H]⁺=536.

Synthesis of (R)-4-(2-chloro-4-fluorophenyl)-7-((3-hydroxy-1-oxo-1-(piperidin-1-yl)propan-2-yl)oxy)-2H-chromen-2-one, Example 15 [Step 4]: To a stirred solution of (R)-7-((3-(benzyloxy)-1-oxo-1-(piperidin-1-yl)propan-2-yl)oxy)-4-(2-chloro-4-fluorophenyl)-2H-chromen-2-one (7, 40 mg, 0.1 mmol) in ethanol (5 mL) was bubbled nitrogen for 5 min. 10% Pd-C (10 mg) was added under an inert atmosphere and the reaction mixture was stirred under a hydrogen balloon pressure at ambient temperature for 2 h. The reaction mixture was filtered, concentrated under reduced pressure, purified by RP prep-HPLC, and lyophilized to give (R)-4-(2-chloro-4-fluorophenyl)-7-((3-hydroxy-1-oxo-1-(piperidin-1-yl)propan-2-yl)oxy)-2H-chromen-2-one (Example 15, 2.5 mg). LCMS (ESI) Calcd. for C₂₃H₂₁ClFNO₅: 445, found [M+H]⁺=446. ¹H NMR (at 80° C.) (400 MHz, DMSO-d₆): δ 7.65 (d, 1H), 7.55 (t, 1H), 7.41 (t, 1H), 6.97-6.92 (m, 1H), 6.91 (s, 1H), 6.85-6.83 (m, 1H), 6.28 (s, 1H), 5.28 (br s, 1H), 5.08 (br s, 1H), 3.79 (s, 2H), 3.50 (br s, 6H), 1.59 (s, 2H), 1.47 (s, 2H).

### Examples 16-20: 4-(4-fluorophenyl)-7-hydroxy-5-methyl-2H-cheromen-2-one, ethyl (R)-2-((4-(4-fluorophenyl)-5-methyl-2-oxo-2H-chromen-7-yl)oxy)propanoate, (R)-2-((4-(4-fluorophenyl)-5-methyl-2-oxo-2H-chromen-7-yl)oxy)propanoic acid, 4-(4-fluorophenyl)-5-methyl-7-[(1R)-1-methyl-2-oxo-2-(1-piperidyl)ethoxy]chromen-2-one, and (R)-2-((4-(4-fluorophenyl)-5-methyl-2-oxo-2H-chromen-7-yl)oxy)-N,N-dimethylpropanamide

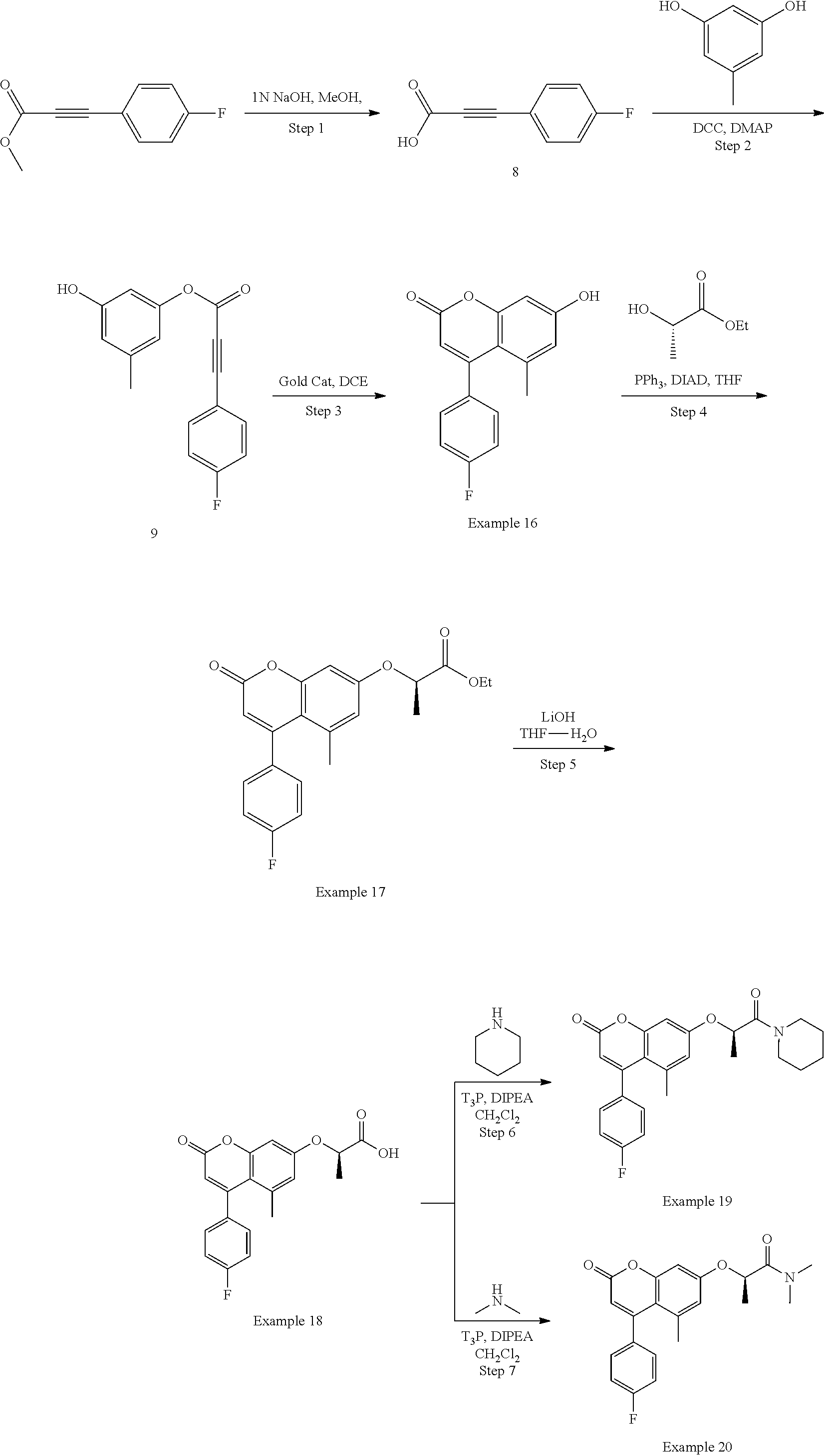

Synthesis of 3-(4-fluorophenyl)propiolic acid, 8 [Step 1]: To a stirred solution of methyl 3-(4-fluorophenyl)propiolate (1.00 eq, 750 mg, 4.2 mmol) in ethanol (15 mL) at ambient temperature was added 1N aqueous NaOH solution (7.5 mL), and the reaction mixture stirred for 2.5 h. The reaction mixture was concentrated under reduced pressure and the product was dissolved in water and extracted with EtOAc. The aqueous layer was separated and acidified with 1N aqueous HCl solution and extracted with EtOAc. The organic extract was washed with water, brine, dried over anhydrous sodium sulfate, filtered, and concentrated under reduced pressure to afford 3-(4-fluorophenyl)propiolic acid (8, 680 mg). LCMS (ESI) Calcd. for C₉H₅FO₂: 164, found [M+H]⁺=165. ¹H NMR (400 MHz, DMSO-d₆) δ 13.93 (br s, 1H), 7.72-7.68 (m, 2H), 7.34-7.30 (m, 2H).

Synthesis of 3-hydroxy-5-methylphenyl 3-(4-fluorophenyl)propiolate, 9 [Step 2]: To a stirred solution of 5-methylbenzene-1,3-diol (1.00 eq, 500 mg, 4.0 mmol) in CH₂Cl₂ (15 mL) at ambient temperature was added 3-(4-fluorophenyl)propiolic acid (8, 1.10 eq, 727 mg, 4.4 mmol), followed by DCC (1.50 eq, 1247 mg, 6.0 mmol) in a mixture of CH₂Cl₂ (10 mL) and DMAP (0.100 eq, 49 mg, 0.40 mmol). The resulting mixture was stirred at ambient temperature for 16 h. The reaction mixture was filtered and the filtrate was diluted with CH₂Cl₂, washed with water, brine, dried over anhydrous sodium sulfate, filtered, and concentrated under reduced pressure. The product was purified by flash column chromatography to afford 3-hydroxy-5-methylphenyl 3-(4-fluorophenyl)propiolate (9, 480 mg). LCMS (ESI) Calcd. for Cl₆H₁₁FO₃: 270, found [M+H]⁺=271. ¹H NMR (400 MHz, DMSO-d₆) δ 9.72 (s, 1H), 7.83-7.79 (m, 2H), 7.38 (t, 2H), 6.54 (s, 1H), 6.50 (s, 1H), 6.44 (s, 1H), 2.24 (s, 3H).

Synthesis of 4-(4-fluorophenyl)-7-hydroxy-5-methyl-2H-chromen-2-one, Example 16 [Step 3]: A solution of 3-hydroxy-5-methylphenyl 3-(4-fluorophenyl)propiolate (9, 1.00 eq, 487 mg, 1.8 mmol) in DCE (35 mL) was purged with argon for 10 min. (Acetonitrile)[(2-biphenyl)di-tert-butyl phosphine]gold(I) hexafluoroantimonate (0.100 eq, 140 mg, 0.18 mmol) was added and the reaction mixture was stirred at ambient temperature under an argon atmosphere for 16 h. The reaction mixture was filtered through a celite bed and the filtrate was concentrated under reduced pressure. The product was purified by flash column chromatography to afford 4-(4-fluorophenyl)-7-hydroxy-5-methyl-2H-chromen-2-one (Example 16, 300 mg, 1.1 mmol). LCMS (ESI) Calcd. for Cl₆H₁₁FO₃: 270, found [M+H]⁺=271. ¹H NMR (400 MHz, DMSO-d₆) δ 10.56 (s, 1H), 7.44-7.40 (m, 2H), 7.34-7.29 (m, 2H), 6.66 (d, 1H), 6.56 (d, 1H), 5.95 (s, 1H), 1.68 (s, 3H).

Synthesis of ethyl (R)-2-((4-(4-fluorophenyl)-5-methyl-2-oxo-2H-chromen-7-yl)oxy)propanoate, Example 17 [Step 4]: To a stirred solution of 4-(4-fluorophenyl)-7-hydroxy-5-methyl-2H-chromen-2-one (Example 16, 1.00 eq, 180 mg, 0.7 mmol) in dry THF (3 mL) at ambient temperature and under an argon atmosphere was added ethyl (S)-2-hydroxypropanoate (1.50 eq, 118 mg, 1.0 mmol), PPh₃ (3.00 eq, 524 mg, 2.0 mmol), and DIAD (3.00 eq, 0.39 mL, 2.00 mmol). The reaction mixture was heated to 50° C. for 16 h. and quenched with water and extracted with EtOAc. The organic layer was separated, dried over anhydrous sodium sulfate, filtered, and concentrated under reduced pressure. The product was purified by flash column chromatography to afford ethyl (R)-2-((4-(4-fluorophenyl)-5-methyl-2-oxo-2H-chromen-7-yl)oxy)propanoate (Example 17, 180 mg, 0.5 mmol). LCMS (ESI) Calcd. for C₂₁H₁₉FO₅: 370, found [M+H]⁺=371. ¹H NMR (400 MHz, DMSO-d₆) δ 7.45-7.42 (m, 2H), 7.32 (t, 2H), 6.86 (d, 1H), 6.73 (d, 1H), 6.06 (s, 1H), 5.20-5.15 (m, 1H), 4.19-4.13 (m, 2H), 1.72 (s, 3H), 1.53 (d, 3H), 1.18 (t, 3H).

Synthesis of (R)-2-((4-(4-fluorophenyl)-5-methyl-2-oxo-2H-chromen-7-yl)oxy)propanoic acid, Example 18 [Step 5]: To a stirred solution of ethyl (R)-2-((4-(4-fluorophenyl)-5-methyl-2-oxo-2H-chromen-7-yl)oxy)propanoate (Example 17, 1.00 eq, 200 mg, 0.5 mmol) in THF (4 mL) and water (1 mL) at ambient temperature was added LiOH·H₂O (3.00 eq, 68 mg, 1.6 mmol). The reaction mixture was stirred for 16 h., and concentrated under reduced pressure. The product was dissolved in water and washed with EtOAc. The aqueous layer was separated and acidified with citric acid to pH-5 and extracted with EtOAc. The organic extract was washed with water, brine, dried over anhydrous sodium sulfate, filtered, and concentrated under reduced pressure to give (R)-2-((4-(4-fluorophenyl)-5-methyl-2-oxo-2H-chromen-7-yl)oxy)propanoic acid (Example 18, 180 mg, 0.5 mmol). LCMS (ESI) Calcd. for C₁₉H₁₅FO₅: 342, found [M+H]⁺=343.

Synthesis of 4-(4-fluorophenyl)-5-methyl-7-[(1R)-1-methyl-2-oxo-2-(1-piperidyl)ethoxy]chromen-2-one, Example 19 [Step 6]: To a stirred solution of (R)-2-((4-(4-fluorophenyl)-5-methyl-2-oxo-2H-chromen-7-yl)oxy)propanoic acid (Example 18, 1.00 eq, 75 mg, 0.22 mmol) in CH₂Cl₂ (3 mL) at ambient temperature was added piperidine (1.20 eq, 22 mg, 0.3 mmol), DIPEA (3.00 eq, 0.11 mL, 0.7 mmol), T₃P (1.50 eq, 0.19 mL, 0.33 mmol) and the reaction mixture stirred for 16 h. The mixture was concentrated under reduced pressure and the product purified by RP prep-HPLC to afford 4-(4-fluorophenyl)-5-methyl-7-[(1R)-1-methyl-2-oxo-2-(1-piperidyl)ethoxy]chromen-2-one (Example 19, 30 mg, 0.0731 mmol). LCMS (ESI) Calcd. for C₂₄H₂₄FNO₄: 409, found [M+H]⁺=410. ¹H NMR (400 MHz, DMSO-d₆) δ 7.45-7.42 (m, 2H), 7.32 (t, 2H), 6.79 (d, 1H), 6.69 (d, 1H), 6.05 (s, 1H), 5.48-5.43 (m, 1H), 3.54-3.47 (m, 3H), 3.39-3.35 (m, 1H), 1.72 (s, 3H), 1.60-1.54 (m, 4H), 1.43-1.41 (m, 5H).

Synthesis of (R)-2-((4-(4-fluorophenyl)-5-methyl-2-oxo-2H-chromen-7-yl)oxy)-N,N-dimethylpropanamide, Example 20 [Step 7]: To a stirred solution of (R)-2-((4-(4-fluorophenyl)-5-methyl-2-oxo-2H-chromen-7-yl)oxy)propanoic acid (Example 18, 1.00 eq, 75 mg, 0.22 mmol) in CH₂Cl₂ (3 mL) at ambient temperature was added N-methylmethanamine HCl (1.20 eq, 21 mg, 0.26 mmol), DIPEA (3.00 eq, 0.11 mL, 0.66 mmol), and T₃P (1.50 eq, 0.19 mL, 0.34 mmol). The reaction mixture was stirred at ambient temperature for 16 h., and concentrated under reduced pressure. The product was purified by RP prep-HPLC to afford (R)-2-((4-(4-fluorophenyl)-5-methyl-2-oxo-2H-chromen-7-yl)oxy)-N,N-dimethylpropanamide (Example 20, 30 mg, 0.08 mmol). LCMS (ESI) Calcd. for C₂₁H₂₀FNO₄: 369, found [M+H]⁺=370. ¹H NMR (400 MHz, DMSO-d₆) δ 7.45-7.42 (m, 2H), 7.32 (t, 2H), 6.77 (d, 1H), 6.69 (d, 1H), 6.04 (s, 1H), 5.43-5.42 (m, 1H), 3.09 (s, 3H), 2.85 (s, 0.3H), 1.72 (s, 3H), 1.42 (d, 3H).

### Example 21: 4-(2-chloro-4-fluorophenyl)-7-methoxy-2H-chromen-2-one

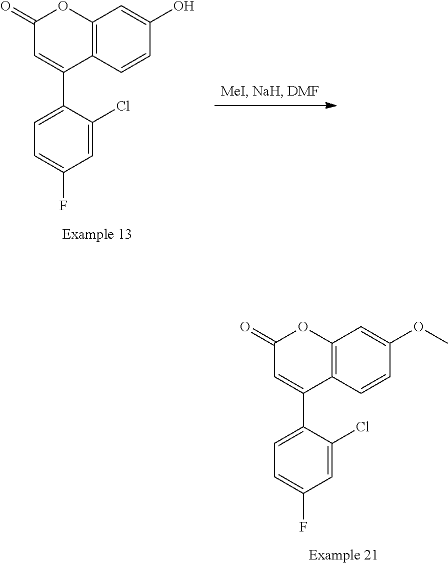

Synthesis of 4-(2-chloro-4-fluorophenyl)-7-methoxy-2H-chromen-2-one, Example 21: To a stirred solution of compound 4-(2-chloro-4-fluorophenyl)-7-hydroxy-2H-chromen-2-one (Example 13, 200 mg, 0.7 mmol) in dry DMF (5 mL) at 0° C. and under a nitrogen atmosphere was added NaH (60% dispersion in mineral oil) (20 mg, 0.8 mmol) portion wise. The reaction mixture was slowly warmed to ambient temperature and stirred for 30 min. Methyl iodide (0.9 mL, 14 mmol) was added and mixture stirred for an additional 16 h. The reaction was quenched with saturated aqueous NH₄Cl solution and extracted with EtOAc (thrice). The combined organic extracts were washed with brine, dried over anhydrous sodium sulfate, filtered, then concentrated under reduced pressure. The product was purified by flash column chromatography on silica gel to give 4-(2-chloro-4-fluorophenyl)-7-methoxy-2H-chromen-2-one (Example 21, 96 mg). LCMS (ESI) Calcd. for Cl₆H₁₀ClFO₃: 304, found [M+H]⁺=305. ¹H NMR (400 MHz, DMSO-d₆) δ 7.70 (dd, 1H), 7.59-7.55 (m, 1H), 7.44 (dt, 1H), 7.11 (d, 1H), 6.95 (d, 1H), 6.90 (dd, 1H), 6.31 (s, 1H), 3.87 (s, 3H).

### Examples 22-24: 7-hydroxy-5-methyl-4-phenyl-2H-chromen-2-one, 7-isopropoxy-5-methyl-4-phenyl-2H-chromen-2-one, and 7-isobutoxy-5-methyl-4-phenyl-2H-chromen-2-one

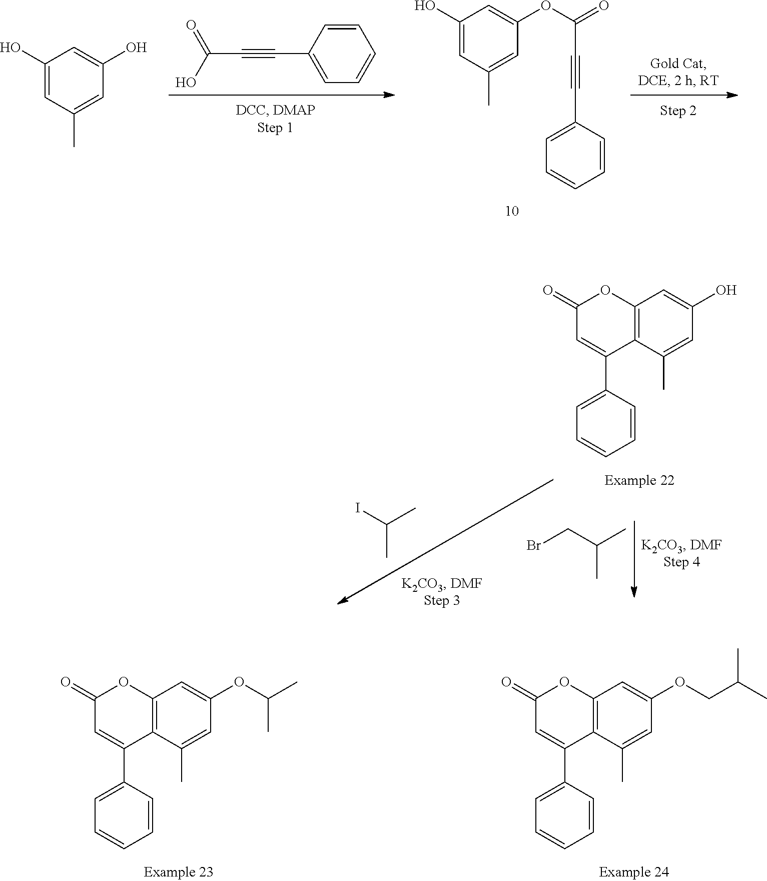

Synthesis of 3-hydroxy-5-methylphenyl-3-phenylpropiolate, 10 [Step 1]: To a stirred solution of 3-phenylpropiolic acid (194 mg, 1.33 mmol) and 5-methylbenzene-1,3-diol (150 mg, 1.21 mmol) in CH₂Cl₂ (6 mL) at 0° C. was added a mixture of DCC (374 mg, 1.81 mmol) and DMAP (15 mg, 0.121 mmol) in CH₂Cl₂ (4 mL) dropwise. The mixture was gradually warmed to ambient temperature and stirred for 12 h. The mixture was concentrated under reduced pressure and the product was diluted with water and extracted with CH₂Cl₂ (twice). The combined organic extracts were washed with water, brine, dried over anhydrous sodium sulfate, filtered, and concentrated under reduced pressure to give 3-hydroxy-5-methylphenyl 3-phenylpropiolate (10, 200 mg). LCMS (ESI) Calcd. for C₁₆H₁₂O₃: 252, found [M+H]⁺=253.

Synthesis of 7-hydroxy-5-methyl-4-phenyl-2H-chromen-2-one, Example 22 [Step 2]: To a stirred solution of 3-hydroxy-5-methylphenyl 3-phenylpropiolate (10, 200 mg, 0.793 mmol) in DCE (5 mL) at ambient temperature and under an argon atmosphere was added (acetonitrile)[(2-biphenyl)di-tert-butyl phosphine]gold(I) hexafluoroantimonate (61 mg, 0.0793 mmol). The resulting mixture was stirred for 18 h and diluted with CH₂Cl₂, washed with water, brine, dried over anhydrous sodium sulfate, filtered, and concentrated under reduced pressure. The product was purified by RP prep-HPLC and lyophilized to give 7-hydroxy-5-methyl-4-phenyl-2H-chromen-2-one (Example 22, 90 mg). LCMS (ESI) Calcd. for C₁₆H₁₂O₃: 252, found [M+H]⁺=253. ¹H NMR (400 MHz, DMSO-d₆) δ 10.54 (br s, 1H), 7.49-7.47 (m, 3H), 7.37-7.35 (m, 2H), 6.67 (d, 1H), 6.55 (s, 1H), 5.93 (s, 1H), 1.67 (s, 3H).

Synthesis of 7-isopropoxy-5-methyl-4-phenyl-2H-chromen-2-one, Example 23 [Step 3]: To a stirred solution of 7-hydroxy-5-methyl-4-phenyl-2H-chromen-2-one (Example 22, 40 mg, 0.159 mmol) in DMF (0.5 mL) was added K₂CO₃ (44 mg, 0.317 mmol) followed by 2-iodopropane (0.024 mL, 0.238 mmol). The resulting mixture was stirred at ambient temperature for 16 h. The reaction mixture was diluted with EtOAc, washed with water (thrice), brine, dried over anhydrous sodium sulfate, filtered, and concentrated under reduced pressure. The product was purified by RP prep-HPLC and lyophilized to give 7-isopropoxy-5-methyl-4-phenyl-2H-chromen-2-one (Example 23, 38 mg). LCMS (ESI) Calcd. for C₁₉H₁₈O₃: 294, found [M+H]⁺=295. ¹H NMR (400 MHz, DMSO-d₆) δ 7.49-7.47 (m, 3H), 7.38-7.36 (m, 2H), 6.93 (d, 1H), 6.69 (br s, 1H), 6.00 (s, 1H), 4.79-4.76 (m, 1H), 1.70 (s, 3H), 1.29 (d, 6H).

Synthesis of 7-isobutoxy-5-methyl-4-phenyl-2H-chromen-2-one, Example 24 [Step 4]: To a stirred solution of 7-hydroxy-5-methyl-4-phenyl-2H-chromen-2-one (Example 22, 40 mg, 0.159 mmol) in DMF (0.5 mL) was added K₂CO₃ (44 mg, 0.317 mmol) followed by isobutyl bromide (0.026 mL, 0.238 mmol). The mixture was stirred at ambient temperature for 16 h and diluted with EtOAc. The mixture was washed with water (thrice), brine, dried over anhydrous sodium sulfate, filtered, and concentrated under reduced pressure. The product was purified by RP prep-HPLC and lyophilized to afford 7-isobutoxy-5-methyl-4-phenyl-2H-chromen-2-one (Example 24, 23 mg). LCMS (ESI) Calcd. for C₂₀H₂₀O₃: 308, found [M+H]⁺=309. ¹H NMR (400 MHz, DMSO-d₆) δ 7.49-7.48 (m, 3H), 7.38-7.36 (m, 2H), 6.93 (d, 1H), 6.73 (d, 1H), 6.01 (s, 1H), 3.86 (d, 2H), 2.05-2.02 (m, 1H), 1.71 (s, 3H), 0.98 (d, 6H).

### Examples 25-27: 7-((1-methoxypropan-2-yl)oxy)-4-(o-tolyl)-2H-chromen-2-one, racemic and purified chiral analogs

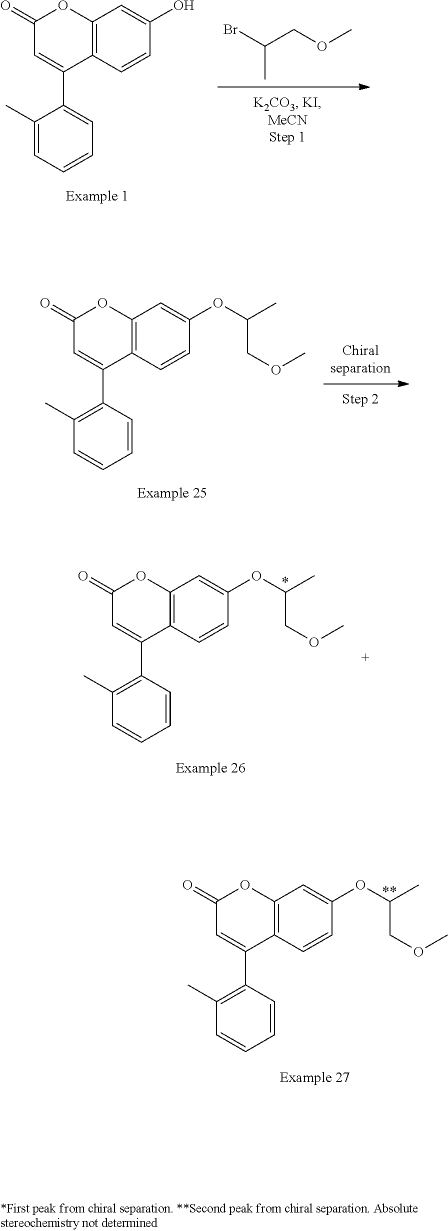

Synthesis of 7-((1-methoxypropan-2-yl)oxy)-4-(o-tolyl)-2H-chromen-2-one, Example 25 [Step 1]: To a stirred solution of 2-bromo-1-methoxy-propane (180 mg, 1.2 mmol) in MeCN (7 mL) was added K₂CO₃ (270 mg, 2.0 mmol) and KI (200 mg, 1.2 mmol) followed by 7-hydroxy-4-(o-tolyl)-2H-chromen-2-one (Example 1, 250 mg, 1.0 mmol). The mixture was stirred at 70° C. for 48 h., and cooled to ambient temperature. The mixture was concentrated under reduced pressure and the product was partitioned between EtOAc and water. The organic layer was separated, washed with water (twice), brine, dried over anhydrous sodium sulfate, filtered, and concentrated under reduced pressure. The product was purified by RP prep-HPLC and lyophilized to afford 7-((1-methoxypropan-2-yl)oxy)-4-(o-tolyl)-2H-chromen-2-one (Example 25, 100 mg). LCMS (ESI) Calcd. for C₂₀H₂₀O₄: 323, found [M+H]⁺=325. ¹H NMR (400 MHz, DMSO-d₆) δ 7.43-7.34 (m, 2H), 7.35 (t, 1H), 7.24 (d, 1H), 7.11 (d, 1H), 6.89-6.83 (m, 2H), 6.17 (s, 1H), 4.81-4.77 (m, 1H), 3.50 (t, 2H), 3.29 (d, 3H), 2.11 (s, 3H), 1.24 (d, 3H).

Synthesis of chiral 7-((1-methoxypropan-2-yl)oxy)-4-(o-tolyl)-2H-chromen-2-one, Examples 26 and 27 [Step 2]: 7-((1-methoxypropan-2-yl)oxy)-4-(o-tolyl)-2H-chromen-2-one (Example 25, 80 mg) was purified by chiral prep-HPLC separation and the first product was isolated as 7-((1-methoxypropan-2-yl)oxy)-4-(o-tolyl)-2H-chromen-2-one, Peak 1 (Example 26, 30 mg) and the second product as 7-((1-methoxypropan-2-yl)oxy)-4-(o-tolyl)-2H-chromen-2-one, Peak 2 (Example 27, 25 mg). The absolute stereochemistries of these compounds were not determined.

Example 26: [7-((1-methoxypropan-2-yl)oxy)-4-(o-tolyl)-2H-chromen-2-one, Peak 1]: LCMS (ESI) Calcd. for C₂₀H₂₀O₄: 324, found [M+H]⁺=325. ¹H NMR (400 MHz, DMSO-d₆) δ 7.43-7.34 (m, 2H), 7.35 (t, 1H), 7.24 (d, 1H), 7.11 (s, 1H), 6.89-6.83 (m, 2H), 6.17 (s, 1H), 4.79 (d, 1H), 3.50 (t, 2H), 3.29 (d, 3H), 2.11 (s, 3H), 1.24 (d, 3H).

Example 27: [(7-((1-methoxypropan-2-yl)oxy)-4-(o-tolyl)-2H-chromen-2-one, Peak 2]: LCMS (ESI) Calcd. for C₂₀H₂₀O₄: 324, found [M+H]⁺=325. ¹H NMR (400 MHz, DMSO-d₆) δ 7.43-7.34 (m, 2H), 7.35 (t, 1H), 7.24 (d, 1H), 7.11 (s, 1H), 6.89-6.83 (m, 2H), 6.17 (s, 1H), 4.79 (d, 1H), 3.50 (t, 2H), 3.29 (d, 3H), 2.11 (s, 3H), 1.24 (d, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 0.1% isopropylamine in a mixture of 70% hexane, 15% of CH₂Cl₂ and 15% ethanol, held isocratic up to 25 min. with detection at 326 nm wavelength.

### Examples 28-29: 7-((tetrahydro-2H-pyran-4-yl)oxy)-4-(o-tolyl)-2H-chromen-2-one and 7-((tetrahydro-2H-pyran-4-yl)methoxy)-4-(o-tolyl)-2H-chromen-2-one

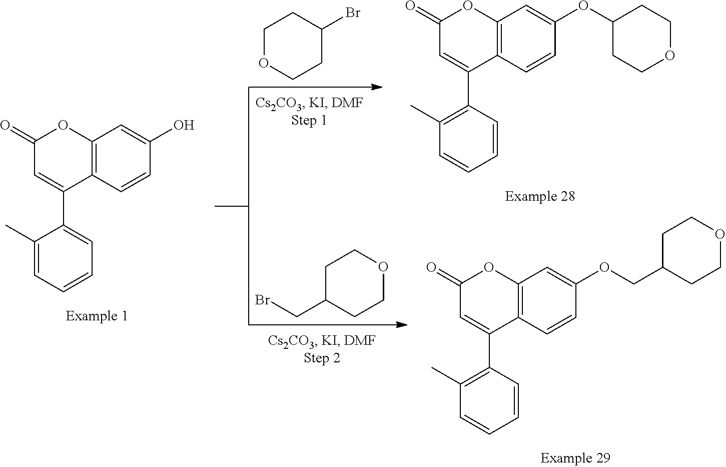 [SMILES: Cc1ccccc1-c1cc(=O)oc2cc(O)ccc12 ; BrC1CCOCC1 ; BrCC1CCOCC1 ; Cc1ccccc1-c1cc(=O)oc2cc(OC3CCOCC3)ccc12 ; Cc1ccccc1-c1cc(=O)oc2cc(OCC3CCOCC3)ccc12]

Synthesis of 7-((tetrahydro-2H-pyran-4-yl)oxy)-4-(o-tolyl)-2H-chromen-2-one, Example 28 [Step 1]: To a solution of 7-hydroxy-4-(o-tolyl)-2H-chromen-2-one (Example 1, 300 mg, 1.2 mmol) in DMF (7 mL) was added Cs₂CO₃ (780 mg, 2.4 mmol) and KI (200 mg, 1.2 mmol) followed by 4-bromotetrahydropyran (300 mg, 1.8 mmol). The resulting mixture was stirred at 80° C. for 48 h. The mixture was cooled, concentrated under reduced pressure, and partitioned between EtOAc and water. The organic layer was separated, washed with water (twice), brine, dried over anhydrous sodium sulfate, filtered, and concentrated under reduced pressure. The product was purified by RP prep-HPLC and lyophilized to afford 7-((tetrahydro-2H-pyran-4-yl)oxy)-4-(o-tolyl)-2H-chromen-2-one (Example 28, 130 mg). LCMS (ESI) Calcd. for C₂₁H₂₀O₄: 336, found [M+H]⁺=337. ¹H NMR (400 MHz, DMSO-d₆) δ 7.45-7.39 (m, 2H), 7.35 (t, 1H), 7.24 (d, 1H), 7.18 (d, 1H), 6.91-6.84 (m, 2H), 6.17 (s, 1H), 4.77-4.72 (m, 1H), 3.88-3.82 (m, 2H), 3.53-3.48 (m, 2H), 2.11 (S, 3H), 1.99 (d, 2H), 1.64-1.57 (m, 2H).

Synthesis of 7-((tetrahydro-2H-pyran-4-yl)methoxy)-4-(o-tolyl)-2H-chromen-2-one, Example 29 [Step 2]: To a stirred solution of 7-hydroxy-4-(o-tolyl)-2H-chromen-2-one (Example 1, 200 mg, 0.8 mmol) in DMF (7 mL) was added Cs₂CO₃ (780 mg, 2.4 mmol) and KI (200 mg, 1.2 mmol) followed by 4-(bromomethyl)tetrahydropyran (210 mg, 1.2 mmol). The resulting mixture was stirred at 80° C. for 48 h, cooled, and concentrated under reduced pressure. The product was partitioned between EtOAc and water and the organic layer was separated, washed with water (twice), brine, dried over anhydrous sodium sulfate, filtered, and concentrated under reduced pressure. The product was purified by RP prep-HPLC and lyophilized to afford 7-((tetrahydro-2H-pyran-4-yl)methoxy)-4-(o-tolyl)-2H-chromen-2-one (Example 29, 100 mg). LCMS (ESI) Calcd. for C₂₂H₂₂O₄: 350, found [M+H]⁺=351. ¹H NMR (400 MHz, DMSO-d₆) δ 7.45-7.33 (m, 3H), 7.24 (d, 1H), 7.10 (s, 1H), 6.87 (t, 2H), 6.18 (s, 1H), 4.02 (d, 2H), 3.96-3.85 (m, 2H), 3.35-3.28 (m, 2H), 2.11 (s, 3H), 2.07-1.94 (m, 1H), 1.66 (d, 2H), 1.38-1.31 (m, 2H).

### Examples 30-36: 7-propoxy-4-(2-(trifluoromethyl)phenyl)-2H-chromen-2-one, 4-(2-chloro-4-fluorophenyl)-7-propoxy-2H-chromen-2-one, 7-propoxy-4-(4-(trifluoromethyl)pyridin-3-yl)-2H-chromen-2-one, 4-(4-methylthiophen-3-yl)-7-propoxy-2H-chromen-2-one, 4-(4-hydroxy-2-methylphenyl)-7-propoxy-2H-chromen-2-one, 4-(2-methoxyphenyl)-7-propoxy-2H-chromen-2-one, and 4-(2,3-dihydrobenzo[b][1,4]dioxin-5-yl)-7-propoxy-2H-chromen-2-one

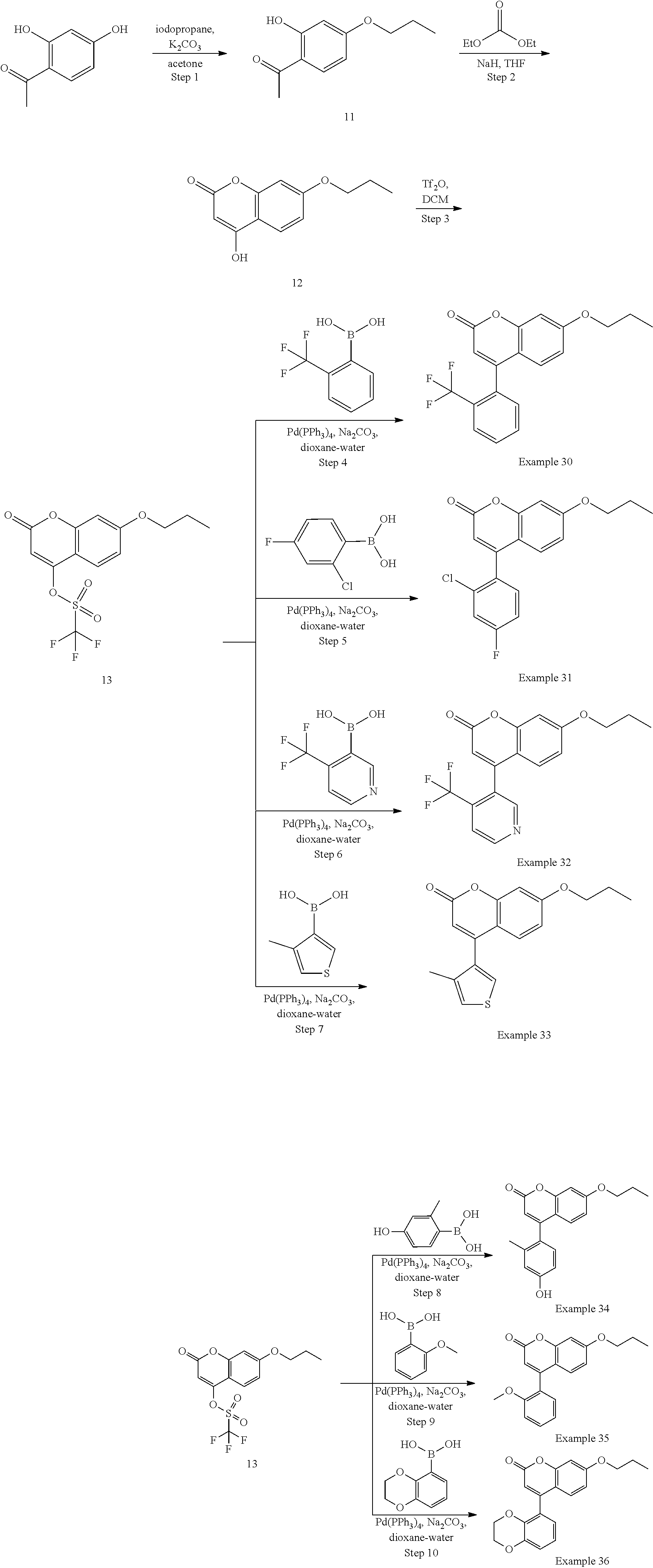

Synthesis of 1-(2-hydroxy-4-propoxyphenyl)ethan-1-one, 11 [Step 1]: To a solution of 1-(2,4-dihydroxyphenyl)ethan-1-one (8.0 g, 52.6 mmol) in acetone (520 mL) was added potassium carbonate (24 g, 174 mmol) followed by 1-iodopropane (5.1 mL, 52.6 mmol). The mixture was heated to 50° C. for 16 h., cooled, and concentrated under reduced pressure. The product was acidified using 2M aqueous HCl solution (pH=3) and extracted with ethyl acetate (twice). The combined organic extracts were dried over anhydrous sodium sulfate, filtered, and concentrated under reduced pressure. The product was purified by flash column chromatography on silica gel to afford 1-(2-hydroxy-4-propoxyphenyl)ethan-1-one (11, 7.5 g). LCMS (ESI) Calcd. for C₁₁H₁₄O₃: 194, found [M+H]⁺=195.

Synthesis of 4-hydroxy-7-propoxy-2H-chromen-2-one, 12 [Step 2]: To a stirred solution of 1-(2-hydroxy-4-propoxyphenyl)ethanone (11, 6 g, 30.9 mmol) in anhydrous toluene (90 mL) at 0° C. under an inert atmosphere was added diethyl carbonate (5.6 mL, 46.3 mmol), and sodium hydride (60% dispersion in mineral oil) (3.7 g, 154 mmol). The mixture was heated to 100° C. and stirred for 4 h, then cooled to 0° C. and quenched with saturated aqueous ammonium chloride solution. The mixture was washed with diethyl ether (twice), and acidified with 2M aqueous HCl solution (pH=3). The resulting precipitate was collected by filtration, washed with water, triturated with hexane, and dried to afford 4-hydroxy-7-propoxy-2H-chromen-2-one (12, 4.5 g). LCMS (ESI) Calcd. for C₁₂H₁₂O₄: 220, found [M+H]⁺=221.

Synthesis of 2-oxo-7-propoxy-2H-chromen-4-yl trifluoromethanesulfonate, 13 [Step 3]: To a stirred solution of 4-hydroxy-7-propoxy-2H-chromen-2-one (12, 2.0 g, 9.1 mmol) in CH₂Cl₂ (40 mL) was added triethylamine (1.6 mL, 11.8 mmol) followed by trifluoromethanesulfonic anhydride (2.1 mL, 11.8 mmol). The mixture stirred at 0° C. for 1 h., then diluted with CH₂Cl₂, washed with water (twice), brine, dried over anhydrous sodium sulfate, filtered, and concentrated under reduced pressure. The product was purified by flash column chromatography on silica gel to afford 2-oxo-7-propoxy-2H-chromen-4-yl trifluoromethanesulfonate (13, 2 g). LCMS (ESI) Calcd. for C₁₃H₁₁F₃O₆S: 352, found [M+H]⁺=353. ¹H NMR (400 MHz, DMSO-d₆): δ 7.60-7.57 (m, 1H), 7.17 (d, 1H), 7.11 (dd, 1H), 6.65 (s, 1H), 4.11-4.07 (m, 2H), 1.79-1.73 (m, 2H), 1.40-0.82 (m, 3H).

General procedure for Suzuki coupling: An oven-dried tube was charged with 2-oxo-7-propoxy-2H-chromen-4-yl trifluoromethanesulfonate (13, 1.0 eq) followed by aryl boronic acid or aryl boronate ester (1.5 eq) and sodium carbonate (2.0 eq). 1,4-Dioxane and water (4:1) were added and the mixture degassed with nitrogen for 10 min. by bubbling. Pd(PPh₃)₄ (0.1 eq) was then added and the tube was sealed and stirred at 80° C. for 1 h. The reaction mixture was then cooled to ambient temperature and partitioned between EtOAc and water. The organic layer was separated, washed with brine, dried over anhydrous sodium sulfate, filtered, and concentrated under reduced pressure. The product was purified by RP prep-HPLC and lyophilized to afford the desired product.

Synthesis of 7-propoxy-4-(2-(trifluoromethyl)phenyl)-2H-chromen-2-one, Example 30 [Step 4]: This compound was synthesized following general Suzuki coupling procedure from 2-oxo-7-propoxy-2H-chromen-4-yl trifluoromethanesulfonate (13, 200 mg, 0.6 mmol) and [2-(trifluoromethyl)phenyl]boronic acid (160 mg, 0.9 mmol).

Purification by RP prep-HPLC and lyophilization gave 7-propoxy-4-(2-(trifluoromethyl)phenyl)-2H-chromen-2-one (Example 30, 110 mg). LCMS (ESI) Calcd. for C₁₉H₁₅F₃O₃: 348, found [M+H]⁺=349. ¹H NMR (400 MHz, DMSO-d₆): δ 7.97-7.94 (d, 1H), 7.87-7.82 (t, 1H), 7.80-7.75 (t, 1H), 7.55-7.52 (d, 1H), 7.09-7.08 (m, 1H), 6.89-6.85 (dd, 1H), 6.79-6.76 (d, 1H), 6.26 (s, 1H), 4.06-4.02 (m, 2H), 1.80-1.70 (m, 2H), 1.00-0.96 (m, 3H).

Synthesis of 4-(2-chloro-4-fluorophenyl)-7-propoxy-2H-chromen-2-one, Example 31 [Step 5]: This compound was synthesized following general Suzuki coupling procedure from 2-oxo-7-propoxy-2H-chromen-4-yl trifluoromethanesulfonate (13, 200 mg, 0.6 mmol) and (2-chloro-4-fluoro-phenyl)boronic acid (150 mg, 0.9 mmol). Purification by RP prep-HPLC and lyophilization gave 4-(2-chloro-4-fluorophenyl)-7-propoxy-2H-chromen-2-one (Example 31, 69 mg). LCMS (ESI) Calcd. for C₁₈H₁₄ClFO₃: 332, found [M+H]⁺=333. ¹H NMR (400 MHz, DMSO-d₆): δ 7.70 (dd, 1H), 7.56 (dd, 1H), 7.43 (dt, 1H), 7.08 (d, 1H), 6.94-6.90 (m, 2H), 6.30 (s, 1H), 4.05 (t, 2H), 1.75 (sextet, 2H), 0.98 (t, 3H).

Synthesis of 7-propoxy-4-(4-(trifluoromethyl)pyridin-3-yl)-2H-chromen-2-one, Example 32 [Step 6]: This compound was synthesized following general Suzuki coupling procedure from 2-oxo-7-propoxy-2H-chromen-4-yl trifluoromethanesulfonate (13, 30 mg, 0.08 mmol) and (4-(trifluoromethyl)pyridin-3-yl)boronic acid (30 mg, 0.1 mmol). Purification by RP prep-HPLC and lyophilization gave 7-propoxy-4-(4-(trifluoromethyl)pyridin-3-yl)-2H-chromen-2-one (Example 32, 11 mg). LCMS (ESI) Calcd. for C₁₈H₁₄F₃NO₃: 349, found [M+H]⁺=350. ¹H NMR (400 MHz, DMSO-d₆): δ 9.03 (d, 1H), 8.80 (s, 1H), 8.00 (d, 1H), 7.11 (s, 1H), 6.87 (s, 2H), 6.42 (s, 1H), 4.06 (t, 2H), 1.74 (sextet, 2H), 0.98 (t, 3H).

Synthesis of 4-(4-methylthiophen-3-yl)-7-propoxy-2H-chromen-2-one, Example 33 [Step 7]: This compound was synthesized following general Suzuki coupling procedure from 2-oxo-7-propoxy-2H-chromen-4-yl trifluoromethanesulfonate (13, 250 mg, 0.7 mmol) and (4-methylthiophen-3-yl)boronic acid (151 mg, 1.06 mmol). Purification by RP prep-HPLC and lyophilization gave 4-(4-methylthiophen-3-yl)-7-propoxy-2H-chromen-2-one (Example 33, 77 mg). LCMS (ESI) Calcd. for C₁₇H₁₆O₃S: 300, found [M+H]⁺=301. ¹H NMR (400 MHz, DMSO-d₆): δ 7.69 (d, 1H), 7.43 (d, 1H), 7.13 (d, 1H), 7.06 (d, 1H), 6.92 (dd, 1H), 6.19 (s, 1H), 4.05 (t, 2H), 2.07 (s, 3H), 1.76 (sextet, 2H), 0.98 (t, 3H).

Synthesis of 4-(4-hydroxy-2-methylphenyl)-7-propoxy-2H-chromen-2-one, Example 34 [Step 8]: This compound was synthesized following general Suzuki coupling procedure from 2-oxo-7-propoxy-2H-chromen-4-yl trifluoromethanesulfonate (13, 200 mg, 0.6 mmol) and (4-hydroxy-2-methylphenyl)boronic acid (130 mg, 0.9 mmol). Purification by RP prep-HPLC and lyophilization gave 4-(4-hydroxy-2-methylphenyl)-7-propoxy-2H-chromen-2-one (Example 34, 69 mg). LCMS (ESI) Calcd. for C₁₉H₁₈O₄: 310, found [M+H]⁺=311. ¹H NMR (400 MHz, DMSO-d₆): δ 9.68 (s, 1H), 7.04-7.02 (m, 2H), 6.97-6.94 (d, 1H), 6.89-6.85 (dd, 1H), 6.77-6.71 (m, 2H), 6.10 (s, 1H), 4.06-4.02 (m, 2H), 2.03 (s, 3H), 1.80-1.70 (m, 2H), 1.00-0.96 (m, 3H).

Synthesis of 4-(2-methoxyphenyl)-7-propoxy-2H-chromen-2-one, Example 35 [Step 9]: This compound was synthesized following general Suzuki coupling procedure from 2-oxo-7-propoxy-2H-chromen-4-yl trifluoromethanesulfonate (13, 200 mg, 0.56 mmol) and (2-methoxyphenyl)boronic acid (130 mg, 0.9 mmol). Purification by RP prep-HPLC and lyophilization gave 4-(2-methoxyphenyl)-7-propoxy-2H-chromen-2-one (Example 35, 58 mg). LCMS (ESI) Calcd. for C₁₉H₁₉O₄: 310, found [M+H]⁺=311. ¹H NMR (400 MHz, DMSO-d₆): δ 7.53 (t, 1H), 7.30-7.28 (m, 1H), 7.22 (d, 1H), 7.11 (t, 1H), 7.03-6.98 (m, 2H), 6.88-6.85 (m, 1H), 6.17 (s, 1H), 4.04 (t, 2H), 3.72 (s, 3H), 1.75 (sextet, 2H), 0.98 (t, 3H).

Synthesis of 4-(2,3-dihydrobenzo[b][1,4]dioxin-5-yl)-7-propoxy-2H-chromen-2-one, Example 36 [Step 10]: This compound was synthesized following general Suzuki coupling procedure from 2-oxo-7-propoxy-2H-chromen-4-yl trifluoromethanesulfonate (13, 50 mg, 0.14 mmol) and (2,3-dihydrobenzo[b][1,4]dioxin-5-yl)boronic acid (38 mg, 0.2 mmol), Purification by RP prep-HPLC and lyophilization gave 4-(2,3-dihydrobenzo[b][1,4]dioxin-5-yl)-7-propoxy-2H-chromen-2-one (Example 36, 33 mg). LCMS (ESI) Calcd. for C₂₀H₁₈O₅: 338, found [M+H]⁺=339. ¹H NMR (400 MHz, DMSO-d₆): δ 7.17-7.13 (d, 1H), 7.05-7.02 (m, 2H), 7.00-6.94 (t, 1H), 6.90-6.84 (m, 2H), 6.20 (s, 1H), 4.29-4.27 (d, 2H), 4.21-4.18 (m, 2H), 4.06-4.02 (m, 2H), 1.80-1.70 (m, 2H), 1.00-0.96 (m, 3H).

### Example 37: 7-hydroxy-5-methyl-4-(o-tolyl)-2H-chromen-2-one

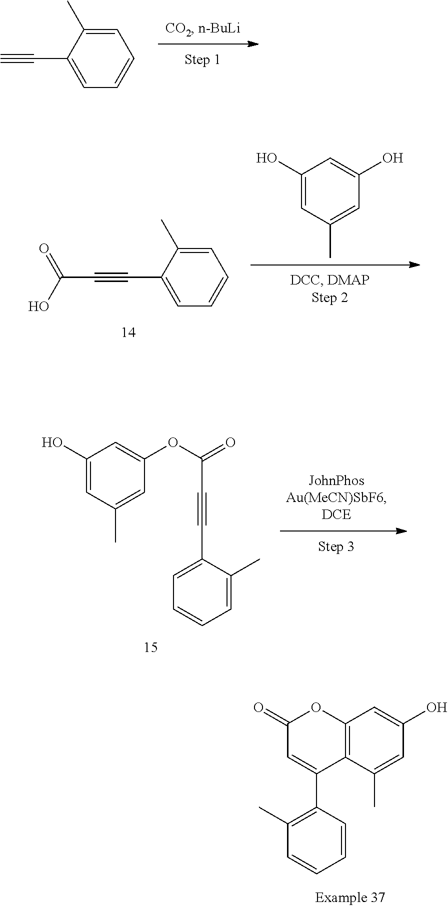

Synthesis of 3-(o-tolyl)propiolic acid, 14 [Step 1]: To a stirred solution of 1-ethynyl-2-methylbenzene (2.4 g, 21 mmol) in THF (25 mL) at −78° C. was added n-butyl lithium, 1.8M in THF (13 mL, 23 mmol) dropwise. Carbon dioxide gas was then bubbled through the reaction mixture for 1 h. at −78° C. The reaction mixture was then gradually warmed to ambient temperature and stirred for 16 h. The reaction was quenched with saturated aqueous NH₄Cl solution and extracted with EtOAc. The aqueous layer was separated, acidified with 1N aqueous HCl solution, and extracted with EtOAc (thrice). Combined organic extracts were washed with brine, dried over anhydrous sodium sulfate, filtered, and concentrated under reduced pressure to give 3-(o-tolyl)propiolic acid (14, 1.8 g). LCMS Calcd. for C₁₀H₈O₂: 160, found [M+H]⁺=161. ¹H NMR (400 MHz, DMSO-d₆) δ 7.54 (d, 1H), 7.41 (t, 1H), 7.35 (d, 1H), 7.26 (t, 1H), 2.41 (s, 3H).

Synthesis of 3-hydroxy-5-methylphenyl 3-(o-tolyl)propiolate, 15 [Step 2]: To a stirred solution of 5-methylbenzene-1,3-diol (330 mg, 2.6 mmol) and 3-(o-tolyl)prop-2-ynoic acid (15, 450 mg, 2.5 mmol) in CH₂Cl₂ (15 mL) was added a solution of DCC (826 mg, 4 mmol) in CH₂Cl₂ (10 mL) followed by DMAP (33 mg, 0.3 mmol). The resulting mixture was stirred at ambient temperature for 16 h., then filtered. The filtrate was washed with water, brine, dried over anhydrous sodium sulfate, filtered, and concentrated under reduced pressure. The product was purified by flash column chromatography on silica gel to afford 3-hydroxy-5-methylphenyl 3-(o-tolyl)propiolate (15, 230 mg). LCMS Calcd. for C₁₇H₁₄O₃: 266, found [M+H]⁺=267. ¹H NMR (400 MHz, DMSO-d₆) δ 9.70 (s, 1H), 7.63 (d, 1H), 7.49 (t, 1H), 7.40 (d, 1H), 7.31 (t, 1H), 6.53 (d, 2H), 6.45 (s, 1H), 2.40 (s, 3H), 2.23 (s, 3H).

Synthesis of 7-hydroxy-5-methyl-4-(o-tolyl)-2H-chromen-2-one, Example 37 [Step 3]: A solution of 3-hydroxy-5-methylphenyl 3-(o-tolyl)propiolate (15, 240 mg, 1 mmol) in DCE (15 mL) was degassed with nitrogen for 10 min. (Acetonitrile)[(2-biphenyl)di-tert-butylphosphine]gold(I) hexafluoroantimonate (70 mg, 0.1 mmol) was added and the mixture stirred at ambient temperature for 16 h. The mixture was filtered and the filtrate was concentrated under reduced pressure. Purification by flash column chromatography on silica gel gave 7-hydroxy-5-methyl-4-(o-tolyl)-2H-chromen-2-one (Example 37, 110 mg). LCMS Calcd. for C₁₇H₁₄O₃: 266, found [M+H]⁺=267. ¹H NMR (400 MHz, DMSO-d₆) δ 10.54 (s, 1H), 7.37-7.35 (m, 1H), 7.32-7.28 (m, 2H), 7.20 (d, 1H), 6.68 (d, 1H), 6.53 (d, 1H), 5.89 (s, 1H), 2.07 (s, 3H), 1.58 (s, 3H).

### Examples 38-40: 7-((tetrahydro-2H-pyran-2-yl)methoxy)-4-(o-tolyl)-2H-chromen-2-one, racemic and purified chiral analogs

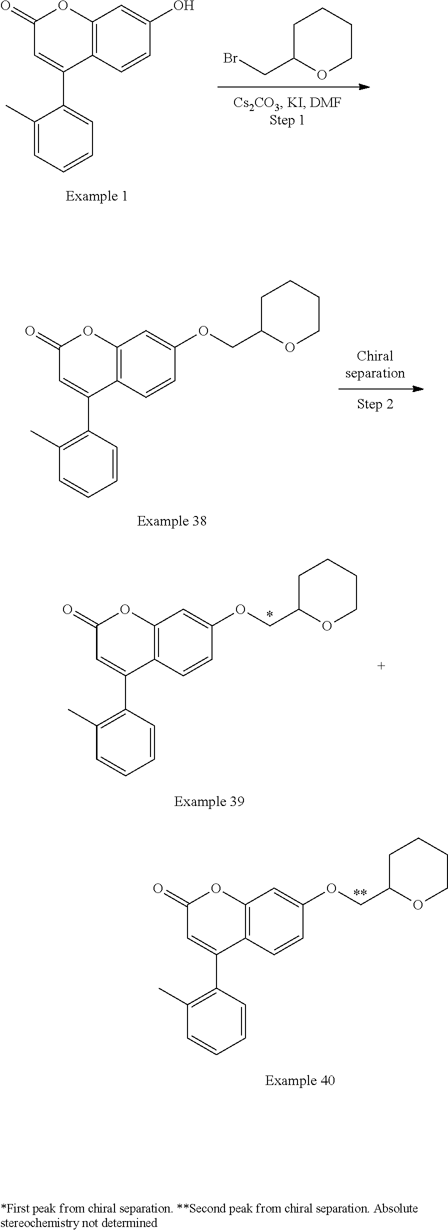 [SMILES: Cc1ccccc1-c1cc(=O)oc2cc(O)ccc12 ; BrCC1CCCCO1 ; Cc1ccccc1-c1cc(=O)oc2cc(OCC3CCCCO3)ccc12 ; Cc1ccccc1-c1cc(=O)oc2cc(OCC3CCCCO3)ccc12 ; Cc1ccccc1-c1cc(=O)oc2cc(OCC3CCCCO3)ccc12]

Synthesis of 7-((tetrahydro-2H-pyran-2-yl)methoxy)-4-(o-tolyl)-2H-chromen-2-one, Example 38 [Step 1]: To a solution of 7-hydroxy-4-(o-tolyl)-2H-chromen-2-one (Example 1, 200 mg, 0.8 mmol) in DMF (5 mL) was added Cs₂CO₃ (780 mg, 2.4 mmol) and KI (200 mg, 1.2 mmol) followed by 2-(bromomethyl)tetrahydropyran (210 mg, 1.2 mmol). The mixture stirred at 80° C. for 12 h, and then cooled, diluted with ethyl acetate, washed water (thrice), brine, dried over anhydrous sodium sulfate, filtered, and concentrated under reduced pressure. The product was purified by RP prep-HPLC and lyophilized to afford 7-((tetrahydro-2H-pyran-2-yl) methoxy)-4-(o-tolyl)-2H-chromen-2-one (Example 38, 100 mg). LCMS (ESI) Calcd. for C₂₂H₂₂O₄: 349, found [M+H]⁺=351. ¹H NMR (400 MHz, DMSO-d₆) δ 7.45-7.33 (m, 3H), 7.24 (d, 1H), 7.09 (d, 1H), 6.91-6.84 (m, 2H), 6.18 (s, 1H), 4.03 (d, 2H), 3.89 (d, 1H), 3.64 (t, 1H), 3.41-3.35 (m, 1H), 2.11 (s, 3H), 1.82 (d, 1H), 1.65 (d, 1H), 1.52-1.14 (m, 3H), 1.34 (t, 1H).

Synthesis of chiral 7-((tetrahydro-2H-pyran-2-yl)methoxy)-4-(o-tolyl)-2H-chromen-2-one, Examples 39 and 40 [Step 2]: 7-((tetrahydro-2H-pyran-2-yl) methoxy)-4-(o-tolyl)-2H-chromen-2-one (Example 38, 90 mg) was purified by chiral prep-HPLC and lyophilized to afford the first product as 7-((tetrahydro-2H-pyran-2-yl)methoxy)-4-(o-tolyl)-2H-chromen-2-one, Peak 1 (Example 39, 22 mg) and the second product as 7-((tetrahydro-2H-pyran-2-yl)methoxy)-4-(o-tolyl)-2H-chromen-2-one, Peak 2 (Example 40, 17 mg). The absolute stereochemistries of the enantiomers were not determined.

Example 39: 7-((tetrahydro-2H-pyran-2-yl)methoxy)-4-(o-tolyl)-2H-chromen-2-one, Peak 1: LCMS (ESI) Calcd. for C₂₂H₂₂O₄: 350, found [M+H]⁺=351. ¹H NMR (400 MHz, DMSO-d₆) δ 7.43-7.33 (m, 3H), 7.24 (d, 1H), 7.09 (d, 1H), 6.91-6.84 (m, 2H), 6.18 (s, 1H), 4.03 (d, 2H), 3.89 (d, 1H), 3.64 (t, 1H), 3.41-3.35 (m, 1H), 2.11 (s, 3H), 1.82 (d, 1H), 1.65 (d, 1H), 1.52-1.14 (m, 3H), 1.34 (t, 1H).

Example 40: 7-((tetrahydro-2H-pyran-2-yl)methoxy)-4-(o-tolyl)-2H-chromen-2-one, Peak 2: LCMS (ESI) Calcd. for C₂₂H₂₂O₄: 350, found [M+H]⁺=351. ¹H NMR (400 MHz, DMSO-d₆) δ 7.43-7.33 (m, 3H), 7.24 (d, 1H), 7.09 (d, 1H), 6.91-6.86 (m, 2H), 6.18 (s, 1H), 4.03 (d, 2H), 3.89 (d, 1H), 3.64 (t, 1H), 3.41-3.35 (m, 1H), 2.11 (s, 3H), 1.82 (d, 1H), 1.65 (d, 1H), 1.52-1.14 (m, 3H), 1.34 (t, 1H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 0.1% isopropylamine in a mixture of 60% hexane, 20% of CH₂Cl₂ and 20% ethanol, held isocratic up to 30 min. with detection at 326 nm wavelength.

### Examples 41-43: 2-((2-oxo-4-(o-tolyl)-2H-chromen-7-yl)oxy)propanenitrile, racemic and purified chiral analogs

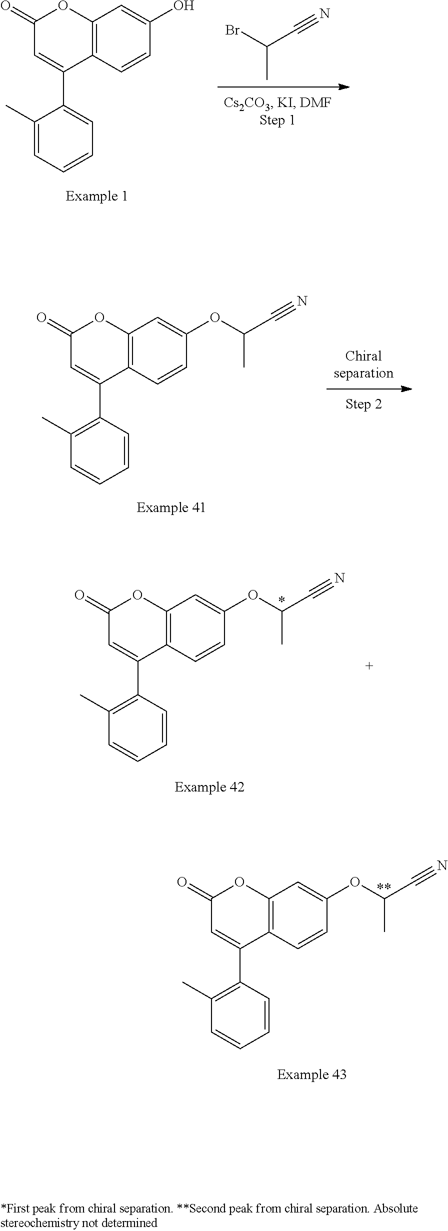

Synthesis of 2-((2-oxo-4-(o-tolyl)-2H-chromen-7-yl)oxy)propanenitrile, Example 41 [Step 1]: To a solution of 7-hydroxy-4-(o-tolyl)-2H-chromen-2-one (Example 1, 400 mg, 1.6 mmol) in DMF (8 mL) was added Cs₂CO₃ (1550 mg, 4.8 mmol) and KI (390 mg, 2.4 mmol) followed by 2-bromopropanenitrile (320 mg, 2.4 mmol). The mixture was stirred at 80° C. for 48 h., then cooled and diluted with ethyl acetate. The mixture was washed water (thrice), brine, dried over anhydrous sodium sulfate, filtered, and concentrated under reduced pressure. The product was purified by RP prep-HPLC and lyophilized to afford 2-((2-oxo-4-(o-tolyl)-2H-chromen-7-yl)oxy)propanenitrile (Example 41, 100 mg). LCMS (ESI) Calcd. for C₁₉H₁₅NO₃: 305, found [M+H]⁺=306. ¹H NMR (400 MHz, DMSO-d₆) δ 7.46-7.34 (m, 3H), 7.31 (s, 1H), 7.26 (d, 1H), 7.02-6.94 (m, 2H), 6.28 (s, 1H), 5.63 (q, 1H), 2.12 (s, 3H), 1.73 (d, 3H).

Synthesis of chiral 2-((2-oxo-4-(o-tolyl)-2H-chromen-7-yl)oxy)propanenitrile, Examples 42 and 43 [Step 2]: 2-((2-oxo-4-(o-tolyl)-2H-chromen-7-yl)oxy)propanenitrile (Example 41, 70 mg) was purified by chiral prep-HPLC and lyophilized to afford the first product as 2-((2-oxo-4-(o-tolyl)-2H-chromen-7-yl)oxy)propanenitrile, Peak 1 (Example 42, 35 mg) and the second product as 2-((2-oxo-4-(o-tolyl)-2H-chromen-7-yl)oxy)propanenitrile, Peak 2 (Example 43, 30 mg). The absolute stereochemistries of the enantiomers were not determined.

Example 42: 2-((2-oxo-4-(o-tolyl)-2H-chromen-7-yl)oxy)propanenitrile, Peak 1: LCMS (ESI) Calcd. for C₁₉H₁₅NO₃: 305, found [M+H]⁺=306. ¹H NMR (400 MHz, DMSO-d₆) δ 7.46-7.40 (m, 2H), 7.36 (t, 1H) 7.31-7.26 (m, 2H), 7.02-6.94 (m, 2H), 6.28 (s, 1H), 5.63 (q, 1H), 2.12 (s, 3H), 1.73 (d, 3H).

Example 43: 2-((2-oxo-4-(o-tolyl)-2H-chromen-7-yl)oxy)propanenitrile, Peak 2: LCMS (ESI) Calcd. for C₁₉H₁₅NO₃: 305, found [M+H]⁺=306. ¹H NMR (400 MHz, DMSO-d₆) δ 7.43-7.37 (m, 2H), 7.33 (t, 1H) 7.28-7.22 (m, 2H), 7.00-6.92 (m, 2H), 6.25 (s, 1H), 5.60 (q, 1H), 2.10 (s, 3H), 1.71 (d, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 0.1% isopropylamine in a mixture of 90% hexane, 10% of CH₂Cl₂ and 20% ethanol, held isocratic up to 30 min. with detection at 210 nm wavelength.

### Examples 44-46: 7-((1-methoxybutan-2-yl)oxy)-4-(o-tolyl)-2H-chromen-2-one, racemic and purified chiral analogs

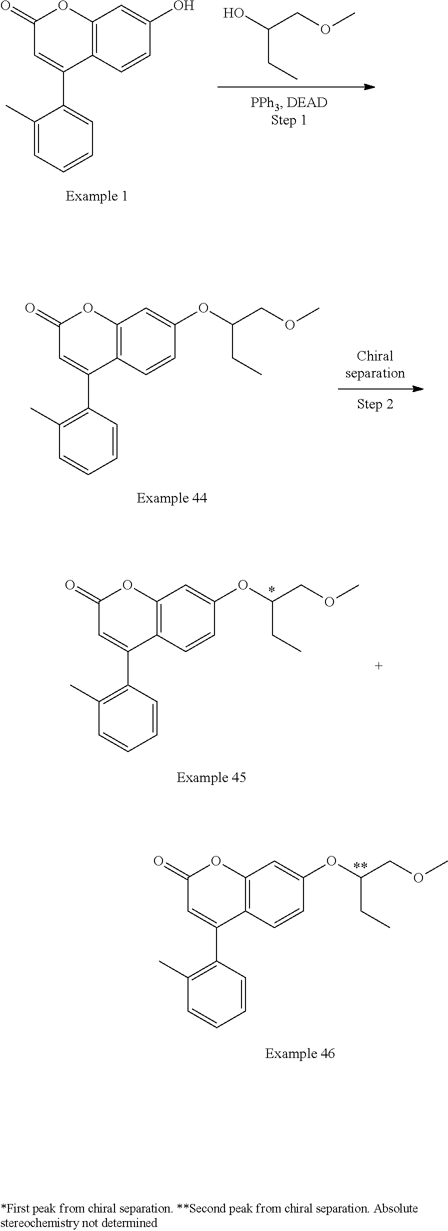

Synthesis of 7-((1-methoxybutan-2-yl)oxy)-4-(o-tolyl)-2H-chromen-2-one, Example 44 [Step 1]: To a solution of 7-hydroxy-4-(o-tolyl)-2H-chromen-2-one (Example 1, 300 mg, 1.2 mmol) in dry THF (7 mL) at 0° C. under inert atmosphere was added 1-methoxybutan-2-ol (140 mg, 1.3 mmol) and PPh₃ (340 mg, 1.3 mmol) followed by 4 Å molecular sieves (500 mg). DEAD (diethyl azodicarboxylate) (0.2 mL, 1.3 mmol) was then added dropwise and the mixture stirred for 30 min. The mixture was gradually warmed to 50° C. and stirred for 12 h. The reaction mixture was diluted with ethyl acetate, washed water, brine, dried over anhydrous sodium sulfate, filtered, and concentrated under reduced pressure. The product was purified by RP prep-HPLC and lyophilized to afford 7-((1-methoxybutan-2-yl)oxy)-4-(o-tolyl)-2H-chromen-2-one (Example 44, 140 mg). LCMS (ESI) Calcd. for C₂₁H₂₂O₄: 337, found [M+H]⁺=339. ¹H NMR (400 MHz, DMSO-d₆) δ 7.43-7.33 (m, 3H), 7.24 (d, 1H), 7.12 (d, 1H), 6.90-6.83 (m, 2H), 6.17 (s, 1H), 4.61 (t, 1H), 3.51 (t, 2H), 3.28 (d, 3H), 2.12 (s, 3H), 1.68-1.63 (m, 2H), 0.91 (t, 3H).

Synthesis of chiral 7-((1-methoxybutan-2-yl)oxy)-4-(o-tolyl)-2H-chromen-2-one, Examples 45 and 46 [Step 2]: 7-((1-methoxybutan-2-yl)oxy)-4-(o-tolyl)-2H-chromen-2-one (Example 44, 90 mg) was purified by chiral prep-HPLC and lyophilized to afford the first product as 7-((1-methoxybutan-2-yl)oxy)-4-(o-tolyl)-2H-chromen-2-one, Peak 1 (Examples 45, 32 mg) and the second product as 7-((1-methoxybutan-2-yl)oxy)-4-(o-tolyl)-2H-chromen-2-one, Peak 2 (Examples 46, 28 mg). The absolute stereochemistries of the enantiomers were not determined.

Example 45: 7-((1-methoxybutan-2-yl)oxy)-4-(o-tolyl)-2H-chromen-2-one, Peak 1: LCMS (ESI) Calcd. for C₂₁H₂₂O₄: 338, found [M+H]⁺=339. ¹H NMR (400 MHz, DMSO-d₆) δ 7.43-7.39 (m, 2H), 7.35 (t, 1H), 7.24 (d, 1H), 7.12 (d, 1H), 6.90-6.83 (m, 2H), 6.17 (s, 1H), 4.61 (t, 1H), 3.51 (t, 2H), 3.28 (d, 3H), 2.12 (s, 3H), 1.68-1.60 (m, 2H), 0.91 (t, 3H).

Example 46: 7-((1-methoxybutan-2-yl)oxy)-4-(o-tolyl)-2H-chromen-2-one, Peak 2: LCMS (ESI) Calcd. for C₂₁H₂₂O₄: 338, found [M+H]⁺=339. ¹H NMR (400 MHz, DMSO-d₆) δ 7.43-7.33 (m, 3H), 7.24 (d, 1H), 7.12 (d, 1H), 6.90-6.83 (m, 2H), 6.17 (s, 1H), 4.61 (t, 1H), 3.51 (t, 2H), 3.28 (d, 3H), 2.12 (s, 3H), 1.68-1.60 (m, 2H), 0.91 (t, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 95% Hexane, 2.5% Ethanol and 2.5% THF, held isocratic up to 30 min. with detection at 325 nm wavelength.

### Examples 47-49: 7-((tetrahydro-2H-pyran-3-yl)oxy)-4-(o-tolyl)-2H-chromen-2-one, racemic and purified chiral analogs

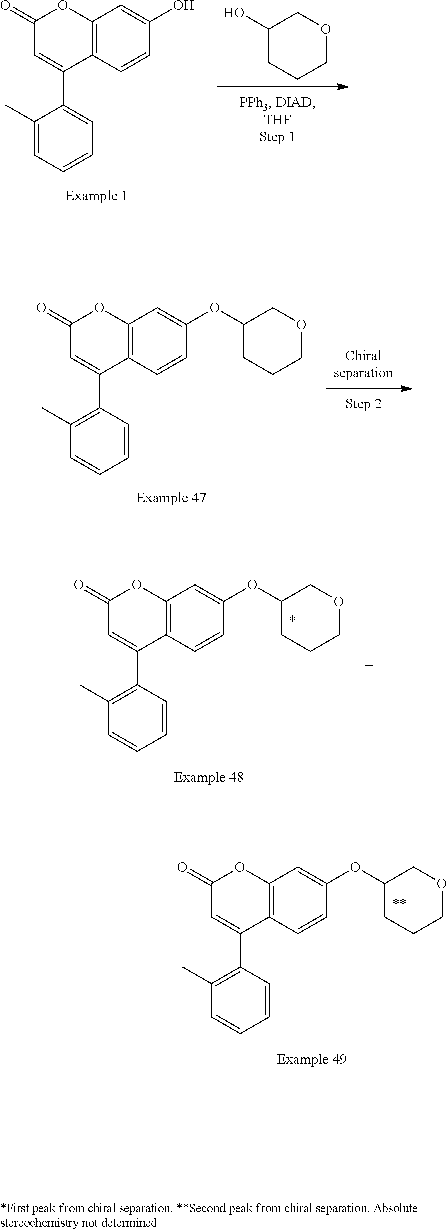 [SMILES: Cc1ccccc1-c1cc(=O)oc2cc(O)ccc12 ; OC1CCCOC1 ; Cc1ccccc1-c1cc(=O)oc2cc(OC3CCCOC3)ccc12 ; Cc1ccccc1-c1cc(=O)oc2cc(OC3CCCOC3)ccc12 ; Cc1ccccc1-c1cc(=O)oc2cc(OC3CCCOC3)ccc12]

Synthesis of 7-((tetrahydro-2H-pyran-3-yl)oxy)-4-(o-tolyl)-2H-chromen-2-one, Example 47 [Step 1]: To a solution 7-hydroxy-4-(o-tolyl)-2H-chromen-2-one (Example 1, 500 mg, 2.0 mmol) in dry THF (10 mL) at 0° C. under inert atmosphere was added tetrahydropyran-3-ol (300 mg, 3.0 mmol) and PPh₃ (780 mg, 3.0 mmol) followed by 4 Å molecular sieves (1 g). Diethyl azodicarboxylate (0.5 mL, 3.0 mmol) was added and the mixture stirred at 50° C. for 12 h. The mixture was then diluted with ethyl acetate, washed with water, brine, dried over anhydrous sodium sulfate, filtered, and concentrated under reduced pressure. The product was purified by RP prep-HPLC and lyophilized to afford 7-((tetrahydro-2H-pyran-3-yl)oxy)-4-(o-tolyl)-2H-chromen-2-one (Example 47, 120 mg). LCMS (ESI) Calcd. for C₂₁H₂₀O₄: 336, found [M+H]⁺=337. ¹H NMR (400 MHz, DMSO-d₆) δ 7.45-7.39 (m, 2H), 7.-7.33 (m, 1H), 7.24 (d, 1H), 7.14 (d, 1H), 6.91-6.84 (m, 2H), 6.18 (s, 1H), 4.58-4.55 (m, 1H), 3.81-3.78 (m, 1H), 3.64-3.52 (m, 3H), 2.11 (s, 3H), 2.05-2.00 (m, 1H), 1.80-1.70 (m, 2H), 1.57-1.51 (m, 1H).

Synthesis of chiral 7-((tetrahydro-2H-pyran-3-yl)oxy)-4-(o-tolyl)-2H-chromen-2-one, Example 48 and Example 49 [Step 2]: 7-((tetrahydro-2H-pyran-3-yl)oxy)-4-(o-tolyl)-2H-chromen-2-one (Example 47, 120 mg) was purified by chiral-HPLC and lyophilized to afford the first product as 7-((tetrahydro-2H-pyran-3-yl)oxy)-4-(o-tolyl)-2H-chromen-2-one, Peak 1 (Example 48, 55 mg) and the second product as 7-((tetrahydro-2H-pyran-3-yl)oxy)-4-(o-tolyl)-2H-chromen-2-one, Peak 2 (Example 49, 50 mg). The absolute stereochemistries of the enantiomers were not determined.

Example 48: 7-((tetrahydro-2H-pyran-3-yl)oxy)-4-(o-tolyl)-2H-chromen-2-one, Peak 1: LCMS (ESI) Calcd. for C₂₁H₂₀O₄: 336, found [M+H]⁺=337. ¹H NMR (400 MHz, DMSO-d₆) δ 7.45-7.33 (m, 3H), 7.24 (d, 1H), 7.14 (d, 1H), 6.91-6.85 (m, 2H), 6.18 (s, 1H), 4.58-4.55 (m, 1H), 3.81-3.78 (m, 1H), 3.64-3.52 (m, 3H), 2.11 (s, 3H), 2.07-2.01 (m, 1H), 1.80-1.71 (m, 2H), 1.57-1.51 (m, 1H).

Example 49: 7-((tetrahydro-2H-pyran-3-yl)oxy)-4-(o-tolyl)-2H-chromen-2-one, Peak 2: LCMS (ESI) Calcd. for C₂₁H₂₀O₄: 336, found [M+H]⁺=337. ¹H NMR (400 MHz, DMSO-d₆) δ 7.45-7.33 (m, 3H), 7.24 (d, 1H), 7.14 (d, 1H), 6.91-6.84 (m, 2H), 6.18 (s, 1H), 4.59-4.55 (m, 1H), 3.82-3.78 (m, 1H), 3.64-3.52 (m, 3H), 2.11 (s, 3H), 2.07-2.00 (m, 1H), 1.80-1.71 (m, 2H), 1.57-1.54 (m, 1H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 0.1% isopropylamine in a mixture of 70% hexane, 15% of CH₂Cl₂ and 15% ethanol, held isocratic up to 25 min. with detection at 326 nm wavelength.

### Example 50: 7-hydroxy-4-(2-methoxyphenyl)-2H-chromen-2-one

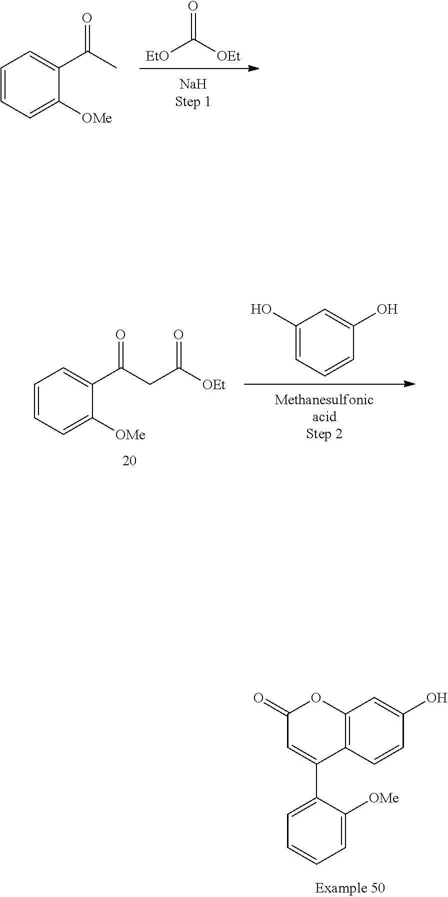 [SMILES: COc1ccccc1C(C)=O ; CCOC(=O)OCC ; CCOC(=O)CC(=O)c1ccccc1OC ; Oc1cccc(O)c1 ; COc1ccccc1-c1cc(=O)oc2cc(O)ccc12]

Synthesis of ethyl 3-(2-methoxyphenyl)-3-oxopropanoate, 20 [Step 1]: To a suspension of NaH (60% dispersion in mineral oil) (2.9 g, 120 mmol) in toluene (40 mL) was added diethyl carbonate (15.8 g, 130 mmol) at ambient temperature. The mixture was stirred for 15 min., then 1-(2-methoxyphenyl)ethanone (5.0 g, 34.0 mmol) was added. The mixture was gradually warmed to 100° C. and stirred at 100° C. for 4 h. The reaction mixture was cooled to 0° C. and quenched with a saturated aqueous NH₄Cl solution. The mixture was extracted with EtOAc (2×100 mL). The combined organic extracts were washed with water, brine, dried over anhydrous sodium sulfate, filtered, and concentrated under reduced pressure. The product was purified by flash column chromatography on silica gel to give ethyl 3-(2-methoxyphenyl)-3-oxopropanoate (20, 4.0 g). LCMS (ESI) Calcd. for C₁₂H₁₄O₄: 222, found [M+H]⁺=223. ¹H NMR (400 MHz, CDCl₃) δ 7.87-7.85 (m, 1H), 7.51-7.47 (m, 1H), 7.02 (t, 1H), 6.96-6.86 (m, 1H), 4.24-4.14 (m, 2H), 3.95 (s, 2H), 3.88 (d, 3H), 1.22 (d, 3H).

Synthesis of 7-hydroxy-4-(2-methoxyphenyl)-2H-chromen-2-one, Example 50 [Step 2]: Methanesulphonic acid (8.0 mL, 120.0 mmol) was added dropwise to a mixture of ethyl 3-oxo-3-(2-methoxyphenyl)-3-oxo-propanoate (20, 4.0 g, 18.0 mmol) and benzene-1,3-diol (2.0 g, 18.0 mmol) at ambient temperature. The reaction mixture was stirred for 16 h. and then quenched with ice water (40 mL). The resulting precipitate was filtered, washed with water (2×40 mL), then pentane. The solid was dried under reduced pressure to give 7-hydroxy-4-(2-methoxyphenyl)-2H-chromen-2-one (Example 50, 4 g). LCMS (ESI) Calcd. for C₁₆H₁₂O₄: 268, found [M+H]⁺=269. ¹H NMR (400 MHz, DMSO-d₆) δ 10.56 (s, 1H), 7.53-7.49 (m, 1H), 7.27 (dd, 1H), 7.20 (d, 1H), 7.09 (t, 1H), 6.92 (d, 1H), 6.76 (t, 1H), 6.73-6.69 (m, 1H), 6.09, (s, 1H), 3.71 (s, 3H).

### Examples 51-55: 4-(4,5-dimethylthiophen-3-yl)-7-propoxy-2H-chromen-2-one, 4-(3,5-dimethylisoxazol-4-yl)-7-propoxy-2H-chromen-2-one, 4-(2-methylpyridin-3-yl)-7-propoxy-2H-chromen-2-one, 4-(3-methylpyridin-4-yl)-7-propoxy-2H-chromen-2-one, of 4-(6-chloro-4-methylpyridin-3-yl)-7-propoxy-2H-chromen-2-one

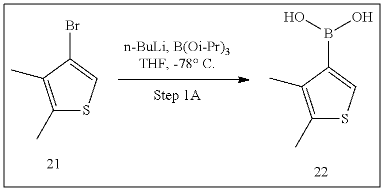

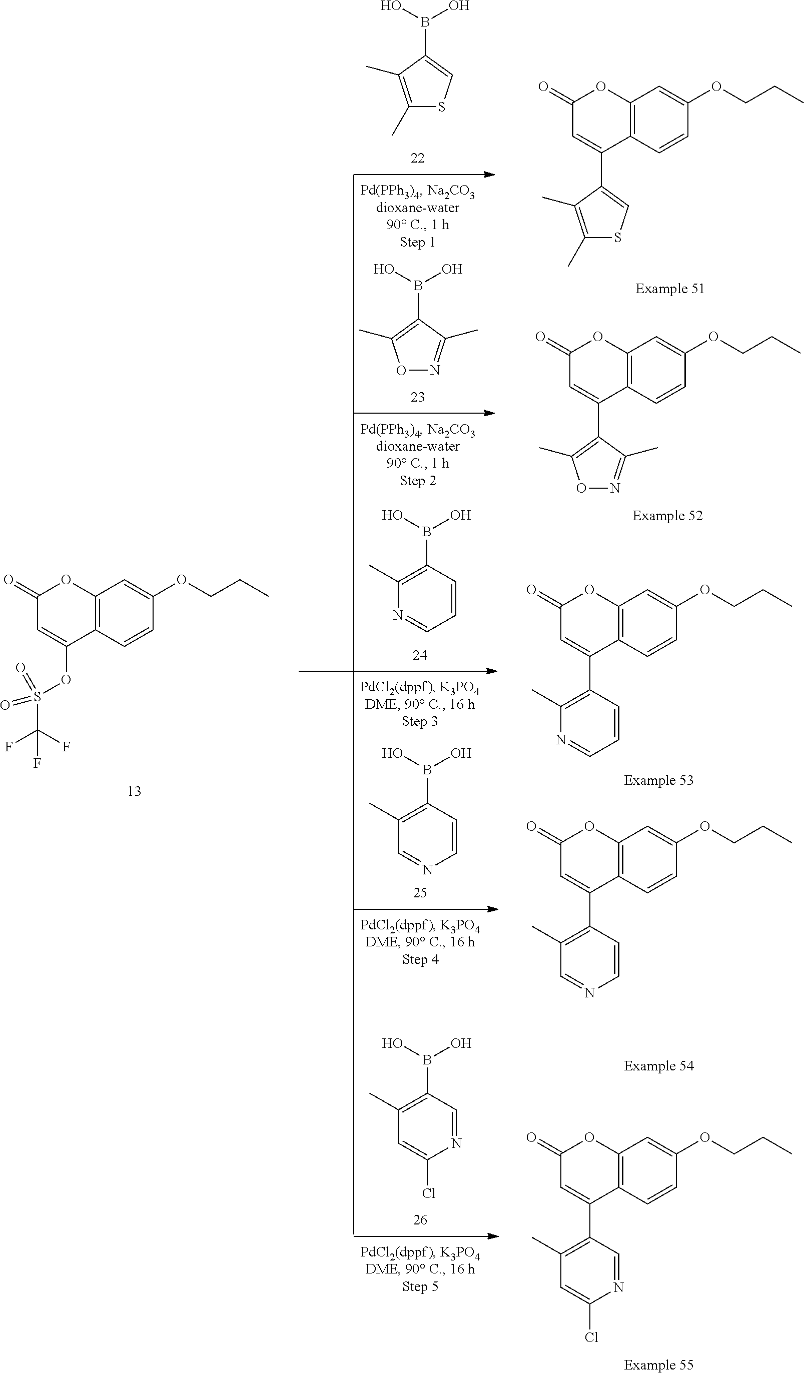

Synthesis of (4,5-dimethylthiophen-3-yl)boronic acid, 22 [Step 1A]: To an oven dried 2-neck round bottle flask was added 4-bromo-2,3-dimethylthiophene (21, 100 mg, 0.5 mmol) followed by triisopropyl borate (128 mg, 0.7 mmol) and THF (0.5 mL). The reaction mixture was treated with n-BuLi (1.6M in hexanes) (0.4 mL, 0.7 mmol) at −78° C. under an argon atmosphere and stirred for 1 h. at the same temperature. The reaction mixture was gradually warmed to room temperature and quenched by the slow addition of 2N aq. HCl. The mixture was diluted with EtOAc, washed with brine and the organic layer was separated, dried over anhydrous Na₂SO₄, and concentrated under reduced pressure. Product 22 was used without further purification.

Synthesis of 4-(4,5-dimethylthiophen-3-yl)-7-propoxy-2H-chromen-2-one, Example 51 [Step 1]: This compound was synthesized following general Suzuki coupling procedure from 2-oxo-7-propoxy-2H-chromen-4-yl trifluoromethanesulfonate (13, 100 mg) and (4,5-dimethylthiophen-3-yl)boronic acid (22, 66 mg). Purification by RP prep-HPLC and lyophilization gave 4-(4,5-dimethylthiophen-3-yl)-7-propoxy-2H-chromen-2-one (Example 51, 33 mg). LCMS (ESI) Calcd. for C₁₈H₁₈O₃S: 314, found [M+H]⁺=315. ¹H NMR (400 MHz, DMSO-d₆): δ 7.43 (s, 1H), 7.14 (d, 1H), 7.05 (d, 1H), 6.91 (dd, 1H), 6.14 (s, 1H), 4.05 (t, 2H), 2.39 (s, 3H), 1.92 (s, 3H), 1.80-1.70 (m, 2H), 0.98 (t, 3H).

Synthesis of 4-(3,5-dimethylisoxazol-4-yl)-7-propoxy-2H-chromen-2-one, Example 52 [Step 2]: This compound was synthesized following general Suzuki coupling procedure from 2-oxo-7-propoxy-2H-chromen-4-yl trifluoromethanesulfonate (13, 100 mg) and (3,5-dimethylisoxazol-4-yl)boronic acid (23, 52 mg). Purification by RP prep-HPLC and lyophilization gave (Example 52, 38 mg). LCMS (ESI) Calcd. for C₁₇H₁₇NO₄: 299, found [M+H]⁺=300. ¹H NMR (400 MHz, DMSO-d₆): δ 7.21 (d, 1H), 7.08-7.07 (m, 1H), 6.93-6.90 (m, 1H), 6.35 (s, 1H), 4.06 (t, 2H), 2.34 (s, 3H), 2.13 (s, 3H), 1.78-1.73 (m, 2H), 0.98 (t, 3H).

Synthesis of 4-(2-methylpyridin-3-yl)-7-propoxy-2H-chromen-2-one, Example 53 [Step 3]: This compound was synthesized following general Suzuki coupling procedure from 2-oxo-7-propoxy-2H-chromen-4-yl trifluoromethanesulfonate (13, 70 mg) and (2-methylpyridin-3-yl)boronic acid (24, 49 mg). Purification by RP prep-HPLC and lyophilization gave 4-(2-methylpyridin-3-yl)-7-propoxy-2H-chromen-2-one (Example 53, 39 mg). LCMS (ESI) Calcd. for C₁₈H₁₇NO₃: 295, found [M+H]⁺=296. ¹H NMR (400 MHz, DMSO-d₆): δ 8.62-8.61 (m, 1H), 7.71-7.69 (m, 1H), 7.42-7.38 (m, 1H), 7.10 (s, 1H), 6.88 (s, 2H), 6.29 (s, 1H), 4.05 (t, 2H), 2.31 (s, 3H), 1.78-1.73 (m, 2H), 0.98 (t, 3H).

Synthesis of 4-(3-methylpyridin-4-yl)-7-propoxy-2H-chromen-2-one, Example 54 [Step 4]: This compound was synthesized following general Suzuki coupling procedure from 2-oxo-7-propoxy-2H-chromen-4-yl trifluoromethanesulfonate (13, 30 mg) and (3-methylpyridin-4-yl)boronic acid (25, 21 mg). Purification by RP prep-HPLC and lyophilization gave 4-(3-methylpyridin-4-yl)-7-propoxy-2H-chromen-2-one, (Example 54, 15 mg). LCMS (ESI) Calcd. for C₁₈H₁₇NO₃: 295, found [M+H]⁺=296. ¹H NMR (400 MHz, DMSO-d₆): δ 8.63 (s, 1H), 8.57-8.55 (m, 1H), 7.31-7.30 (m, 1H), 7.10 (s, 1H), 6.87-6.85 (m, 2H), 6.27 (s, 1H), 4.07-4.03 (m, 2H), 2.12 (s, 3H), 1.79-1.73 (m, 2H), 0.98 (t, 3H).

Synthesis of 4-(6-chloro-4-methylpyridin-3-yl)-7-propoxy-2H-chromen-2-one, Example 55 [Step 5]: This compound was synthesized following general Suzuki coupling procedure from 2-oxo-7-propoxy-2H-chromen-4-yl trifluoromethanesulfonate (13, 100 mg) and 73 mg of (6-chloro-4-methylpyridin-3-yl)boronic acid (26, 73 mg). Purification by RP prep-HPLC and lyophilization gave 4-(6-chloro-4-methylpyridin-3-yl)-7-propoxy-2H-chromen-2-one (Example 55, 54 mg). LCMS (ESI) Calcd. for C₁₈H₁₆ClNO₃: 329, found [M+H]⁺=330. ¹H NMR (400 MHz, DMSO-d₆): δ 8.29 (s, 1H), 7.64 (s, 1H), 7.09 (d, 1H), 6.95 (d, 1H), 6.86 (dd, 1H), 6.33 (s, 1H), 4.05 (t, 2H), 2.15 (s, 3H), 1.79-1.70 (m, 2H), 0.98 (t, 3H).

### Examples 56-57: 7-((1-acetylpiperidin-4-yl)oxy)-4-(2-chloro-4-fluorophenyl)-2H-chromen-2-one, 7-((1-acetylpiperidin-3-yl)oxy)-4-(2-chloro-4-fluorophenyl)-2H-chromen-2-one

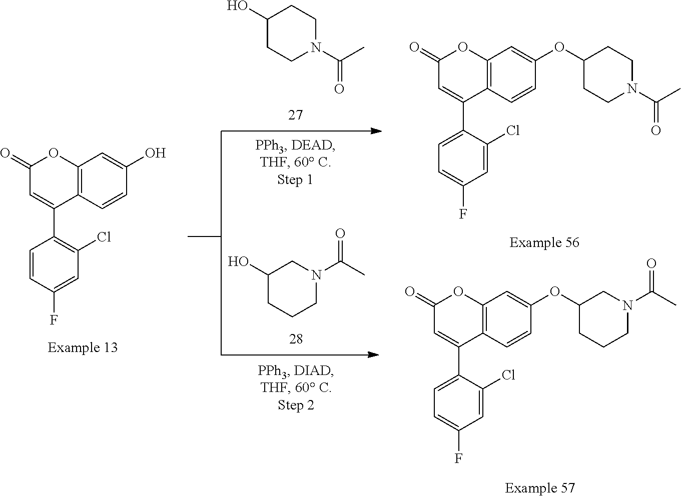

Synthesis of 7-((1-acetylpiperidin-4-yl)oxy)-4-(2-chloro-4-fluorophenyl)-2H-chromen-2-one, Example 56 [Step 1]: In a dried 2-neck 50 mL flask was added a solution of 4-(2-chloro-4-fluoro-phenyl)-7-hydroxy-chromen-2-one (Example 13, 200 mg, 0.7 mmol) in dry THF (7 mL) followed by 1-(4-hydroxy-1-piperidyl)ethanone (27, 150 mg, 1.0 mmol) and PPh₃ (270 mg, 1.0 mmol) along with 4 Å molecular sieves (400 mg) under an inert atmosphere. The reaction mixture was cooled to 0° C., and DEAD (0.2 mL, 1.0 mmol) was added dropwise with stirring for 30 min. The reaction mixture was gradually warmed to ambient temperature and stirring was continued for 12 h., at 60° C. The reaction mixture was diluted with ethyl acetate and the organic layer was washed with water (twice), brine (twice), dried over anhydrous Na₂SO₄, and concentrated under reduced pressure. The product was purified by RP prep-HPLC and lyophilization to afford 7-((1-acetylpiperidin-4-yl)oxy)-4-(2-chloro-4-fluorophenyl)-2H-chromen-2-one (Example 56, 84 mg). LCMS (ESI) Calcd. for C₂₂H₁₉ClFNO₄: 415, found [M+H]⁺=416. ¹H NMR (400 MHz, DMSO-d₆) δ 7.70 (dd, 1H), 7.58-7.55 (m, 1H), 7.46-7.41 (m, 1H), 7.21 (s, 1H), 6.95-6.90 (m, 2H), 6.30 (s, 1H), 4.82-4.76 (m, 1H), 3.89-3.85 (m, 1H), 3.70-3.65 (m, 1H), 3.37-3.35 (m, 1H), 3.25-3.19 (m, 1H), 2.01 (s, 4H), 1.89 (d, 1H), 1.63 (d, 1H), 1.52 (d, 1H).

Synthesis of 7-((1-acetylpiperidin-3-yl)oxy)-4-(2-chloro-4-fluorophenyl)-2H-chromen-2-one, Example 57 [Step 2]: To a solution of 4-(2-chloro-4-fluoro-phenyl)-7-hydroxy-chromen-2-one (Example 13, 150 mg, 0.5 mmol) in dry THF (7 mL) was added 1-(3-hydroxy-1-piperidyl)ethanone (28, 220 mg, 1.6 mmol) and PPh₃ (410 mg, 1.6 mmol) along with 4 Å molecular sieves (300 mg) under inert atmosphere. The solution was cooled to 0° C., and DIAD (270 mg, 1.6 mmol) was added dropwise and the reaction mixture was stirred for 30 min. The reaction mixture was gradually warmed to ambient temperature and continued stirring for 12 h. at 60° C. The reaction mixture was diluted with ethyl acetate and the organic layer was washed with water (twice), brine (twice), dried over anhydrous Na₂SO₄, and concentrated under reduced pressure. The product was purified by RP prep-HPLC and lyophilized to afford 7-((1-acetylpiperidin-3-yl)oxy)-4-(2-chloro-4-fluorophenyl)-2H-chromen-2-one (Example 57, 60 mg). LCMS (ESI) Calcd. for C₂₂H₁₉ClFNO₄: 415, found [M+H]⁺=416. ¹H NMR (400 MHz, DMSO-d₆) (at 100° C.) δ 7.61-7.52 (m, 2H), 7.41-7.37 (m, 1H), 7.01 (s, 1H), 6.96 (d, 1H), 6.90 (t, 1H), 6.24 (s, 1H), 4.61 (d, 1H), 3.52-3.46 (m, 4H), 1.97 (s, 4H), 1.78 (s, 2H), 1.53 (s, 1H).

### Examples 58-60: 7-isopropoxy-5-methyl-4-(o-tolyl)-2H-chromen-2-one, 7-((1-methoxypropan-2-yl)oxy)-5-methyl-4-(o-tolyl)-2H-chromen-2-one, racemic and purified chiral analogs

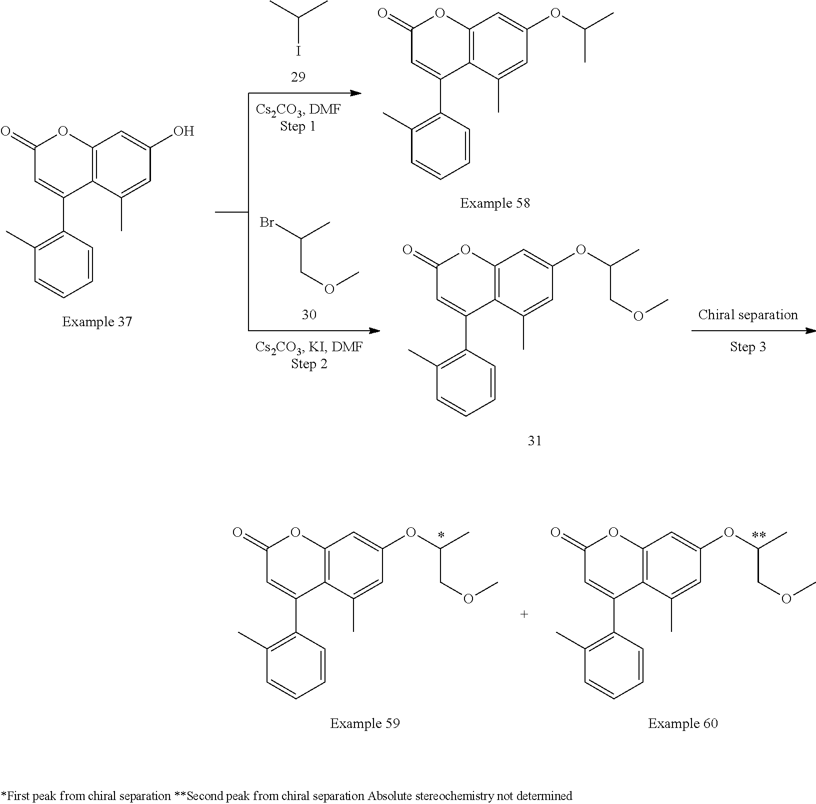

Synthesis of 7-isopropoxy-5-methyl-4-(o-tolyl)-2H-chromen-2-one, Example 58 [Step 1]: To a solution of 7-hydroxy-5-methyl-4-(o-tolyl)chromen-2-one (Example 37, 50 mg, 0.2 mmol) in DMF (5 mL) was added Cs₂CO₃ (120 mg, 0.4 mmol), followed by 2-iodopropane (29, 50 mg, 0.3 mmol). The reaction mixture was stirred 80° C. for 12 h., and then concentration under reduced pressure at ambient temperature to obtain the product, which was extracted with EtOAc. The organic layer was washed twice with cold water, brine, and dried over anhydrous Na₂SO₄. The organic layer was concentrated under reduced pressure and purified via RP prep-HPLC and lyophilized to afford 7-isopropoxy-5-methyl-4-(o-tolyl)-2H-chromen-2-one (Example 58, 29 mg). LCMS (ESI) Calcd. for C₂₀H₂₀O₃: 308, found [M+H]⁺=309. ¹H NMR (400 MHz, DMSO-d₆) δ 7.40-7.36 (m, 1H), 7.31 (t, 2H), 7.22 (d, 1H), 6.94 (d, 1H), 6.67 (d, 1H), 5.96 (s, 1H), 4.79-4.73 (m, 1H), 2.08 (s, 3H), 1.62 (s, 3H), 1.29 (d, 6H).

Synthesis of 7-((1-methoxypropan-2-yl)oxy)-5-methyl-4-(o-tolyl)-2H-chromen-2-one, 31 [Step 2]: To a solution of 7-hydroxy-5-methyl-4-(o-tolyl)chromen-2-one (Example 37, 100 mg, 0.4 mmol) in DMF (5 mL) was added cesium carbonate (490 mg, 1.5 mmol) and KI (130 mg, 0.8 mmol) followed by 2-bromo-1-methoxy-propane (30, 120 mg, 0.8 mmol). The reaction mixture was stirred at 80° C. for 12 h, concentrated under reduced pressure, and the product was extracted with ethyl acetate. The organic layer was washed twice with cold water, brine, dried over anhydrous Na₂SO₄, and filtered. The filtrate was concentrated under reduce pressure and purified by column chromatography to obtain 7-((1-methoxypropan-2-yl)oxy)-5-methyl-4-(o-tolyl)-2H-chromen-2-one (31, 110 mg). LCMS (ESI) Calcd. for C₂₁H₂₂O₄: 338, found [M+H]⁺=339. ¹H NMR (400 MHz, CDCl₃) δ 7.33 (t, 1H), 7.25 (t, 2H), 7.12 (d, 1H), 6.80 (d, 1H), 6.58 (s, 1H), 6.01 (s, 1H), 4.63-4.56 (m, 1H), 3.60-3.56 (m, 1H), 3.52-3.48 (m, 1H), 3.40 (s, 3H), 2.12 (d, 3H), 1.67 (s, 3H), 1.34 (d, 3H).

Synthesis of chiral 7-((1-methoxypropan-2-yl)oxy)-5-methyl-4-(o-tolyl)-2H-chromen-2-one, Examples 59 and 60 [Step 3]: 7-((1-methoxypropan-2-yl)oxy)-5-methyl-4-(o-tolyl)-2H-chromen-2-one (31, 110 mg) was purified by chiral prep-HPLC separation and the first product was isolated as 7-((1-methoxypropan-2-yl)oxy)-5-methyl-4-(o-tolyl)-2H-chromen-2-one, Peak 1 (Example 59, 15 mg) and the second product as 7-((1-methoxypropan-2-yl)oxy)-5-methyl-4-(o-tolyl)-2H-chromen-2-one, Peak 2 (Example 60, 30 mg). The absolute stereochemistries of these compounds were not determined.

Example 59: [7-((1-methoxypropan-2-yl)oxy)-5-methyl-4-(o-tolyl)-2H-chromen-2-one, Peak 1]: LCMS (ESI) Calcd. for C₂₁H₂₂O₄: 338, found [M+H]⁺=339. ¹H NMR (400 MHz, DMSO-d₆) δ 7.38 (t, 1H), 7.31 (t, 2H), 7.21 (d, 1H), 6.91 (d, 1H), 6.69 (s, 1H), 5.94 (s, 1H), 4.75 (q, 1H), 3.56-3.47 (m, 2H), 3.32 (s, 3H), 2.12 (s, 3H), 1.67 (s, 3H), 1.28 (d, 3H).

Example 60: [7-((1-methoxypropan-2-yl)oxy)-5-methyl-4-(o-tolyl)-2H-chromen-2-one, Peak 2]: LCMS (ESI) Calcd. for C₂₁H₂₂O₄: 338, found [M+H]⁺=339. ¹H NMR (400 MHz, DMSO-d₆) δ 7.38 (t, 1H), 7.31 (t, 2H), 7.21 (d, 1H), 6.91 (s, 1H), 6.69 (s, 1H), 5.94 (s, 1H), 4.75 (q, 1H), 3.56-3.47 (m, 2H), 3.32 (s, 3H), 2.12 (s, 3H), 1.67 (s, 3H), 1.28 (d, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 85% hexane and 15% of ethanol, held isocratic up to 40 min. with detection at 210 nm wavelength.

### Examples 61-62: (S)-7-((1-acetylpyrrolidin-3-yl)oxy)-4-(o-tolyl)-2H-chromen-2-one, (R)-7-((1-acetylpyrrolidin-3-yl)oxy)-4-(o-tolyl)-2H-chromen-2-one

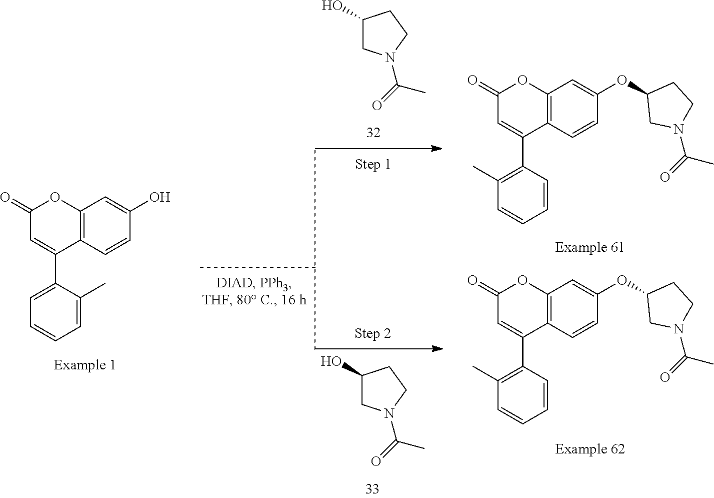

Synthesis of (S)-7-((1-acetylpyrrolidin-3-yl)oxy)-4-(o-tolyl)-2H-chromen-2-one, Example 61 [Step 1]: To a stirred solution of 7-hydroxy-4-(o-tolyl)-2H-chromen-2-one (Example 1, 200 mg, 0.8 mmol) and (R)-1-(hydroxypyrrolidin-1-yl)ethan-1-one (32, 155 mg, 1.2 mmol) in THF (3 mL) was added PPh₃ (625 mg, 2.4 mmol) followed by diisopropyl azodicarboxylate (0.5 mL, 2.4 mmol) and the reaction mixture was heated at 80° C. for 16 h. The reaction mixture was concentrated under reduced pressure to obtain the product, which was partitioned between EtOAc and water. The organic layer was washed with brine, dried over anhydrous Na₂SO₄, and concentrated under reduced pressure to obtain the product, which was purified by RP prep-HPLC chromatography and lyophilized to afford ethyl (S)-7-((1-acetylpyrrolidin-3-yl)oxy)-4-(o-tolyl)-2H-chromen-2-one as solid (Example 61, 159 mg). LCMS (ESI) Calcd. for C₂₂H₂₁NO₄: 363, found [M+H]⁺=364. ¹H NMR (400 MHz, DMSO-d₆) δ 7.43-7.33 (m, 3H), 7.23 (d, 1H), 7.15 (d, 1H), 6.89 (m, 2H), 6.19 (s, 1H), 5.23 (d, 1H), 3.86-3.52 (m, 4H), 2.33-2.11 (m, 5H), 1.97 (s, 3H).

Synthesis of (R)-7-((1-acetylpyrrolidin-3-yl)oxy)-4-(o-tolyl)-2H-chromen-2-one, Example 62 [Step 2]: To a stirred solution of 7-hydroxy-4-(o-tolyl)-2H-chromen-2-one (Example 1, 200 mg, 0.8 mmol) and (S)-1-(hydroxypyrrolidin-1-yl)ethan-1-one (33, 155 mg, 1.2 mmol) in THF (3 mL) was added PPh₃ (625 mg, 2.4 mmol) followed by diisopropyl azodicarboxylate (0.5 mL, 2.4 mmol), and the reaction mixture was heated at 80° C. for 16 h. The reaction mixture was concentrated under reduced pressure and the product was partitioned between EtOAc and water. The organic layer was washed with brine, dried over anhydrous Na₂SO₄, and concentrated under reduced pressure. The product was purified by RP prep-HPLC chromatography and lyophilized to afford ethyl (R)-7-((1-acetylpyrrolidin-3-yl)oxy)-4-(o-tolyl)-2H-chromen-2-one (Example 62, 145 mg). LCMS (ESI) Calcd. for C₂₂H₂₁NO₄: 363, found [M+H]⁺=364. ¹H NMR (400 MHz, DMSO-d₆) δ 7.43-7.38 (m, 3H), 7.23 (d, 1H), 7.15 (d, 1H), 6.89 (m, 2H), 6.19 (s, 1H), 5.23 (d, 1H), 3.86-3.52 (m, 4H), 2.33-2.11 (m, 5H), 1.97 (s, 3H).

### Example 63: (S)-7-((1-methylpyrrolidin-3-yl)oxy)-4-(o-tolyl)-2H-chromen-2-one

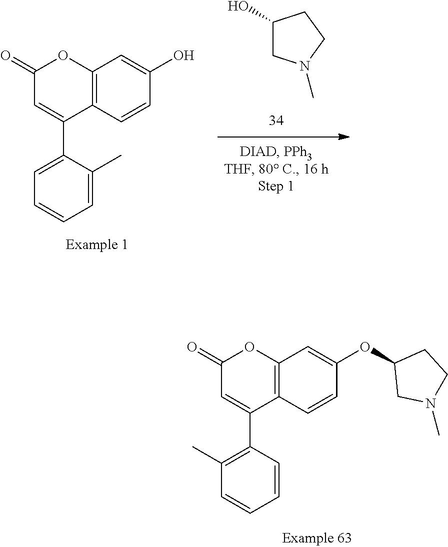

Synthesis of (S)-7-((1-methylpyrrolidin-3-yl)oxy)-4-(o-tolyl)-2H-chromen-2-one, Example 63 [Step 1]: To a stirred solution of 7-hydroxy-4-(o-tolyl)-2H-chromen-2-one (Example 1, 200 mg, 0.8 mmol) in THF (5 mL) was added (R)-1-methylpyrrolidin-3-ol (34, 120 mg, 1.2 mmol) followed by PPh₃ (624 mg, 2.4 mmol). The reaction mixture was stirred at ambient temperature for 15 min., followed by the addition of diisopropyl azodicarboxylate (0.4 mL, 2.4 mmol) at 0° C., and the reaction mixture was stirred at 80° C. for 16 h. The reaction mixture was concentrated under reduced pressure and the product was partitioned between EtOAc and water. The organic layer was washed with brine, dried over anhydrous Na₂SO₄, filtered, and concentrated under reduced pressure. The product was subjected to flash chromatography and then to RP prep-HPLC and lyophilized to afford (S)-7-((1-methylpyrrolidin-3-yl)oxy)-4-(o-tolyl)-2H-chromen-2-one (Example 63, 120 mg). LCMS (ESI) Calcd. for C₂₁H₂₁NO₃: 335, found [M+H]⁺=336. ¹H NMR (400 MHz, DMSO-d₆) δ 7.44-7.32 (m, 3H), 7.24 (d, 1H), 7.01 (d, 1H), 6.86-6.81 (m, 2H), 6.17 (s, 1H), 4.98 (br s, 1H), 2.78-2.74 (m, 1H), 2.69-2.62 (m, 2H), 2.36-2.32 (m, 2H), 2.25 (s, 3H), 2.11 (s, 3H), 1.83 (br s, 1H).

### Example 64: 4-(3-methylpyridin-2-yl)-7-propoxy-2H-chromen-2-one

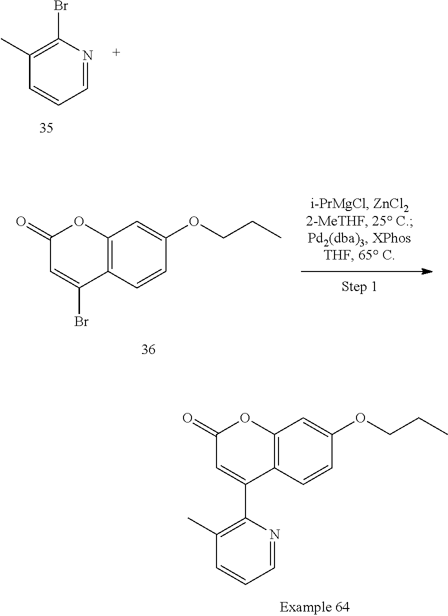

Synthesis of 4-(3-methylpyridin-2-yl)-7-propoxy-2H-chromen-2-one, Example 64 [Step 1]: To a mixture of THF (1.1 mL) and isopropylmagnesium chloride in 2M THF (0.6 mL, 1.3 mmol) under an argon atmosphere was added 2-bromo-3-methylpyridine (35, 200 mg, 1.2 mmol) dropwise at 25° C. After 4 h., zinc chloride solution (0.7 mL, 1.9M in 2-MeTHF, 1.4 mmol) was added dropwise, and the reaction mixture was stirred for another hour at the same temperature. The organozinc reagent was kept under argon, and used in the next step.

To a pressure tube was added Pd₂(dba)₃ (20 mg, 23.2 μmol) and XPhos (17 mg, 34.9 μmol) in THF (1.5 mL) under argon, and the reaction mixture was heated at 65° C. for 10 min. 4-Bromo-7-propoxy-2H-chromen-2-one (36, 200 mg, 0.7 mmol) was added and the reaction mixture was stirred for 15 min. at the same temperature. To the reaction mixture was added the previously prepared organozinc reagent and stirring was continued for 12 h. at 65° C. The reaction mixture was filtered and concentrated under reduced pressure, and the product was purified by RP prep-HPLC to afford 4-(3-methylpyridin-2-yl)-7-propoxy-2H-chromen-2-one (Example 64, 19 mg). LCMS (ESI) Calcd. for C₁₈H₁₇NO₃: 295, found [M+H]⁺=296. ¹H NMR (400 MHz, DMSO-d₆): δ 8.55 (d, 1H), 7.85 (d, 1H), 7.48-7.45 (m, 1H), 7.08 (d, 1H), 6.88-6.86 (m, 2H), 6.29 (s, 1H), 4.06-4.03 (m, 2H) 2.19 (s, 3H) 1.78-1.72 (m, 2H), 0.98 (t, 3H).

### Example 65: 3-methoxy-2-((2-oxo-4-(o-tolyl)-2H-chromen-7-yl)oxy)propanoic acid

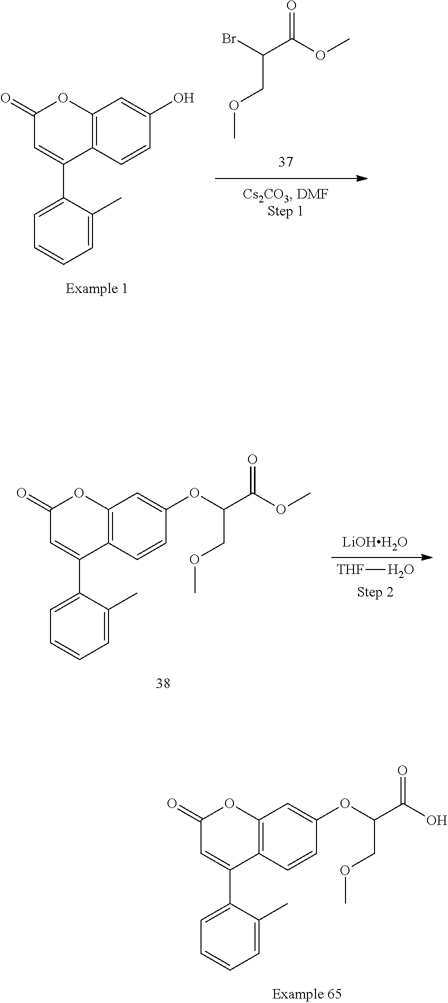

Synthesis of methyl 3-methoxy-2-((2-oxo-4-(o-tolyl)-2H-chromen-7-yl)oxy)propanoate, 38 [Step 1]: To a stirred solution of 7-hydroxy-4-(o-tolyl)chromen-2-one (Example 1, 1.0 g, 4 mmol) in DMF (5 mL) was added Cs₂CO₃ (2.6 g, 8 mmol). The reaction mixture was stirred at ambient temperature for 15 min., and methyl 2-bromo-3-methoxy-propanoate (37, 0.6 mL, 4 mmol) was added and the reaction mixture was stirred at ambient temperature for 18 h. The reaction mixture was diluted with water and extracted with EtOAc. The organic layer was washed with brine, dried over anhydrous Na₂SO₄, and concentrated under reduced pressure. The product was purified by flash column chromatography to obtain methyl 3-methoxy-2-((2-oxo-4-(o-tolyl)-2H-chromen-7-yl)oxy)propanoate (38, 1.3 g). LCMS (ESI) Calcd. for C₂₁H₂₀O₆: 368, found [M+H]=369.

Synthesis of 3-methoxy-2-((2-oxo-4-(o-tolyl)-2H-chromen-7-yl)oxy)propanoic acid, Example 65 [Step 2]: To a stirred solution of methyl 3-methoxy-2-[4-(o-tolyl)-2-oxo-chromen-7-yl]oxy-propanoate (38, 300 mg, 0.8 mmol) in THF (3 mL) and water (0.3 mL) was added at LiOH (60 mg, 2.5 mmol). The reaction mixture was stirred for 16 h. at ambient temperature. The reaction mixture was concentrated under reduced pressure, diluted with water, and extracted with EtOAc. The aqueous layer was acidified to pH 5-6 with aqueous HCl (10M), filtered, and the precipitated solids were collected to afford the product. The product was purified by RP prep-HPLC and lyophilized to yield 3-methoxy-2-((2-oxo-4-(o-tolyl)-2H-chromen-7-yl)oxy)propanoic acid (Example 65, 280 mg). LCMS (ESI) Calcd. for C₂₀H₁₈O₆: 354, found [M+H]⁺=355. ¹H NMR (400 MHz, DMSO-d₆) (at 100° C.) δ 7.44-7.33 (m, 3H), 7.23 (d, 1H), 6.91 (br s, 1H), 6.86-6.81 (m, 2H), 6.1 (s, 1H), 4.78 (s, 1H), 3.79-3.77 (m, 2H), 3.32 (s, 3H), 2.14 (s, 3H).

### Example 66: (R)-7-((1-methylpyrrolidin-3-yl)oxy)-4-(o-tolyl)-2H-chromen-2-one

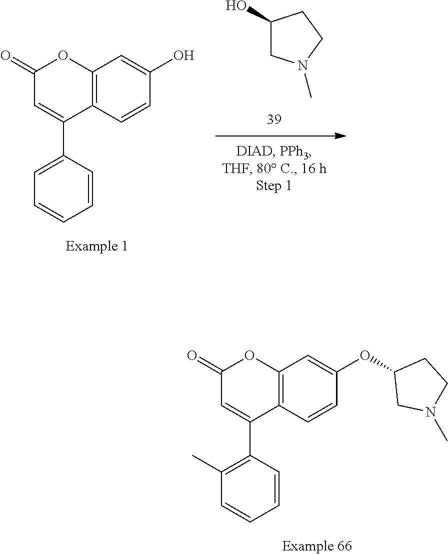

Synthesis of (R)-7-((1-methylpyrrolidin-3-yl)oxy)-4-(o-tolyl)-2H-chromen-2-one, Example 66 [Step 1]: To a stirred solution of 7-hydroxy-4-(o-tolyl)-2H-chromen-2-one (Example 1, 100 mg, 0.4 mmol) and (S)-1-methylpyrrolidin-3-ol (39, 60 mg, 0.6 mmol) in THF (3 mL) was added PPh₃ (310 mg, 1.2 mmol) followed by diisopropyl azodicarboxylate (0.2 mL, 1.2 mmol) at 0° C. The reaction mixture was stirred at 80° C. for 16 h. and then concentrated under reduced pressure. The product was partitioned between EtOAc and water, and the organic layer was washed with brine, dried over anhydrous Na₂SO₄, and concentrated under reduced pressure. The product was purified by RP prep-HPLC and lyophilized to afford (R)-7-((1-methylpyrrolidin-3-yl)oxy)-4-(o-tolyl)-2H-chromen-2-one (Example 66, 30 mg). LCMS (ESI) Calcd. for C₂₁H₂₁NO₃: 335, found [M+H]⁺=336. ¹H NMR (400 MHz, DMSO-d₆) δ 7.43-7.38 (m, 2H), 7.36-7.33 (m, 1H), 7.24 (d, 1H), 7.01 (d, 1H), 6.87-6.81 (m, 2H), 6.17 (s, 1H), 5.00-4.98 (m, 1H), 2.78-2.74 (m, 1H), 2.69-2.62 (m, 2H), 2.33-2.31 (m, 2H), 2.25 (s, 3H), 2.11 (s, 3H), 1.77 (d, 1H).

### Examples 67-68: 3-methoxy-2-((2-oxo-4-(o-tolyl)-2H-chromen-7-yl)oxy)propanamide, racemic and purified chiral analogs

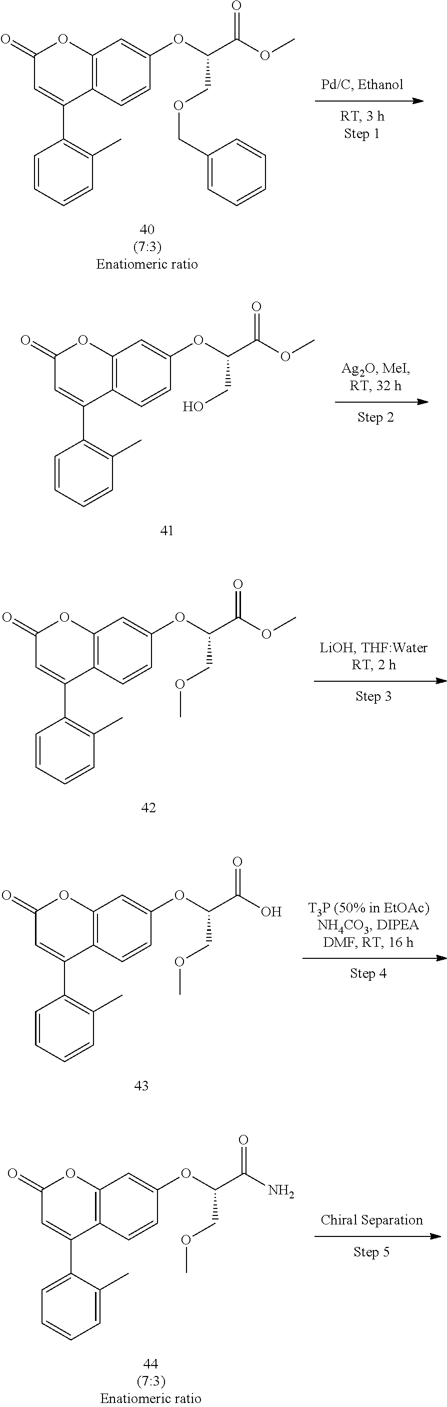 [SMILES: COC(=O)[C@H](COCc1ccccc1)Oc1ccc2c(-c3ccccc3C)cc(=O)oc2c1 ; COC(=O)[C@H](CO)Oc1ccc2c(-c3ccccc3C)cc(=O)oc2c1 ; COC[C@H](Oc1ccc2c(-c3ccccc3C)cc(=O)oc2c1)C(=O)OC ; COC[C@H](Oc1ccc2c(-c3ccccc3C)cc(=O)oc2c1)C(=O)O ; COC[C@H](Oc1ccc2c(-c3ccccc3C)cc(=O)oc2c1)C(N)=O]

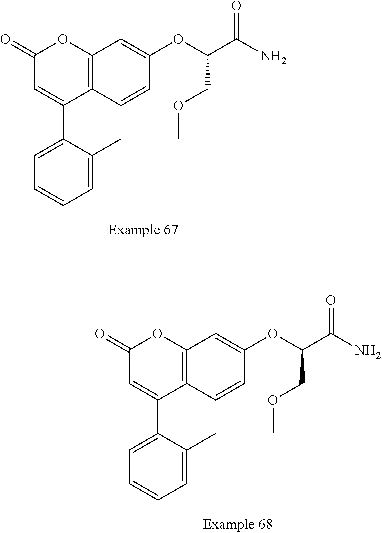 [SMILES: COC[C@H](Oc1ccc2c(-c3ccccc3C)cc(=O)oc2c1)C(N)=O ; COC[C@@H](Oc1ccc2c(-c3ccccc3C)cc(=O)oc2c1)C(N)=O]

Synthesis of methyl (S)-3-hydroxy-2-((2-oxo-4-(o-tolyl)-2H-chromen-7-yl)oxy)propanoate, 41 [Step 1]: A stirred solution of methyl (S)-3-(benzyloxy)-2-((2-oxo-4-(o-tolyl)-2H-chromen-7-yl)oxy)propanoate (40, 400 mg, 0.9 mmol) (7:3 (S:R) enantiomeric ratio) in ethanol (8 mL) was degassed with argon for 5 min. To the solution was added Pd/C (40 mg, 0.9 mmol) followed by hydrogen gas and stirring was continued at ambient temperature for 3 h. The reaction mixture was filtered through a celite bed using EtOH as eluent and concentrated under reduced pressure to afford methyl (S)-3-hydroxy-2-((2-oxo-4-(o-tolyl)-2H-chromen-7-yl)oxy)propanoate (41, 290 mg). LCMS (ESI) Calcd. for C₂₀H₁₈O₆: 354, found [M+H]⁺=355.

Synthesis of methyl (S)-3-methoxy-2-((2-oxo-4-(o-tolyl)-2H-chromen-7-yl)oxy)propanoate, 42 [Step 2]: To a stirred solution of methyl (S)-3-hydroxy-2-((2-oxo-4-(o-tolyl)-2H-chromen-7-yl)oxy)propanoate (41, 350 mg, 1.0 mmol) was added methyl iodide (1.8 mL, 30 mmol) and Ag₂O (460 mg, 2.0 mmol), and the reaction mixture was stirred at ambient temperature for 32 h. The reaction mixture was filtered through a celite bed and the filtrate was concentrated under reduced pressure to give the product that was purified by flash chromatography to afford methyl (S)-3-methoxy-2-((2-oxo-4-(o-tolyl)-2H-chromen-7-yl)oxy)propanoate (42, 210 mg). LCMS (ESI) Calcd. for C₂₁H₂₀O₆: 368, found [M+H]⁺=369.

Synthesis of (S)-3-methoxy-2-((2-oxo-4-(o-tolyl)-2H-chromen-7-yl)oxy)propanoic acid, 43 [Step 3]: To a stirred solution of methyl (S)-3-methoxy-2-((2-oxo-4-(o-tolyl)-2H-chromen-7-yl)oxy)propanoate (42, 210 mg, 0.6 mmol) in THF (4 mL) was added dropwise a solution of LiOH·H₂O (50 mg, 1.1 mmol) in water (1 mL) and the reaction mixture was stirred at ambient temperature for 2 h. The reaction mixture was concentrated under reduced pressure, diluted with water, acidified with citric acid, extracted with EtOAc, washed with brine, and dried over anhydrous Na₂SO₄. After filtration, the filtrate was concentrated under reduced pressure to afford the (S)-3-methoxy-2-((2-oxo-4-(o-tolyl)-2H-chromen-7-yl)oxy)propanoic acid (43, 200 mg). LCMS (ESI) Calcd. for C₂₀H₁₈O₆: 354, found [M+H]⁺=355.

Synthesis of (S)-3-methoxy-2-((2-oxo-4-(o-tolyl)-2H-chromen-7-yl)oxy)propenamide, 44 [Step 4]: To a stirred solution of (S)-3-methoxy-2-((2-oxo-4-(o-tolyl)-2H-chromen-7-yl)oxy)propanoic acid (43, 200 mg, 0.6 mmol) in DMF (3 mL) was added DIPEA (0.5 mL, 2.8 mmol) and (NH₄)₂CO₃ (270 mg, 2.8 mmol), followed by the addition of T₃P (50% in EtOAc) (0.5 mL, 0.8 mmol) and the reaction mixture was stirred at ambient temperature for 16 h. The reaction mixture was quenched with ice-cold water, extracted with EtOAc, and the organic layer was washed with brine, dried over anhydrous Na₂SO₄, and concentrated under reduced pressure. The product was purified by RP prep-HPLC to afford (S)-3-methoxy-2-((2-oxo-4-(o-tolyl)-2H-chromen-7-yl)oxy)propanamide (44, 55 mg). LCMS (ESI) Calcd. for C₂₀H₁₉NO₅: 353, found [M+H]⁺=354.

Synthesis of chiral 3-methoxy-2-((2-oxo-4-(o-tolyl)-2H-chromen-7-yl)oxy)propenamide, Examples 67 and 68 [Step 5]: (S)-3-methoxy-2-((2-oxo-4-(o-tolyl)-2H-chromen-7-yl)oxy)propenamide (44) (7:3 (S:R) enantiomeric ratio) was purified by chiral prep-HPLC separation and the first product was isolated as (S)-3-methoxy-2-((2-oxo-4-(o-tolyl)-2H-chromen-7-yl)oxy)propenamide, Peak 1 (Example 67, 30 mg) and the second product as (R)-3-methoxy-2-((2-oxo-4-(o-tolyl)-2H-chromen-7-yl)oxy)propenamide, Peak 2 (Example 68, 10 mg).

Example 67: [(S)-3-methoxy-2-((2-oxo-4-(o-tolyl)-2H-chromen-7-yl)oxy)propenamide, Peak 1]: LCMS (ESI) Calcd. for C₂₀H₁₉NO₅: 353, found [M+H]⁺=354. ¹H NMR (400 MHz, DMSO-d₆) δ 7.66 (br s, 1H), 7.44-7.40 (m, 3H), 7.38-7.34 (t, 1H), 7.24-7.22 (d, 1H), 7.03 (m, 1H), 6.91-6.86 (m, 2H), 6.20 (s, 1H), 4.92 (m, 1H), 3.75-3.71 (m, 2H), 3.30 (s, 3H), 2.11 (s, 3H).

Example 68: [(R)-3-methoxy-2-((2-oxo-4-(o-tolyl)-2H-chromen-7-yl)oxy)propenamide, Peak 2]: LCMS (ESI) Calcd. for C₂₀H₁₉NO₅: 353, found [M+H]⁺=354. ¹H NMR (400 MHz, DMSO-d₆) δ 7.66 (br s, 1H), 7.44-7.40 (m, 3H), 7.38-7.33 (m, 1H), 7.24-7.22 (d, 1H), 7.03 (m, 1H), 6.93-6.86 (m, 2H), 6.20 (s, 1H), 4.92 (m, 1H), 3.77-3.69 (m, 2H), 3.30 (s, 3H), 2.11 (s, 3H).

Chiral prep-HPLC: Chiral separation was performed on an Agilent 1200 series instrument. Column: Chiralpak IC (250×20 mm), 5p, operating at ambient temperature with flow rate of 18.0 mL/min. Mobile phase: Hexane, dichloromethane, and ethanol (70/15/15), held isocratic up to 33 min. with detection at 322 nm wavelength.

### Examples 69-70: 3-methoxy-N-methyl-2-((2-oxo-4-(o-tolyl)-2H-chromen-7-yl)oxy)propanamide, racemic and purified chiral analogs

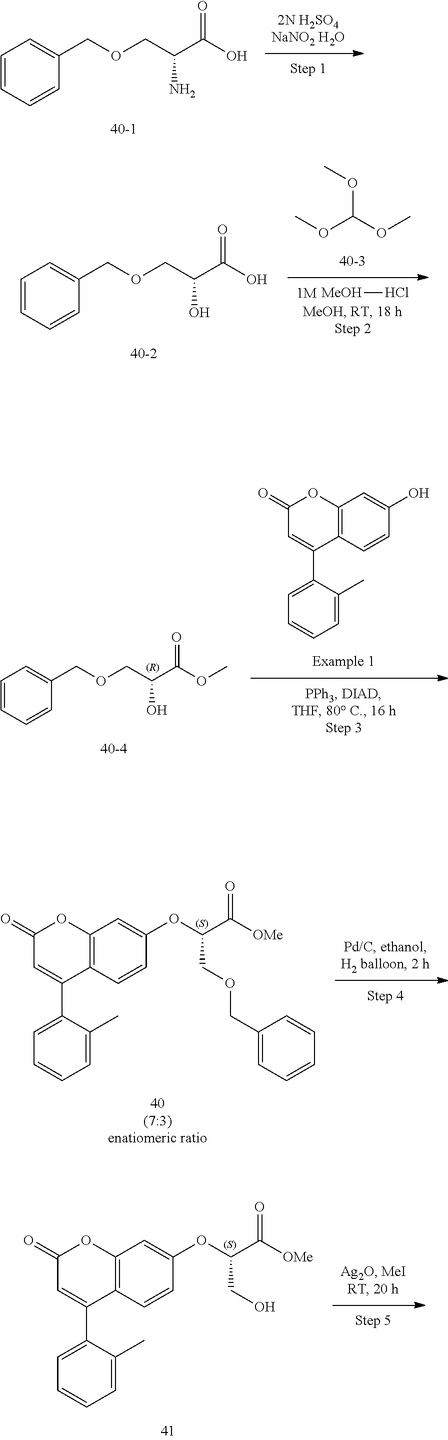

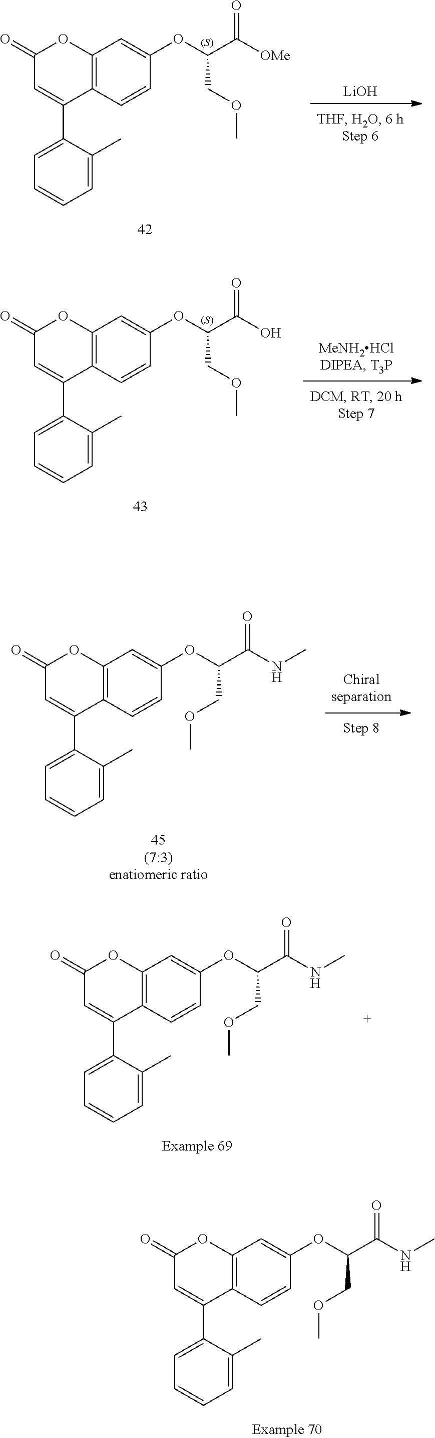

Synthesis of (R)-3-(benzyloxy)-2-hydroxypropanoic acid, 40-2 [Step 1]: A stirred solution of (2R)-2-amino-3-benzyloxy-propanoic acid (40-1, 2.0 g, 10.2 mmol) in 2N H₂SO₄ (10 mL) was cooled to 0° C. Then a solution of NaNO₂ (1.28 g, 18.6 mmol) in water was added to the mixture over 1.5 h. and the temperature was maintained at 0 to 5° C. The reaction mixture was stirred at 5° C. for 6 h. and it was gradually warmed to ambient temperature and stirred for another 3 h. A solution of 50% NaOH was added dropwise to the reaction mixture at 0° C. until pH 4, and the mixture was extracted with ethyl acetate. The aqueous layer was acidified to pH 2 with the addition of 2N H₂SO₄ and extracted several times with EtOAc. The combined organic layer was dried over anhydrous Na₂SO₄, filter, and concentrated under reduced pressure to obtain (R)-3-(benzyloxy)-2-hydroxypropanoic acid (40-2, 1.4 g). LCMS (ESI): Calcd. for C₁₀H₁₂O₄: 196, found [M+NH₄]⁺=214.

Synthesis of methyl (R)-3-(benzyloxy)-2-hydroxypropanoate, 40-4 [Step 2]: To a stirred mixture of (R)-3-(benzyloxy)-2-hydroxypropanoic acid (40-2, 5.3 g, 27.0 mmol) in methanol (35 mL) was added a freshly prepared solution of methanolic HCl 1M (25 mL) at ambient temperature and the mixture was stirred for 1 h. To the mixture was added trimethyl orthoformate (40-3, 17.2 g, 162 mmol) and the mixture was stirred for 18 h. at ambient temperature. The mixture was concentrated under reduced pressure, and the product was purified by column chromatography to afford methyl (R)-3-(benzyloxy)-2-hydroxypropanoate (40-4, 3.4 g). ¹H NMR (400 MHz, DMSO-d₆) δ 7.34-7.24 (m, 5H), 4.61-4.51 (m, 2H), 4.33-4.30 (m, 1H), 3.77 (s, 3H), 3.74 (m, 2H).

Synthesis of methyl 3-(benzyloxy)-2-((2-oxo-4-(o-tolyl)-2H-chromen-7-yl)oxy)propanoate, 40 [Step 3]: To a stirred solution of 7-hydroxy-4-(o-tolyl)-2H-chromen-2-one (Example 1, 1 g, 4 mmol) and methyl (R)-3-(benzyloxy)-2-hydroxypropanoate (40-4, 1.2 g, 6 mmol) in THF (25 mL) was added PPh₃ (3.1 g, 12 mmol). The reaction mixture was cooled to 0° C., and DIAD (2.3 mL, 12 mmol) was added and the reaction mixture was stirred for 5 min., and gradually heated to 80° C. and stirred for 16 h. The reaction mixture was diluted with ethyl acetate, washed with water, brine, dried over anhydrous Na₂SO₄, and concentrated under reduced pressure. The product was purified through combi-flash chromatography to afford methyl 3-(benzyloxy)-2-((2-oxo-4-(o-tolyl)-2H-chromen-7-yl)oxy)propanoate (40, 582 mg). LCMS Calcd. for C₂₇H₂₄O₆: 444, found [M+H]⁺=445. ¹H NMR (400 MHz, DMSO-d₆) δ 7.39-7.27 (m, 8H), 7.15 (d, 1H), 6.97 (d, 1H), 6.83-6.81 (m, 1H), 6.79-6.75 (m, 1H), 6.16 (s, 1H), 4.98 (m, 1H), 4.67 (s, 2H), 3.99-3.92 (m, 2H), 3.79 (s, 3H), 2.16 (s, 3H).

Synthesis of methyl (S)-3-hydroxy-2-((2-oxo-4-(o-tolyl)-2H-chromen-7-yl)oxy)propanoate, 41 [Step 4]: To a stirred solution of methyl (S)-3-benzyloxy-2-[4-(o-tolyl)-2-oxo-chromen-7-yl]oxy-propanoate (40, 520 mg, 1.17 mmol) in ethanol (10 mL) under argon, was added Pd/C (30 mg, 0.117 mmol) and the reaction mixture was subjected to hydrogenolysis using a H₂ balloon for 2 h. The reaction mixture was filtered through a celite bed and concentrated under reduced pressure. The product was purified through column chromatography to obtain methyl (S)-3-hydroxy-2-[4-(o-tolyl)-2-oxo-chromen-7-yl]oxy-propanoate (41, 280 mg). LCMS Calcd. for C₂₀H₁₈O₆: 354, found [M+H]⁺=355.

Synthesis of methyl (S)-3-methoxy-2-((2-oxo-4-(o-tolyl)-2H-chromen-7-yl)oxy)propanoate, 42 [Step 5]: To a stirred solution of methyl (S)-3-hydroxy-2-[4-(o-tolyl)-2-oxo-chromen-7-yl]oxy-propanoate (41, 210 mg, 0.593 mmol) in methyl iodide (1.1 mL, 17.8 mmol) at 0° C. under argon, was added Ag₂O (275 mg, 1.19 mmol) portion wise, and the reaction mixture was stirred at ambient temperature overnight. The reaction mixture was diluted with ethyl acetate and filtered through a celite bed, and concentrated under reduced pressure to afford methyl (S)-3-methoxy-2-((2-oxo-4-(o-tolyl)-2H-chromen-7-yl)oxy)propanoate (42, 215 mg) which was used for next without further purification. LCMS Calcd. for C₂₁H₂₀O₆: 368, found [M+H]⁺=369.

Synthesis of (S)-3-methoxy-2-((2-oxo-4-(o-tolyl)-2H-chromen-7-yl)oxy)propanoic acid, 43 [Step 6]: To a stirred solution of methyl (S)-3-methoxy-2-[4-(o-tolyl)-2-oxo-chromen-7-yl]oxy-propanoate (42, 200 mg, 0.543 mmol) in THF (6 mL) and water (1.5 mL) was added LiOH·H₂O (68 mg, 1.63 mmol). The reaction mixture was stirred for 6 h. at ambient temperature, and concentrated under reduced pressure to obtain (S)-3-methoxy-2-((2-oxo-4-(o-tolyl)-2H-chromen-7-yl)oxy)propanoic acid (43, 160 mg). The product was used in the next step without further purification. LCMS Calcd. for C₂₀H₁₈O₆: 354, found [M+H]⁺=355.

Synthesis of (S)-3-methoxy-N-methyl-2-((2-oxo-4-(o-tolyl)-2H-chromen-7-yl)oxy)propenamide, 45 [Step 7]: To a stirred solution of (S)-3-methoxy-2-[4-(o-tolyl)-2-oxo-chromen-7-yl]oxy-propanoic acid (43, 150 mg, 0.423 mmol) and methylamine hydrochloride (171 mg, 2.54 mmol) in DCM (10 mL) was added N,N-diisopropylethylamine (0.52 mL, 2.96 mmol) at 0° C. under argon, and the reaction mixture was stirred for 10 min. A solution of T₃P (0.20 mL, 0.635 mmol) was added dropwise in ice-cold conditions and the mixture was stirred for 18 h. at ambient temperature. The reaction mixture was diluted with DCM, washed with water, concentrated under reduced pressure, and subjected to RP prep-HPLC to afford (S)-3-methoxy-N-methyl-2-[4-(o-tolyl)-2-oxo-chromen-7-yl]oxy-propanamide (45, 110 mg). LCMS Calcd. for C₂₁H₂₁NO₅: 367, found [M+H]⁺=368. Enantiomeric ratio was 7:3.

Synthesis of (S)-3-methoxy-N-methyl-2-((2-oxo-4-(o-tolyl)-2H-chromen-7-yl)oxy)propenamide, Example 69, and (R)-3-methoxy-N-methyl-2-((2-oxo-4-(o-tolyl)-2H-chromen-7-yl)oxy)propenamide, Example 70 [Step 8]: Enantio-enriched (7:3) (S)-3-methoxy-N-methyl-2-((2-oxo-4-(o-tolyl)-2H-chromen-7-yl)oxy)propanamide (45, 110 mg) was purified by chiral prep-HPLC separation and the first product was isolated as (S)-3-methoxy-N-methyl-2-((2-oxo-4-(o-tolyl)-2H-chromen-7-yl)oxy)propenamide, Peak 1 (Example 69, 59 mg) and the second product as (R)-3-methoxy-N-methyl-2-((2-oxo-4-(o-tolyl)-2H-chromen-7-yl)oxy)propenamide, Peak 2 (Example 70, 21 mg).

Example 69: [(S)-3-methoxy-N-methyl-2-((2-oxo-4-(o-tolyl)-2H-chromen-7-yl)oxy)propenamide, Peak 1]: LCMS (ESI) Calcd. for C₂₁H₂₁NO₅: 367, found [M+H]⁺=368. ¹H NMR (400 MHz, DMSO-d₆) δ 8.16 (br s, 1H), 7.44-7.41 (m, 2H), 7.35 (t, 1H), 7.24 (d, 1H), 7.05-7.04 (m, 1H), 6.92-6.89 (m, 2H), 6.21 (s, 1H), 4.98 (s, 1H), 3.74-3.71 (m, 2H), 3.28 (s, 3H), 2.61 (d, 3H), 2.12 (s, 3H).

Example 70: [(R)-3-methoxy-N-methyl-2-((2-oxo-4-(o-tolyl)-2H-chromen-7-yl)oxy)propenamide, Peak 2]: LCMS (ESI) Calcd. for C₂₁H₂₁NO₅: 367, found [M+H]⁺=368. ¹H NMR (400 MHz, DMSO-d₆) δ 8.16 (br s, 1H), 7.44-7.41 (m, 2H), 7.35 (t, 1H), 7.24 (d, 1H), 7.05-7.04 (m, 1H), 6.92-6.89 (m, 2H), 6.21 (s, 1H), 4.97 (s, 1H), 3.74-3.71 (m, 2H), 3.28 (s, 3H), 2.61 (d, 3H), 2.12 (s, 3H).

Chiral prep-HPLC: Chiral separation was performed on an Agilent 1200 series instrument. Column: Chiralpak IC (250×20 mm), 5p, operating at ambient temperature with flow rate of 18.0 mL/min. Mobile phase: Hexane, dichloromethane, and ethanol (65/17.5/17.5), held isocratic up to 20 min. with detection at 322 nm wavelength.

### Examples 71-72: 3-methoxy-N,N-dimethyl-2-((2-oxo-4-(o-tolyl)-2H-chromen-7-yl)oxy)propenamide, racemic and purified chiral analogs

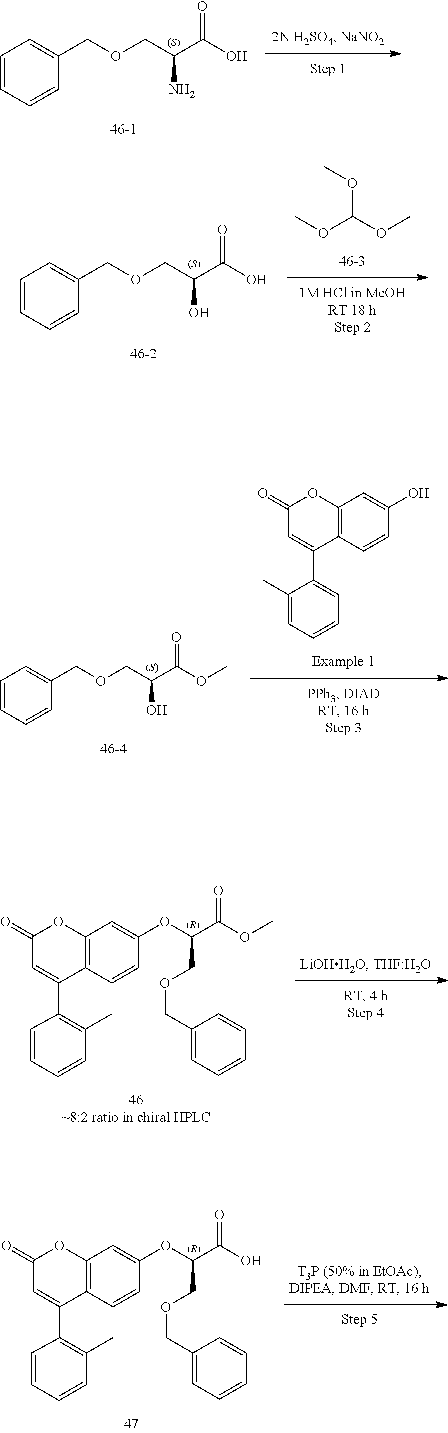

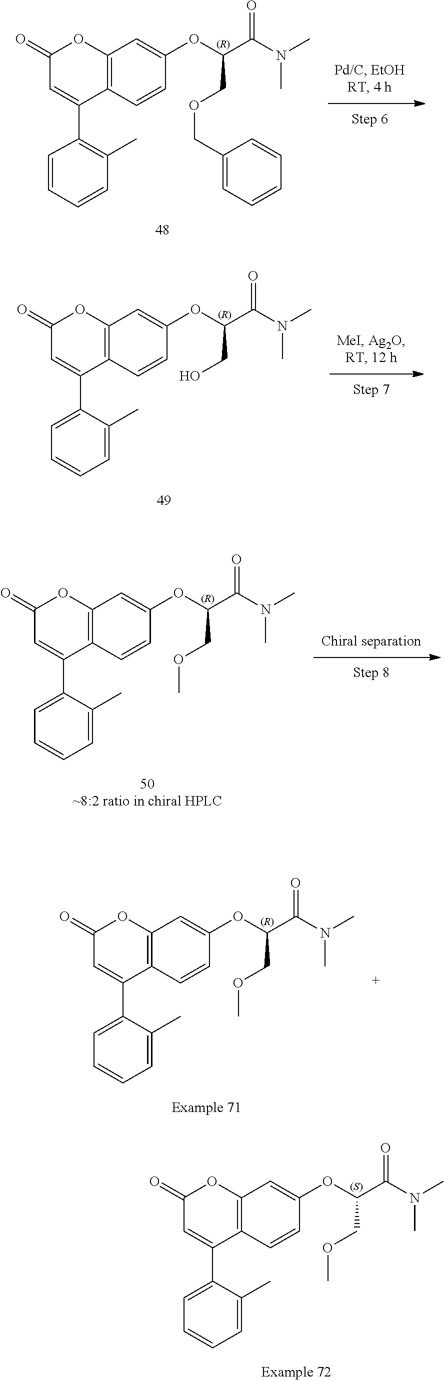

Synthesis of (S)-3-(benzyloxy)-2-hydroxypropanoic acid, 46-2 [Step 1]: To stirred a solution of O-benzyl-L-serine (46-1, 10 g, 51.2 mmol) in 2N H₂SO₄ (30 mL) at 0° C. was added an aqueous solution of NaNO₂ (6.3 g, 92 mmol) over a period of 2 h. The reaction mixture was stirred at the same temperature for 6 h. and then allowed to warm to ambient temperature and stirred for 12 h. The reaction mixture was cooled and a solution of 50% NaOH was added slowly to adjust to pH ~4, and the reaction mixture was stirred for 10 min. EtOAc (100 mL) was added and the reaction mixture was stirred for another 10 min., cooled to 0° C., and acidified with 2N H₂SO₄ to pH ~2. The organic layer was separated and the aqueous layer was re-extracted with EtOAc (twice). The combined organic layer was washed with cold water, brine, dried over anhydrous Na₂SO₄, and concentrated under reduced pressure to afford (S)-3-(benzyloxy)-2-hydroxypropanoic acid (46-2, 7 g). ¹H NMR (400 MHz, DMSO-d₆) δ 12.51 (s, 1H), 7.33-7.22 (m, 5H), 5.32 (s, 1H), 4.54-4.47 (m, 2H), 4.15 (t, 1H), 3.60 (d, 2H).

Synthesis of methyl (S)-3-(benzyloxy)-2-hydroxypropanoate, 46-4 [Step 2]: To a stirred solution of (S)-3-(benzyloxy)-2-hydroxypropanoic acid (46-2, 4.5 g, 13.7 mmol) in MeOH (50 mL) was added 1M methanolic HCl (8.1 mL, 8.1 mmol), and the reaction mixture was allowed to stir for 1.5 h. at ambient temperature. To the reaction mixture was added trimethyl orthoformate (46-3, 4.9 g, 46 mmol) and stirring was continued for 18 h. at ambient temperature. The reaction mixture was concentrated under reduced pressure and the product was purified by flash column chromatography to afford methyl (S)-3-(benzyloxy)-2-hydroxypropanoate (46-4, 2.2 g). ¹H NMR (400 MHz, DMSO-d₆) δ 7.36-7.26 (m, 5H), 5.58 (d, 1H), 4.53-4.45 (m, 2H), 4.27-4.23 (m, 1H), 3.66-3.60 (m, 5H).

Synthesis of methyl (R)-3-(benzyloxy)-2-((2-oxo-4-(o-tolyl)-2H-chromen-7-yl)oxy)propanoate, 46 [Step 3]: To a stirred solution of methyl (S)-3-(benzyloxy)-2-hydroxypropanoate (46-4, 1.8 g, 8.7 mmol) in THF (10 mL) was added 7-hydroxy-4-(o-tolyl)chromen-2-one (Example 1, 1 g, 4 mmol), PPh₃ (3.1 g, 11.9 mmol), and the reaction mixture was cooled 0° C. To the reaction mixture was added DIAD (2.4 mL, 11.9 mmol) and the reaction mixture was allowed to warm to ambient temperature and then heated at 80° C. for 16 h. The reaction mixture was concentrated under reduced pressure and the product was diluted with EtOAc, washed with water, brine and dried over anhydrous Na₂SO₄, and concentrated under reduced pressure. The product was purified through combi-flash chromatography to afford methyl (R)-3-(benzyloxy)-2-((2-oxo-4-(o-tolyl)-2H-chromen-7-yl)oxy)propanoate (46, 1.20 g). LCMS (ESI) Calcd. for C₂₇H₂₄O₆: 444, found [M+H]⁺=445. The enantiomeric ratio was determined to be approximately 8:2 of R:S enantiomers.

Synthesis of methyl (R)-3-(benzyloxy)-2-(2-oxo-4-(o-tolyl)-2H-chromen-7-yl)oxy)propanoic acid, 47 [Step 4]: To stirred a solution of methyl (R)-3-(benzyloxy)-2-((2-oxo-4-(o-tolyl)-2H-chromen-7-yl)oxy)propanoate (46, 200 mg, 0.45 mmol) in THF (4 mL) and water (1 mL), was added LiOH·H₂O (75 mg, 1.79 mmol) and the reaction mixture was stirred for 16 h. at ambient temperature. The reaction mixture was acidified with 1N HCl to pH ~5 and extracted with EtOAc. The organic layer was washed with water, brine, dried over anhydrous Na₂SO₄, and concentrated under reduced pressure. The product was purified by combi-flash chromatography to afford methyl (R)-3-(benzyloxy)-2-(2-oxo-4-(o-tolyl)-2H-chromen-7-yl)oxy)propanoic acid (47, 150 mg, 75%). LCMS (ESI) Calcd. for C₂₆H₂₂O₆: 430, found [M+H]⁺=431.

Synthesis of (R)-3-(benzyloxy)-N,N-dimethyl-2-((2-oxo-4-(o-tolyl)-2H-chromen-7-yl)oxy)propenamide, 48 [Step 5]: To a stirred solution of methyl (R)-3-(benzyloxy)-2-(2-oxo-4-(o-tolyl)-2H-chromen-7-yl)oxy)propanoic acid (47, 1.4 g, 3.2 mmol) in DCM (10 mL) was added N-methylmethanamine hydrochloride (1.6 g, 19.5 mmol) and DIPEA (3.1 mL, 22.8 mmol). The reaction mixture was cooled to 0° C. and T₃P (50% in EtOAc) (1.3 mL, 4.9 mmol) was added, and the reaction mixture was allowed to warm to ambient temperature and stirred for 16 h. The reaction mixture was diluted with DCM and washed with water, brine, dried over anhydrous Na₂SO₄, and concentrated under reduced pressure. The product was purified by flash column chromatography to afford (R)-3-(benzyloxy)-N,N-dimethyl-2-((2-oxo-4-(o-tolyl)-2H-chromen-7-yl)oxy)propanamide (48, 0.5 g). LCMS (ESI) Calcd. for C₂₈H₂₇NO₅: 457, found [M+H]⁺=458.

Synthesis of (R)-3-hydroxy-N,N-dimethyl-2-((2-oxo-4-(o-tolyl)-2H-chromen-7-yl)oxy)propenamide, 49 [Step 6]: A stirred solution of (R)-3-(benzyloxy)-N,N-dimethyl-2-((2-oxo-4-(o-tolyl)-2H-chromen-7-yl)oxy)propanamide (48, 600 mg, 1.3 mmol) in ethanol (7 mL) was degassed with argon for 10 min. and then Pd/C (70 mg, 0.1 mmol) was added under an argon atmosphere. The reaction mixture was subjected to hydrogenolysis using a hydrogen balloon for 1 h., filtered through a celite bed, and the filtrate was concentrated under reduced pressure. The product was purified by flash column chromatography to afford (R)-3-hydroxy-N,N-dimethyl-2-((2-oxo-4-(o-tolyl)-2H-chromen-7-yl)oxy)propanamide (49, 80 mg). LCMS (ESI) Calcd. for C₂₁H₂₁NO₅: 367, found [M+H]⁺=368.

Synthesis of (R)-3-methoxy-N,N-dimethyl-2-((2-oxo-4-(o-tolyl)-2H-chromen-7-yl)oxy)propenamide, 50 [Step 7]: To a stirred solution of (R)-3-hydroxy-N,N-dimethyl-2-((2-oxo-4-(o-tolyl)-2H-chromen-7-yl)oxy)propanamide (49, 80 mg, mmol) was added MeI (0.41 mL, 6.5 mmol) at 0° C. under an argon atmosphere. Ag₂O (101 mg, 0.4 mmol) was added to the reaction mixture and stirring was continued at ambient temperature for 12 h. The reaction mixture was filtered through a celite bed and concentrated under reduce pressure. The product was purified by RP prep-HPLC to afford (R)-3-methoxy-N,N-dimethyl-2-((2-oxo-4-(o-tolyl)-2H-chromen-7-yl)oxy)propanamide (50, 60 mg). LCMS (ESI) Calcd. for C₂₂H₂₃NO₅: 381, found [M+H]⁺=382. The enantiomeric ratio was approximately 8:2 R:S enantiomers.

Synthesis of chiral 3-methoxy-N,N-dimethyl-2-((2-oxo-4-(o-tolyl)-2H-chromen-7-yl)oxy)propenamide, Examples 71 and 72 [Step 8]: Enantio-enriched (8:2) (R)-3-methoxy-N,N-dimethyl-2-((2-oxo-4-(o-tolyl)-2H-chromen-7-yl)oxy)propanamide (50, 60 mg) was purified by chiral prep-HPLC separation and the first product was isolated as (R)-3-methoxy-N,N-dimethyl-2-((2-oxo-4-(o-tolyl)-2H-chromen-7-yl)oxy)propenamide, Peak 1 (Example 71, 27 mg) and the second peak as (S)-3-methoxy-N,N-dimethyl-2-((2-oxo-4-(o-tolyl)-2H-chromen-7-yl)oxy)propenamide, Peak 2 (Example 72, 6 mg).

Example 71: (R)-3-methoxy-N,N-dimethyl-2-((2-oxo-4-(o-tolyl)-2H-chromen-7-yl)oxy)propenamide, Peak 1: LCMS (ESI) Calcd. for C₂₂H₂₃NO₅: 382, found [M+H]⁺=382. ¹H NMR (400 MHz, DMSO-d₆) δ 7.40-7.32 (m, 3H), 7.24 (m, 1H), 6.91-6.80 (m, 3H), 6.19 (s, 1H), 5.53 (m, 1H), 3.73 (m, 2H), 3.32 (s, 3H), 3.13 (s, 3H), 2.83 (s, 3H), 2.11 (s, 3H).

Example 72: (S)-3-methoxy-N,N-dimethyl-2-((2-oxo-4-(o-tolyl)-2H-chromen-7-yl)oxy)propenamide, Peak 2: LCMS (ESI) Calcd. for C₂₂H₂₃NO₅: 382, found [M+H]⁺ 382. ¹H NMR (400 MHz, DMSO-d₆) δ 7.43-7.34 (m, 3H), 7.24 (m, 1H), 6.91-6.83 (m, 3H), 6.19 (s, 1H), 5.50 (m, 1H), 3.73 (m, 2H), 3.31 (s, 3H), 3.13 (s, 3H), 2.83 (s, 3H), 2.11 (s, 3H).

Chiral prep-HPLC: Chiral separation was performed on an Agilent 1200 series instrument. Column: Chiralpak IC (250×20 mm) 5p, operating at ambient temperature with flow rate of 18.0 mL/min. Mobile phase: 0.1% isopropylamine in a mixture of hexane, dichloromethane, and ethanol (70/15/15), held isocratic up to 17 min. with detection at 324 nm wavelength.

### Examples 73-75: 2-[4-(2-chloro-4-fluoro-phenyl)-2-oxo-2H-chromen-7-yl]oxy-3-methoxy-propanoic acid, and chiral analogs of 2-((4-(2-chloro-4-fluorophenyl)-2-oxo-2H-chromen-7-yl)oxy)-3-methoxypropanamide

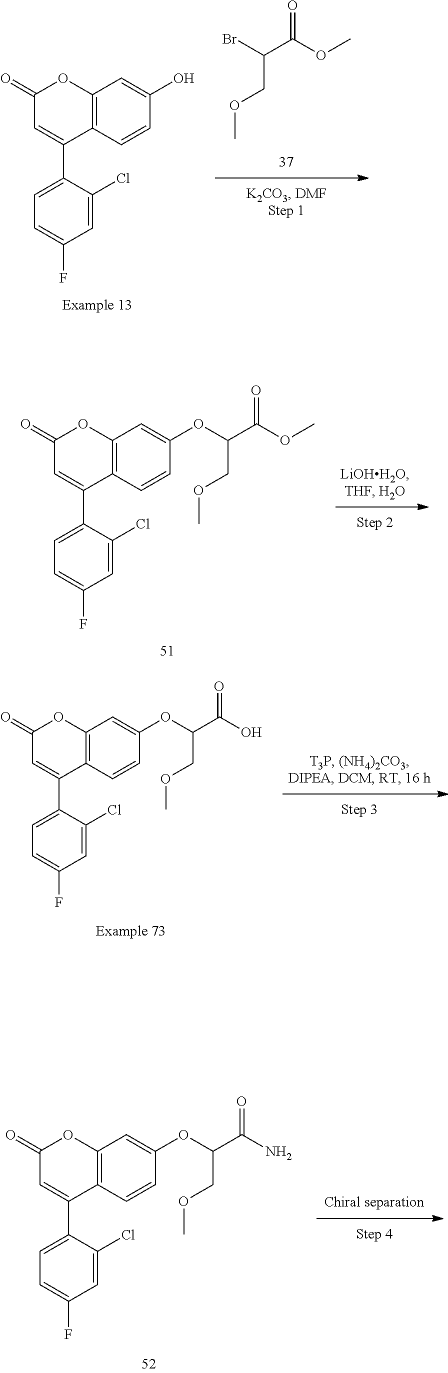

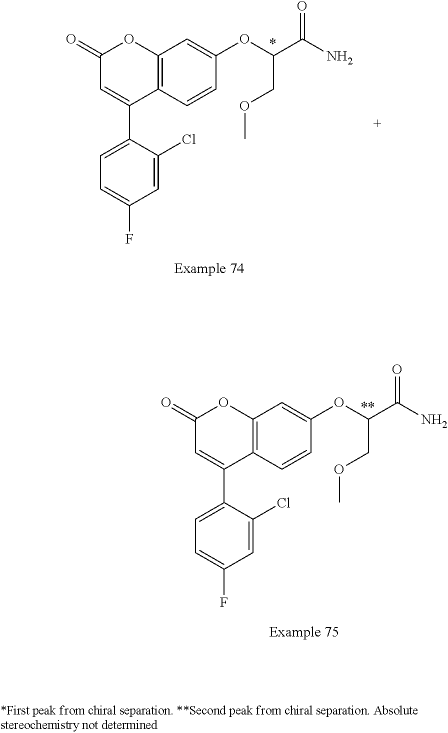 [SMILES: COCC(Oc1ccc2c(-c3ccc(F)cc3Cl)cc(=O)oc2c1)C(N)=O ; COCC(Oc1ccc2c(-c3ccc(F)cc3Cl)cc(=O)oc2c1)C(N)=O]

Synthesis of methyl 3-(4-(2-chloro-4-fluorophenyl)-2-oxo-2H-chromen-7-yl)-2-(methoxymethyl)propanoate, 51 [Step 1]: To a stirred solution of 4-(2-chloro-4-fluorophenyl)-7-hydroxy-2H-chromen-2-one (Example 13, 200 mg, 0.7 mmol) in DMF (5 mL) was added K₂CO₃ (561 mg, 1.7 mmol) followed by methyl 2-bromo-3-methoxypropanoate (37, 0.1 mL, 0.83 mmol). To the reaction mixture was added EtOAc and water, and the organic layer was extracted and washed with brine, ice-cold water, dried over anhydrous Na₂SO₄, and concentrated under reduce pressure. The product was purified by combi-flash chromatography to afford methyl 3-(4-(2-chloro-4-fluorophenyl)-2-oxo-2H-chromen-7-yl)-2-(methoxymethyl)propanoate (51, 260 mg). LCMS (ESI) Calcd. for C₂₀H₁₆ClFO₆: 406, found [M+H]⁺=407.

Synthesis of 2-((4-(2-chloro-4-fluorophenyl)-2-oxo-2H-chromen-7-yl)oxy)-3-methoxypropanoic acid, Example 73 [Step 2]: To a stirred solution of methyl 2-[4-(2-chloro-4-fluoro-phenyl)-2-oxo-2H-chromen-7-yl]oxy-3-methoxy-propanoate (51, 600 mg, 1.5 mmol) in THF (3 mL) and water (1 mL) was added LiOH·H₂O (153 mg, 6.4 mmol) at ambient temperature and the reaction mixture was allowed to stir at the same temperature for 2 h. The reaction mixture was concentrated under reduced pressure and the product was diluted with a minimum amount of water and slowly acidified with citric acid until pH 4-5. The product was extracted with ethyl acetate and the combined organic layer was washed with brine, dried over anhydrous Na₂SO₄, and concentrated under reduced pressure. The product was purified by RP prep-HPLC to afford 2-[4-(2-chloro-4-fluoro-phenyl)-2-oxo-2H-chromen-7-yl]oxy-3-methoxy-propanoic acid (Example 73, 300 mg). LCMS (ESI) Calcd. for C₁₉H₁₄ClFO₆: 392, found [M+H]⁺=393. ¹H NMR (400 MHz, DMSO-d₆) δ 7.71-7.68 (m, 1H), 7.57-7.53 (m, 1H), 7.44-7.40 (m, 1H), 6.90-6.87 (m, 2H), 6.84-6.81 (m, 1H), 6.26 (s, 1H), 4.79 (m, 1H), 3.74 (m, 2H), 3.32 (s, 3H).

Synthesis of 2-((4-(2-chloro-4-fluorophenyl)-2-oxo-2H-chromen-7-yl)oxy)-3-methoxypropanamide, 52 [Step 3]: To a stirred solution of 2-[4-(2-chloro-4-fluoro-phenyl)-2-oxo-2H-chromen-7-yl]oxy-3-methoxy-propanoic acid (Example 73, 260 mg, mmol) in DMF (5 mL) was added (NH₄)₂CO₃ (254 mg, 2.7 mmol), DIPEA (0.58 mL, 3.3 mmol), and then T₃P (0.58 mL, 1 mmol). The reaction mixture was allowed to stir at ambient temperature for 4 h., and then concentrated under reduced pressure. Water was added to the product and the mixture was extracted with EtOAc (three times). The combined organic layers were washed with aq. NaHCO₃ solution, brine, dried over anhydrous Na₂SO₄, filtered, and concentrated under reduced pressure. The product was purified by flash chromatography to afford 2-((4-(2-chloro-4-fluorophenyl)-2-oxo-2H-chromen-7-yl)oxy)-3-methoxypropanamide (52, 150 mg).

Synthesis of chiral 2-((4-(2-chloro-4-fluorophenyl)-2-oxo-2H-chromen-7-yl)oxy)-3-methoxypropanamide, Examples 74 and 75 [Step 4]: 2-[4-(2-chloro-4-fluoro-phenyl)-2-oxo-2H-chromen-7-yl]oxy-3-methoxy-propanamide (52, 150 mg, 0.4 mmol) was purified by chiral prep-HPLC separation and the first product was isolated as 2-((4-(2-chloro-4-fluorophenyl)-2-oxo-2H-chromen-7-yl)oxy)-3-methoxypropanamide, Peak 1 (Example 74, 50 mg) and the second peak as 2-((4-(2-chloro-4-fluorophenyl)-2-oxo-2H-chromen-7-yl)oxy)-3-methoxypropanamide, Peak 2 (Example 75, 40 mg). The absolute stereochemistries of these compounds were not determined.

Example 74: 2-((4-(2-chloro-4-fluorophenyl)-2-oxo-2H-chromen-7-yl)oxy)-3-methoxypropanamide, Peak 1: LCMS (ESI) Calcd. for C₁₉H₁₅ClFNO₅: 391, found [M+H]⁺=392. ¹H NMR (400 MHz, DMSO-d₆) δ 7.72-7.67 (m, 2H), 7.57-7.54 (m, 1H), 7.46-7.41 (m, 2H), 7.04 (s, 1H), 6.97-6.91 (m, 2H), 6.33 (s, 1H), 4.94-4.92 (m, 1H), 3.78-3.69 (m, 2H), 3.31 (s, 3H).

Example 75: 2-((4-(2-chloro-4-fluorophenyl)-2-oxo-2H-chromen-7-yl)oxy)-3-methoxypropanamide, Peak 2: LCMS (ESI) Calcd. for C₁₉H₁₅ClFNO₅: 391, found [M+H]⁺=392. ¹H NMR (400 MHz, DMSO-d₆) δ 7.72-7.67 (m, 2H), 7.57-7.54 (m, 1H), 7.46-7.41 (m, 2H), 7.04 (s, 1H), 6.97-6.91 (m, 2H), 6.33 (s, 1H), 4.94-4.92 (m, 1H), 3.78-3.69 (m, 2H), 3.31 (s, 3H).

Chiral prep-HPLC: Chiral separation was performed on an Agilent 1200 series instrument. Column: Chiralpak IC (250×21 mm) 5p, operating at ambient temperature with flow rate of 21.0 mL/min. Mobile phase: 0.1% isopropylamine in hexane, dichloromethane, and ethanol (50/25/25), held isocratic up to 24 min. with detection at 325 nm wavelength.

### Examples 76-77: 2-((4-(2-chloro-4-fluorophenyl)-2-oxo-2H-chromen-7-yl)oxy)-3-methoxypropanamide, racemic and purified chiral analogs

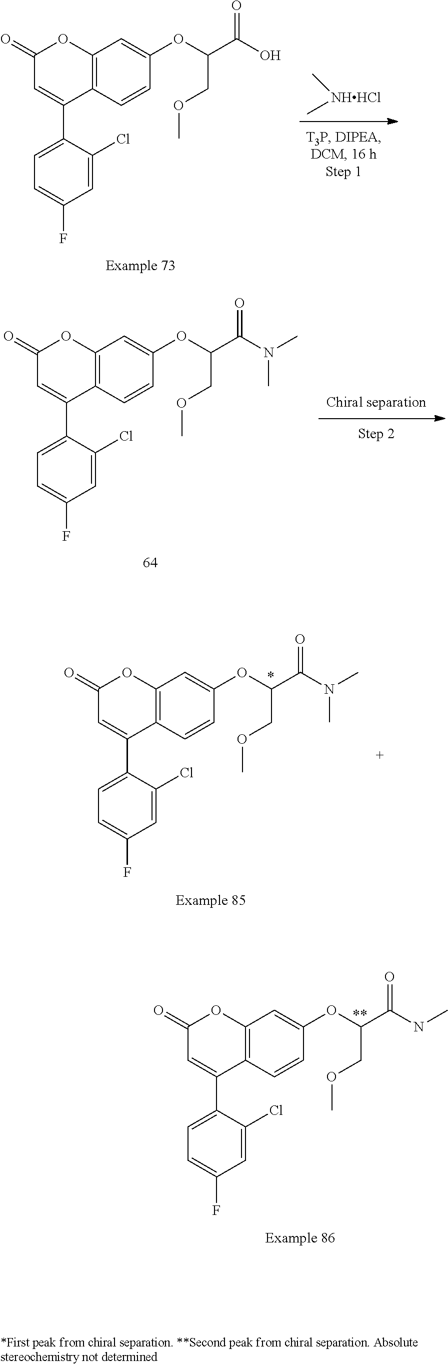

Synthesis of 2-((4-(2-chloro-4-fluorophenyl)-2-oxo-2H-chromen-7-yl)oxy)-3-methoxypropanamide, 53 [Step 1]: To a stirred solution of 2-[4-(4-chloro-2-fluoro-phenyl)-2-oxo-2H-chromen-7-yl]oxy-3-methoxy-propanoic acid (Example 73, 261 mg, mmol) in DCM (5 mL) was added DIPEA (0.58 mL, 3.3 mmol), methylamine hydrochloride (224 mg, 3.3 mmol), T₃P (0.59 mL, 1 mmol), and the reaction mixture was stirred at ambient temperature for 16 h. The reaction mixture was diluted with DCM and washed with water, brine, dried over anhydrous Na₂SO₄, and concentrated under reduced pressure to obtain the product. The product was purified by flash chromatography to afford 2-((4-(2-chloro-4-fluorophenyl)-2-oxo-2H-chromen-7-yl)oxy)-3-methoxypropanamide (53, 220 mg). LCMS Calcd. for C₂₀H₁₇ClFNO₅: 405, found [M+H]⁺=406.

Synthesis of chiral 2-((4-(2-chloro-4-fluorophenyl)-2-oxo-2H-chromen-7-yl)oxy)-3-methoxypropanamide, Examples 76 and 77 [Step 2]: 2-((4-(2-chloro-4-fluorophenyl)-2-oxo-2H-chromen-7-yl)oxy)-3-methoxypropanamide (53, 250 mg, 0.616 mmol) was purified by chiral prep-HPLC separation and the first product was isolated as 2-((4-(2-chloro-4-fluorophenyl)-2-oxo-2H-chromen-7-yl)oxy)-3-methoxypropanamide, Peak 1 (Example 76, 90 mg) and the second peak as 2-((4-(2-chloro-4-fluorophenyl)-2-oxo-2H-chromen-7-yl)oxy)-3-methoxypropanamide, Peak 2 (Example 77, 100 mg). The absolute stereochemistries of these compounds were not determined.

Example 76: 2-((4-(2-chloro-4-fluorophenyl)-2-oxo-2H-chromen-7-yl)oxy)-3-methoxypropanamide, Peak 1: LCMS (ESI) Calcd. for C₂₀H₁₇ClFNO₅: 405, found [M+H]⁺=406. ¹H NMR (400 MHz, DMSO-d₆) δ 8.17-8.15 (m, 1H), 7.72-7.69 (m, 1H), 7.57-7.54 (m, 1H), 7.46-7.41 (m, 1H), 7.06-7.05 (m, 1H), 6.97-6.92 (m, 2H), 6.33 (s, 1H), 4.97 (m, 1H), 3.77-3.68 (m, 2H), 3.31 (s, 3H), 2.61 (d, 3H).

Example 77: 2-((4-(2-chloro-4-fluorophenyl)-2-oxo-2H-chromen-7-yl)oxy)-3-methoxypropanamide, Peak 2: LCMS (ESI) Calcd. for C₂₀H₁₇ClFNO₅: 405, found [M+H]⁺=406. ¹H NMR (400 MHz, DMSO-d₆) δ 8.18-8.16 (m, 1H), 7.71-7.69 (m, 1H), 7.57-7.54 (m, 1H), 7.46-7.41 (m, 1H), 7.06-7.05 (m, 1H), 6.97-6.92 (m, 2H), 6.33 (s, 1H), 4.99 (m, 1H), 3.77-3.68 (m, 2H), 3.31 (s, 3H), 2.61 (d, 3H).

Chiral prep-HPLC: Chiral separation was performed on an Agilent 1200 series instrument. Column: Chiralpak IC (250×21 mm) 5p, operating at ambient temperature with flow rate of 21.0 mL/min. Mobile phase: 0.1% isopropylamine in a mixture of hexane, dichloromethane, and ethanol (50/25/25), held isocratic up to 40 min. with detection at 325 nm wavelength.

### Examples 78-79: 3-hydroxy-N,N-dimethyl-2-((2-oxo-4-(o-tolyl)-2H-chromen-7-yl)oxy)propanamide, racemic and purified chiral analogs

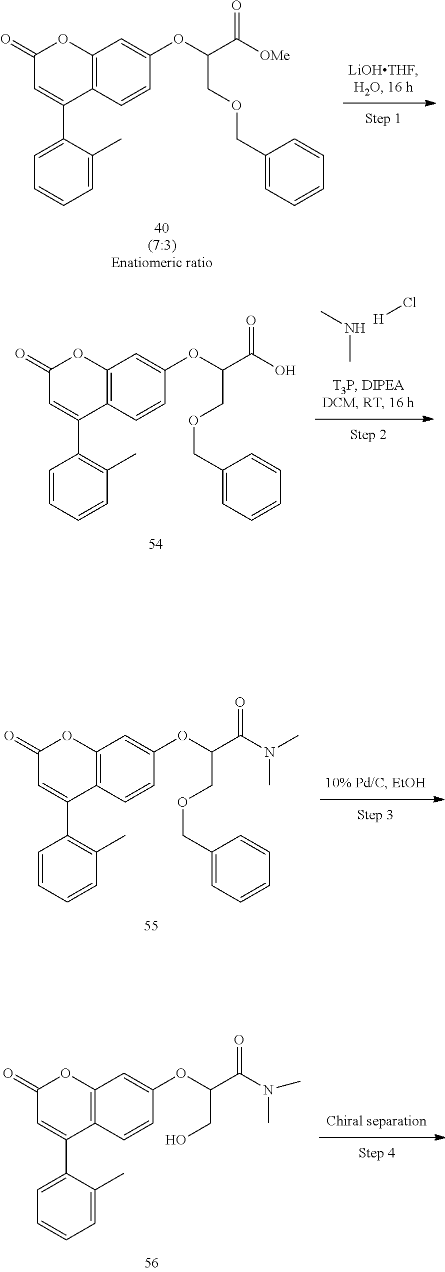

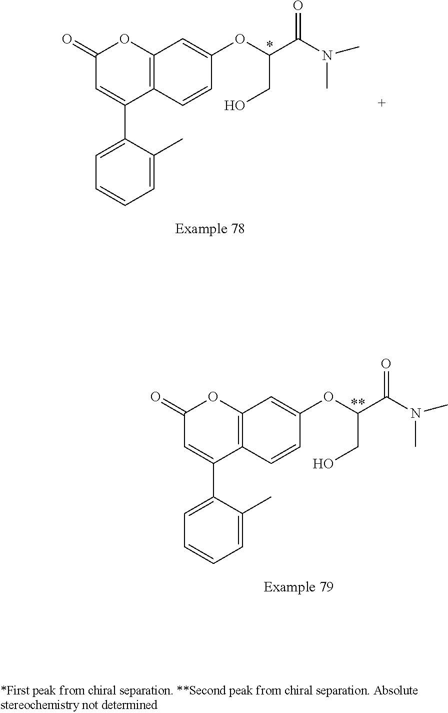 [SMILES: Cc1ccccc1-c1cc(=O)oc2cc(OC(CO)C(=O)N(C)C)ccc12 ; Cc1ccccc1-c1cc(=O)oc2cc(OC(CO)C(=O)N(C)C)ccc12]

Synthesis of 3-(benzyloxy)-2-((2-oxo-4-(o-tolyl)-2H-chromen-7-yl)oxy)propanoic acid, 54 [Step 1]: To a stirred solution of methyl 3-(benzyloxy)-2-((2-oxo-4-(o-tolyl)-2H-chromen-7-yl)oxy)propanoate (40, 400 mg, 0.9 mmol) in THF (6 mL) and water (1.5 mL) was added LiOH·H₂O (113 mg, 2.7 mmol) at ambient temperature and the reaction mixture was stirred for 16 h. The mixture was concentrated under reduced pressure to obtain the product, to which water was added and the mixture was acidified with 1N HCl to pH 5. The mixture was extracted with EtOAc, and the organic layer was washed with water, brine, dried over anhydrous Na₂SO₄ and concentrated under reduced pressure to afford 3-(benzyloxy)-2-((2-oxo-4-(o-tolyl)-2H-chromen-7-yl)oxy)propanoic acid (54, 320 mg). LCMS Calcd. for C₂₆H₂₂O₆: 430, found [M+H]⁺=431.

Synthesis of 3-(benzyloxy)-N,N-dimethyl-2-((2-oxo-4-(o-tolyl)-2H-chromen-7-yl)oxy)propenamide, 55 [Step 2]: To a stirred solution of 3-(benzyloxy)-2-((2-oxo-4-(o-tolyl)-2H-chromen-7-yl)oxy)propanoic acid (54, 320 mg, 0.7 mmol) and N-methylmethanamine hydrochloride (364 mg, 4.5 mmol) in DCM (10 mL), was added DIPEA (0.7 mL, 5.2 mmol) and the reaction mixture was cooled to 0° C. Then T₃P (0.3 mL, 1.1 mmol) was added dropwise at 0° C. and the reaction mixture was allowed to warm up to ambient temperature and stirred for 16 h. The reaction mixture was diluted with DCM and washed with water and brine. The organic layer was dried over anhydrous Na₂SO₄ and concentrated under reduced pressure to obtain the product, which was purified by combi-flash chromatography to afford 3-(benzyloxy)-N,N-dimethyl-2-((2-oxo-4-(o-tolyl)-2H-chromen-7-yl)oxy)propanamide (55, 170 mg). LCMS Calcd. for C₂₈H₂₇NO₅: 457, found [M+H]⁺=458. ¹H NMR (400 MHz, DMSO-d₆) δ 7.43-7.22 (m, 9H), 6.94 (m, 1H), 6.86 (m, 2H), 6.19 (s, 1H), 5.58 (m, 1H), 4.59 (s, 2H), 3.83 (m, 2H), 3.09 (s, 3H), 2.83 (s, 3H), 2.11 (s, 3H).

Synthesis of 3-hydroxy-N,N-dimethyl-2-((2-oxo-4-(o-tolyl)-2H-chromen-7-yl)oxy)propenamide, 56 [Step 3]: A stirred solution of 3-benzyloxy-N,N-dimethyl-2-[4-(o-tolyl)-2-oxo-2H-chromen-7-yl]oxy-propanamide (55, 170 mg, 0.372 mmol) in ethanol (5 mL) was purged with argon for 5 min. To the solution was added Pd-C (25 mg, 0.04 mmol) and the reaction mixture was subjected to hydrogenolysis using a hydrogen balloon for 4 h. The reaction mixture was filtered through a celite bed and the filtrate was concentrated under reduced pressure to obtain the product, which was purified by RP prep-LC to afford 3-hydroxy-N,N-dimethyl-2-((2-oxo-4-(o-tolyl)-2H-chromen-7-yl)oxy)propanamide (56, 60 mg). LCMS Calcd. for C₂₁H₂₁NO₅: 367, found [M+H]⁺=368.

Synthesis of chiral 3-hydroxy-N,N-dimethyl-2-((2-oxo-4-(o-tolyl)-2H-chromen-7-yl)oxy)propenamide, Examples 78 and 79 [Step 4]: 3-hydroxy-N,N-dimethyl-2-[4-(o-tolyl)-2-oxo-2H-chromen-7-yl]oxy-propanamide (56, 60 mg, 0.163 mmol) was purified by chiral prep-HPLC separation and the first product was isolated as 3-hydroxy-N,N-dimethyl-2-[4-(o-tolyl)-2-oxo-2H-chromen-7-yl]oxy-propanamide (56, 60 mg, 0.163 mmol), Peak 1 (Example 78, 10 mg) and the second product as 3-hydroxy-N,N-dimethyl-2-[4-(o-tolyl)-2-oxo-2H-chromen-7-yl]oxy-propanamide, Peak 2 (Example 79, 25 mg). The absolute stereochemistries of these compounds were not determined.

Example 78: [3-hydroxy-N,N-dimethyl-2-[4-(o-tolyl)-2-oxo-2H-chromen-7-yl]oxy-propanamide, Peak 1]: LCMS Calcd. for C₂₁H₂₁NO₅: 367, found [M+H]⁺=368. ¹H NMR (400 MHz, DMSO-d₆) δ 7.43-7.33 (m, 3H), 7.24 (m, 1H), 6.88 (m, 2H), 6.82-6.80 (m, 1H), 6.19 (s, 1H), 5.28-5.22 (m, 2H), 3.77 (m, 2H), 3.14 (s, 3H), 2.84 (s, 3H), 2.11 (s, 3H).

Example 79: [3-hydroxy-N,N-dimethyl-2-[4-(o-tolyl)-2-oxo-2H-chromen-7-yl]oxy-propanamide, Peak 2]: LCMS Calcd. for C₂₁H₂₁NO₅: 367, found [M+H]⁺=368. ¹H NMR (400 MHz, DMSO-d₆) δ 7.43-7.34 (m, 3H), 7.24 (m, 1H), 6.88-6.86 (m, 2H), 6.82 (m, 1H), 6.19 (s, 1H), 5.28-5.23 (m, 2H), 3.77 (m, 2H), 3.14 (s, 3H), 2.83 (s, 3H), 2.11 (s, 3H).

Chiral prep-HPLC: Chiral separation was performed on an Agilent 1200 series instrument. Column: Chiralpak IC (250×20 mm), 5p, operating at ambient temperature with flow rate of 18.0 mL/min. Mobile phase: 0.1% isopropylamine in a mixture of 60% hexane, 20% of CH₂Cl₂ and 20% ethanol, held isocratic up to 15 min. with detection at 324 nm wavelength.

### Examples 80-81: N-ethyl-3-methoxy-2-((2-oxo-4-(o-tolyl)-2H-chromen-7-yl)oxy)propanamide, racemic and purified chiral analogs

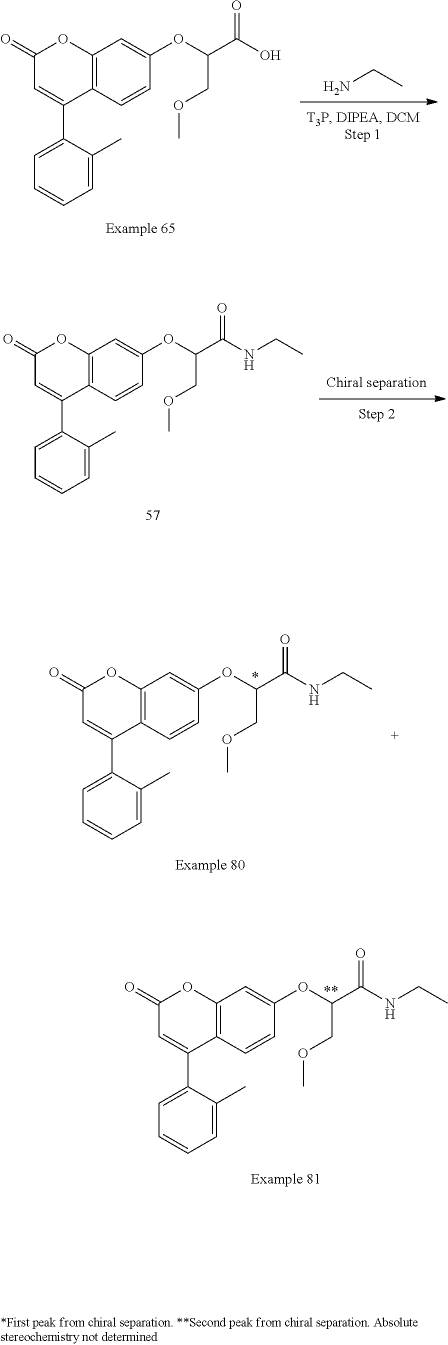

Synthesis of N-ethyl-3-methoxy-2-((2-oxo-4-(o-tolyl)-2H-chromen-7-yl)oxy)propenamide, 57 [Step 1]: To a stirred solution of 3-methoxy-2-[4-(o-tolyl)-2-oxo-2H-chromen-7-yl]oxy-propanoic acid (Example 65, 150 mg, 0.4 mmol) and ethylamine (29 mg, 0.6 mmol) in DCM (4 mL) was added DIPEA (0.2 mL, 1.3 mmol) followed by T₃P, 50% in EtOAc (0.2 mL, 0.6 mmol) at 0° C., and the reaction mixture was stirred at ambient temperature for 2 h. The reaction mixture was diluted with DCM, washed with water, brine, dried over anhydrous Na₂SO₄, and concentrated under reduced pressure. The product was purified by flash column chromatography to afford N-ethyl-3-methoxy-2-((2-oxo-4-(o-tolyl)-2H-chromen-7-yl)oxy)propanamide (57, 120 mg).

Synthesis of chiral N-ethyl-3-methoxy-2-((2-oxo-4-(o-tolyl)-2H-chromen-7-yl)oxy)propanamide, Examples 80 and 81 [Step 2]: N-ethyl-3-methoxy-2-((2-oxo-4-(o-tolyl)-2H-chromen-7-yl)oxy)propanamide (57, 120 mg) was purified by chiral prep-HPLC separation and the first product was isolated as N-ethyl-3-methoxy-2-((2-oxo-4-(o-tolyl)-2H-chromen-7-yl)oxy)propanamide, Peak 1 (Example 80, 48 mg) and the second product as N-ethyl-3-methoxy-2-((2-oxo-4-(o-tolyl)-2H-chromen-7-yl)oxy)propanamide, Peak 2 (Example 81, 36 mg). The absolute stereochemistries of these compounds were not determined.

Example 80: [N-ethyl-3-methoxy-2-((2-oxo-4-(o-tolyl)-2H-chromen-7-yl)oxy)propanamide, Peak 1]: LCMS (ESI) Calcd. for C₂₂H₂₃NO₅: 381, found [M+H]⁺=382. ¹H NMR (400 MHz, DMSO-d₆) δ 8.25 (s, 1H), 7.43-7.33 (m, 3H), 7.24 (d, 1H), 7.04 (d, 1H), 6.93-6.87 (m, 2H), 6.21 (s, 1H), 4.94 (t, 1H), 3.76-3.67 (m, 2H), 3.32 (s, 3H), 3.12 (q, 2H), 2.11 (s, 3H), 1.01 (q, 3H).

Example 81: [N-ethyl-3-methoxy-2-((2-oxo-4-(o-tolyl)-2H-chromen-7-yl)oxy)propanamide, Peak 2]: LCMS (ESI) Calcd. for C₂₂H₂₃NO₅: 381, found [M+H]⁺=382. ¹H NMR (400 MHz, DMSO-d₆) δ 8.25 (s, 1H), 7.43-7.33 (m, 3H), 7.24 (d, 1H), 7.04 (d, 1H), 6.93-6.87 (m, 2H), 6.21 (s, 1H), 4.94 (t, 1H), 3.76-3.67 (m, 2H), 3.32 (s, 3H), 3.12 (q, 2H), 2.11 (s, 3H), 1.01 (q, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 0.1% isopropylamine in a mixture of 40% hexane, 30% of CH₂Cl₂ and 30% ethanol, held isocratic up to 12 min. with detection at 322 nm wavelength.

### Examples 82-83: 3-methoxy-2-((2-oxo-4-(o-tolyl)-2H-chromen-7-yl)oxy)-N-(2,2,2-trifluoroethyl)propanamide, racemic and purified chiral analogs

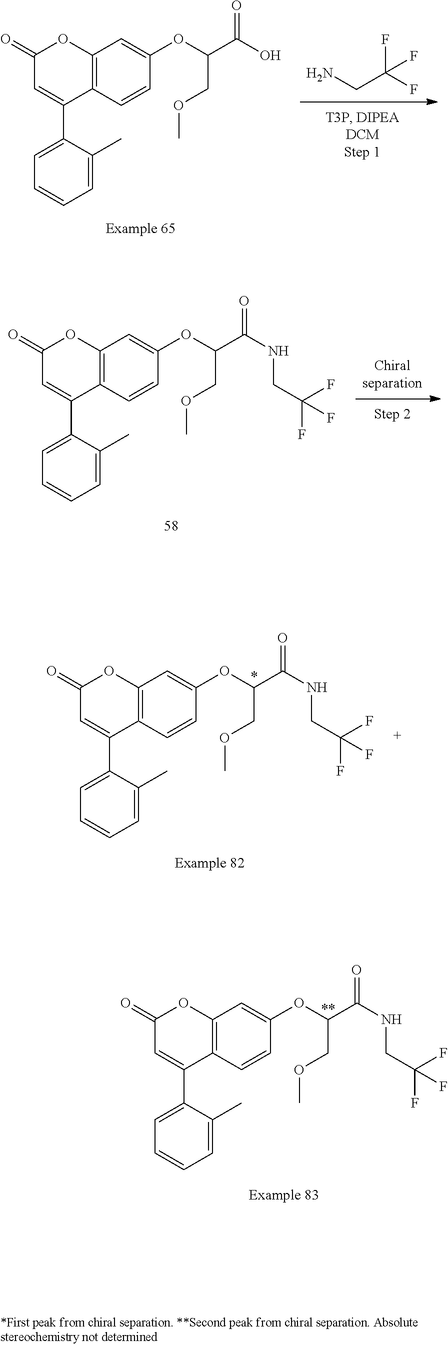 [SMILES: COCC(Oc1ccc2c(-c3ccccc3C)cc(=O)oc2c1)C(=O)O ; NCC(F)(F)F ; COCC(Oc1ccc2c(-c3ccccc3C)cc(=O)oc2c1)C(=O)NCC(F)(F)F ; COCC(Oc1ccc2c(-c3ccccc3C)cc(=O)oc2c1)C(=O)NCC(F)(F)F ; COCC(Oc1ccc2c(-c3ccccc3C)cc(=O)oc2c1)C(=O)NCC(F)(F)F]

Synthesis of 3-methoxy-2-((2-oxo-4-(o-tolyl)-2H-chromen-7-yl)oxy)-N-(2,2,2 trifluoroethyl) propenamide, 58 [Step 1]: To a stirred solution of 3-methoxy-2-[4-(o-tolyl)-2-oxo-2H-chromen-7-yl]oxy-propanoic acid (Example 65, 150 mg, 0.4 mmol) and 2,2,2-trifluoroethan-1-amine (63 mg, 0.6 mmol) in DCM (4 mL) was added DIPEA (0.3 mL, 1.3 mmol) followed by T₃P, 50% in EtOAc (0.2 mL, 0.6 mmol) at 0° C., and the reaction mixture was stirred at ambient temperature for 2 h. The reaction mixture was diluted with DCM, washed with water, brine, dried over anhydrous Na₂SO₄, and concentrated under reduced pressure. The product was purified by flash chromatography to afford 3-methoxy-2-((2-oxo-4-(o-tolyl)-2H-chromen-7-yl)oxy)-N-(2,2,2-trifluoroethyl)propanamide (58, 120 mg).

Synthesis of chiral 3-methoxy-2-((2-oxo-4-(o-tolyl)-2H-chromen-7-yl)oxy)-N-(2,2,2-trifluoroethyl)propanamide, Examples 82 and 83 [Step 2]: 3-methoxy-2-((2-oxo-4-(o-tolyl)-2H-chromen-7-yl)oxy)-N-(2,2,2-trifluoroethyl)propanamide (58, 120 mg) was purified by chiral prep-HPLC separation and the first product was isolated as 3-methoxy-2-((2-oxo-4-(o-tolyl)-2H-chromen-7-yl)oxy)-N-(2,2,2-trifluoroethyl)propanamide, Peak 1 (Example 82, 40 mg) and the second product as 3-methoxy-2-((2-oxo-4-(o-tolyl)-2H-chromen-7-yl)oxy)-N-(2,2,2-trifluoroethyl)propanamide, Peak 2 (Example 83, 40 mg). The absolute stereochemistries of these compounds were not determined.

Example 82: [3-methoxy-2-((2-oxo-4-(o-tolyl)-2H-chromen-7-yl)oxy)-N-(2,2,2-trifluoroethyl)propanamide, Peak 1]: LCMS (ESI) Calcd. for C₂₂H₂₀F₃NO₅: 435, found [M+H]⁺=436. ¹H NMR (400 MHz, DMSO-d₆) (at 100° C.) δ 8.57 (br s, 1H), 7.43-7.33 (m, 3H), 7.24 (d, 1H), 7.03 (s, 1H), 6.91 (s, 2H), 6.15 (s, 1H), 5.05 (s, 1H), 3.96-3.89 (m, 2H), 3.83-3.75 (m, 2H), 3.33 (s, 3H), 2.13 (s, 3H).

Example 83: [3-methoxy-2-((2-oxo-4-(o-tolyl)-2H-chromen-7-yl)oxy)-N-(2,2,2-trifluoroethyl)propanamide, Peak 2]: LCMS (ESI) Calcd. for C₂₂H₂₀F₃NO₅: 435, found [M+H]⁺=436. ¹H NMR (400 MHz, DMSO-d₆) (at 100° C.) δ 8.56 (br s, 1H), 7.45-7.33 (m, 3H), 7.24 (d, 1H), 7.03 (s, 1H), 6.91 (s, 2H), 6.15 (s, 1H), 5.05 (s, 1H), 3.96-3.89 (m, 2H), 3.80-3.75 (m, 2H), 3.33 (s, 3H), 2.13 (s, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: 0.1% isopropylamine in a mixture of 60% hexane, 20% of CH₂Cl₂ and 20% ethanol, held isocratic up to 24 min. with detection at 322 nm wavelength.

### Example 84: 2-((4-(2-chlorophenyl)-2-oxo-2H-chromen-7-yl)oxy)-3-methoxypropanoic acid

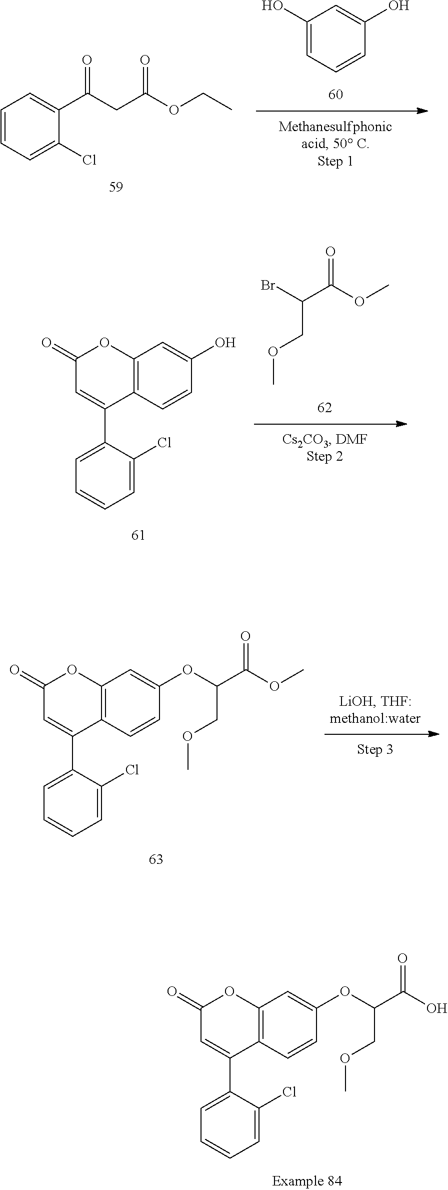

Synthesis of 4-(2-chlorophenyl)-7-hydroxy-2H-chromen-2-one, 61 [Step 1]: To a mixture of ethyl 3-(2-chlorophenyl)-3-oxo-propanoate (59, 2.0 g, 8.8 mmol) and benzene-1,3-diol (60, 1 g, 8.8 mmol) was added methanesulphonic acid (5 mL) and the reaction mixture was heated at 50° C. for 16 h. The reaction mixture was cooled and poured into ice-water and the precipitate was filtered and dried to yield 4-(2-chlorophenyl)-7-hydroxy-2H-chromen-2-one (61, 2.0 g). LCMS (ESI): Calcd. for C₁₅H₉ClO₃: 272, found [M+H]⁺=272.

Synthesis of methyl 2-((4-(2-chlorophenyl)-2-oxo-2H-chromen-7-yl)oxy)-3-methoxypropanoate, 63 [Step 2]: To a stirred solution of 4-(2-chlorophenyl)-7-hydroxy-2H-chromen-2-one (61, 1.0 g, 3.7 mmol) in DMF (5 mL) was added Cs₂CO₃ (2.4 g, 7.3 mmol) and the reaction mixture was stirred at ambient temperature for 15 min. To the reaction mixture was added methyl 2-bromo-3-methoxypropanoate (62, 0.5 mL, 4.0 mmol) and the reaction mixture was stirred at ambient temperature for 16 h. The reaction mixture was diluted with EtOAc and washed with water, brine, dried over anhydrous Na₂SO₄, filtered, and concentrated under reduced pressure to yield methyl 2-((4-(2-chlorophenyl)-2-oxo-2H-chromen-7-yl)oxy)-3-methoxypropanoate (63, 1.30 g). LCMS (ESI): Calcd. for C_{2N}H₁₇ClO₆: 388, found [M+H]⁺=389.

Synthesis of 2-((4-(2-chlorophenyl)-2-oxo-2H-chromen-7-yl)oxy)-3-methoxypropanoic acid, Example 84 [Step 3]: To an ice-cooled solution of methyl 2-((4-(2-chlorophenyl)-2-oxo-2H-chromen-7-yl)oxy)-3-methoxypropanoate (63, 150 mg, mmol) in a mixture of THF:methanol:water (5:3:1, 1 mL) was added LiOH·H₂O (15 mg, 0.6 mmol) and the reaction mixture was allowed to warm to ambient temperature and stirred for 16 h. The reaction mixture was concentrated under reduced pressure and the resulting material was dissolved in water, acidified with 1N HCl (pH=3), and extracted with EtOAc (3×30 mL). The organic phase was combined and washed with brine, dried over anhydrous Na₂SO₄, filtered, and concentrated under reduced pressure to afford the product, which was purified by RP prep-HPLC and lyophilized to yield 2-((4-(2-chlorophenyl)-2-oxo-2H-chromen-7-yl)oxy)-3-methoxypropanoic acid (Example 84, 80 mg). LCMS (ESI): Calcd. for C₁₉H₁₅ClO₆: 374, found [M+H]⁺=375. ¹H NMR (400 MHz, DMSO-d₆) at δ 7.66 (d, 1H), 7.59-7.47 (m, 3H), 7.21 (br s, 1H), 6.93 (br s, 1H), 6.88-6.83 (m, 2H), 6.26 (br s, 1H), 4.90 (br s, 1H), 3.76 (d, 2H), 3.29 (s, 3H).

### Examples 85-86: 2-((4-(2-chloro-4-fluorophenyl)-2-oxo-2H-chromen-7-yl)oxy)-3-methoxy-N,N-dimethylpropanamide, racemic and purified chiral analogs

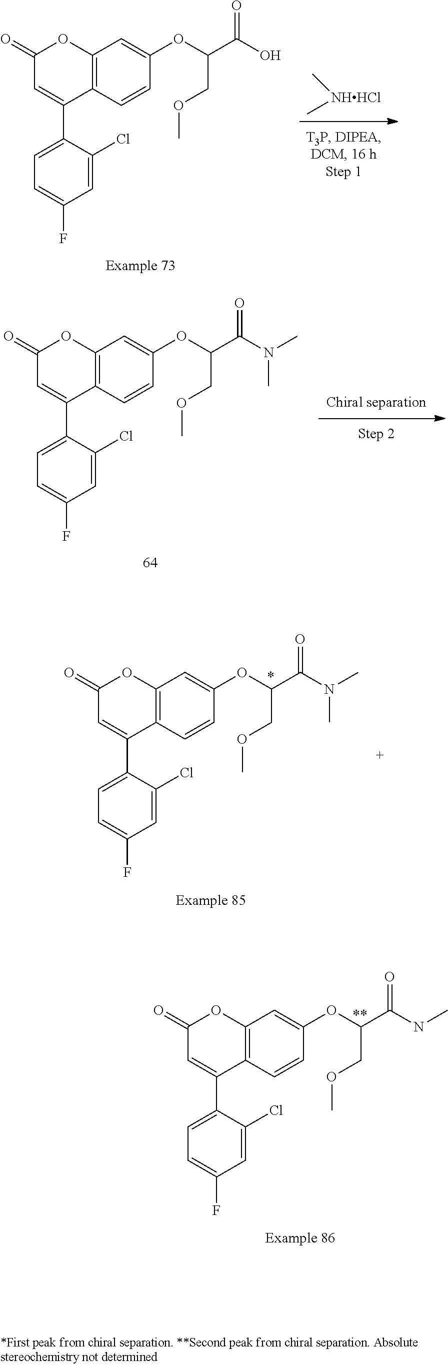

Synthesis of 2-((4-(2-chloro-4-fluorophenyl)-2-oxo-2H-chromen-7-yl)oxy)-3-methoxy-N,N-dimethylpropanamide, 64 [Step 1]: To a stirred solution of 2-[4-(4-chloro-2-fluoro-phenyl)-2-oxo-2H-chromen-7-yl]oxy-3-methoxy-propanoic acid (Example 73, 261 mg, 0.7 mmol) in DCM (5 mL) was added DIPEA (0.7 mL, 4 mmol), N-methylmethanamine hydrochloride (270 mg, 3.3 mmol) and T₃P (0.6 mL, 1 mmol), and the reaction mixture was stirred at ambient temperature for 16 h. The reaction mixture was diluted with DCM and washed with water, brine, dried over anhydrous Na₂SO₄, and concentrated under reduced pressure. The resulting product was purified by flash chromatography to afford 2-[4-(4-chloro-2-fluoro-phenyl)-2-oxo-2H-chromen-7-yl]oxy-3-methoxy-N,N-dimethyl-propanamide (64, 150 mg). LCMS Calcd. for C₂₁H₁₉ClFNO₅: 419, found [M+H]⁺=420.

Synthesis of chiral 2-((4-(2-chloro-4-fluorophenyl)-2-oxo-2H-chromen-7-yl)oxy)-3-methoxy-N,N-dimethylpropanamide, Examples 85 and 86 [Step 2]: 2-((4-(2-chloro-4-fluorophenyl)-2-oxo-2H-chromen-7-yl)oxy)-3-methoxy-N,N-dimethylpropanamide (64, 150 mg) was purified by chiral prep-HPLC separation and the first product was isolated as 2-((4-(2-chloro-4-fluorophenyl)-2-oxo-2H-chromen-7-yl)oxy)-3-methoxy-N,N-dimethylpropanamide, Peak 1 (Example 85, 65 mg) and the second product as 2-((4-(2-chloro-4-fluorophenyl)-2-oxo-2H-chromen-7-yl)oxy)-3-methoxy-N,N-dimethylpropanamide, Peak 2 (Example 86, 70 mg). The absolute stereochemistries of these compounds were not determined.

Example 85: [2-((4-(2-chloro-4-fluorophenyl)-2-oxo-2H-chromen-7-yl)oxy)-3-methoxy-N,N-dimethylpropanamide, Peak 1]: LCMS (ESI) Calcd. for C₂₁H₁₉ClFNO₅: 419, found [M+H]⁺=420. ¹H NMR (400 MHz, DMSO-d₆) δ 7.71-7.69 (m, 1H), 7.58-7.53 (m, 1H), 7.45-7.41 (m, 1H), 6.94-6.92 (m, 2H), 6.86-6.82 (m, 1H), 6.31 (s, 1H), 5.54 (m, 1H), 3.76-3.72 (m, 2H), 3.33 (s, 3H), 3.13 (s, 3H), 2.84 (s, 3H).

Example 86: [2-((4-(2-chloro-4-fluorophenyl)-2-oxo-2H-chromen-7-yl)oxy)-3-methoxy-N,N-dimethylpropanamide, Peak 2]: LCMS (ESI) Calcd. for C₂₁H₁₉ClFNO₅: 419, found [M+H]⁺=420. ¹H NMR (400 MHz, DMSO-d₆) δ 7.71-7.69 (m, 1H), 7.58-7.53 (m, 1H), 7.45-7.41 (m, 1H), 6.94-6.92 (m, 2H), 6.88-6.82 (m, 1H), 6.32 (s, 1H), 5.54 (m, 1H), 3.73 (m, 2H), 3.33 (s, 3H), 3.13 (s, 3H), 2.84 (s, 3H).

Chiral prep-HPLC: Chiral separation was performed on an Agilent 1200 series instrument. Column: Chiralpak IC (250×21 mm), 5p, operating at ambient temperature with flow rate of 21.0 mL/min. Mobile phase: mixture of 65% hexane, 17.5% of CH₂Cl₂ and 17.5% ethanol, held isocratic up to 30 min. with detection at 325 nm wavelength.

### Examples 87-88: 3-methoxy-N-(2-methoxyethyl)-2-((2-oxo-4-(o-tolyl)-2H-chromen-7-yl)oxy)propanamide, racemic and purified chiral analogs

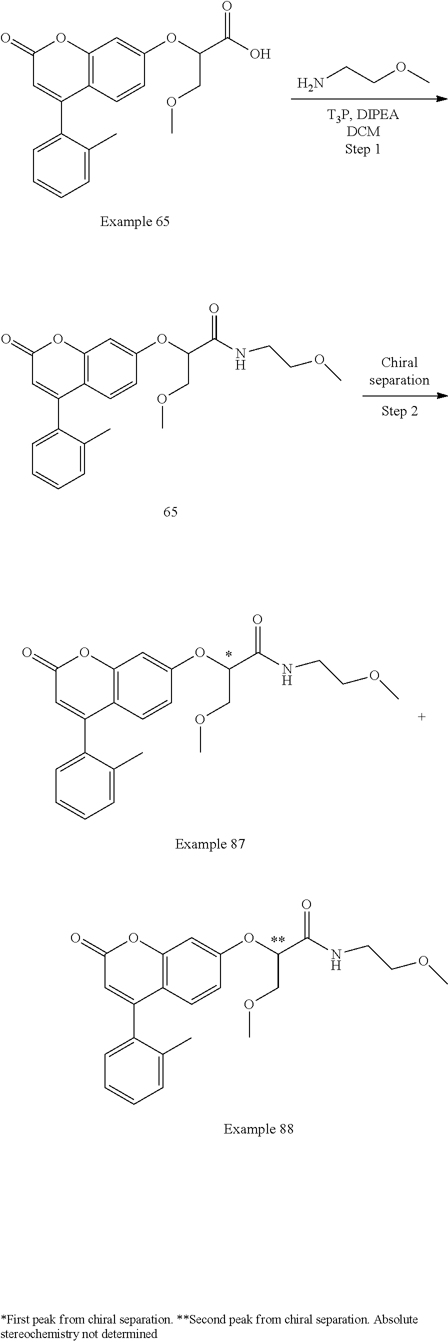

Synthesis of 3-methoxy-N-(2-methoxyethyl)-2-((2-oxo-4-(o-tolyl)-2H-chromen-7-yl)oxy)propenamide, 65 [Step 1]: To a stirred solution of 3-methoxy-2-[4-(o-tolyl)-2-oxo-2H-chromen-7-yl]oxy-propanoic acid (Example 65, 130 mg, 0.4 mmol) and 2-methoxyethylamine (0.06 mL, 0.7 mmol) in DCM (3 mL) was added DIPEA (0.3 mL, 1.5 mmol) followed by T₃P (50% in EtOAc) (0.2 mL, 0.6 mmol) at 0° C. and the reaction mixture was stirred at ambient temperature for 4 h. The reaction mixture was diluted with DCM and washed with water. The organic layer was collected, washed with brine, dried over anhydrous Na₂SO₄, and concentrated under reduced pressure. The product was purified by flash column chromatography to afford 3-methoxy-N-(2-methoxyethyl)-2-((2-oxo-4-(o-tolyl)-2H-chromen-7-yl)oxy)propanamide (65, 120 mg).

Synthesis of chiral 3-methoxy-N-(2-methoxyethyl)-2-((2-oxo-4-(o-tolyl)-2H-chromen-7-yl)oxy)propanamide, Examples 87 and 88 [Step 2]: 3-methoxy-N-(2-methoxyethyl)-2-((2-oxo-4-(o-tolyl)-2H-chromen-7-yl)oxy)propanamide (65, 120 mg) was purified by chiral prep-HPLC separation and the first product was isolated as 3-methoxy-N-(2-methoxyethyl)-2-((2-oxo-4-(o-tolyl)-2H-chromen-7-yl)oxy)propanamide, Peak 1 (Example 87, 60 mg) and the second product as 3-methoxy-N-(2-methoxyethyl)-2-((2-oxo-4-(o-tolyl)-2H-chromen-7-yl)oxy)propanamide, Peak 2 (Example 88, 55 mg). The absolute stereochemistries of these compounds were not determined.

Example 87: [3-methoxy-N-(2-methoxyethyl)-2-((2-oxo-4-(o-tolyl)-2H-chromen-7-yl)oxy)propanamide, Peak 1]: LCMS (ESI) Calcd. for C₂₃H₂₅NO₆: 411, found [M+H]⁺=412. ¹H NMR (400 MHz, DMSO-d₆) δ 8.34 (t, 1H), 7.43-7.33 (m, 3H), 7.24 (d, 1H), 7.04-7.02 (m, 1H), 6.92-6.86 (m, 2H), 6.20 (s, 1H), 4.99 (s, 1H), 3.76-3.71 (m, 1H), 3.69-3.66 (m, 1H), 3.32 (s, 1H), 3.29 (s, 4H), 3.27-3.24 (m, 2H), 3.19 (s, 3H), 2.10 (s, 3H).

Example 88: [3-methoxy-N-(2-methoxyethyl)-2-((2-oxo-4-(o-tolyl)-2H-chromen-7-yl)oxy)propanamide, Peak 2]: LCMS (ESI) Calcd. for C₂₃H₂₅NO₆: 411, found [M+H]⁺=412. ¹H NMR (400 MHz, DMSO-d₆) δ 8.34 (t, 1H), 7.43-7.33 (m, 3H), 7.24 (d, 1H), 7.04-7.02 (m, 1H), 6.92-6.86 (m, 2H), 6.20 (s, 1H), 4.99 (s, 1H), 3.76-3.71 (m, 1H), 3.69-3.66 (m, 1H), 3.32 (s, 1H), 3.29 (s, 4H), 3.27-3.24 (m, 2H), 3.19 (s, 3H), 2.10 (s, 3H).

Chiral prep-HPLC: Chiral separation was performed on an Agilent 1200 series instrument. Column: Chiralpak IC (250×20 mm), 5p, operating at ambient temperature with flow rate of 18.0 mL/min. Mobile phase: mixture of 60% hexane, 20% of CH₂Cl₂ and 20% ethanol, held isocratic up to 25 min. with detection at 332 nm wavelength.

### Examples 89-90: 7-((1-(azetidin-1-yl)-3-methoxy-1-oxopropan-2-yl)oxy)-4-(o-tolyl)-2H-chromen-2-one, racemic and purified chiral analogs

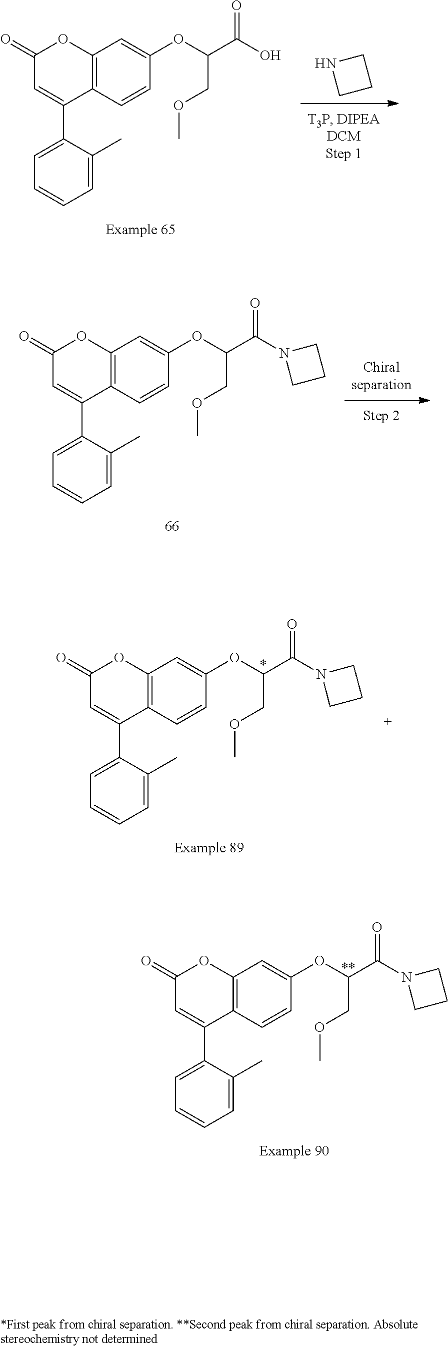

Synthesis of 7-((1-(azetidin-1-yl)-3-methoxy-1-oxopropan-2-yl)oxy)-4-(o-tolyl)-2H-chromen-2-one, 66 [Step 1]: To a stirred solution of 3-methoxy-2-[4-(o-tolyl)-2-oxo-2H-chromen-7-yl]oxy-propanoic acid (Example 65, 150 mg, 0.4 mmol) and azetidine hydrochloride (59 mg, 0.7 mmol) in DCM (4 mL) was added DIPEA (0.3 mL, 1.7 mmol) followed by T₃P, 50% in EtOAc (0.2 mL, 0.7 mmol) and the reaction mixture was stirred at ambient temperature for 4 h. The reaction mixture was diluted with DCM and washed with water. The organic layer was collected, washed with brine, dried over anhydrous Na₂SO₄, and concentrated under reduced pressure. The product was purified by flash column chromatography to afford 7-((1-(azetidin-1-yl)-3-methoxy-1-oxopropan-2-yl)oxy)-4-(o-tolyl)-2H-chromen-2-one (66, 120 mg).

Synthesis of chiral 7-((1-(azetidin-1-yl)-3-methoxy-1-oxopropan-2-yl)oxy)-4-(o-tolyl)-2H-chromen-2-one, Examples 89 and 90 [Step 2]: 7-((1-(azetidin-1-yl)-3-methoxy-1-oxopropan-2-yl)oxy)-4-(o-tolyl)-2H-chromen-2-one (66, 120 mg) was purified by chiral prep-HPLC separation and the first product was isolated as 7-((1-(azetidin-1-yl)-3-methoxy-1-oxopropan-2-yl)oxy)-4-(o-tolyl)-2H-chromen-2-one, Peak 1 (Example 89, 30 mg) and the second product as 7-((1-(azetidin-1-yl)-3-methoxy-1-oxopropan-2-yl)oxy)-4-(o-tolyl)-2H-chromen-2-one, Peak 2 (Example 90, 22 mg). The absolute stereochemistries of these compounds were not determined.

Example 89: [7-((1-(azetidin-1-yl)-3-methoxy-1-oxopropan-2-yl)oxy)-4-(o-tolyl)-2H-chromen-2-one, Peak 1]: LCMS (ESI) Calcd. for C₂₃H₂₃NO₅: 393, found [M+H]⁺=394. ¹H NMR (400 MHz, DMSO-d₆): δ 7.46-7.36 (m, 3H), 7.28 (d, 1H), 7.02-7.00 (m, 1H), 6.94-6.89 (m, 2H), 6.24 (s, 1H), 5.10-5.08 (m, 1H), 4.49 (t, 1H), 4.16 (t, 1H), 3.95 (d, 1H), 3.87 (d, 1H), 3.79-3.71 (m, 2H), 3.34 (s, 3H), 2.25-2.18 (m, 2H), 2.15 (s, 3H).

Example 90: [7-((1-(azetidin-1-yl)-3-methoxy-1-oxopropan-2-yl)oxy)-4-(o-tolyl)-2H-chromen-2-one, Peak 2]: LCMS (ESI) Calcd. for C₂₃H₂₃NO₅: 393, found [M+H]⁺=394. ¹H NMR (400 MHz, DMSO-d₆): δ 7.46-7.36 (m, 3H), 7.28 (d, 1H), 7.02-7.00 (m, 1H), 6.94-6.89 (m, 2H), 6.24 (s, 1H), 5.10-5.08 (m, 1H), 4.49 (t, 1H), 4.16 (t, 1H), 3.95 (d, 1H), 3.87 (d, 1H), 3.79-3.71 (m, 2H), 3.34 (s, 3H), 2.25-2.18 (m, 2H), 2.15 (s, 3H).

Chiral prep-HPLC: Chiral separation was performed on an Agilent 1200 series instrument. Column: Chiralpak IC (250×20 mm), 5p, operating at ambient temperature with flow rate of 18.0 mL/min. Mobile phase: 0.1% isopropylamine in a mixture of 65% hexane, 17.5% of CH₂Cl₂ and 17.5% ethanol, held isocratic up to 40 min. with detection at 324 nm wavelength.

### Examples 91-92: 2-((4-(2-chlorophenyl)-2-oxo-2H-chromen-7-yl)oxy)-3-methoxy-N-methylpropanamide, racemic and purified chiral analogs

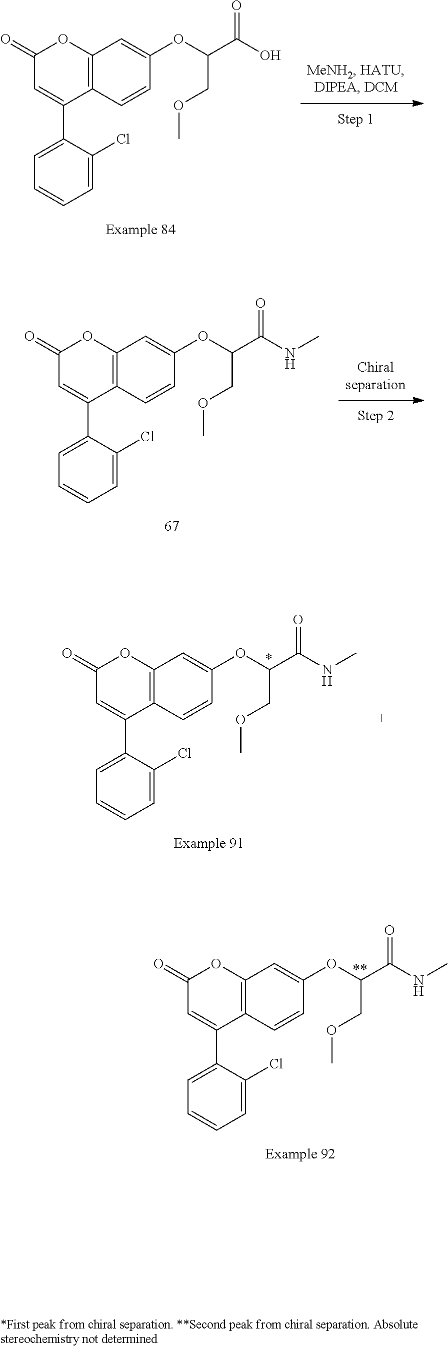 [SMILES: COCC(Oc1ccc2c(-c3ccccc3Cl)cc(=O)oc2c1)C(=O)O ; CNC(=O)C(COC)Oc1ccc2c(-c3ccccc3Cl)cc(=O)oc2c1 ; CNC(=O)C(COC)Oc1ccc2c(-c3ccccc3Cl)cc(=O)oc2c1 ; CNC(=O)C(COC)Oc1ccc2c(-c3ccccc3Cl)cc(=O)oc2c1]

Synthesis of 2-((4-(2-chlorophenyl)-2-oxo-2H-chromen-7-yl)oxy)-3-methoxy-N-methylpropanamide, 67 [Step 1]: To a stirred solution of 2-((4-(2-chlorophenyl)-2-oxo-2H-chromen-7-yl)oxy)-3-methoxypropanoic acid (Example 84, 300 mg, 0.8 mmol) in DCM (10 mL), was added HATU (610 mg, 1.6 mmol) and N,N-diisopropylethylamine (0.7 mL, 4.0 mmol) at 0° C. and the reaction mixture was stirred for 30 min. Methanamine (25 mg, 0.8 mmol) was added and the reaction mixture was stirred at ambient temperature for 16 h. The reaction mixture was diluted with EtOAc and washed with 5% aq. Na₂CO₃, water, brine, dried over anhydrous Na₂SO₄, filtered, and concentrated under reduced pressure. The product was purified by RP prep-HPLC and lyophilized to yield 2-((4-(2-chlorophenyl)-2-oxo-2H-chromen-7-yl)oxy)-3-methoxy-N-methylpropanamide (67, 200 mg). LCMS (ESI): Calcd. for C₂₀H₁₈ClNO₅: 387, found [M+H]⁺=387.

Synthesis of chiral 2-((4-(2-chlorophenyl)-2-oxo-2H-chromen-7-yl)oxy)-3-methoxy-N-methylpropanamide, Examples 91 and 92 [Step 2]: 2-((4-(2-chlorophenyl)-2-oxo-2H-chromen-7-yl)oxy)-3-methoxy-N-methylpropanamide (67, 200 mg) was purified by chiral prep-HPLC separation and the first product was isolated as 2-((4-(2-chlorophenyl)-2-oxo-2H-chromen-7-yl)oxy)-3-methoxy-N-methylpropanamide, Peak 1 (Example 91, 92 mg) and the second product as 2-((4-(2-chlorophenyl)-2-oxo-2H-chromen-7-yl)oxy)-3-methoxy-N-methylpropanamide, Peak 2 (Example 92, 90 mg). The absolute stereochemistries of these compounds were not determined.

Example 91: [2-((4-(2-chlorophenyl)-2-oxo-2H-chromen-7-yl)oxy)-3-methoxy-N-methylpropanamide, Peak 1]: LCMS (ESI): Calcd. for C₂₀H₁₈ClNO₅: 387, found [M+H]⁺=388. ¹H NMR (400 MHz, DMSO-d₆) δ 8.16 (t, 1H), 7.67 (d, 1H), 7.61-7.47 (m, 3H), 7.59 (q, 1H), 6.96-6.90 (m, 2H), 6.32 (s, 1H), 4.98 (br s, 1H), 3.77-3.68 (m, 2H), 3.28 (s, 3H), 2.62 (d, 3H).

Example 92: [2-((4-(2-chlorophenyl)-2-oxo-2H-chromen-7-yl)oxy)-3-methoxy-N-methylpropanamide, Peak 2: LCMS (ESI): Calcd. for C₂₀H₁₈ClNO₅: 387, found [M+H]⁺=388. ¹H NMR (400 MHz, DMSO-d₆) δ 8.16 (t, 1H), 7.67 (d, 1H), 7.61-7.47 (m, 3H), 7.59 (q, 1H), 6.96-6.90 (m, 2H), 6.32 (s, 1H), 4.98 (br s, 1H), 3.77-3.68 (m, 2H), 3.28 (s, 3H), 2.62 (d, 3H).

Chiral prep-HPLC: Chiral separation was performed on an Agilent 1200 series instrument. Column: (R,R) WHELK-01 (250×21.1 mm), 5p, operating at 35° C. with flow rate of 60 g/min. Mobile phase: 65% CO₂+35% (0.3% Ipamine in MeOH) at 100 bar with detection at 320 nm wavelength.

Examples 93-94: 2-((4-(2-chlorophenyl)-2-oxo-2H-chromen-7-yl)oxy)-3-methoxy-N,N-dimethylpropanamide, racemic and purified chiral analogs

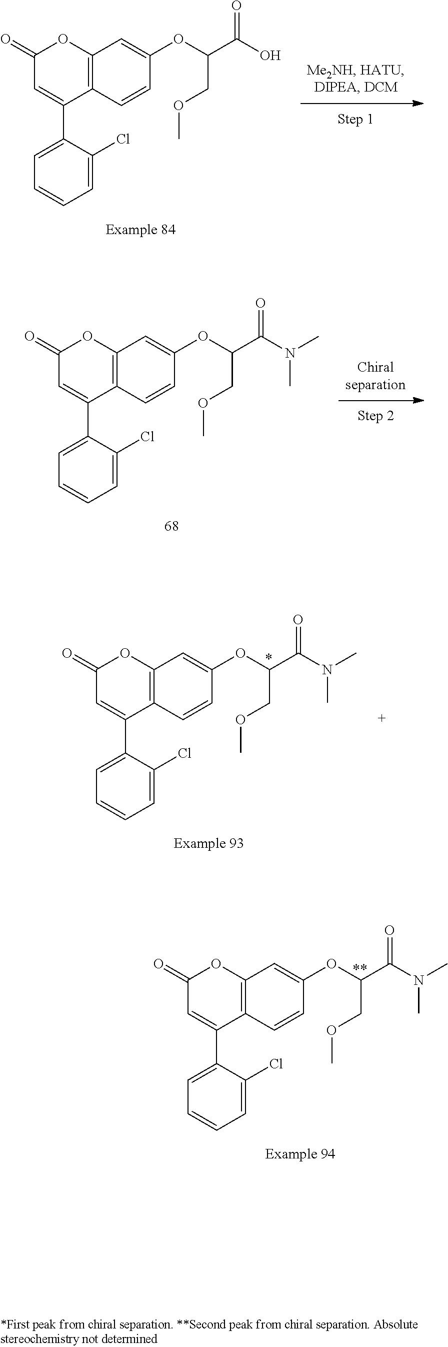 [SMILES: COCC(Oc1ccc2c(-c3ccccc3Cl)cc(=O)oc2c1)C(=O)O ; COCC(Oc1ccc2c(-c3ccccc3Cl)cc(=O)oc2c1)C(=O)N(C)C ; COCC(Oc1ccc2c(-c3ccccc3Cl)cc(=O)oc2c1)C(=O)N(C)C ; COCC(Oc1ccc2c(-c3ccccc3Cl)cc(=O)oc2c1)C(=O)N(C)C]

Synthesis of 2-((4-(2-chlorophenyl)-2-oxo-2H-chromen-7-yl)oxy)-3-methoxy-N,N-dimethylpropanamide, 68 [Step 1]: To a stirred solution of 2-((4-(2-chlorophenyl)-2-oxo-2H-chromen-7-yl)oxy)-3-methoxypropanoic acid (Example 84, 300 mg, 0.8 mmol) in DCM (10 mL) was added HATU (610 mg, 1.6 mmol) and N,N-diisopropylethylamine (0.7 mL, 4.0 mmol) at 0° C. and the reaction mixture was stirred for 30 min. To the reaction mixture was added N-methylmethanamine (1M in THF) (0.8 mL, mmol) and the reaction mixture was stirred at ambient temperature for 16 h. The reaction mixture was diluted with EtOAc and washed with 5% aq. Na₂CO₃, water, brine, dried over anhydrous Na₂SO₄, filtered, and concentrated under reduced pressure. The product was purified by RP prep-HPLC chromatography and lyophilized to yield 2-((4-(2-chlorophenyl)-2-oxo-2H-chromen-7-yl)oxy)-3-methoxy-N,N-dimethylpropanamide (68, 200 mg). LCMS Calcd. for C₂₁H₂₀ClNO₅: 402, found [M+H]⁺=403.

Synthesis of chiral 2-((4-(2-chlorophenyl)-2-oxo-2H-chromen-7-yl)oxy)-3-methoxy-N,N-dimethylpropanamide, Examples 93 and 94 [Step 2]: 2-((4-(2-chlorophenyl)-2-oxo-2H-chromen-7-yl)oxy)-3-methoxy-N,N-dimethylpropanamide (68, 200 mg) was purified by chiral prep-HPLC separation and the first product was isolated as 2-((4-(2-chlorophenyl)-2-oxo-2H-chromen-7-yl)oxy)-3-methoxy-N,N-dimethylpropanamide, Peak 1 (Example 93, 60 mg) and the second product as 2-((4-(2-chlorophenyl)-2-oxo-2H-chromen-7-yl)oxy)-3-methoxy-N,N-dimethylpropanamide, Peak 2 (Example 94, 72 mg). The absolute stereochemistries of these compounds were not determined.

Example 93: [2-((4-(2-chlorophenyl)-2-oxo-2H-chromen-7-yl)oxy)-3-methoxy-N,N-dimethylpropanamide, Peak 1]: LCMS (ESI): Calcd. for C₂₁H₂₀ClNO₅: 401, found [M+H]⁺=402. ¹H NMR (400 MHz, DMSO-d₆) δ 7.67 (d, 1H), 7.60-7.47 (m, 3H), 6.94-6.85 (m, 3H), 6.31 (s, 1H), 5.53 (q, 1H), 3.73 (d, 2H), 3.33 (s, 3H), 3.13 (s, 3H), 2.84 (s, 3H).

Example 94: [2-((4-(2-chlorophenyl)-2-oxo-2H-chromen-7-yl)oxy)-3-methoxy-N,N-dimethylpropanamide, Peak 2]: LCMS (ESI): Calcd. for C₂₁H₂₀ClNO₅: 402, found [M+H]⁺=402. ¹H NMR (400 MHz, DMSO-d₆) δ 7.67 (d, 1H), 7.60-7.47 (m, 3H), 6.94-6.85 (m, 3H), 6.31 (s, 1H), 5.53 (q, 1H), 3.73 (d, 2H), 3.33 (s, 3H), 3.13 (s, 3H), 2.84 (s, 3H).

Chiral prep-HPLC: Chiral separation was performed on an Agilent 1200 series instrument. Column: (R,R) WHELK-01 (250×21.1 mm), 5p, operating at 35° C. with flow rate of 60 g/min. Mobile phase: 65% CO₂+35% (0.3% isopropylamine in MeOH) at 100 bar with detection at 320 nm wavelength.

### Examples 95-96: 2-((4-(2-chlorophenyl)-2-oxo-2H-chromen-7-yl)oxy)-3-methoxypropanamide, racemic and purified chiral analogs

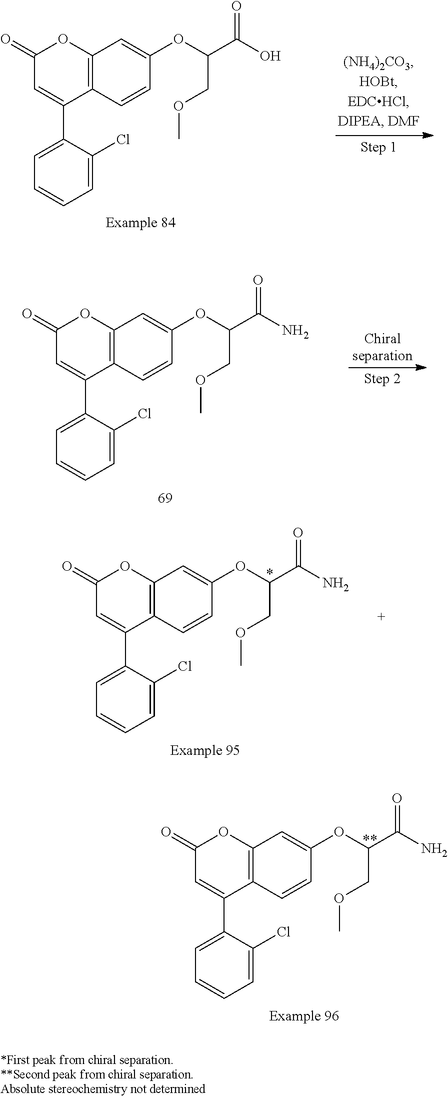 [SMILES: COCC(Oc1ccc2c(-c3ccccc3Cl)cc(=O)oc2c1)C(=O)O ; COCC(Oc1ccc2c(-c3ccccc3Cl)cc(=O)oc2c1)C(N)=O ; COCC(Oc1ccc2c(-c3ccccc3Cl)cc(=O)oc2c1)C(N)=O ; COCC(Oc1ccc2c(-c3ccccc3Cl)cc(=O)oc2c1)C(N)=O]

Synthesis of 2-((4-(2-chlorophenyl)-2-oxo-2H-chromen-7-yl)oxy)-3-methoxypropanamide, 69 [Step 1]: To a stirred solution of 2-((4-(2-chlorophenyl)-2-oxo-2H-chromen-7-yl)oxy)-3-methoxypropanoic acid (Example 84, 300 mg, 0.8 mmol) in DMF (3 mL) was added HOBt (160 mg, 1.2 mmol), EDC HCl (230 mg, 1.2 mmol), DIPEA (0.4 mL, 3.2 mmol), and (NH₄)₂CO₃ (310 mg, 3.2 mmol) and the reaction mixture was stirred at ambient temperature for 16 h. The reaction mixture was diluted with water and extracted with EtOAc. The organic layer was washed with brine, dried over anhydrous Na₂SO₄, filtered, and concentrated under reduced pressure. The product was purified by RP prep-HPLC and lyophilized to yield 2-((4-(2-chlorophenyl)-2-oxo-2H-chromen-7-yl)oxy)-3-methoxypropanamide (69, 200 mg). LCMS (ESI): Calcd. for C₁₉H₁₆ClNO₅: 374, found [M+H]⁺=374.

Synthesis of chiral 2-((4-(2-chlorophenyl)-2-oxo-2H-chromen-7-yl)oxy)-3-methoxypropanamide, Examples 95 and 96 [Step 2]: 2-((4-(2-chlorophenyl)-2-oxo-2H-chromen-7-yl)oxy)-3-methoxypropanamide (69, 160 mg) was purified by chiral prep-HPLC separation and the first product was isolated as 2-((4-(2-chlorophenyl)-2-oxo-2H-chromen-7-yl)oxy)-3-methoxypropanamide, Peak 1 (Example 95, 60 mg) and the second product as 2-((4-(2-chlorophenyl)-2-oxo-2H-chromen-7-yl)oxy)-3-methoxypropanamide, Peak 2 (Example 96, 55 mg). The absolute stereochemistries of these compounds were not determined.

Example 95: [2-((4-(2-chlorophenyl)-2-oxo-2H-chromen-7-yl)oxy)-3-methoxypropanamide, Peak 1]: LCMS (ESI): Calcd. for C₁₉H₁₆ClNO₅: 374, found [M+H]⁺=374. ¹H NMR (400 MHz, DMSO-d₆) δ 7.67 (d, 2H), 7.61-7.46 (m, 4H), 7.05-7.04 (m, 1H), 6.96-6.90 (m, 2H), 6.32 (s, 1H), 4.95-4.92 (m, 1H), 3.78-3.69 (m, 2H), 3.31 (d, 3H).

Example 96: [2-((4-(2-chlorophenyl)-2-oxo-2H-chromen-7-yl)oxy)-3-methoxypropanamide, Peak 2]: LCMS (ESI): Calcd. for C₁₉H₁₆ClNO₅: 374, found [M+H]⁺=374. ¹H NMR (400 MHz, DMSO-d₆) δ 7.67 (d, 2H), 7.61-7.46 (m, 4H), 7.05-7.04 (m, 1H), 6.96-6.90 (m, 2H), 6.32 (s, 1H), 4.95-4.92 (m, 1H), 3.78-3.69 (m, 2H), 3.31 (d, 3H).

### Examples 97-98: 3-hydroxy-N-methyl-2-((2-oxo-4-(o-tolyl)-2H-chromen-7-yl)oxy)propenamide, racemic and chiral forms

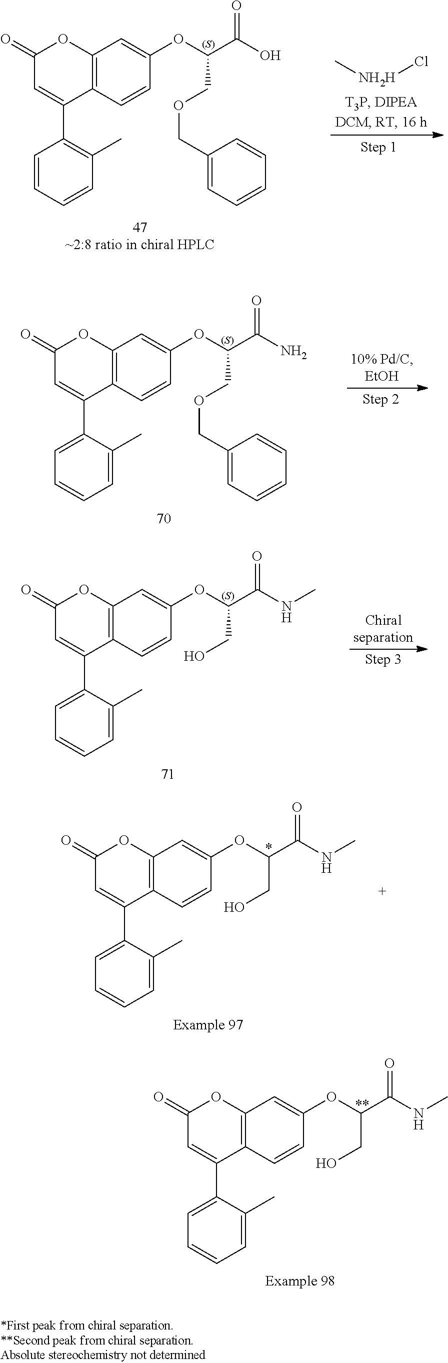

Synthesis (S)-3-(benzyloxy)-N-methyl-2-((2-oxo-4-(o-tolyl)-2H-chromen-7-yl)oxy)propenamide, 70 [Step 1]: To a stirred solution of (S)-3-(benzyloxy)-2-((2-oxo-4-(o-tolyl)-2H-chromen-7-yl)oxy)propanoic acid (47, 180 mg, 0.4 mmol) in DCM (7 mL) was added DIPEA (0.46 mL, 3.3 mmol) and methanamine hydrochloride (198 mg, 2.9 mmol), and the reaction mixture was cooled to 0° C. T₃P (50% in EtOAc, 0.4 mL, 0.6 mmol) was added, and the reaction mixture was allowed to warm to room temperature and stirred for 16 h. The reaction mixture was diluted with DCM, washed with water, brine, dried over anhydrous Na₂SO₄, and concentrated to afford (S)-3-(benzyloxy)-N-methyl-2-((2-oxo-4-(o-tolyl)-2H-chromen-7-yl)oxy)propanamide (70, 130 mg). LCMS (ESI): Calcd. for C₂₇H₂₅NO₅: 443, found: [M+H]⁺=444.

Synthesis of (S)-3-hydroxy-N-methyl-2-((2-oxo-4-(o-tolyl)-2H-chromen-7-yl)oxy)propenamide, 71 [Step 2]: A stirred solution of (S)-3-(benzyloxy)-N-methyl-2-((2-oxo-4-(o-tolyl)-2H-chromen-7-yl)oxy)propanamide (70, 130 mg, 0.3 mmol) in ethanol (5 mL) was purged with argon for 5 min. To the mixture was added Pd/C (10 mg, 0.03 mmol) and the reaction mixture was subjected to hydrogenolysis for 3 h. The reaction mixture was filtered through a celite bed and the filtrate was concentrated to obtain the product that was purified by RP prep HPLC to afford (S)-3-hydroxy-N-methyl-2-((2-oxo-4-(o-tolyl)-2H-chromen-7-yl)oxy)propanamide (71, 70 mg). LCMS (ESI): Calcd. for C₂₀H₁₉NO₅: 353, found: [M+H]⁺=354.

Synthesis of chiral 3-hydroxy-N-methyl-2-((2-oxo-4-(o-tolyl)-2H-chromen-7-yl)oxy)propenamide, Examples 97 and 98 [Step 3]: (S)-3-hydroxy-N-methyl-2-((2-oxo-4-(o-tolyl)-2H-chromen-7-yl)oxy)propanamide (71, 70 mg, 0.2 mmol) was purified by chiral prep-HPLC separation and the first product isolated as 3-hydroxy-N-methyl-2-((2-oxo-4-(o-tolyl)-2H-chromen-7-yl)oxy)propenamide, Peak 1 (Example 97, 12 mg) and the second product as 3-hydroxy-N-methyl-2-((2-oxo-4-(o-tolyl)-2H-chromen-7-yl)oxy)propenamide, Peak 2 (Example 98, 28 mg). The absolute stereochemistries of these compounds were not determined.

Example 97: [3-hydroxy-N-methyl-2-[4-(o-tolyl)-2-oxo-chromen-7-yl]oxy-propanamide, Peak 1]: LCMS (ESI) Calcd. for C₂₀H₁₉NO₅: 353, found: [M+H]⁺=354. ¹H NMR (400 MHz, DMSO-d₆) δ 8.11 (m, 1H), 7.43-7.39 (m, 2H), 7.36-7.33 (m, 1H), 7.24 (d, 1H), 7.02 (m, 1H), 6.91-6.87 (m, 2H), 6.20 (s, 1H), 5.15-5.12 (m, 1H), 4.76 (m, 1H), 3.76 (m, 2H), 2.61 (d, 3H), 2.11 (s, 3H).

Example 98: [3-hydroxy-N-methyl-2-[4-(o-tolyl)-2-oxo-chromen-7-yl]oxy-propanamide, Peak 2]: LCMS (ESI) Calcd. for C₂₀H₁₉NO₅: 353, found: [M+H]⁺=354. ¹H NMR (400 MHz, DMSO-d₆) δ 8.11 (m, 1H), 7.43-7.40 (m, 2H), 7.38-7.35 (m, 1H), 7.24 (d, 1H), 7.02 (m, 1H), 6.91-6.87 (m, 2H), 6.20 (s, 1H), 5.15-5.12 (m, 1H), 4.76 (m, 1H), 3.76 (m, 2H), 2.61 (d, 3H), 2.11 (s, 3H).

Chiral prep-HPLC: Chiral separation was performed on an Agilent 1200 series instrument. Column: CHIRALPAK IC (250×20 mm), 5p, operating at ambient temperature with flow of 18.0 mL/min. Mobile phase: 65% hexane, 17.5% dichloromethane, and 17.5% ethanol with run time up to 20 min. and detection at 324 nm wavelength.

### Examples 99-100: 3-ethoxy-2-((2-oxo-4-(o-tolyl)-2H-chromen-7-yl)oxy)propenamide, racemic and purified chiral analogs

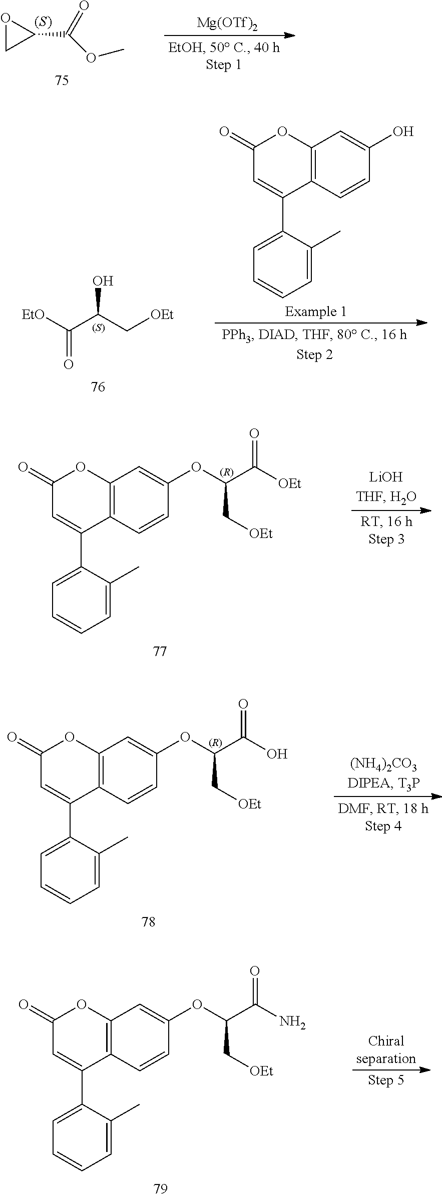

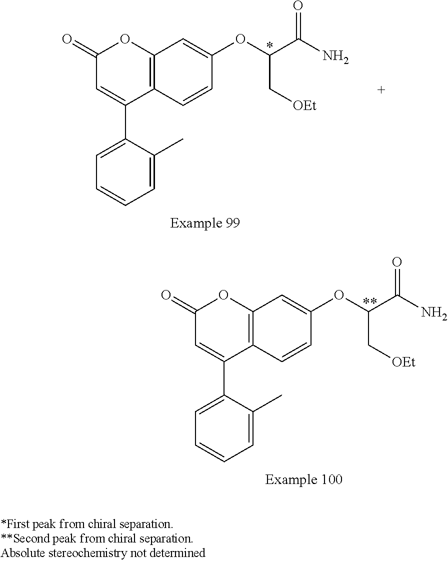 [SMILES: CCOCC(Oc1ccc2c(-c3ccccc3C)cc(=O)oc2c1)C(N)=O ; CCOCC(Oc1ccc2c(-c3ccccc3C)cc(=O)oc2c1)C(N)=O]

Synthesis of ethyl (S)-3-ethoxy-2-hydroxypropanoate, 76 [Step 1]: To a stirred solution of methyl (S)-oxirane-2-carboxylate (75, 2.0 g, 19.6 mmol) in ethanol (10 mL) was added magnesium triflate (1.6 g, 4.9 mmol) at ambient temperature. The reaction mixture was stirred at 50° C. for 40 h., and then concentrated under reduced pressure and diluted with ethyl acetate. The organic layer was washed with brine, dried over anhydrous Na₂SO₄, filtered, and the filtrate was concentrated under reduced pressure. The product was purified by column chromatography to afford ethyl (S)-3-ethoxy-2-hydroxypropanoate (76, 950 mg). ¹H NMR (400 MHz, CDCl₃) δ 4.28-4.20 (m, 3H), 3.73-3.67 (m, 2H), 3.59-3.47 (m, 2H), 3.01 (d, 1H), 1.29 (t, 3H), 1.17 (t, 3H).

Synthesis of ethyl (R)-3-ethoxy-2-((2-oxo-4-(o-tolyl)-2H-chromen-7-yl)oxy)propanoate, 77 [Step 2]: To a stirred solution of 7-hydroxy-4-(o-tolyl)-2H-chromen-2-one (76, 0.5 g, 2.0 mmol) and ethyl (S)-3-ethoxy-2-hydroxypropanoate (0.5 g, 3.0 mmol) in THF was added PPh₃ (1.6 g, 6.0 mmol). The reaction mixture was cooled to 0° C. and DIAD (1.2 mL, 6.0 mmol) was added. The reaction mixture was stirred for 5 min., and then gradually heated to 80° C. and stirred at this temperature for 16 h. The reaction mixture was diluted with ethyl acetate, washed with water, brine, dried over anhydrous Na₂SO₄, and concentrated under reduced pressure. The product was purified by column chromatography to afford ethyl (R)-3-ethoxy-2-((2-oxo-4-(o-tolyl)-2H-chromen-7-yl)oxy)propanoate (77, 350 mg). LCMS (ESI) Calcd. for C₂₃H₂₄O₆=396, found [M+H]⁺=397.

Synthesis of (R)-3-ethoxy-2-((2-oxo-4-(o-tolyl)-2H-chromen-7-yl)oxy)propanoic acid, 78 [Step 3]: To a stirred solution of ethyl (R)-3-ethoxy-2-((2-oxo-4-(o-tolyl)-2H-chromen-7-yl)oxy)propanoate (77, 350 mg, 0.9 mmol) in THF (6 mL) and water (1.5 mL), was added LiOH·H₂O (111 mg, 2.7 mmol). The reaction mixture was stirred for 16 h. at ambient temperature, and concentrated under reduced pressure to give (R)-3-ethoxy-2-((2-oxo-4-(o-tolyl)-2H-chromen-7-yl)oxy)propanoic acid (78, 300 mg). The product was carried forward without further purification. LCMS (ESI) Calcd. for C₂₁H₂₀O₆=368, found [M+H]⁺=369.

Synthesis of (R)-3-ethoxy-2-((2-oxo-4-(o-tolyl)-2H-chromen-7-yl)oxy)propenamide, 79 [Step 4]: To a stirred solution of (R)-3-ethoxy-2-((2-oxo-4-(o-tolyl)-2H-chromen-7-yl)oxy)propanoic acid (78, 200 mg, 0.6 mmol) and ammonium carbonate (380 mg, 4.0 mmol) in DMF was added N,N-diisopropylethylamine (0.8 mL, 4.5 mmol) at 0° C. under an argon atmosphere. The reaction was stirred for 10 min., followed by the dropwise addition of T₃P in EtOAc (50%, 0.3 mL, 0.8 mmol). The reaction mixture was then warmed to ambient temperature and stirred for 18 h. The product was diluted with DCM, washed with water, and concentrated under reduced pressure. The product was purified by RP prep HPLC to afford (R)-3-ethoxy-2-((2-oxo-4-(o-tolyl)-2H-chromen-7-yl)oxy)propanamide (79, 50 mg).

Synthesis of chiral 3-ethoxy-2-((2-oxo-4-(o-tolyl)-2H-chromen-7-yl)oxy)propenamide, Examples 99 and 100 [Step 5]: 3-ethoxy-2-((2-oxo-4-(o-tolyl)-2H-chromen-7-yl)oxy)propanamide (50 mg) was purified by chiral prep-HPLC and the first product isolated as 3-ethoxy-2-((2-oxo-4-(o-tolyl)-2H-chromen-7-yl)oxy)propenamide, Peak 1 (Example 99, 7 mg) and the second product as 3-ethoxy-2-((2-oxo-4-(o-tolyl)-2H-chromen-7-yl)oxy)propenamide, Peak 2 (Example 100, 32 mg). The absolute stereochemistries of these compounds were not determined.

Example 99: [3-ethoxy-2-((2-oxo-4-(o-tolyl)-2H-chromen-7-yl)oxy)propenamide, Peak 1]: LCMS (ESI) Calcd. for C₂₁H₂₁NO₅=367, found [M+H]⁺=368. ¹H NMR (400 MHz, DMSO-d₆) δ 7.67 (s, 1H), 7.45-7.39 (m, 3H), 7.35 (t, 1H), 7.24 (d, 1H), 7.03 (t, 1H), 6.94-6.87 (m, 2H), 6.20 (s, 1H), 4.90 (s, 1H), 3.77 (d, 2H), 3.52 (q, 2H), 2.12 (s, 3H), 1.09 (t, 3H).

Example 100: [3-ethoxy-2-((2-oxo-4-(o-tolyl)-2H-chromen-7-yl)oxy)propenamide, Peak 2]: LCMS (ESI) Calcd. for C₂₁H₂₁NO₅=367, found [M+H]⁺=368. ¹H NMR (400 MHz, DMSO-d₆) δ 7.67 (s, 1H), 7.45-7.39 (m, 3H), 7.35 (t, 1H), 7.23 (d, 1H), 7.03 (s, 1H), 6.94-6.87 (m, 2H), 6.20 (s, 1H), 4.88 (t, 1H), 3.80 (d, 2H), 3.54 (q, 2H), 2.14 (s, 3H), 1.09 (t, 3H).

Chiral prep-HPLC: Chiral separation was performed on an Agilent 1200 series instrument. Column: CHIRALPAK IC (250×20 mm), 5p, operating at ambient temperature with flow of 18.0 mL/min. Mobile phase: 60% hexane, 20% dichloromethane, and 20% ethanol with run time up to 18 min. and detection at 322 nm wavelength.

### Examples 101: (R)-3-methoxy-2-((2-oxo-4-(o-tolyl)-2H-chromen-7-yl)oxy)propanoic acid

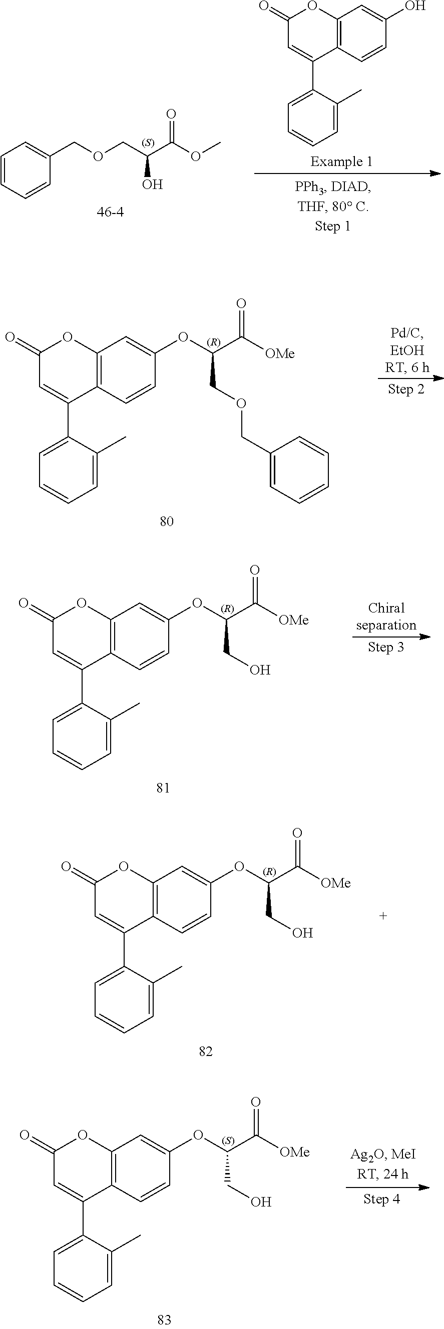

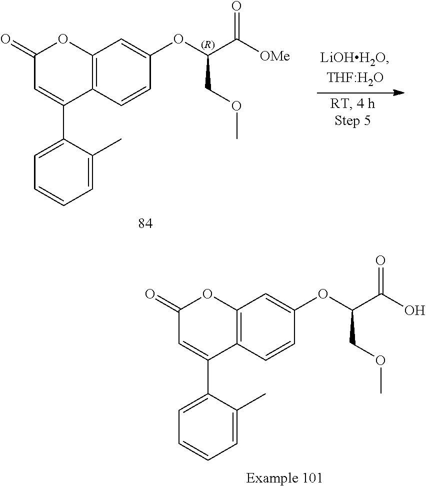

Synthesis of methyl (R)-3-(benzyloxy)-2-((2-oxo-4-(o-tolyl)-2H-chromen-7-yl)oxy)propanoate, 80 [Step 1]: To a stirred solution of methyl (S)-3-(benzyloxy)-2-hydroxypropanoate (46-4, 1.83 g, 8.7 mmol) in THF (10 mL) was added 7-hydroxy-4-(o-tolyl)-2H-chromen-2-one (Example 1, 1.00 g, 4 mmol) and PPh₃ (3.12 g, 11.9 mmol). To the reaction mixture was slowly added DIAD (2.4 mL, 11.9 mmol) at 0° C., and the reaction mixture was stirred for 10 min. before it was gradually heated to 80° C. and stirred for 16 h. The reaction mixture was diluted with ethyl acetate, washed with water, brine, and dried over anhydrous Na₂SO₄. The mixture was concentrated under reduced pressure to get the product that was purified by combi-flash chromatography to afford methyl (R)-3-(benzyloxy)-2-((2-oxo-4-(o-tolyl)-2H-chromen-7-yl)oxy)propanoate (80, 1.20 g). LCMS (ESI) Calcd. for C₂₇H₂₄O₆: 444, found [M+H]⁺=445.

Synthesis of methyl (R)-3-hydroxy-2-((2-oxo-4-(o-tolyl)-2H-chromen-7-yl)oxy)propanoate, 81 [Step 2]: A stirred solution of methyl (R)-3-(benzyloxy)-2-((2-oxo-4-(o-tolyl)-2H-chromen-7-yl)oxy)propanoate (80, 700 mg, 1.6 mmol) in ethanol (7 mL) was degassed for 10 min., followed by the addition of Pd-C (17 mg, 0.2 mmol), and the reaction mixture was subjected to hydrogenolysis for 2 h. The reaction mixture was filtered through a celite bed and the filtrate was concentrated under reduced pressure to obtain the product, which was purified by combi-flash chromatography to afford methyl (R)-3-hydroxy-2-((2-oxo-4-(o-tolyl)-2H-chromen-7-yl)oxy)propanoate (81, 200 mg) as a liquid. LCMS (ESI) Calcd. for C₂₀H₁₈O₆: 354, found: [M+H]⁺=355. ¹H NMR (400 MHz, DMSO-d₆) δ 7.41-7.33 (m, 3H), 7.25-7.23 (m, 1H), 7.03 (s, 1H), 6.88 (m, 2H), 6.20 (s, 1H), 5.33-5.20 (m, 1H), 5.12 (m, 1H), 3.88-3.86 (m, 2H), 3.68 (s, 3H), 2.11 (s, 3H).

Synthesis of methyl (R)-3-hydroxy-2-((2-oxo-4-(o-tolyl)-2H-chromen-7-yl)oxy)propanoate, 82, and methyl (S)-3-hydroxy-2-((2-oxo-4-(o-tolyl)-2H-chromen-7-yl)oxy)propanoate, 83 [Step 3]: Methyl (R)-3-hydroxy-2-((2-oxo-4-(o-tolyl)-2H-chromen-7-yl)oxy)propanoate (81, 200 mg, 0.6 mmol) was subjected to normal phase chiral prep chromatography to afford methyl (R)-3-hydroxy-2-((2-oxo-4-(o-tolyl)-2H-chromen-7-yl)oxy)propanoate, 82, and methyl (S)-3-hydroxy-2-((2-oxo-4-(o-tolyl)-2H-chromen-7-yl)oxy)propanoate, 83. The major product was assumed to have the desired R-stereochemistry.

Methyl (R)-3-hydroxy-2-((2-oxo-4-(o-tolyl)-2H-chromen-7-yl)oxy)propanoate (82, 120 mg). LCMS (ESI) Calcd. for C₂₀H₁₈O₆: 354, found: [M+H]⁺=355. Chiral purity: 100% ee.

Methyl (S)-3-hydroxy-2-((2-oxo-4-(o-tolyl)-2H-chromen-7-yl)oxy)propanoate (83, 60 mg). LCMS (ESI) Calcd. for C₂₀H₁₈O₆: 354, found: [M+H]⁺=355. Chiral purity: 99% ee.

Synthesis of methyl (R)-3-methoxy-2-((2-oxo-4-(o-tolyl)-2H-chromen-7-yl)oxy)propanoate, 84 [Step 4]: A stirred solution of methyl (R)-3-hydroxy-2-((2-oxo-4-(o-tolyl)-2H-chromen-7-yl)oxy)propanoate (82, 120 mg, 0.3 mmol) in MeI (0.63 mL, 10.2 mmol) was cooled to 0° C., and Ag₂O (157 mg, 0.7 mmol) was added portion wise under an argon atmosphere. The reaction mixture was allowed to warm to ambient temperature and stirred for 12 h. The reaction mixture was filtered through a celite bed and the filtrate was evaporated under reduced pressure to afford methyl (R)-3-methoxy-2-((2-oxo-4-(o-tolyl)-2H-chromen-7-yl)oxy)propanoate (84, 110 mg). LCMS (ESI) Calcd. for C₂₁H₂₀O₆: 368, found: [M+H]⁺=369.

Synthesis of (R)-3-methoxy-2-((2-oxo-4-(o-tolyl)-2H-chromen-7-yl)oxy)propanoic acid, Example 101 [Step 5]: To a stirred solution of methyl (R)-3-methoxy-2-((2-oxo-4-(o-tolyl)-2H-chromen-7-yl)oxy)propanoate (84, 110 mg, 0.3 mmol) in THF (4 mL) and water (1 mL) was added LiOH H₂O (50 mg, 1.2 mmol), and the reaction mixture was stirred at ambient temperature for 4 h. The reaction mixture was concentrated under reduced pressure and the product was dissolved in water and acidified with 1N HCl, and then extracted with EtOAc. The organic layer was washed with water, brine, dried over anhydrous Na₂SO₄, and concentrated under reduced pressure. The product was purified by RP prep-HPLC and lyophilized to afford (R)-3-methoxy-2-((2-oxo-4-(o-tolyl)-2H-chromen-7-yl)oxy)propanoic acid (Example 101, 69 mg). LCMS (ESI) Calcd. for C₂₀H₁₈O₆: 354, found [M+H]⁺=355. ¹H NMR (400 MHz, DMSO-d₆) δ 7.44-7.38 (m, 2H), 7.36-7.32 (m, 1H), 7.24-7.22 (d, 1H), 6.90 (m, 1H), 6.82 (m, 2H), 6.14 (s, 1H), 4.84 (m, 1H), 3.75-3.73 (d, 2H), 3.28 (s, 3H), 2.11 (s, 3H).

### Example 102-103: 3-hydroxy-2-((2-oxo-4-(o-tolyl)-2H-chromen-7-yl)oxy)propanoic acid, racemic and purified chiral analogs

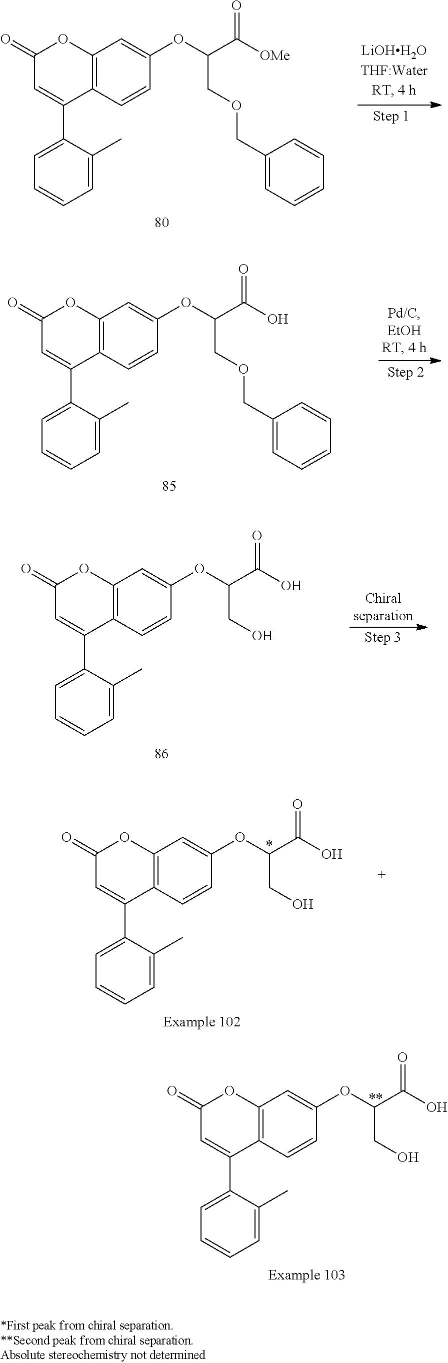 [SMILES: COC(=O)C(COCc1ccccc1)Oc1ccc2c(-c3ccccc3C)cc(=O)oc2c1 ; Cc1ccccc1-c1cc(=O)oc2cc(OC(COCc3ccccc3)C(=O)O)ccc12 ; Cc1ccccc1-c1cc(=O)oc2cc(OC(CO)C(=O)O)ccc12 ; Cc1ccccc1-c1cc(=O)oc2cc(OC(CO)C(=O)O)ccc12 ; Cc1ccccc1-c1cc(=O)oc2cc(OC(CO)C(=O)O)ccc12]

Synthesis of (R)-3-(benzyloxy)-2-((2-oxo-4-(o-tolyl)-2H-chromen-7-yl)oxy)propanoic acid, 85 [Step 1]: To a stirred solution of methyl (R)-3-(benzyloxy)-2-((2-oxo-4-(o-tolyl)-2H-chromen-7-yl)oxy)propanoate (80, 500 mg, 1.1 mmol) in THF (4 mL) and water (1 mL), was added LiOH H₂O (190 mg, 4.5 mmol) at ambient temperature. The reaction mixture was allowed to stir for 4 h., and then concentrated under reduced pressure. The product was diluted with EtOAc, washed with water, brine, dried over Na₂SO₄, and concentrated under reduced pressure to afford (R)-3-(benzyloxy)-2-((2-oxo-4-(o-tolyl)-2H-chromen-7-yl)oxy)propanoic acid (85, 450 mg). LCMS (ESI) Calcd. for C₂₆H₂₂O₆: 430, found [M+H]⁺=431.

Synthesis of (R)-3-hydroxy-2-((2-oxo-4-(o-tolyl)-2H-chromen-7-yl)oxy)propanoic acid, 86 [Step 2]: A stirred solution of (R)-3-(benzyloxy)-2-((2-oxo-4-(o-tolyl)-2H-chromen-7-yl)oxy)propanoic acid (85, 300 mg, 0.7 mmol) in ethanol (7 mL) was degassed with argon for 10 min. To the reaction mixture was added Pd-C 10% (50 mg) and the reaction mixture was subjected to hydrogenolysis at ambient temperature for 2 h. The reaction mixture was filtered through a celite bed and the filtrate was concentrated to obtain the product that was purified by RP prep-HPLC and lyophilized to afford (R)-3-hydroxy-2-((2-oxo-4-(o-tolyl)-2H-chromen-7-yl)oxy)propanoic acid (86, 104 mg). LCMS (ESI) Calcd. for C₁₉H₁₆O₆: 340 found: [M+H]⁺=341.

Synthesis of chiral 3-hydroxy-2-((2-oxo-4-(o-tolyl)-2H-chromen-7-yl)oxy)propanoic acid, Example 102 and 103 [Step 3]: 3-hydroxy-2-((2-oxo-4-(o-tolyl)-2H-chromen-7-yl)oxy)propanoic acid (86, 104 mg) was separated by normal phase chiral prep-HPLC to afford 3-hydroxy-2-((2-oxo-4-(o-tolyl)-2H-chromen-7-yl)oxy)propanoic acid (Example 102, 45 mg) and 3-hydroxy-2-((2-oxo-4-(o-tolyl)-2H-chromen-7-yl)oxy)propanoic acid (Example 103, 9.0 mg). The absolute stereochemistries of these compounds were not determined.

Example 102: 3-hydroxy-2-((2-oxo-4-(o-tolyl)-2H-chromen-7-yl)oxy)propanoic acid, Peak 1: LCMS (ESI) Calcd. for C₁₉H₁₆O₆: 340, found: [M−H]⁻: 339. ¹H NMR (400 MHz, DMSO-d₆) δ 13.18 (br s, 1H), 7.45-7.33 (m, 3H), 7.25 (d, 1H), 6.98 (s, 1H), 6.88 (s, 2H), 6.19 (s, 1H), 4.98 (br s, 1H), 3.86 (br s, 2H), 2.11 (s, 3H).

Example 103: (S)-3-hydroxy-2-((2-oxo-4-(o-tolyl)-2H-chromen-7-yl)oxy)propanoic acid, Peak 2: LCMS (ESI) Calcd. for C₁₉H₁₆O₆: 340, found [M−H]⁻: 339. ¹H NMR (400 MHz, DMSO-d₆) δ 13.18 (br s, 1H), 7.45-7.33 (m, 3H), 7.25 (d, 1H), 6.98 (s, 1H), 6.88 (s, 2H), 6.19 (s, 1H), 4.98 (br s, 1H), 3.86 (br s, 2H), 2.11 (s, 3H).

Chiral prep-HPLC: Chiral separation was performed on an Agilent 1200 series instrument. Column: CHIRALPAK IG (250×21 mm), 5p, operating at ambient temperature with flow of 21.0 mL/min. Mobile phase: 70% hexane, 15% dichloromethane, 15% ethanol, and 0.1% trifluoroacetic acid, with a run time up to 28 min. and detection at 324 nm wavelength.

### Example 104: (S)-3-methoxy-2-((2-oxo-4-(o-tolyl)-2H-chromen-7-yl)oxy)propanoic acid

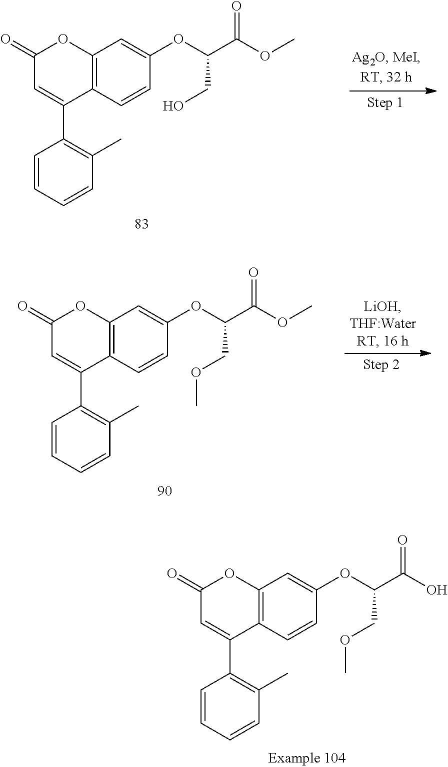 [SMILES: COC(=O)[C@H](CO)Oc1ccc2c(-c3ccccc3C)cc(=O)oc2c1 ; COC[C@H](Oc1ccc2c(-c3ccccc3C)cc(=O)oc2c1)C(=O)OC ; COC[C@H](Oc1ccc2c(-c3ccccc3C)cc(=O)oc2c1)C(=O)O]

Synthesis of methyl (S)-3-methoxy-2-((2-oxo-4-(o-tolyl)-2H-chromen-7-yl)oxy)propanoate, 90 [Step 1]: To a stirred solution of methyl (S)-3-hydroxy-2-((2-oxo-4-(o-tolyl)-2H-chromen-7-yl)oxy)propanoate (83, 60 mg, 0.2 mmol) in methyl iodide (0.3 mL, 5.1 mmol) was added Ag₂O (78 mg, 0.4 mmol), and the reaction mixture stirred at ambient temperature for 32 h. The reaction mixture was filtered through a celite bed and the filtrate was concentrated under reduced pressure to afford the product that was purified by flash chromatography to afford methyl (S)-3-methoxy-2-((2-oxo-4-(o-tolyl)-2H-chromen-7-yl)oxy)propanoate (90, 60 mg). LCMS (ESI) Calcd. for C₂₁H₂₀O₆: 368, found [M+H]⁺=369.

Synthesis of (S)-3-methoxy-2-((2-oxo-4-(o-tolyl)-2H-chromen-7-yl)oxy)propanoic acid, Example 104 [Step 2]: To a stirred solution of methyl (S)-3-methoxy-2-((2-oxo-4-(o-tolyl)-2H-chromen-7-yl)oxy)propanoate (90, 60 mg, 0.2 mmol) in THF (3 mL) was dropwise added an aqueous solution of LiOH H₂O (21 mg, 0.5 mmol, 0.75 mL). The reaction mixture was stirred for 2 h. at ambient temperature and concentrated under reduced pressure to afford the product that was diluted with water, acidified with citric acid, and extracted with EtOAc. The organic layer was washed with brine, dried over anhydrous Na₂SO₄, concentrated under reduced pressure and purified by RP prep-HPLC to afford (S)-3-methoxy-2-((2-oxo-4-(o-tolyl)-2H-chromen-7-yl)oxy)propanoic acid (Example 104, 20 mg). LCMS (ESI) Calcd. for C₂₀H₁₈O₆: 354, found [M+H]⁺=355. ¹H NMR (400 MHz, DMSO-d₆) δ 13.48 (br s, 1H), 7.44-7.32 (m, 3H), 7.24-7.22 (d, 1H), 6.97 (s, 1H), 6.85 (m, 2H), 6.17 (s, 1H), 5.05 (m, 1H), 3.80-3.75 (m, 2H), 3.34 (s, 3H), 2.11 (s, 3H).

### Examples 105-106: Methyl 2-((2-oxo-4-(o-tolyl)-2H-chromen-7-yl)oxy)-3-(trifluoromethoxy)propanoate, 2-((2-oxo-4-(o-tolyl)-2H-chromen-7-yl)oxy)-3-(trifluoromethoxy)propanoic acid, ammonia salt

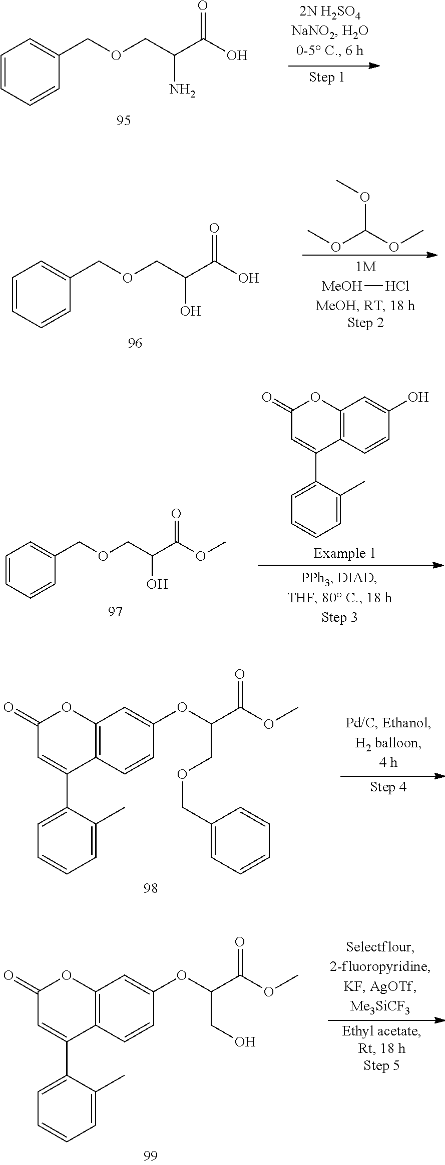

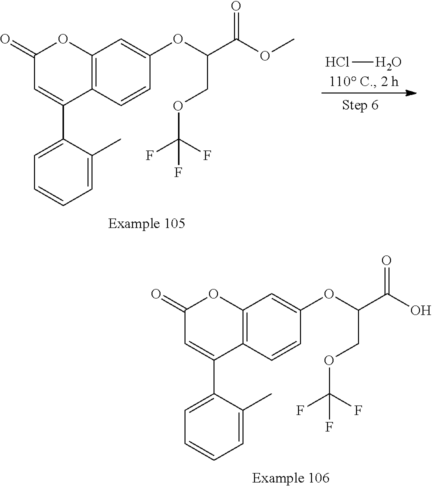

Synthesis of 3-(benzyloxy)-2-hydroxypropanoic acid, 96 [Step 1]: A stirred solution of O-benzylserine (95, 10.0 g, 51.2 mmol) in 2N H₂SO₄ (50 mL) was cooled to 0° C. Then a solution of NaNO₂ (6.4 g, 92.2 mmol) in water (10 mL) was added to the mixture over 1.5 h. at a temperature of 0-5° C. The reaction mixture was stirred at 5° C. for 6 h., and gradually warmed to ambient temperature and stirred for another 6 h. The reaction mixture was adjusted to pH 4 with 50% NaOH solution at 0° C. Ethyl acetate (300 mL) was added and the reaction was stirred vigorously and the aqueous layer was adjusted to pH 2 with the addition of 2N H₂SO₄. The aqueous layer was extracted with EtOAc and the combined organic layer was dried over anhydrous Na₂SO₄, filtered, and concentrated under reduced pressure to obtain 3-(benzyloxy)-2-hydroxypropanoic acid (96, 9.5 g). LCMS (ESI): Calcd. for C₁₀H₁₂O₄: 196, found [M−H]⁻=195.

Synthesis of methyl 3-(benzyloxy)-2-hydroxypropanoate, 97 [Step 2]: To a stirred mixture of 3-(benzyloxy)-2-hydroxypropanoic acid (96, 5.0 g, 25.5 mmol) in methanol (50 mL), was added freshly prepared methanolic HCl (25 mL, 1 M) at ambient temperature over 1.5 h. To the reaction mixture was added trimethyl orthoformate (17.2 g, 162 mmol) and the reaction mixture was stirred at ambient temperature for 18 h. The reaction mixture was concentrated under reduced pressure and purified by column chromatography to afford methyl 3-(benzyloxy)-2-hydroxypropanoate (97, 5.0 g). ¹H NMR (400 MHz, DMSO-d₆) δ 7.36-7.28 (m, 5H), 5.59 (br s, 1H), 4.54-4.46 (m, 2H), 4.25 (br s, 1H), 3.64-3.60 (m, 5H).

Synthesis of methyl 3-(benzyloxy)-2-((2-oxo-4-(o-tolyl)-2H-chromen-7-yl)oxy)propanoate, 98 [Step 3]: To a stirred solution of 7-hydroxy-4-(o-tolyl)-2H-chromen-2-one (Example 1, 1.0 g, 3.9 mmol) and methyl 3-(benzyloxy)-2-hydroxypropanoate (97, 1.25 g, 5.9 mmol) in THF (30 mL) was added PPh₃ (3.1 g, 11.9 mmol). The reaction mixture was cooled to 0° C. and DIAD (2.3 mL, 11.9 mmol) was added and the reaction mixture was stirred for 5 min., then gradually heated to 80° C. and stirred for 18 h. The reaction mixture was diluted with ethyl acetate, washed with water, brine, dried over anhydrous Na₂SO₄, and concentrated under reduced pressure. The product was purified by column chromatography to afford methyl 3-(benzyloxy)-2-((2-oxo-4-(o-tolyl)-2H-chromen-7-yl)oxy)propanoate (98, 950 mg). LCMS (ESI) Calcd. for C₂₇H₂₄O₆: 444, found [M+H]⁺=445.

Synthesis of methyl 3-hydroxy-2-((2-oxo-4-(o-tolyl)-2H-chromen-7-yl)oxy)propanoate, 99 [Step 4]: A stirred solution of methyl 3-(benzyloxy)-2-((2-oxo-4-(o-tolyl)-2H-chromen-7-yl)oxy)propanoate, (98, 950 mg, 2.1 mmol) in ethanol (20 mL) was purged with argon for 5 min. To the mixture was added 10% Pd/C (200 mg, 0.2 mmol) and the mixture was subjected to hydrogenolysis at ambient temperature for 4 h. The reaction mixture was filtered through a celite bed and concentrated under reduced pressure, and the product was purified by column chromatography to afford methyl 3-hydroxy-2-((2-oxo-4-(o-tolyl)-2H-chromen-7-yl)oxy)propanoate (99, 400 mg). LCMS (ESI) Calcd. for C₂₀H₁₈O₆: 354, found [M+H]⁺=355.

Synthesis of methyl 2-((2-oxo-4-(o-tolyl)-2H-chromen-7-yl)oxy)-3-(trifluoromethoxy)propanoate, Example 105 [Step 5]: In a sealed vessel, dry potassium fluoride (490 mg, 8.5 mmol), silver trifluoromethanesulphonate (1.7 g, 6.8 mmol), 1-chloromethyl-4-fluoro-1,4-diazoniabicyclo[2.2.2]octane bis(tetrafluoroborate) (1.2 g, 3.4 mmol), and methyl 3-hydroxy-2-((2-oxo-4-(o-tolyl)-2H-chromen-7-yl)oxy)propanoate (99, 600 mg, 1.7 mmol) were added successively under an argon atmosphere. To the mixture was successively added dry ethyl acetate (10 mL), 2-fluoropyridine (0.6 mL, 6.8 mmol), and (trifluoromethyl)trimethylsilane (1.0 mL, 6.8 mmol) under argon and the reaction mixture was stirred at ambient temperature for 18 h. The reaction mixture was diluted with ethyl acetate and filtered through a celite bed and the filtrate was dried over anhydrous Na₂SO₄, and concentrated under reduced pressure. The product was purified by column chromatography to give methyl 2-((2-oxo-4-(o-tolyl)-2H-chromen-7-yl)oxy)-3-(trifluoromethoxy)propanoate (Example 105, 170 mg). LCMS (ESI) Calcd for C₂₁H₁₇F₃O₆: 422, found [M+H]⁺=423. ¹H NMR (400 MHz, DMSO-d₆) δ 7.46-7.39 (m, 2H), 7.35 (t, 1H), 7.25 (d, 1H), 7.18 (s, 1H), 6.95 (d, 1H), 6.90 (t, 1H), 6.23 (s, 1H), 5.61 (t, 1H), 4.58 (d, 2H), 3.75 (s, 3H), 2.15 (s, 3H).

Synthesis of 2-((2-oxo-4-(o-tolyl)-2H-chromen-7-yl)oxy)-3-(trifluoromethoxy)propanoic acid, ammonia salt, Example 106 [Step 6]: Methyl 2-((2-oxo-4-(o-tolyl)-2H-chromen-7-yl)oxy)-3-(trifluoromethoxy)propanoate (Example 105, 90 mg, 0.2 mmol), water (0.6 mL, 33.3 mmol), and 12N HCl (4.0 mL, 132 mmol) were added to a sealed tube. The reaction mixture was heated to 110° C. for 2 h, and then concentrated under reduced pressure. The product was purified by RP prep-HPLC to afford 2-((2-oxo-4-(o-tolyl)-2H-chromen-7-yl)oxy)-3-(trifluoromethoxy)propanoic acid, ammonia salt (Example 106, 30 mg). LCMS (ESI) Calcd. for C₂₀H₁₅F₃O₆: 408, found [M+H]⁺=409. ¹H NMR (400 MHz, DMSO-d₆) δ 7.45-7.38 (m, 2H), 7.35 (t, 1H), 7.23 (d, 1H), 7.14 (br s, 3H), 6.91 (s, 1H), 6.83 (s, 2H), 6.15 (s, 1H), 4.99-4.88 (m, 1H), 4.48 (d, 1H), 4.41-4.36 (m, 1H), 2.15 (s, 3H).

### Example 107: 2-((4-(2,6-dimethylphenyl)-2-oxo-2H-chromen-7-yl)oxy)acetonitrile

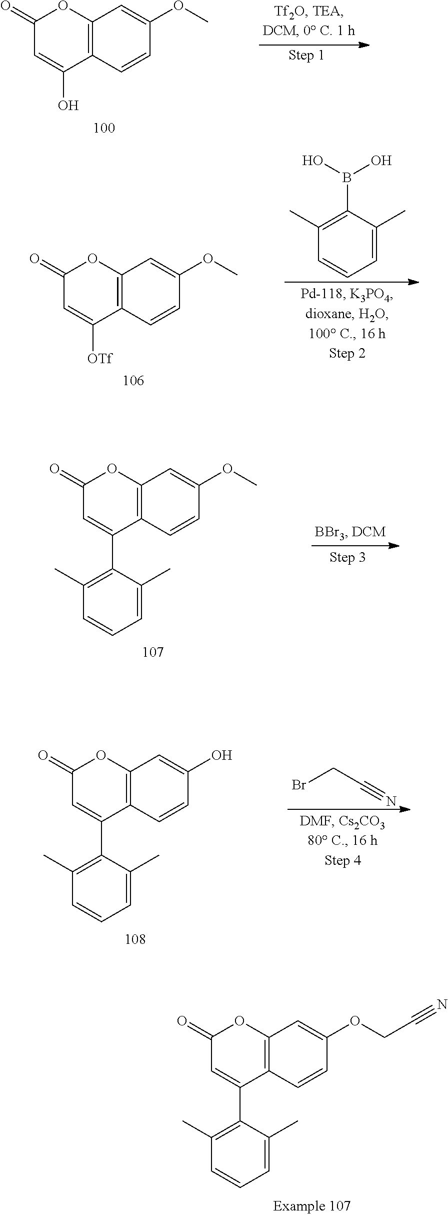

Synthesis of 7-methoxy-2-oxo-2H-chromen-4-yl trifluoromethanesulfonate, 106 [Step 1]: A solution of 4-hydroxy-7-methoxy-2H-chromen-2-one (100, 500 mg, 2.6 mmol) in DCM (3 mL) was cooled to 0° C. and triethylamine (0.7 mL, 5.2 mmol) and trifluoromethanesulfonic anhydride (0.7 mL, 3.9 mmol) were added at 0° C. The reaction mixture was allowed to warm to ambient temperature and stirred for another 3 h. The reaction mixture was quenched with water and extracted with DCM (3×50 mL). The organic layer was washed with water, brine, dried over anhydrous Na₂SO₄, and concentrated under reduced pressure. The product was purified by flash chromatography to afford 7-methoxy-2-oxo-2H-chromen-4-yl trifluoromethanesulfonate (106, 350 mg). LCMS (ESI) Calcd. for C₁₁H₇F₃O₆S: 324, found [M+H]⁺=325. ¹H NMR (400 MHz, DMSO-d₆) δ 7.61 (d, 1H), 7.19 (br s, 1H), 7.13-7.10 (m, 1H), 6.65 (s, 1H), 3.90 (s, 3H).

Synthesis of 4-(2,6-dimethylphenyl)-7-methoxy-2H-chromen-2-one, 107 [Step 2]: To a stirred solution of K₃PO₄ (420 mg, 2 mmol) and 7-methoxy-2-oxo-2H-chromen-4-yl trifluoromethanesulfonate (106, 100 mg, 0.3 mmol) in 1,4-dioxane-water (5 mL, 4:1) mixture was added (2,6-dimethylphenyl)boronic acid (70 mg, 0.5 mmol) and the reaction mixture was degassed with argon for 10 min. To the reaction mixture was added Pd-118 (20 mg, 0.03 mmol) and the reaction mixture was heated to reflux at 100° C. for 16 h. The reaction mixture was cooled, filtered through a celite bed, and the filtrate was evaporated under reduced pressure. The product was purified by flash column chromatography to afford 4-(2,6-dimethylphenyl)-7-methoxy-2H-chromen-2-one (107, 22 mg). LCMS (ESI) Calcd. for C₁₈H₁₆O₃: 280, found [M+H]⁺=281.

Synthesis of 4-(2,6-dimethylphenyl)-7-hydroxy-2H-chromen-2-one, 108 [Step 3]: To a stirred solution 4-(2,6-dimethylphenyl)-7-methoxy-2H-chromen-2-one (107, 105 mg, 0.4 mmol) in DCM (3 mL) was added BBr₃ (1.1 mL, 1.1 mmol, 1M in DCM,) dropwise at 0° C. The reaction mixture was allowed to warm to ambient temperature and stirred for 16 h. The mixture was concentrated under reduced pressure and quenched with methanol and ice cold water. The mixture was extracted with EtOAc (3×50 mL) and washed with brine, dried over anhydrous Na₂SO₄, and concentrated under reduced pressure. The product was purified by flash column chromatography to afford 4-(2,6-dimethylphenyl)-7-hydroxy-2H-chromen-2-one (108, 92 mg). LCMS (ESI) Calcd. for C₁₇H₁₄O₃: 266, found [M+H]⁺=267.

Synthesis of 2-((4-(2,6-dimethylphenyl)-2-oxo-2H-chromen-7-yl)oxy)acetonitrile, Example 107 [Step 4]: To a stirred solution of 2-bromoacetonitrile (55 mg, 0.5 mmol) and 4-(2,6-dimethylphenyl)-7-hydroxy-2H-chromen-2-one (108, 100 mg, 0.4 mmol) in DMF (2 mL) was added cesium carbonate (365 mg, 1.1 mmol) at ambient temperature and the reaction mixture was heated up to 100° C. for 16 h. The reaction mixture was quenched with water, extracted with EtOAc (3×30 mL), washed with brine, dried over anhydrous Na₂SO₄, and concentrated under reduced pressure. The product was purified by RP prep-HPLC and lyophilized to afford 2-((4-(2,6-dimethylphenyl)-2-oxo-2H-chromen-7-yl)oxy)acetonitrile (Example 107, 15 mg). LCMS (ESI) Calcd. for C₁₉H₁₅NO₃: 305, found [M+H]⁺=306. ¹H NMR (400 MHz, DMSO-d₆) δ 7.33-7.30 (m, 2H), 7.23-7.21 (m, 2H), 6.98-6.96 (m, 1H), 6.82-6.80 (d, 1H), 6.26 (s, 1H), 5.30 (s, 2H), 2.03 (s, 6H).

### Example 108: 2-((2-oxo-4-(o-tolyl)-2H-chromen-7-yl)oxy)acetamide

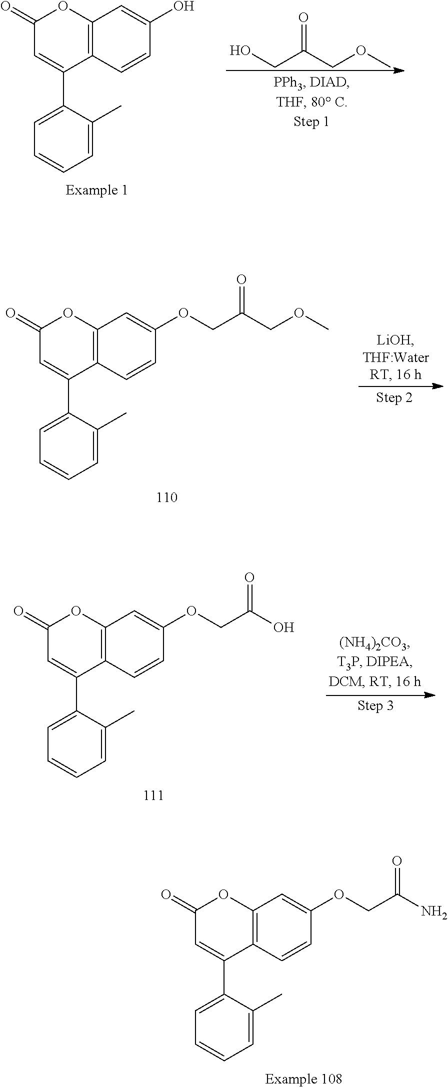

Synthesis of ethyl 2-((2-oxo-4-(o-tolyl)-2H-chromen-7-yl)oxy)acetate, 110 [Step 1]: To a stirred solution of 7-hydroxy-4-(o-tolyl)chromen-2-one (Example 1, 250 mg, 1 mmol) and ethyl 2-hydroxyacetate (225 mg, 2.2 mmol) in THF (5 mL) was added PPh₃ (780 mg, 3 mmol) and the mixture was cooled in an ice bath. DIAD (0.6 mL, 3 mmol) was added and the reaction mixture was allowed to warm up to ambient temperature and then gradually to 80° C. for 16 h. The reaction mixture was diluted with ethyl acetate and washed with water, brine, dried over anhydrous Na₂SO₄, and concentrated under reduced pressure. The product was purified by combi-flash chromatography to afford ethyl 2-((2-oxo-4-(o-tolyl)-2H-chromen-7-yl)oxy)acetate (110, 270 mg). LCMS (ESI) Calcd. for C₂₀H₁₈O₅: 338, found [M+H]⁺=339. ¹H NMR (400 MHz, CDCl₃) δ 7.39-7.29 (m, 3H), 7.16 (d, 1H), 6.98 (m, 1H), 6.83 (m, 1H), 6.76-6.73 (m, 1H), 6.16 (s, 1H), 4.66 (s, 2H), 4.30-4.23 (m, 2H), 2.14 (s, 3H), 3.09 (m, 1H).

Synthesis of 2-((2-oxo-4-(o-tolyl)-2H-chromen-7-yl)oxy)acetic acid, 111 [Step 2]: To a stirred solution of ethyl 2-((2-oxo-4-(o-tolyl)-2H-chromen-7-yl)oxy)acetate (110, 270 mg, 0.8 mmol) in a mixture of THF (4 mL): water (1 mL) was added LiOH H₂O (135 mg, 3.2 mmol) and the reaction mixture was stirred at ambient temperature for 16 h. The reaction mixture was diluted with water, acidified with 1N HCl and extracted with EtOAc (3×30 mL). The combined organic part was dried over anhydrous Na₂SO₄ and concentrated under reduced pressure to afford 2-((2-oxo-4-(o-tolyl)-2H-chromen-7-yl)oxy)acetic acid (111, 220 mg). LCMS (ESI) Calcd. for C₁₈H₁₄O₅: 310, found [M+H]⁺=311.

Synthesis of 2-((2-oxo-4-(o-tolyl)-2H-chromen-7-yl)oxy)acetamide, Example 108 [Step 3]: A stirred solution of 2-((2-oxo-4-(o-tolyl)-2H-chromen-7-yl)oxy)acetic acid (111, 230 mg, 0.7 mmol) in DMF (3 mL) was cooled to 0-5° C. and (NH₄)₂CO₃ (355 mg, 3.7 mmol), DIPEA (0.64 mL, 3.7 mmol), and T₃P (50% in EtOAc) (0.7 mL, 1.1 mmol) were added and the reaction mixture was stirred at ambient temperature for 16 h. The reaction mixture was diluted with ice-cold water, extracted with EtOAc, washed with brine, dried over anhydrous Na₂SO₄, and concentrated under reduced pressure. The product was purified by RP prep-chromatography and lyophilization to afford 2-((2-oxo-4-(o-tolyl)-2H-chromen-7-yl)oxy)acetamide (Example 108, 40 mg). LCMS (ESI) Calcd. for C₁₈H₁₅ClFNO₄: 309, found: [M+H]⁺=310. ¹H NMR (400 MHz, DMSO-d₆) δ 7.61 (br s, 1H), 7.45-7.33 (m, 4H), 7.25 (d, 1H), 7.05 (d, 1H), 6.93-6.87 (m, 2H), 6.20 (s, 1H), 4.56 (s, 2H), 2.11 (s, 3H).

### Example 109: 4-(o-tolyl)-7-((1,1,1-trifluoropropan-2-yl)oxy)-2H-chromen-2-one

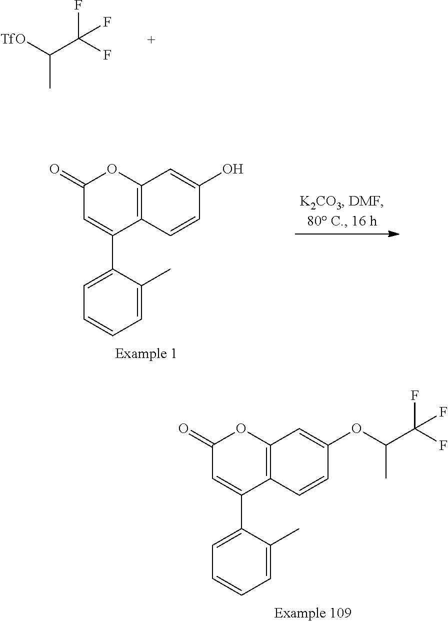 [SMILES: CC(OS(=O)(=O)C(F)(F)F)C(F)(F)F ; Cc1ccccc1-c1cc(=O)oc2cc(O)ccc12 ; Cc1ccccc1-c1cc(=O)oc2cc(OC(C)C(F)(F)F)ccc12]

Synthesis of 4-(o-tolyl)-7-((1,1,1-trifluoropropan-2-yl)oxy)-2H-chromen-2-one, Example 109: To a stirred solution of 7-hydroxy-4-(o-tolyl)-2H-chromen-2-one (Example 1, 150 mg, 0.6 mmol) in DMF (3 mL) was added K₂CO₃ (205 mg, 1.5 mmol) and a DCM solution of (2,2,2-trifluoro-1-methyl-ethyl) trifluoromethanesulfonate (860 mg, 3.5 mmol) and the reaction mixture was stirred at 80° C. for 16 h. The reaction was quenched with ice-cold water, extracted with EtOAc, washed with brine, dried over anhydrous Na₂SO₄, and purified by flash chromatography to afford 4-(o-tolyl)-7-((1,1,1-trifluoropropan-2-yl)oxy)-2H-chromen-2-one (Example 109, 140 mg). LCMS (ESI) Calcd. for C₁₉H₁₅F₃O₃: 348, [M+H]⁺=349. ¹H NMR (400 MHz, DMSO-d₆) δ 7.42-7.32 (m, 4H), 7.25-7.23 (d, 1H), 7.00-6.98 (dd, 1H), 6.91-6.89 (d, 1H), 6.25 (s, 1H), 5.49-5.43 (m, 1H), 2.11 (s, 3H), 1.46-1.44 (d, 3H).

### Example 110: (R)-4-(2-chloro-4-fluorophenyl)-7-((1-(1,1-dioxidothiomorpholino)-1-oxopropan-2-yl)oxy)-2H-chromen-2-one

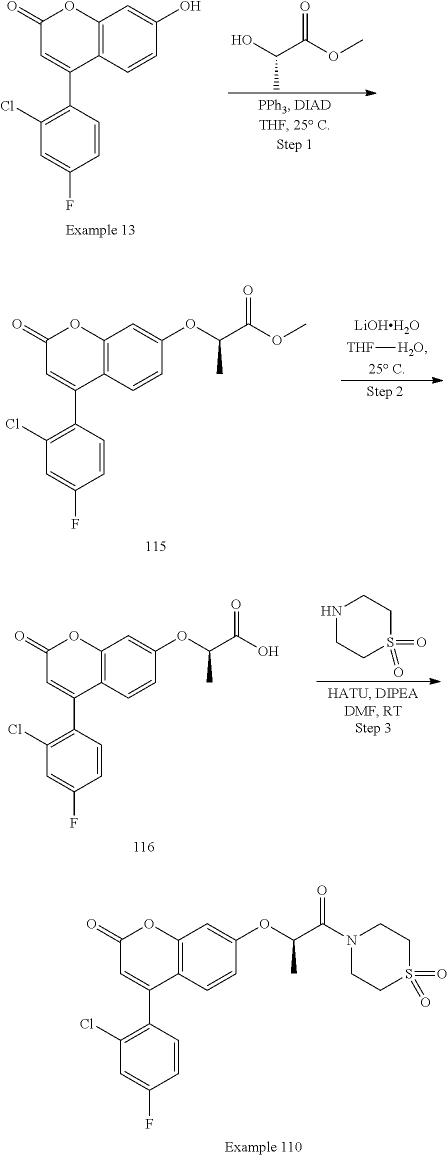

Synthesis of methyl (R)-2-((4-(2-chloro-4-fluorophenyl)-2-oxo-2H-chromen-7-yl)oxy)propanoate, 115 [Step 1]: To an ice-cold solution of 4-(2-chloro-4-fluorophenyl)-7-hydroxy-2H-chromen-2-one (Example 13, 1.0 g, 3.44 mmol), methyl (S)-2-hydroxypropanoate (0.5 mL, 5.2 mmol) and triphenylphosphine (1.4 g, 5.2 mmol) in THF (20 mL) was added diisopropyl azodicarboxylate (1.0 mL, 5.2 mmol), and the reaction mixture was stirred at 25° C. After 16 h., the reaction mixture was concentrated under reduced pressure, and purified by column chromatography to afford methyl (R)-2-((4-(2-chloro-4-fluorophenyl)-2-oxo-2H-chromen-7-yl)oxy)propanoate (115, 1.0 g). LCMS (ESI) Calcd. for C₁₉H₁₄ClFO₅: 376, found [M+H]⁺=377. ¹H NMR (400 MHz, DMSO-d₆) δ 7.71-7.69 (m, 1H), 7.58-7.55 (m, 1H), 7.46-7.42 (m, 1H), 7.04-7.03 (m, 1H), 6.96-6.94 (m, 1H), 6.90-6.87 (m, 1H), 6.33 (s, 1H), 5.25-5.20 (m, 1H), 3.70 (s, 3H), 1.54 (d, 3H).

Synthesis of (R)-2-((4-(2-chloro-4-fluorophenyl)-2-oxo-2H-chromen-7-yl)oxy)propanoic acid, 116 [Step 2]: To a solution of methyl (R)-2-((4-(2-chloro-4-fluorophenyl)-2-oxo-2H-chromen-7-yl)oxy)propanoate (115, 150 mg, 0.4 mmol) in THF (2 mL)-water (0.5 mL) was added lithium hydroxide monohydrate (40 mg, 1.0 mmol) and the reaction mixture was stirred at 25° C. After 16 h., the reaction mixture was concentrated under reduced pressure and was diluted with water, acidified with 1N HCl to pH 2, and extracted with 20% methanol in dichloromethane (twice). The combined organic extract was washed with brine, dried over anhydrous Na₂SO₄, filtered, and concentrated under reduced pressure to afford (R)-2-((4-(2-chloro-4-fluorophenyl)-2-oxo-2H-chromen-7-yl)oxy)propanoic acid (116, 100 mg). LCMS (ESI) Calcd. for C₁₈H₁₂ClFO₅: 362, found [M+H]⁺=363.

Synthesis of (R)-4-(2-chloro-4-fluorophenyl)-7-((1-(1,1-dioxidothiomorpholino)-1-oxopropan-2-yl)oxy)-2H-chromen-2-one, Example 110 [Step 3]: To a stirred solution of (R)-2-((4-(2-chloro-4-fluorophenyl)-2-oxo-2H-chromen-7-yl)oxy)propanoic acid (116, 100 mg, 0.3 mmol) and thiomorpholine 1,1-dioxide (45 mg, 0.3 mmol) in DMF (3 mL) was added HATU (157 mg, 0.4 mmol) and DIPEA (0.1 mL, 0.7 mmol) successively at 25° C. After 16 h., the reaction mixture was quenched with cold water, and extracted with ethyl acetate (twice). The combined organic extract was washed with brine, dried over anhydrous Na₂SO₄, filtered, and concentrated under reduced pressure. The product was purified by RP prep-HPLC and lyophilized to afford (R)-4-(2-chloro-4-fluorophenyl)-7-((1-(1,1-dioxidothiomorpholino)-1-oxopropan-2-yl)oxy)-2H-chromen-2-one (Example 110, 50 mg). LCMS (ESI) Calcd. for C₂₂H₁₉ClFNO₆S: 479, found [M+H]⁺=480. ¹H NMR (400 MHz, DMSO-d₆) (at 100° C.) δ 7.62-7.59 (m, 1H), 7.55-7.52 (m, 1H), 7.41-7.37 (m, 1H), 7.05-7.04 (m, 1H), 6.99-6.97 (m, 1H), 6.88-6.86 (m, 1H), 6.25 (s, 1H), 5.51-5.46 (m, 1H), 3.97-3.95 (m, 4H), 3.20-3.15 (m, 4H), 1.52 (d, 3H).

### Example 111: (R)-7-((1-(1,1-dioxidothiomorpholino)-1-oxopropan-2-yl)oxy)-4-(o-tolyl)-2H-chromen-2-one

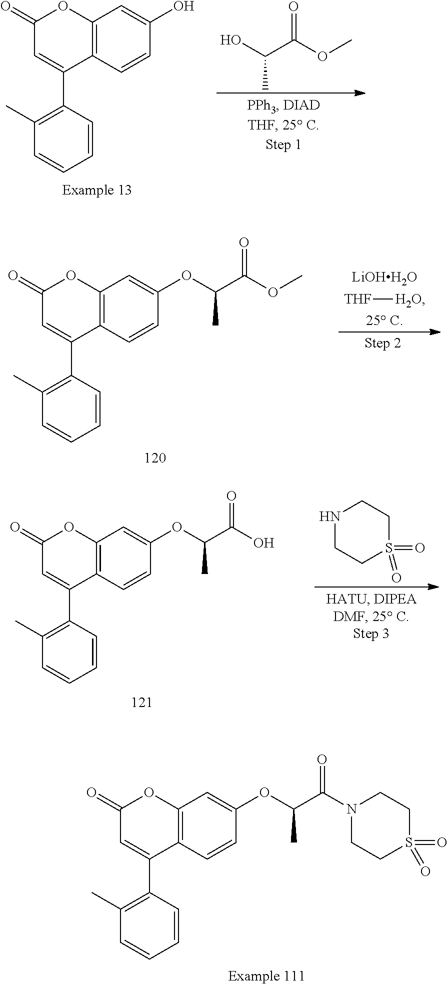

Synthesis of methyl (R)-2-((2-oxo-4-(o-tolyl)-2H-chromen-7-yl)oxy)propanoate, 120 [Step 1]: To an ice-cold solution of 7-hydroxy-4-(o-tolyl)-2H-chromen-2-one (Example 13, 1.0 g, 3.9 mmol), methyl (S)-2-hydroxypropanoate (0.6 mL, 5.9 mmol), and triphenylphosphine (1.6 g, 5.9 mmol) in THF (20 mL) was added dropwise diisopropyl azodicarboxylate (1.2 mL, 5.9 mmol). The reaction mixture was stirred at ambient temperature for 16 h., concentrated under reduced pressure, and purified by column chromatography to afford methyl (R)-2-((2-oxo-4-(o-tolyl)-2H-chromen-7-yl)oxy)propanoate (120, 800 mg). LCMS (ESI) Calcd. for C₂₀H₁₈O₅: 338, found [M+H]⁺=339. ¹H NMR (400 MHz, DMSO-d₆) δ 7.43-7.39 (m, 2H), 7.37-7.33 (m, 1H), 7.25-7.23 (m, 1H), 7.02 (s, 1H), 6.90-6.84 (m, 2H), 6.21 (s, 1H), 5.22-5.20 (m, 1H), 3.70 (s, 3H), 2.12 (d, 3H), 1.54 (d, 3H).

Synthesis of (R)-2-((2-oxo-4-(o-tolyl)-2H-chromen-7-yl)oxy)propanoic acid, 121 [Step 2]: To a solution of methyl (R)-2-((2-oxo-4-(o-tolyl)-2H-chromen-7-yl)oxy)propanoate (120, 150 mg, 0.4 mmol) in THF (2 mL)-water (0.5 mL) mixture, was added lithium hydroxide monohydrate (45 mg, 1.1 mmol), and the reaction mixture was stirred at ambient temperature for 16 h. The mixture was concentrated under reduced pressure, acidified with 1N aqueous HCl to pH 2, and extracted with ethyl acetate (twice). The combined organic extract was washed with brine, dried over anhydrous Na₂SO₄, filtered, and concentrated under reduced pressure to afford (R)-2-((2-oxo-4-(o-tolyl)-2H-chromen-7-yl)oxy)propanoic acid (121, 120 mg). LCMS (ESI) Calcd. for C₁₉H₁₆O₅: 324, found [M+H]⁺=325.

Synthesis of (R)-7-((1-(1,1-dioxidothiomorpholino)-1-oxopropan-2-yl)oxy)-4-(o-tolyl)-2H-chromen-2-one, Example 111 [Step 3]: To a stirred solution of (R)-2-((2-oxo-4-(o-tolyl)-2H-chromen-7-yl)oxy)propanoic acid (121, 100 mg, 0.5 mmol) in DMF (3 mL), was added HATU (265 mg, 0.7 mmol) followed by N,N-diisopropylethylamine (0.2 mL, 1.2 mmol) at 0° C. After stirring for 10 min. at ambient temperature, thiomorpholine 1,1-dioxide (75 mg, 0.5 mmol) was added and the reaction mixture was stirred at ambient temperature for 4 h. The reaction mixture was diluted with ice-cold water, and extracted with ethyl acetate (twice). The combined organic extract was washed with brine, dried over anhydrous Na₂SO₄, filtered, and concentrated under reduced pressure. The product was purified by RP prep-HPLC and lyophilized to afford (R)-7-((1-(1,1-dioxidothiomorpholino)-1-oxopropan-2-yl)oxy)-4-(o-tolyl)-2H-chromen-2-one (Example 111, 55 mg). LCMS (ESI) Calcd. for C₂₃H₂₃NO₆S: 441, found [M+H]⁺=442. ¹H NMR (400 MHz, DMSO-d₆) δ 7.45-7.33 (m, 2H), 7.36-7.33 (m, 1H), 7.25-7.22 (m, 1H), 7.05-7.04 (m, 1H), 6.90-6.88 (m, 1H), 6.83-6.79 (m, 1H), 6.15 (s, 1H), 5.49-5.44 (m, 1H), 3.95-3.94 (m, 4H), 3.16-3.15 (m, 4H), 2.14 (s, 3H), 1.52 (d, 3H).

### Example 112: Synthesis of (R)-4-(2,6-dimethylphenyl)-7-((1-(1,1-dioxidothiomorpholino)-1-oxopropan-2-yl)oxy)-2H-chromen-2-one

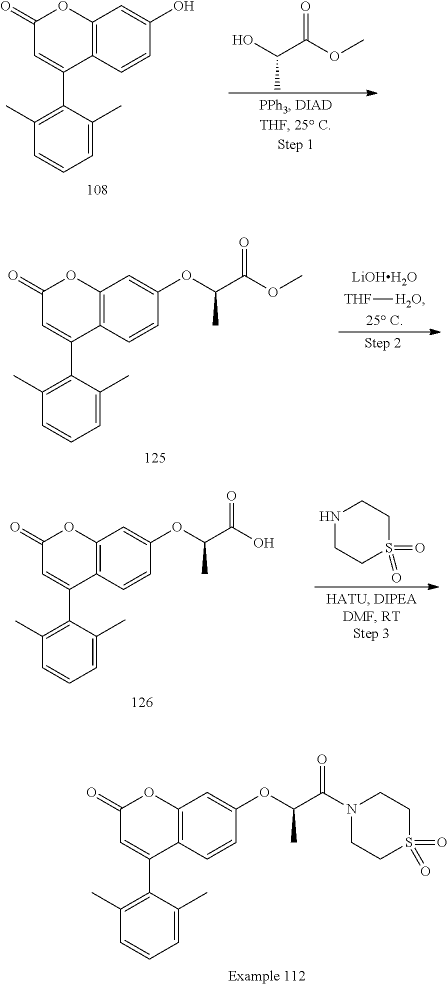

Synthesis of methyl (R)-2-((4-(2,6-dimethylphenyl)-2-oxo-2H-chromen-7-yl)oxy)propanoate, 125 [Step 1]: To an ice-cold solution of 4-(2,6-dimethylphenyl)-7-hydroxy-2H-chromen-2-one (108, 1.0 g, 3.8 mmol), methyl (S)-2-hydroxypropanoate (4, 0.5 mL, 5.6 mmol) and triphenylphosphine (1.5 g, 5.6 mmol) in THF (20 mL), was added diisopropyl azodicarboxylate (1.1 mL, 5.6 mmol) and the reaction mixture was stirred at ambient temperature. After 16 h., the reaction mixture was concentrated under reduced pressure, and purified by Combi-Flash column chromatography to afford methyl (R)-2-((4-(2,6-dimethylphenyl)-2-oxo-2H-chromen-7-yl)oxy)propanoate (125, 1.0 g). LCMS (ESI) Calcd. for C₂₁H₂₀O₅: 352, found [M+H]⁺=352.

Synthesis of (R)-2-((4-(2,6-dimethylphenyl)-2-oxo-2H-chromen-7-yl)oxy)propanoic acid, 126 [Step 2]: To a solution of methyl (R)-2-((4-(2,6-dimethylphenyl)-2-oxo-2H-chromen-7-yl)oxy)propanoate (125, 200 mg, 0.57 mmol) in THF (4 mL)-water (1 mL) was added lithium hydroxide monohydrate (35 mg, 0.8 mmol), and the reaction mixture was stirred at ambient temperature. After 16 h., the reaction mixture was concentrated under reduced pressure, acidified with 1N aqueous HCl to pH 2, and extracted with 20% methanol in dichloromethane (twice). The combined organic extract was washed with brine, dried over anhydrous Na₂SO₄, filtered, and concentrated under reduced pressure to afford (R)-2-((4-(2,6-dimethylphenyl)-2-oxo-2H-chromen-7-yl)oxy)propanoic acid (126, 160 mg). The product was used in the next step without further purification. LCMS (ESI) Calcd. for C₂₀H₁₃O₅: 338, found [M+H]⁺=339.

Synthesis of (R)-4-(2,6-dimethylphenyl)-7-((1-(1,1-dioxidothiomorpholino)-1-oxopropan-2-yl)oxy)-2H-chromen-2-one, Example 112 [Step 3]: To a solution of (R)-2-((4-(2,6-dimethylphenyl)-2-oxo-2H-chromen-7-yl)oxy)propanoic acid (126, 160 mg, 0.5 mmol) and thiomorpholine 1,1-dioxide (77 mg, mmol) in DMF (5 mL), was added HATU (270 mg, 0.7 mmol) followed by DIPEA (0.2 mL, 1.2 mmol), and the reaction mixture was stirred at ambient temperature. After 16 h., the reaction mixture was quenched with cold water and extracted with ethyl acetate (twice). The combined organic extract was washed with brine, dried over anhydrous Na₂SO₄, filtered, and concentrated under reduced pressure. The product was purified by RP prep-HPLC followed by lyophilization to afford (R)-4-(2,6-dimethylphenyl)-7-((1-(1,1-dioxidothiomorpholino)-1-oxopropan-2-yl)oxy)-2H-chromen-2-one (Example 112, 80 mg). LCMS (ESI) Calcd. for C₂₄H₂₅NO₆S: 455, found [M+H]⁺=456. ¹H NMR (400 MHz, DMSO-d₆) (at 100° C.) δ 8.05-8.03 (m, 1H), 7.33-7.31 (m, 1H), 7.21-7.19 (m, 2H), 7.12-7.09 (m, 2H), 6.27 (s, 1H), 5.51 (q, 1H), 3.98-3.95 (m, 4H), 3.16-3.14 (m, 4H), 2.27 (s, 6H), 1.53 (d, 3H).

### Example 113: Synthesis of 4-phenyl-7-propoxy-2H-chromen-2-one

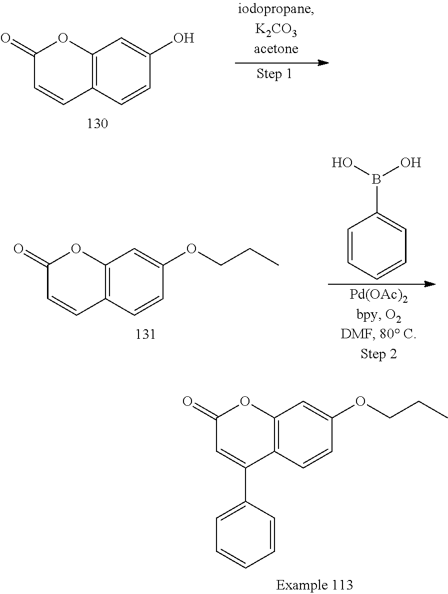 [SMILES: O=c1ccc2ccc(O)cc2o1 ; CCCOc1ccc2ccc(=O)oc2c1 ; OB(O)c1ccccc1 ; CCCOc1ccc2c(-c3ccccc3)cc(=O)oc2c1]

Synthesis of 7-propoxy-2H-chromen-2-one, 131 [Step 1]: To a solution of 7-hydroxy-2H-chromen-2-one (130, 1.0 g, 6.2 mmol) in acetone (30 mL) was added potassium carbonate (1.7 g, 12.3 mmol) followed by 1-iodopropane (0.7 mL, 7.4 mmol) at 25° C., and the reaction mixture was stirring for 16 h. The reaction mixture was concentrated under reduced pressure, and purified on a silica gel column to afford 7-propoxy-2H-chromen-2-one (131, 1.0 g). LCMS (ESI) Calcd. for C₁₂H₁₂O₃: 204, found [M+H]⁺=205. ¹H NMR (400 MHz, DMSO-d₆): δ 7.62 (d, 1H), 7.34 (d, 1H), 6.84-6.79 (m, 2H), 6.22 (d, 1H), 3.97 (t, 2H), 1.83 (sextet, 2H), 1.05 (t, 3H).

Synthesis of 4-phenyl-7-propoxy-2H-chromen-2-one, Example 113 [Step: To a mixture of 7-propoxy-2H-chromen-2-one (131, 200 mg, 1.0 mmol), phenylboronic acid (350 mg, 2.9 mmol), Pd(OAc)₂ (22 mg, 0.1 mmol) and 2,2′-Bipyridyl (23 mg, 0.15 mmol) in dry DMF (2 mL) was bubbled O₂ gas, and the reaction mixture was stirred at 80° C. for 16 h. The resulting mixture was allowed to warm to ambient temperature, quenched with water, and extracted with EtOAc (x2). The organic layers were combined and washed with brine, dried with anhydrous Na₂SO₄, filtered, and concentrated under reduced pressure. The product was purified by RP prep-HPLC and lyophilized to afford 4-phenyl-7-propoxy-2H-chromen-2-one (Example 113, 49 mg, 0.2 mmol). LCMS (ESI) Calcd. for C₁₈H₁₆O₃: 280, found [M+H]⁺=281. ¹H NMR (400 MHz, DMSO-d₆): δ 7.58-7.51 (m, 5H), 7.34 (d, 1H), 7.08 (d, 1H), 6.94 (dd, 1H), 6.23 (s, 1H), 4.06 (t, 2H), 1.76 (sextet, 2H), 0.99 (t, 3H).

### Example 114: Synthesis of chiral

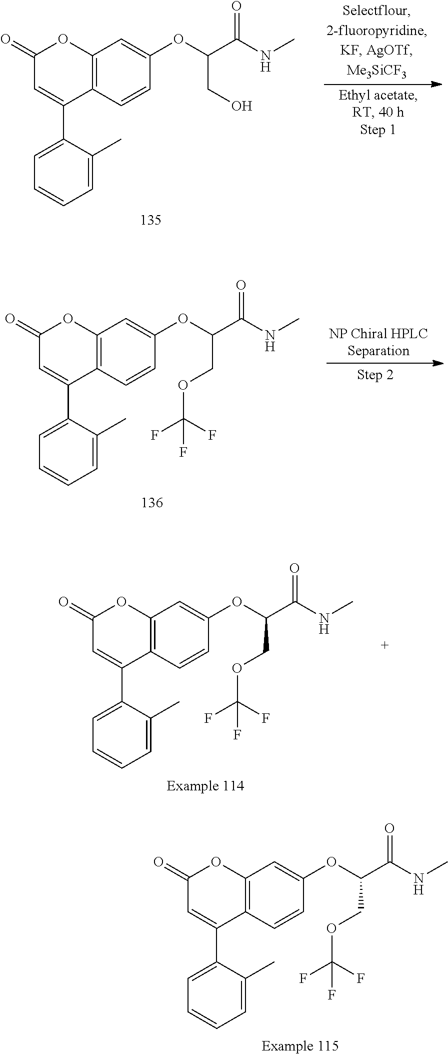 [SMILES: CNC(=O)C(CO)Oc1ccc2c(-c3ccccc3C)cc(=O)oc2c1 ; CNC(=O)C(COC(F)(F)F)Oc1ccc2c(-c3ccccc3C)cc(=O)oc2c1 ; CNC(=O)[C@@H](COC(F)(F)F)Oc1ccc2c(-c3ccccc3C)cc(=O)oc2c1 ; CNC(=O)[C@H](COC(F)(F)F)Oc1ccc2c(-c3ccccc3C)cc(=O)oc2c1]

Synthesis of N-methyl-2-((2-oxo-4-(o-tolyl)-2H-chromen-7-yl)oxy)-3-(trifluoromethoxy)propanamide, 136 [Step 1]: In a sealed tube was added dry potassium fluoride (410 mg, 7.1 mmol), silver trifluoromethanesulphonate (1.5 g, 5.7 mmol), 1-chloromethyl-4-fluoro-1,4-diazoniabicyclo[2.2.2]octane bis(tetrafluoroborate) (1.0 g, 2.8 mmol) and 3-hydroxy-N-methyl-2-((2-oxo-4-(o-tolyl)-2H-chromen-7-yl)oxy)propanamide (135, 500 mg, 1.4 mmol) under argon. To the mixture was added dry ethyl acetate (20 mL), 2-fluoropyridine (0.5 mL, 5.7 mmol) and (trifluoromethyl)trimethylsilane (0.8 mL, 5.7 mmol) under argon and the reaction mixture was stirred at ambient temperature for 40 h. The reaction mixture was diluted with ethyl acetate and filtered through a celite bed. The filtrate was dried over anhydrous Na₂SO₄ and concentrated under reduced pressure. The product was purified by column chromatography followed by RP prep-HPLC to afford N-methyl-2-((2-oxo-4-(o-tolyl)-2H-chromen-7-yl)oxy)-3-(trifluoromethoxy)propanamide (136, 250 mg). LCMS (ESI) Calcd for C₂₁H₁₈F₃NO₅: 421, found [M+H]⁺=422. ¹H NMR (400 MHz, DMSO-d₆) δ 8.31 (br s, 1H) 7.46-7.34 (m, 3H), 7.24 (d, 1H), 7.15 (d, 1H), 6.99 (d, 1H), 6.91 (d, 1H), 6.24 (s, 1H), 5.23 (br s, 1H), 4.47 (br s, 2H), 2.65 (d, 3H), 2.12 (s, 3H).

Synthesis of chiral N-methyl-2-((2-oxo-4-(o-tolyl)-2H-chromen-7-yl)oxy)-3-(trifluoromethoxy)propanamide, Example 114 and Example 115 [Step 2]: The racemic N-methyl-2-((2-oxo-4-(o-tolyl)-2H-chromen-7-yl)oxy)-3-(trifluoromethoxy)propanamide (136, 30 mg) was used for normal phase chiral HPLC separation to afford Peak 1 as chiral N-methyl-2-((2-oxo-4-(o-tolyl)-2H-chromen-7-yl)oxy)-3-(trifluoromethoxy)propanamide (Example 114, 10 mg) and Peak 2 as chiral N-methyl-2-((2-oxo-4-(o-tolyl)-2H-chromen-7-yl)oxy)-3-(trifluoromethoxy)propanamide (Example 115, 11 mg). The absolute stereochemistries of these compounds were not determined.

Example 114, N-methyl-2-((2-oxo-4-(o-tolyl)-2H-chromen-7-yl)oxy)-3-(trifluoromethoxy)propanamide, Peak 1: LCMS (ESI) Calcd. for C₂₁H₁₈F₃NO₅: 421, found [M+H]⁺=422. ¹H NMR (400 MHz, DMSO-d₆) δ 8.30 (b rs, 1H), 7.43-7.41 (m, 2H), 7.37 (t, 1H), 7.24 (d, 1H), 7.15 (d, 1H), 6.99 (d, 1H), 6.91 (d, 1H), 6.23 (s, 1H), 5.23 (br s, 1H), 4.46 (br s, 2H), 2.65 (d, 3H), 2.12 (s, 3H).

Example 115, N-methyl-2-((2-oxo-4-(o-tolyl)-2H-chromen-7-yl)oxy)-3-(trifluoromethoxy)propanamide, Peak 2: LCMS (ESI) Calcd. for C₂₁H₁₈F₃NO₅: 421, found [M+H]⁺=422. ¹H NMR (400 MHz, DMSO-d₆) δ 8.30 (br s, 1H), 7.44-7.39 (m, 2H), 7.35 (t, 1H), 7.24 (d, 1H), 7.15 (d, 1H), 6.99 (d, 1H), 6.91 (d, 1H), 6.23 (s, 1H), 5.23 (br s, 1H), 4.46 (br s, 2H), 2.65 (d, 3H), 2.11 (s, 3H).

Prep Normal phase HPLC Chiral Method: Chiral separation was performed on an Agilent 1200 series instrument. Column was a CHIRALCEL OD-H (250×20 mm), 5μ, operating at ambient temperature and a flow rate of 18.0 mL/min. The mobile phase was mixture of 70% Hexane and 30% EtOH, held isocratic for 13 min. with detection at a wavelength of 322 nm.

### Biological/Biochemical Evaluation

### General Protocol for In Vitro Analysis of Compounds

The inhibitory activity of the compounds of the present invention against POLRMT were determined by assays based on Bergbrede, T., et al., “An adaptable high-throughput technology enabling the identification of specific transcription modulators,” SLAC Discov., 22, 378-386 (2017).

The ability of some compounds of the present invention to inhibit POLRMT were determined in a homogeneous TR-FRET Assay using high-throughput screening in a 384-well plate format. This method is used to monitor the activity of mitochondrial transcription through measurement of its product, a 407 bp long RNA transcript. Detection of the product is facilitated by hybridization of two DNA-oligonucleotide probes to specific and adjacent sequences within the RNA product sequence. Upon annealing of the probes, two fluorophores are coupled directly to an acceptor nucleotide probe (ATT0647, 5′), or introduced via a coupled streptavidin with a biotinylated donor nucleotide probe (Europium cryptate) that is brought into sufficient proximity to serve as a fluorescence-donor-acceptor pair. Thus, a FRET signal at 665 nm is generated upon excitation at 340 nm.

Proteins used as transcription factors (POLRMT: NP_005026.3, TFAM: NP_003192.1, TFB2M: NP_071761.1) are diluted from their stocks to working concentrations of 1 μM, 20 μM and 4 μM respectively, in a dilution buffer containing 20 mM Tris-HCl (pH 8.0), 200 mM NaCl, 10% (v/v) glycerol, 1 mM Dithiothreitol (DTT), mM EDTA.

DNA template is a pUC18 plasmid with the mitochondrial light strand promotor sequence (1-477) cloned between HindIII and BamHI sites. The DNA template is restriction linearized proximal to the promotor 3′-end (pUC-LSP). The reaction mixture (10 μL) containing 7.5 nM POLRMT, 15 nM of TFB2M, 30 nM of TFAM, 0.5 nM of DNA template and 500 μM nucleotide triphosphate mix (NTPs) in a reaction buffer (containing 10 mM Tris-HCl (pH 7.5), 10 mM MgCl₂, 40 mM NaCl, 10 mM DTT, 0.005% (w/v) Tween-20, 160 units/ml Rnase inhibitor and 0.1 mg/mL BSA) are dispensed to compounds in microplates, using a Thermo Multidrop® dispenser, and incubated at 37° C. in a VWR INCU-Line incubator for 60 minutes after mixing. No nucleotide triphosphate mix is added to negative control samples. Microplates with compounds to be tested in the assay are prepared from 10 mM compound stocks in 100% DMSO, equal amounts of DMSO without any compound are added to positive control and negative control samples.

During the incubation, a mix of the detection reagents is prepared in a buffer such that the enzymatic reaction is terminated due to chelating of Mg-ions and increased ionic strength, containing 50 mM Tris-HCl (pH 7.5), 700 mM NaCl, 20 mM EDTA, and 0.01% (w/v) Tween-20. Europium-streptavidin is pre-incubated with a 200-fold molar excess of a random sequence oligonucleotide to block unspecific binding of oligo, for two hours at ambient temperature in the dark. Afterwards, the blocked Europium-streptavidin is kept on ice until use.

At the end of the enzymatic reaction time, 5 μL detection oligo mix in the detection buffer is added, and assay plates are mixed and kept at ambient temperature for one hour, protected from light. The concentration of the Acceptor nucleotide oligo (e.g., ATTO647N-5′-ACAAAGAACCCTAACACCAG-3′) and Donor nucleotide oligo (e.g., bio-5′-AACACATCTCT(-bio)GCCAAACCCCA-bio-3′) in each assay well is 1 nM, and 3 nM, respectively.

After incubation with oligo mix, 5 μL of pre-blocked Europium-streptavidin reagent is dispensed to each assay well, assay plates are again mixed and kept at ambient temperature for one hour, protected from light.

The generated signal is measured with BMG Pherastar microtiter plate reader with a TRF light unit, using excitation at 340 nm, an integration time of 200 ρs, and a delay time of 100 ρs, before detection at 620 nm and 665 nm. The ratio of donor- and acceptor-fluorescence is used as a measure of the generated transcript product (i.e. enzymatic activity).

The IC₅₀ values are summarized in Table 1.

TABLE 1

| Examples | IC₅₀ (μM) |
|---|---|
| Example 01 | 3 |
| Example 02 | 0.09 |
| Example 03 | 2 |
| Example 04 | 2 |
| Example 05 | 0.1 |
| Example 06 | 0.6 |
| Example 07 | 4 |
| Example 08 | 94 |
| Example 09 | 0.9 |
| Example 12 | 7 |
| Example 13 | 3 |
| Example 14 | 0.2 |
| Example 15 | 0.4 |
| Example 16 | >100 |
| Example 19 | 0.5 |
| Example 20 | 2 |
| Example 21 | 4 |
| Example 22 | 34 |
| Example 23 | 87 |
| Example 24 | 93 |
| Example 25 | 0.3 |
| Example 26 | 0.3 |
| Example 27 | 0.6 |
| Example 28 | 0.5 |
| Example 29 | 1 |
| Example 30 | 1 |
| Example 31 | 0.2 |
| Example 32 | 23 |
| Example 33 | 0.2 |
| Example 34 | 4 |
| Example 35 | 6 |
| Example 36 | 50 |
| Example 37 | 2 |
| Example 39 | 0.9 |
| Example 40 | 3 |
| Example 42 | 0.05 |
| Example 43 | 1 |
| Example 45 | 0.1 |
| Example 46 | 0.3 |
| Example 48 | 3 |
| Example 49 | 1 |
| Example 50 | 10 |
| Example 51 | 0.4 |
| Example 52 | 17 |
| Example 53 | 0.8 |
| Example 54 | 3 |
| Example 55 | 5 |
| Example 56 | 8 |
| Example 57 | 4 |
| Example 58 | 0.4 |
| Example 59 | 0.6 |
| Example 60 | 0.5 |
| Example 61 | 28 |
| Example 62 | 19 |
| Example 63 | 90 |
| Example 64 | 54 |
| Example 65 | 0.8 |
| Example 66 | 42 |
| Example 67 | 16 |
| Example 68 | 0.7 |
| Example 69 | 17 |
| Example 70 | 0.9 |
| Example 71 | 0.1 |
| Example 72 | 10 |
| Example 73 | 3 |
| Example 74 | 13 |
| Example 75 | 2 |
| Example 76 | 44 |
| Example 77 | 2 |
| Example 78 | 0.5 |
| Example 79 | 32 |
| Example 80 | 15 |
| Example 81 | 0.2 |
| Example 82 | 5 |
| Example 83 | 0.2 |
| Example 84 | 2 |
| Example 85 | 0.2 |
| Example 86 | >100 |
| Example 87 | 36 |
| Example 88 | 0.5 |
| Example 89 | 0.2 |
| Example 90 | 10 |
| Example 91 | 14 |
| Example 92 | 0.4 |
| Example 93 | 18 |
| Example 94 | 0.09 |
| Example 95 | 14 |
| Example 96 | 0.5 |
| Example 97 | 5 |
| Example 98 | >100 |
| Example 99 | 4 |
| Example 100 | 0.9 |
| Example 101 | 0.4 |
| Example 102 | 1 |
| Example 103 | 16 |
| Example 104 | 2 |
| Example 105 | 0.04 |
| Example 106 | 0.03 |
| Example 107 | 0.03 |
| Example 108 | 0.2 |
| Example 109 | 0.3 |
| Example 110 | 0.5 |
| Example 111 | 0.2 |
| Example 112 | 65 |
| Example 113 | 4 |

### General Protocol for In Vivo AML (Acute Myeloid Leukemia) Efficacy Experiment

### Determination of Maximum Tolerated Dose of Test Compound

Immunocompromised mice (6-10-week-old, female NSG mice, strain NOD.Cg-Prkdc^{scid} I/2rg^{tm1wjl}/Szj, Jackson Laboratories) are treated orally with test compound ranging from 75 to 150 mg/kg, once or twice per day for the duration of 14 days. Total body weight is measured, and the general condition of mice is monitored routinely. Mice with severe symptoms and moribund are excluded from study. Submental blood collection method (no anesthesia) is used for all samplings. Plasma levels of test compound are determined at intervals ranging from 0.5 to 4 hours post first and last doses in all dosing groups. From these data pharmacokinetic analysis are conducted.

### In Vivo Efficacy Study in AML Mouse Model

MV4-11 AML cell lines (ATCC) are labelled with luciferase tag by viral transduction procedure (MV4-11-luc).

For an AML cell line xenograft efficacy experiment, female NSG mice are given intravenously ~1×10⁷ MV4-11-luc cells. Mice are flux sorted and randomized into treatment groups 14 days post transplantation. Mice are then treated with vehicle (50 mM Na₂HPO₄), or test compound at a tolerable dose determined from the above study, once or twice per day for 21 days. Tumor progression/regression is monitored by imaging of mice using luciferin as a substrate (150 mg/kg). Images are taken on a total of 9 time points i.e., one flux sort and once weekly to end date (8 time points). Imaging is performed under anesthesia and using in vivo imaging equipment IVIS. The treatment efficacy is also measured based on proportion of human AML cells, determined by flow cytometry analysis of viable human CD45 positive cell population in peripheral blood of mice one week post last dose. Plasma levels of test compound are determined at intervals ranging from 0.5 to 4 hours post last dose. Animals are monitored individually, and total body weight is measured routinely. The endpoint of the experiment is moribundity. In addition, mice demonstrating tumor-associated symptoms including impairment of hind limb function, ocular proptosis, and weight loss are considered for euthanasia. The remaining mice are euthanized on day 60 of the study.

1. A compound, or a pharmaceutically acceptable salt thereof, according to formula (I): 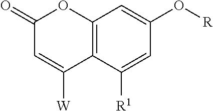
wherein:
W is C₆-C₁₀ aryl, C₆-C₁₀ cycloaryl, or 5-10 membered heteroaryl, any of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, CH₂CH₂R², 5-or 6-membered heterocyclyl optionally substituted with C₁-C₄ alkyl or C(O)R³, or C₁-C₆ alkyl optionally substituted with one or more groups, each independently selected from the group consisting of C₁-C₄ alkyl, 5-or 6-membered heterocyclyl, hydroxyl, cyano, fluoro, C₁-C₄ alkoxyl, C₁-C₄ haloalkoxyl, and NR³R⁴,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and C(O)R³, provided that when R is hydrogen, then R¹ is methyl and W is substituted, and further provided that when R is methyl, then W is disubstituted;
R¹ is hydrogen, fluoro, chloro, hydroxyl, cyano, C₃-C₄ cycloalkyl, C₁-C₂ alkoxyl, C₁-C₂ haloalkoxyl, or C₁-C₃ alkyl optionally substituted with one or more fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl, OR³, NR³R⁴, C(O)R³, or C(O)NR³R⁴;
each R³ is independently hydrogen or C₁-C₄ alkyl optionally substituted with one or more groups selected from the group consisting of fluoro, hydroxyl, C₁-C₄ haloalkoxyl, and C₁-C₄ alkoxyl;
each R⁴ is independently R³, C(O)C₁-C₄ alkyl optionally substituted with one or more fluoro groups, or C(O)NHC₁-C₄ alkyl optionally substituted with one or more fluoro groups;
or if R³ and R⁴ are attached to the same nitrogen atom, R³ and R⁴ together with their connecting nitrogen form a 5-or 6-membered heterocyclic ring optionally containing another heteroatom that is N, O, S, S(O), or S(O)NR³, and such heterocyclic ring is optionally substituted with one or more groups each independently selected from the group consisting of fluoro, chloro, C₁-C₄ alkyl, C₁-C₄ alkoxyl, C₁-C₄ haloalkoxyl, carboxyl, and C₁-C₄ alkylcarboxylate; and
R⁵ is hydrogen or C₁-C₄ alkyl.

2. The compound according to claim 1, or a pharmaceutically acceptable salt thereof,
wherein:
W is C₆ aryl, C₆ cycloaryl, or a 5-or 6-membered heteroaryl, any of which is optionally substituted with one or more groups, each independently selected from the group consisting of fluoro, chloro, C₁ alkyl, trifluoromethyl, hydroxyl, and C₁ alkoxyl;
R is hydrogen, CH₂CH₂R², C₆ aryl, 5-or 6-membered heterocyclyl optionally substituted with C₁ alkyl or C(O)R³, or C₁-C₄ alkyl optionally substituted with one or more groups, each independently selected from the group consisting of 6-membered heterocyclyl, hydroxyl, cyano, fluoro, C₁ alkoxyl, and NR³R⁴;
R¹ is hydrogen or C₁ alkyl;
R² is C₆ aryl;
each R³ is independently hydrogen or C₁ alkyl; and
each R⁴ is independently R³.

3. The compound according to claim 2, or a pharmaceutically acceptable salt thereof, wherein:
W is C₆ aryl or 5-membered heteroaryl, any of which is optionally substituted with one or more groups, each independently selected from the group consisting of methyl, chloro, and fluoro;
R is 6-membered heterocyclyl or C₁-C₄ alkyl optionally substituted with one or more groups independently selected from the group consisting of cyano, fluoro, and methoxyl; and
R¹ is hydrogen.

4. The compound according to claim 1, or a pharmaceutically acceptable salt thereof,
wherein:
W is C₆ aryl substituted with one or more methyl;
R is C₁-C₄ alkyl optionally substituted with one or more groups independently selected from the group consisting of cyano and methoxyl; and
R¹ is hydrogen.

5. The compound according to claim 1, or a pharmaceutically acceptable salt thereof, wherein:
W is C₆ aryl that is substituted with one or more methyl;
R is C₁-C₂ alkyl that is substituted with cyano; and
R¹ is hydrogen.

6. The compound according to claim 1, or a pharmaceutically acceptable salt thereof, wherein:
W is C₆ aryl that is substituted with two methyl;
R is C₁ alkyl substituted with cyano; and
R¹ is hydrogen.

7. A pharmaceutical composition comprising a compound according to claim 2, and a pharmaceutically acceptable excipient.

8. A pharmaceutical composition comprising a compound according to claim 3, and a pharmaceutically acceptable excipient.

9. A compound, or a pharmaceutically acceptable salt thereof, selected from the group consisting of

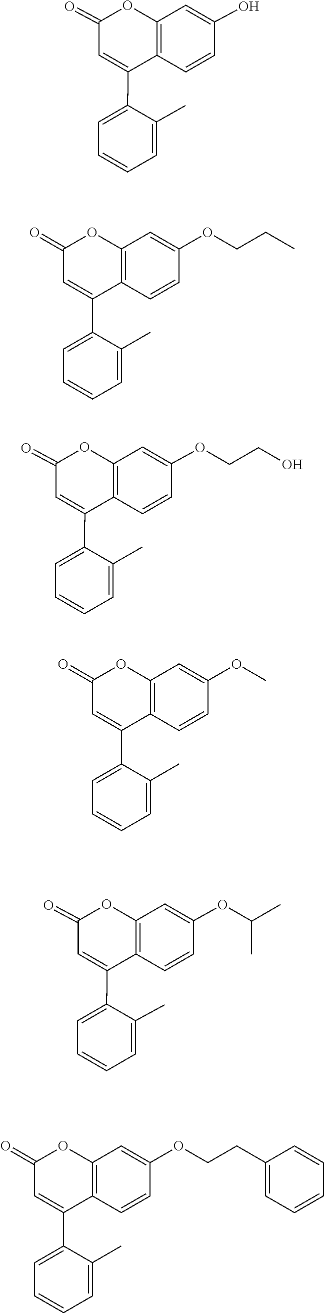 [SMILES: Cc1ccccc1-c1cc(=O)oc2cc(O)ccc12 ; CCCOc1ccc2c(-c3ccccc3C)cc(=O)oc2c1 ; Cc1ccccc1-c1cc(=O)oc2cc(OCCO)ccc12 ; COc1ccc2c(-c3ccccc3C)cc(=O)oc2c1 ; Cc1ccccc1-c1cc(=O)oc2cc(OC(C)C)ccc12 ; Cc1ccccc1-c1cc(=O)oc2cc(OCCc3ccccc3)ccc12]

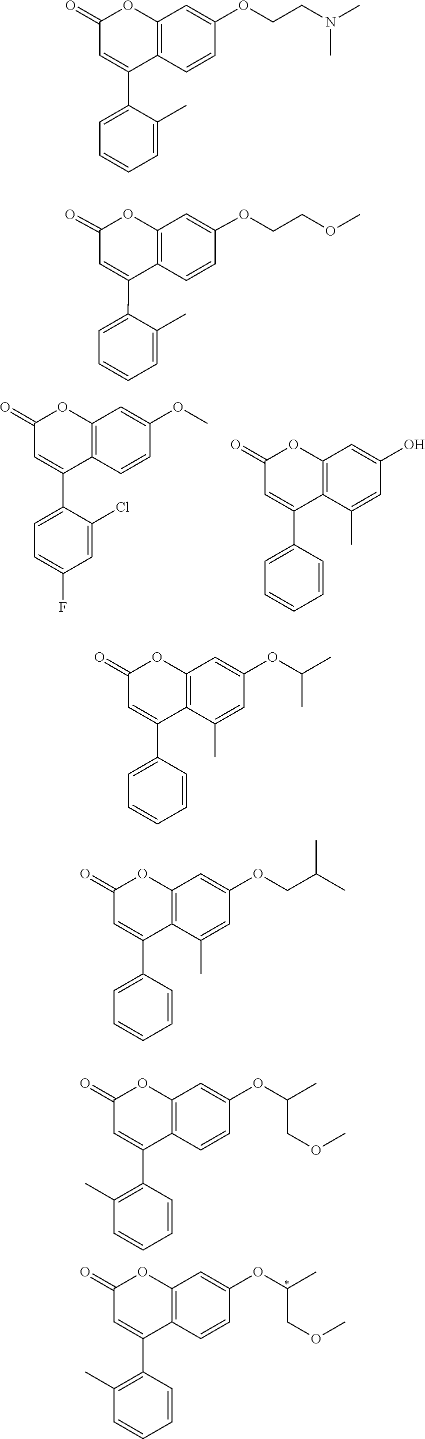

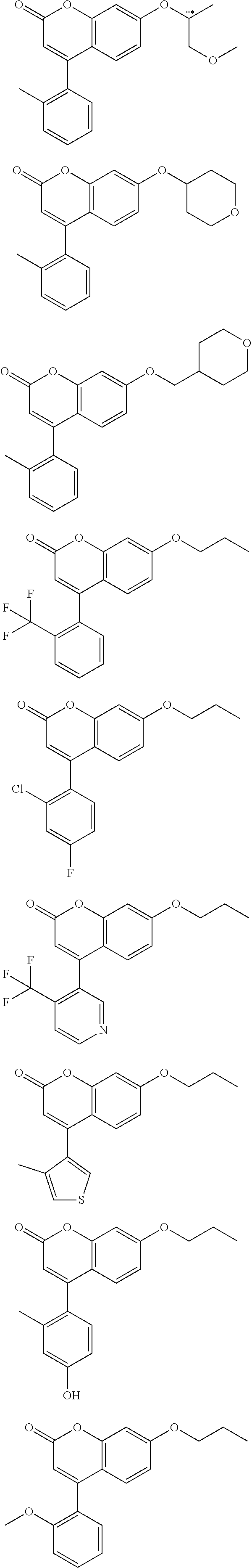

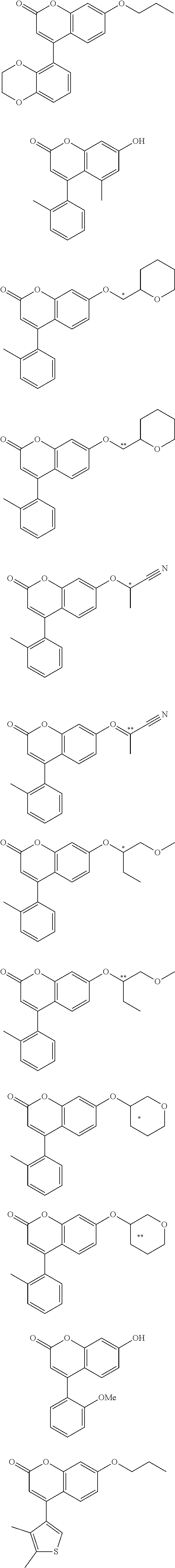

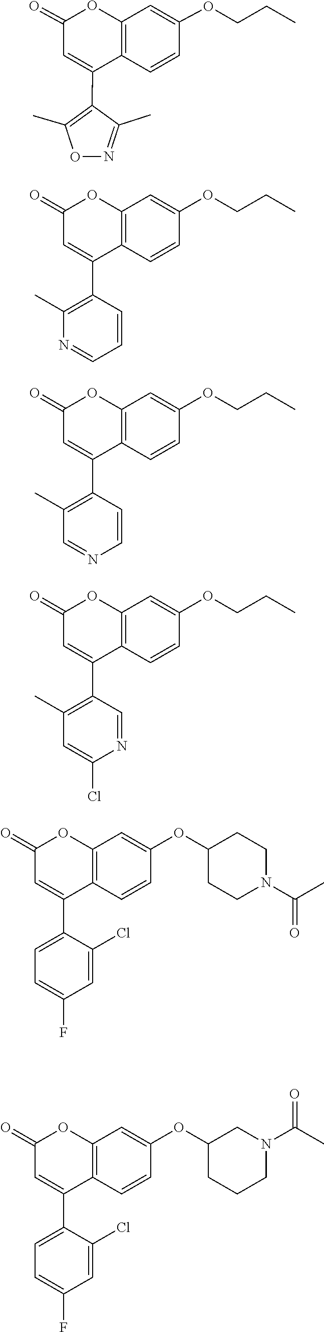

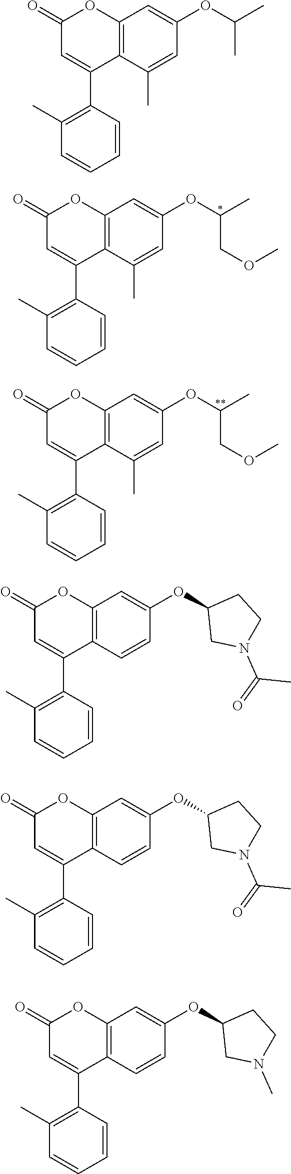

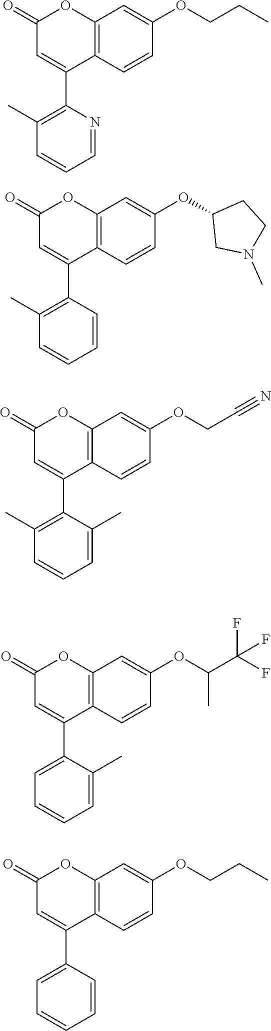

10. The compound according to claim 9, or a pharmaceutically acceptable salt thereof, wherein the compound is selected from the group consisting of:

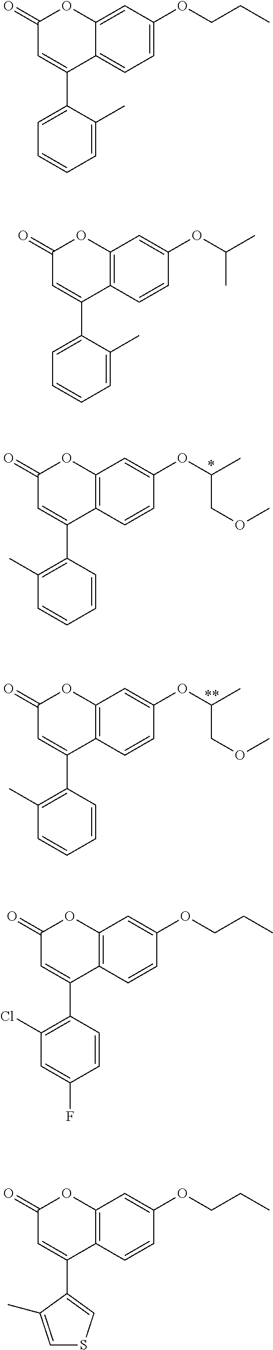

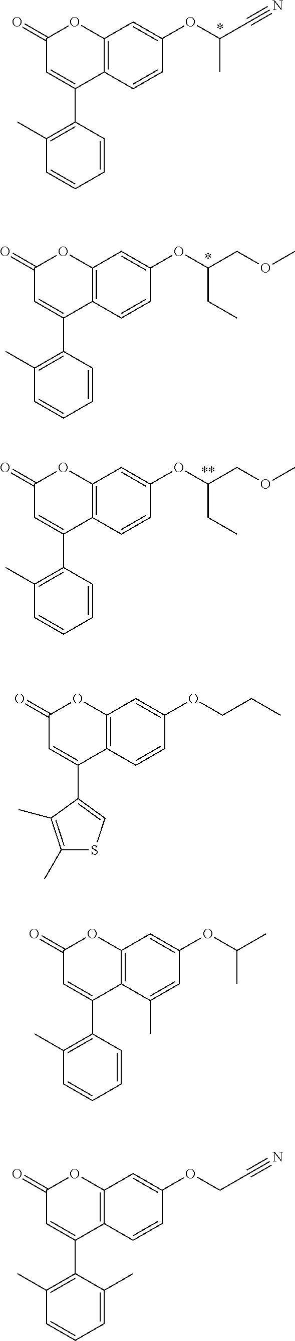

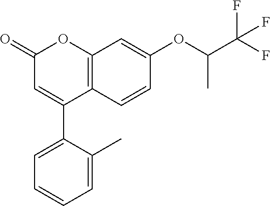 [SMILES: Cc1ccccc1-c1cc(=O)oc2cc(OC(C)C(F)(F)F)ccc12]

11. The compound according to claim 9, or a pharmaceutically acceptable salt thereof, wherein the compound is:

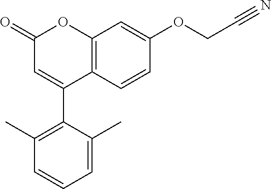 [SMILES: Cc1cccc(C)c1-c1cc(=O)oc2cc(OCC#N)ccc12]

12. The compound according to claim 9, or a pharmaceutically acceptable salt thereof, wherein the compound is:

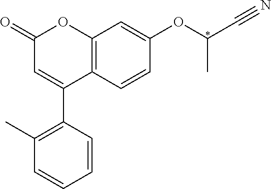 [SMILES: Cc1ccccc1-c1cc(=O)oc2cc(OC(C)C#N)ccc12]

13. The compound according to claim 9, or a pharmaceutically acceptable salt thereof, wherein the compound is:

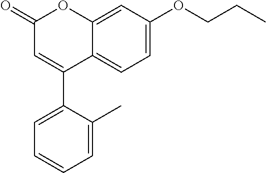 [SMILES: CCCOc1ccc2c(-c3ccccc3C)cc(=O)oc2c1]

14. The compound according to claim 9, or a pharmaceutically acceptable salt thereof, wherein the compound is selected from the group consisting of:

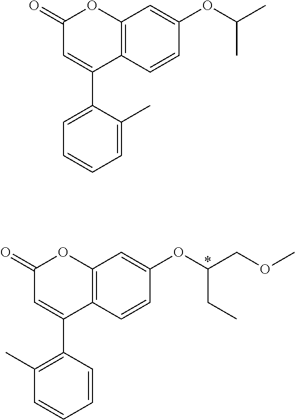 [SMILES: Cc1ccccc1-c1cc(=O)oc2cc(OC(C)C)ccc12 ; CCC(COC)Oc1ccc2c(-c3ccccc3C)cc(=O)oc2c1]

15. A pharmaceutical composition comprising a compound according to claim 10, and a pharmaceutically acceptable excipient.

16. A method of inhibiting the activity of POLRMT with a compound of formula (I), or a pharmaceutically acceptable salt thereof: 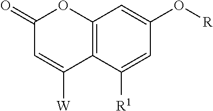
wherein:
W is C₆-C₁₀ aryl, C₆-C₁₀ cycloaryl, or 5-10 membered heteroaryl, any of which is optionally substituted with one or more groups, each independently selected from the group consisting of fluoro, chloro, trifluoromethyl, difluoromethyl, cyano, hydroxyl, C₁-C₄ alkoxyl, and C₁-C₄ alkyl optionally substituted with one or more deuterium or hydroxyl;
R is hydrogen, CH₂CH₂R², 5-or 6-membered heterocyclyl optionally substituted with C₁-C₄ alkyl or C(O)R³, or C₁-C₆ alkyl optionally substituted with one or more groups, each independently selected from the group consisting of C₁-C₄ alkyl, 5-or 6-membered heterocyclyl, hydroxyl, cyano, fluoro, C₁-C₄ alkoxyl, C₁-C₄ haloalkoxyl, and NR³R⁴,
or R is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and C(O)R³, provided that when R is hydrogen, then R¹ is methyl and W is substituted, and further provided that when R is methyl, then W is disubstituted;
R¹ is hydrogen, fluoro, chloro, hydroxyl, cyano, C₃-C₄ cycloalkyl, C₁-C₂ alkoxyl, C₁-C₂ haloalkoxyl, or C₁-C₃ alkyl optionally substituted with one or more fluoro;
R² is C₆-C₁₀ aryl optionally substituted with one or more groups, each independently selected from the group consisting of fluoro, chloro, C₁-C₄ alkyl, trifluoromethyl, difluoromethyl, cyano, hydroxyl, C₁-C₄ haloalkoxyl, and C₁-C₄ alkoxyl, OR³, NR³R⁴, C(O)R³, or C(O)NR³R⁴;
each R³ is independently hydrogen or C₁-C₄ alkyl optionally substituted with one or more groups selected from the group consisting of fluoro, hydroxyl, C₁-C₄ haloalkoxyl, and C₁-C₄ alkoxyl;
each R⁴ is independently R³, C(O)C₁-C₄ alkyl optionally substituted with one or more fluoro groups, or C(O)NHC₁-C₄ alkyl optionally substituted with one or more fluoro groups;
or if R³ and R⁴ are attached to the same nitrogen atom, R³ and R⁴ together with their connecting nitrogen form a 5-or 6-membered heterocyclic ring optionally containing another heteroatom that is N, O, S, S(O), or S(O)NR³, and such heterocyclic ring is optionally substituted with one or more groups each independently selected from the group consisting of fluoro, chloro, C₁-C₄ alkyl, C₁-C₄ alkoxyl, C₁-C₄ haloalkoxyl, carboxyl, and C₁-C₄ alkylcarboxylate; and
R⁵ is hydrogen or C₁-C₄ alkyl.

17. The method according to claim 16, wherein:
W is C₆ aryl, C₆ cycloaryl, or a 5-or 6-membered heteroaryl, any of which is optionally substituted with one or more groups, each independently selected from the group consisting of fluoro, chloro, C₁ alkyl, trifluoromethyl, hydroxyl, and C₁ alkoxyl;
R is hydrogen, CH₂CH₂R², C₆ aryl, 5-or 6-membered heterocyclyl optionally substituted with C₁ alkyl or C(O)R³, or C₁-C₄ alkyl optionally substituted with one or more groups, each independently selected from the group consisting of 6-membered heterocyclyl, hydroxyl, cyano, fluoro, C₁ alkoxyl, and NR³R⁴;
R¹ is hydrogen or C₁ alkyl;
R² is C₆ aryl;
each R³ is independently hydrogen or C₁ alkyl; and
each R⁴ is independently R³.

18. The method according to claim 16, wherein:
W is C₆ aryl or 5-membered heteroaryl, any of which is optionally substituted with one or more groups, each independently selected from the group consisting of methyl, chloro, and fluoro;
R is 6-membered heterocyclyl or C₁-C₄ alkyl optionally substituted with one or more groups independently selected from the group consisting of cyano, fluoro, and methoxyl; and
R¹ is hydrogen.

19. The method according to claim 16, wherein the compound is selected from the group consisting of:

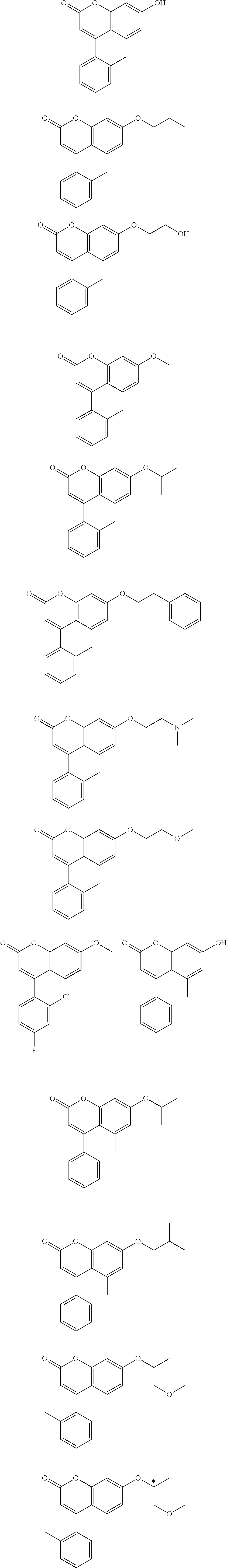

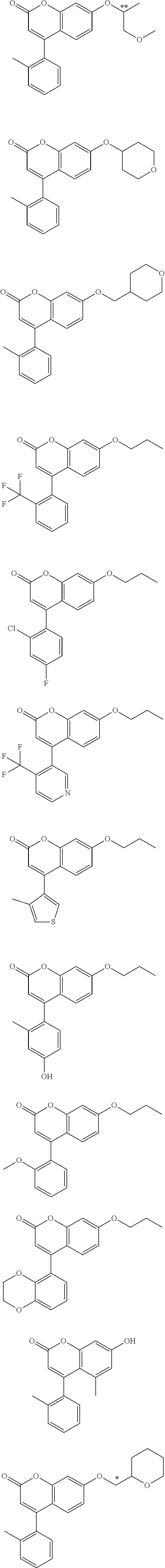

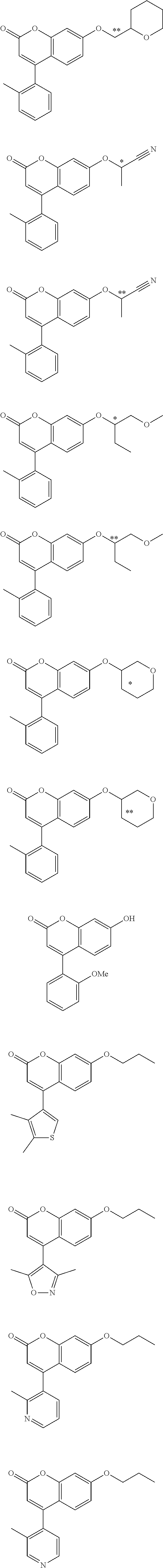

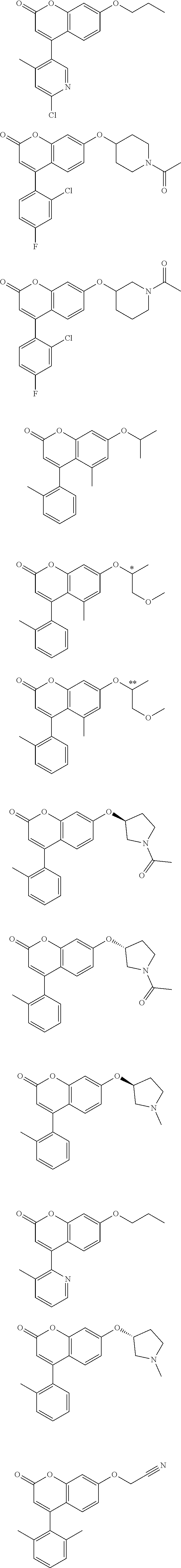

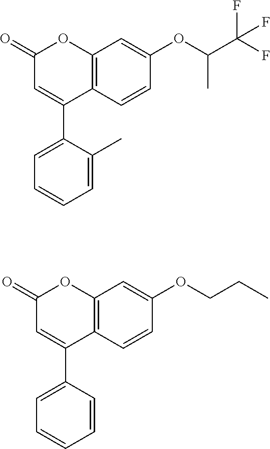 [SMILES: Cc1ccccc1-c1cc(=O)oc2cc(OC(C)C(F)(F)F)ccc12 ; CCCOc1ccc2c(-c3ccccc3)cc(=O)oc2c1]

20. The method according to claim 16, wherein the compound is selected from the group consisting of: 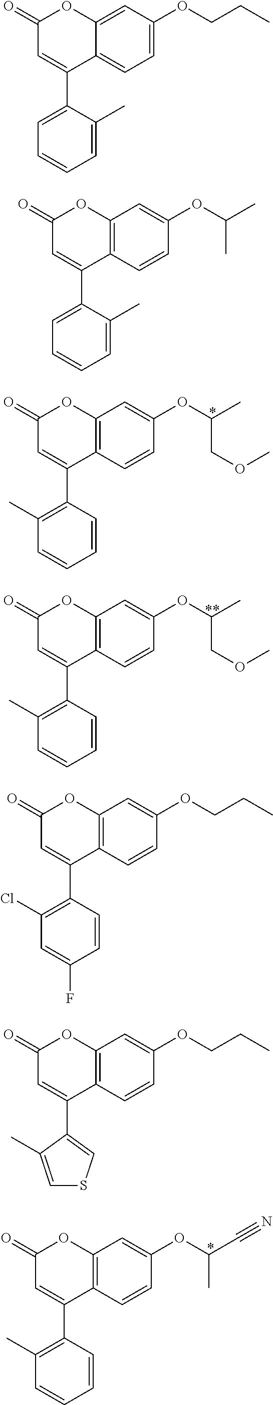 [chemical structure]
